(19) United States
(12) Patent Application Publication
(10) Pub. No.: US 2024/0279281 A1
(43) Pub. Date: Aug. 22, 2024

(54) Peptide Inhibitors of Interleukin-23 Receptor

(71) Applicants: Janssen Pharmaceutica NV, BEERSE (BE); Protagonist Therapeutics, Inc., Newark, CA (US)

(72) Inventors: Stephanie A. BARROS, Chalfont, PA (US); Santhosh Francis Neelamkavil, Edison, NJ (US); Chengzao SUN, Warrington, PA (US); Boying Guo, Warrington, PA (US); Charles Hendrick, North Wales, PA (US); David Marcoux, Newton Centre, MA (US); Sandeep Somani, Princeton, NJ (US); Tho Thieu, North Wales, PA (US); Jing Zhang, Wilmington, DE (US); Danila Branca, Pomezia (IT); Roberto Costante, Pomezia (IT); Ashok Bhandari, Pleasanton, CA (US); Gregory Thomas Bourne, Brisbane (AU); Brian Troy Frederick, Ben Lomond, CA (US)

(21) Appl. No.: 18/412,678

(22) Filed: Jan. 15, 2024

Related U.S. Application Data
(60) Provisional application No. 63/480,038, filed on Jan. 16, 2023.

(51) Int. Cl. C07K 7/64; A61K 38/00

(52) U.S. Cl. CPC C07K 7/64; A61K 38/00

(57) ABSTRACT

The present disclosure relates to peptide inhibitors of the interleukin-23 receptor (IL-23R) or pharmaceutically acceptable salts thereof, corresponding pharmaceutical compositions, methods and/or uses for treatment of autoimmune inflammation and related diseases and disorders.

### CROSS-REFERENCE TO RELATED APPLICATIONS

This application claims the benefit of U.S. Provisional Application No. 63/480,038 filed Jan. 16, 2023, which is herein incorporated by reference in its entirety.

### INCORPORATION OF SEQUENCE LISTING

The sequence listing in ST.26 XML format entitled NTT-4248_SL.xml, created on Jan. 12, 2024, comprising 1,212,736 bytes, prepared according to 37 CFR 1.822 to 1.824, submitted concurrently with the filing of this application, is incorporated herein by reference in its entirety.

### BACKGROUND

The interleukin-23 (IL-23) cytokine is a heterodimer composed of a unique p19 subunit and the p40 subunit shared with IL-12, which is a cytokine involved in the development of interferon-γ (IFN-γ)-producing T helper 1 (T_{H}1) cells. Although IL-23 and IL-12 both contain the p40 subunit, they have different phenotypic properties. For example, animals deficient in IL-12 are susceptible to inflammatory autoimmune diseases, whereas IL-23 deficient animals are resistant to these diseases, presumably due to a reduced number of CD4⁺ T cells producing IL-6, IL-17, and TNF in the CNS of IL-23-deficient animals. IL-23 binds to IL-23R, which is a heterodimeric receptor composed of IL-12Rβ1 and IL-23R subunits. Binding of IL-23 to IL-23R activates the Jak-Stat signaling molecules Jak2, Tyk2, Stat1, Stat 3, Stat 4, and Stat 5, although Stat4 activation is substantially weaker and different DNA-binding Stat complexes form in response to IL-23 as compared with IL-12. IL-23R associates constitutively with Jak2 and in a ligand-dependent manner with Stat3. In contrast to IL-12, which acts mainly on naive CD4(+) T cells, IL-23 preferentially acts on memory CD4(+) T cells.

IL-23 has been implicated as playing a crucial role in the pathogenesis of autoimmune inflammation and related diseases and disorders, such as multiple sclerosis, asthma, rheumatoid arthritis, psoriasis, and inflammatory bowel diseases (IBDs) such as ulcerative colitis and Crohn's disease. Studies in acute and chronic mouse models of IBDs revealed a primary role of interleukin-23 receptor (IL-23R) and downstream effector cytokines in disease pathogenesis. IL-23R is expressed on various adaptive and innate immune cells including Th17 cells, γδ T cells, natural killer (NK) cells, dendritic cells, macrophages, and innate lymphoid cells, which are found abundantly in the intestine. At the intestine mucosal surface, the gene expression and protein levels of IL-23R are found to be elevated in IBD patients. It is believed that IL-23 mediates this effect by promoting the development of a pathogenic CD4⁺ T cell population that produces IL-6, IL-17, and tumor necrosis factor (TNF).

Accordingly, there remains a need for compositions that bind IL-23R to inhibit IL-23 binding and signaling in a patient.

### BRIEF SUMMARY

Provided herein are peptide inhibitors of the interleukin-23 receptor (IL-23R) or pharmaceutically acceptable salts thereof, corresponding pharmaceutical compositions, and methods and/or uses of the IL-23R inhibitors for the treatment of inflammatory diseases, autoimmune diseases, and/or related disorders.

In particular, the present disclosure provides a peptide of Formula (I), comprising the amino acid sequence:

X₃-X₄-X₅-X₆-X₇-X₈-X₉-X₁₀-X₁₁-X₁₂-X₁₃-X₁₄-X₁₅-X₁₆-
X₁₇ (I),

or a pharmaceutically acceptable salt thereof, wherein each of X₃, X₄, X₅, X₆, X₇, X₈, X₉, X₁₀, X₁₁, X₁₂, X₁₃, X₁₄, X₁₅, X₁₆, and X₁₇ are defined herein.

In some embodiments, the present disclosure provides a peptide of Formula (I-B), comprising the amino acid sequence:

R₁-X₃-X₄-X₅-T-X₇-X₈-X₉-X₁₀-X₁₁-X₁₂-X₁₃-N-X₁₅-
X₁₆-X₁₇-R₂ (I-B),

or a pharmaceutically acceptable salt thereof, wherein each of R₁, X₃, X₄, X₅, X₇, X₈, X₉, X₁₀, X₁₁, X₁₂, X₁₃, X₁₅, X₁₆, X₁₇, and R₂ are defined herein.

In some embodiments, the present disclosure provides a peptide of Formula (I-E), comprising the amino acid sequence:

R₁-X₃-Pen-X₅-T-7(3NAcPh)W-X₈-Pen-X₁₀-6OH2Nal-THP-X₁₃-N-3Pya-Sar-X₁₇-R₂ (I-E),

or a pharmaceutically acceptable salt thereof, wherein each of R₁, X₃, X₅, X₈, X₁₀, X₁₃, X₁₇, and R₂ are defined herein.

The present disclosure further provides a pharmaceutical composition comprising a peptide described herein, or a pharmaceutically acceptable salt thereof, and a pharmaceutically acceptable carrier.

The present disclosure still further provides a method for treating a disease or disorder associated with Interleukin 23 (IL-23)/Interleukin 23 Receptor (IL-23R), comprising administering to a subject in need thereof a therapeutically effective amount of a peptide or a pharmaceutical composition described herein. In some embodiments, the disease or disorder is selected from ulcerative colitis (UC), Crohn's disease (CD), psoriasis (PsO), and psoriatic arthritis (PsA).

### DETAILED DESCRIPTION

Provided herein are peptide inhibitors of IL-23R, and pharmaceutically acceptable salts thereof, corresponding pharmaceutical compositions, and methods and/or uses for the treatment of inflammatory diseases, autoimmune diseases, and/or related disorders. The peptide inhibitors of the present disclosure may exhibit enhanced properties, such as longer in vivo half-life, compared to the corresponding cyclic peptide inhibitor of IL-23R without a cyclic structure.

Peptide molecules that modulate the interactions of IL-23R with IL-23 represent a convenient and cost-efficient treatment for autoimmune diseases when used as an oral therapeutic. To achieve high binding interactions with the IL-23 receptor, it is critical to maintain several amino acid residues at selected positions as shown in the peptide molecules described herein. In addition, peptide molecules having a hydroxy substituted naphthalene at the X₁₁ position can provide higher potency than peptide molecules without such a feature. Therefore, the peptide molecules described herein represent a series of more potent and efficient therapeutics for inhibiting IL-23R.

### Definitions

Unless otherwise defined herein, scientific and technical terms used in this application shall have the meanings that are commonly understood by those of ordinary skill in the art.

As used in the specification and in the claims, the “comprise(s),” “comprising,” “include(s),” “having,” “has,” “can,” “contain(s),” and variants thereof, as used herein, are intended to be open-ended transitional phrases, terms, or words that require the presence of the named features, groups, ingredients, or steps and does not exclude the presence of additional features, groups, ingredients, or steps. For example, the language “a peptide of Formula (I), comprising the amino acid sequence: X₃-X₄-X₅-X₆-X₇-X₈-X₉-X₁₀-X₁₁-X₁₂-X₁₃-X₁₄-X₁₅-X₁₆-X₁₇ (I),” means that in addition to amino acids X₃ through Xin, the peptide may include but is not limited to additional amino acids attached to the N-terminus, additional amino acids attached to the C-terminus, N-terminal or C-terminal capping groups, chemical or biological moieties (including but not limited to, for example, lipophilic substituents, antibodies, imaging agents, etc.) conjugated to the peptide at any location, and the like. The term “comprise(s),” “comprising,” “include(s),” “having,” “has,” “can,” or “contain(s),” can include embodiments encompassed by the term “consisting essentially of” or “consisting of.”

The terms “peptide,” “polypeptide,” and “protein” are used interchangeably herein and typically refer to a molecule comprising a chain of two or more amino acids (e.g., L-amino acids, D-amino acids, modified amino acids, amino acid analogs, amino acid mimetics, etc.).

Naturally-occurring L-amino acids are represented by the conventional three-letter or capitalized one-letter amino acid designations of Table 1. The corresponding D-amino acids are represented by lower-case one-letter amino acid designations or by the three-letter or capitalized one letter amino acid designations of Table 1 preceded by the letter “D” (e.g., r, dR, or D-Arg).

TABLE 1
Naturally-occurring amino acids

| | | | | | |
|---|---|---|---|---|---|
| G | Glycine | Gly | P | Proline | Pro |
| A | Alanine | Ala | V | Valine | Val |
| L | Leucine | Leu | I | Isoleucine | Ile |
| M | Methionine | Met | C | Cysteine | Cys |
| F | Phenylalanine | Phe | Y | Tyrosine | Tyr |
| W | Tryptophan | Trp | H | Histidine | His |
| K | Lysine | Lys | R | Arginine | Arg |
| Q | Glutamine | Gln | N | Asparagine | Asn |
| E | Glutamic Acid | Glu | D | Aspartic Acid | Asp |
| S | Serine | Ser | T | Threonine | Thr |

The term “L-amino acid,” as used herein, refers to the “L” isomeric form of an amino acid, and conversely the term “D-amino acid” refers to the “D” isomeric form of an amino acid (e.g., (D)Asp or D-Asp; (D)Phe or D-Phe). D-amino acids may be indicated as customary in lower case when referred to using single-letter abbreviations. For example, D-arginine can be represented as “arg” or “r.” Alternatively, a lower case “d” in front of an amino acid can be used to indicate that it is of the D isomeric form, for example D-lysine can be represented by dK.

In the case of less common or non-naturally occurring amino acids, unless they are referred to by their full name (e.g., sarcosine, omithine, etc.), frequently employed three- or four-character codes are employed for residues thereof, including, Sar or Sarc (sarcosine, i.e., N-methylglycine), Aib (α-aminoisobutyric acid), Dab (2,4-diaminobutanoic acid), Dapa (2,3-diaminopropanoic acid), γ-Glu (γ-glutamic acid), Gaba (γ-aminobutanoic acid), R-Pro (pyrrolidine-3-carboxylic acid), and Abu (2-aminobutyric acid).

Amino acids of the D-isomeric form may be located at any of the positions in the IL-23R inhibitors set forth herein (e.g., any of X₃-X₁₇ appearing in the molecule). In some embodiments, amino acids of the D-isomeric form may be located only at any one or more of X₃, X₅, X₆, X₈, X₁₃, and optionally one additional position. In other embodiments, amino acids of the D-isomeric form may be located only at any one or more of X₃, X₈, X₁₃, and optionally one additional position. In other embodiments, amino acids of the D-isomeric form may be located only at any one or more of X₈, X₁₃, and optionally one additional position. In other embodiments, amino acids of the D-isomeric form may be located only at X₃ and optionally one additional position. In other embodiments, amino acids of the D-isomeric form may be located only at X₃, and optionally two or three additional positions. In other embodiments, amino acids of the D-isomeric form may be located at only one or two of positions X₃ to X₁₇ appearing in the IL-23R inhibitors set forth herein. In other embodiments, amino acids of the D-isomeric form may be located at only three or four of positions X₃ to X₁₇ appearing in the IL-23R inhibitors set forth herein. For example, an IL-23R inhibitor set forth herein having only positions X₃ to X₁₅ present may have amino acids of the D-form present in three or four of those positions. In other embodiments, amino acids of the D-isomeric form may be located at only five or six of positions X₃ to X₁₇ appearing in the IL-23R inhibitors set forth herein.

Peptides may be naturally occurring, synthetically produced, or recombinantly expressed. Peptides may also comprise additional groups modifying the amino acid chain, for example, functional groups added via post-translational modification. Examples of post-translation modifications include, but are not limited to, acetylation, alkylation (including, methylation), biotinylation, glutamylation, glycylation, glycosylation, isoprenylation, lipoylation, phosphopantetheinylation, phosphorylation, selenation, and C-terminal amidation. The term peptide also includes peptides comprising modifications of the amino terminus and/or the carboxy terminus. Modifications of the terminal amino group include, but are not limited to, des-amino, N-lower alkyl, N-di-lower alkyl, and N-acyl modifications. Modifications of the terminal carboxy group include, but are not limited to, amide, lower alkyl amide, dialkyl amide, and lower alkyl ester modifications (e.g., wherein lower alkyl is C1-C4 alkyl). The term peptide also includes modifications, such as but not limited to those described above, of amino acids falling between the amino and carboxy termini.

As is clear to the skilled artisan, the peptide sequences disclosed herein are shown proceeding from left to right, with the left end of the sequence being the N-terminus of the peptide and the right end of the sequence being the C-terminus of the peptide. Among sequences disclosed herein are sequences incorporating either an “—OH” moiety or an “—NH₂” moiety at the carboxy terminus (C-terminus) of the sequence. In such cases, and unless otherwise indicated, an “—OH” or an “—NH₂” moiety at the C-terminus of the sequence indicates a hydroxy group or an amino group, corresponding to the presence of a carboxylic acid (COOH) or an amido (CONH₂) group at the C-terminus, respectively. In each sequence of the disclosure, a C-terminal “—OH” moiety may be substituted for a C-terminal “—NH₂” moiety, and vice-versa.

The phrase “amino acid,” “amino acid residue,” or “residue” as used herein refers to an amino acid, a modified amino acid, an amino acid analog, or an amino acid mimetic that is incorporated into a peptide by an amide bond or an amide bond mimetic.

Unless indicated otherwise the names of naturally occurring and non-naturally occurring aminoacyl residues used herein follow the naming conventions suggested by the IUPAC Commission on the Nomenclature of Organic Chemistry and the IUPAC-IUB Commission on Biochemical Nomenclature as set out in “Nomenclature of α-Amino Acids (Recommendations, 1974)” Biochemistry, 14(2), (1975). To the extent that the names and abbreviations of amino acids and aminoacyl residues employed in this specification and appended claims differ from those suggestions, they will be made clear to the reader. In sequences of amino acids that represent IL-23 inhibitors the individual amino acids are separated by a hyphen “-” or brackets e.g., lysine is shown as [K].

One of skill in the art will appreciate that certain amino acids and other chemical moieties are modified when bound to another molecule. For example, an amino acid side chain may be modified when it forms an intramolecular bridge with another amino acid side chain, e.g., one or more hydrogens may be removed or replaced by the bond.

The term “therapeutically effective amount” or “pharmaceutically effective amount” means that amount of active peptide or pharmaceutical agent that elicits the biological or medicinal response in a tissue system, animal or human, that is being sought by a researcher, veterinarian, medical doctor, or other clinician, which includes preventing, treating or ameliorating the symptoms of a syndrome, disorder or disease being treated.

The term “pharmaceutically acceptable” means approved or approvable by a regulatory agency of Federal or a state government or the corresponding agency in countries other than the United States, or that is listed in the U. S. Pharmcopoeia or other generally recognized pharmacopoeia for use in animals, and more particularly, in humans.

“Pharmaceutically acceptable excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Composition” or “pharmaceutical composition” as used herein is intended to encompass a product comprising the specified active pharmaceutical ingredient (API) (i.e., a peptide of the present disclosure), which may include pharmaceutically acceptable excipients, carriers or diluents as described herein, such as in specified amounts defined throughout the disclosure.

Compositions or pharmaceutical compositions of the present disclosure may be in different pharmaceutically acceptable forms, which may include, but are not limited to a liquid composition, a tablet or matrix composition, a capsule composition, etc. When the composition is a tablet composition, the tablet may include, but is not limited to different layers two or more different phases, including an internal phase and an external phase that can comprise a core. The tablet composition can also include, but is not limited to one or more coatings.

Provided are also pharmaceutically acceptable salts and tautomeric forms of the peptides described herein.

A “pharmaceutically acceptable salt” is intended to mean a salt of a free acid or base of peptides represented by Formula (I) that are non-toxic, biologically tolerable, or otherwise biologically suitable for administration to the subject. It should possess the desired pharmacological activity of the parent compound. See, generally, G. S. Paulekuhn, et al., “Trends in Active Pharmaceutical Ingredient Salt Selection based on Analysis of the Orange Book Database”, J Med. Chem., 2007, 50:6665-72, S. M. Berge, et al., “Pharmaceutical Salts”, J Pharm Sci., 1977, 66:1-19, and Handbook of Pharmaceutical Salts, Properties, Selection, and Use, Stahl and Wermuth, Eds., Wiley-VCH and VHCA, Zurich, 2002. Examples of pharmaceutically acceptable salts are those that are pharmacologically effective and suitable for contact with the tissues of patients without undue toxicity, irritation, or allergic response. A peptide of Formula (I) may possess a sufficiently acidic group, a sufficiently basic group, or both types of functional groups, and accordingly react with a number of inorganic or organic bases, and inorganic and organic acids, to form a pharmaceutically acceptable salt.

The IL-23R inhibitors of the present disclosure, pharmaceutically acceptable salts, and/or other forms thereof may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic and optically pure forms of the IL-23R inhibitors of the present disclosure. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included. Where compounds are represented in their chiral form, it is understood that the aspect encompasses, but is not limited to, the specific diastereomerically or enantiomerically enriched form. Where chirality is not specified but is present, it is understood that the aspect is directed to either the specific diastereomerically or enantiomerically enriched form; or a racemic or scalemic mixture of such compound(s).

“Racemates” refers to a mixture of enantiomers. The mixture can include equal or unequal amounts of each enantiomer.

“Stereoisomer” and “stereoisomers” refer to compounds that differ in the chirality of one or more stereo centers. Stereoisomers include enantiomers and diastereomers. The compounds may exist in stereoisomeric form if they possess one or more asymmetric centers or a double bond with asymmetric substitution and, therefore, can be produced as individual stereoisomers or as mixtures. Unless otherwise indicated, the description is intended to include individual stereoisomers as well as mixtures. The methods for the determination of stereochemistry and the separation of stereoisomers are well-known in the art (see, e.g., Chapter 4 of Advanced Organic Chemistry, 4th ed., J. March, John Wiley and Sons, New York, 1992).

“Diastereoisomers” are stereoisomers that have at least two asymmetric atoms, but which are not mirror images of each other.

“Enantiomers” are a pair of stereoisomers that are non-superimposable mirror images of each other. A “racemic” mixture is a 1:1 mixture of a pair of enantiomers. A “scalemic” mixture of enantiomers is mixture of enantiomers at a ratio other than 1:1.

“Tautomer” refers to alternate forms of a compound that differ in the position of a proton, such as enol-keto and imine-enamine tautomers, or the tautomeric forms of heteroaryl groups containing a ring atom attached to both a ring —NH— and a ring ═N— such as pyrazoles, imidazoles, benzimidazoles, triazoles, and tetrazoles.

The term “administering” with respect to the methods of the present disclosure, means a method for therapeutically or prophylactically preventing, treating or ameliorating a syndrome, disorder or disease as described herein by using a compound of the disclosure, or pharmaceutically acceptable salt thereof, composition thereof, or medicament thereof. Such methods include administering a therapeutically effective amount of a peptide of the disclosure, or pharmaceutically acceptable salt thereof, composition thereof, or medicament thereof, at different times during the course of a therapy or concurrently or sequentially as a combination therapy.

“Patient” or “subject,” which are used interchangeably, refer to a living organism, preferably a mammal, most preferably a human, whom will be or has been treated by a method according to an embodiment of the application. Examples of mammals include, but are not limited to, cows, horses, sheep, pigs, cats, dogs, mice, rats, rabbits, guinea pigs, non-human primates (NHPs) such as monkeys or apes, humans, etc., more preferably a human.

As used herein, the term “treatment” or “treating,” is defined as the application or administration of a therapeutic agent, i.e., a compound of the present disclosure (alone or in combination with another pharmaceutical agent), to a patient, or application or administration of a therapeutic agent to an isolated tissue or cell line from a patient (e.g., for diagnosis or ex vivo applications), who has a disorder or disease as described herein, a symptom thereof; or the potential to develop such disorder or disease, where the purpose of the application or administration is to cure, heal, alleviate, relieve, alter, remedy, ameliorate, improve or affect the disorder or disease, its symptoms, or the potential to develop said disorder or disease. Such treatments may be specifically tailored or modified, based on knowledge obtained from the field of pharmacogenomics.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

Unless defined otherwise, all technical and scientific terms used herein have the same meaning as commonly or conventionally understood by one of ordinary skill in the art. In the chemical arts a dash at the front or end of a chemical group is a matter of convenience; chemical groups may be depicted with or without one or more dashes without losing their ordinary meaning. A wavy line drawn through a line in a structure indicates a point of attachment of a group. A dashed line indicates an optional bond. Unless chemically or structurally required, no directionality is indicated or implied by the order in which a chemical group is written or the point at which it is attached to the remainder of the molecule. For instance, the group “—SO₂CH₂—” is equivalent to “—CH₂SO₂—” and both may be connected in either direction. Similarly, an “arylalkyl” group, for example, may be attached to the remainder of the molecule at either an aryl or an alkyl portion of the group. A prefix such as “Cᵤ₋ᵥ” or (Cᵤ-Cᵥ) indicates that the following group has from u to v carbon atoms. For example, “C₁₋₆alkyl” and “C₁-C₆ alkyl” both indicate that the alkyl group has from 1 to 6 carbon atoms.

The term “alkyl” is a straight or branched saturated hydrocarbon. For example, an alkyl group can have 1 to 10 carbon atoms (i.e., (C₁-C₁₀)alkyl), 1 to 5 carbon atoms (i.e., (C₁-C₅)alkyl), 1 to 4 carbon atoms (i.e., (C₁-C₄)alkyl), or 1 to 3 carbon atoms (i.e., (C₁-C₃)alkyl). Examples of alkyl groups include, but are not limited to, methyl (Me, —CH₃), ethyl (Et, —CH₂CH₃), 1-propyl (n-Pr, n-propyl, —CH₂CH₂CH₃), isopropyl (i-Pr, i-propyl, —CH(CH₃)₂), 1-butyl (n-bu, n-butyl, —CH₂CH₂CH₂CH₃), 2-butyl (s-bu, s-butyl, —CH(CH₃)CH₂CH₃), tert-butyl (t-bu, t-butyl, —CH(CH₃)₃), 1-pentyl (n-pentyl, —CH₂CH₂CH₂CH₂CH₃), 2-pentyl (—CH(CH₃) CH₂CH₂CH₃), neopentyl (—CH₂C(CH₃)₃), 1-hexyl (—CH₂CH₂CH₂CH₂CH₂CH₃), 2-hexyl (—CH(CH₃)CH₂CH₂CH₂CH₃), heptyl (—(CH₂)₆CH₃), octyl (—(CH₂)₇CH₃), 2,2,4-trimethylpentyl (—CH₂C(CH₃)₂CH₂CH(CH₃)₂), nonyl (—(CH₂)₈CH₃), decyl (—(CH₂)₉CH₃), undecyl (—(CH₂)₁₀CH₃), and dodecyl (—(CH₂)₁₁CH₃). In an embodiment, alkyl refers to C₍₁₋₆₎alkyl. In another embodiment, alkyl refers to C₍₁₋₄₎alkyl. In another embodiment, alkyl refers to C₍₁₋₃₎alkyl.

The term “alkylene” refers to a bivalent alkyl group. For example, an alkylene group can have 1 to 10 carbon atoms (i.e., (C₁-C₁₀)alkylene), 1 to 5 carbon atoms (i.e., (C₁-C₅)alkylene), 1 to 2 carbon atoms (i.e., (C₁-C₂)alkylene), or 1 carbon atom (i.e., (C₁)alkylene). Examples of alkylene groups include, but are not limited to, methylene (—CH₂—), ethylene (—CH₂CH₂—), n-propylene (—CH₂CH₂CH₂—), n-butylene (—CH₂CH₂CH₂CH₂—), etc.

The term “halo” or “halogen” refers to bromo (—Br), chloro (—Cl), fluoro (—F), or iodo (—I). In an embodiment, halo refers to fluoro.

The term “haloalkyl” refers to a straight- or branched-chain alkyl group having from 1 to 12 carbon atoms, 1 to 6 carbon atoms, 1 to 4 carbon atoms, or 1 to 3 carbon atoms in the chain optionally substituting one or more H with halo. Examples of “haloalkyl” groups include trifluoromethyl (CF₃), difluoromethyl (CF₂H), monofluoromethyl (CH₂F), pentafluoroethyl (CF₂CF₃), tetrafluoroethyl (CHFCF₃), monofluoroethyl (CH₂CH₂F), trifluoroethyl (CH₂CF₃), tetrafluorotrifluoromethylethyl (CF(CF₃)₂), and groups that in light of the ordinary skill in the art and the teachings provided herein would be considered equivalent to any one of the foregoing examples. In an embodiment, haloalkyl refers to C₍₁₋₆₎haloalkyl. In another embodiment, haloalkyl refers to C₍₁₋₄₎haloalkyl. In another embodiment, alkyl refers to C₍₁₋₃₎haloalkyl.

The term “cycloalkyl” refers to a saturated or partially unsaturated all carbon ring system having 3 to 8 carbon atoms (i.e., C₍₃₋₈₎cycloalkyl), and preferably 3 to 6 carbon atoms (i.e., C₍₃₋₆₎cycloalkyl), wherein the cycloalkyl ring system has a single ring or multiple rings in a spirocyclic or bicyclic form. Exemplary cycloalkyls include, but are not limited to, cyclopropyl, cyclobutyl, cyclopentyl, cyclohexyl, cycloheptyl, and cyclooctyl. Unless otherwise stated specifically in the specification, a cycloalkyl group may be unsubstituted or substituted. Some cycloalkyl groups may exist as spirocycloalkyls, wherein two cycloalkyl rings are fused through a single carbon atom; for example and without limitation, an example of a spiropentyl group is

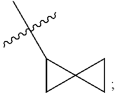 [SMILES: *C1CC12CC2]

for example and without limitation, examples of spirohexyl groups include

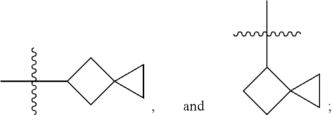 [SMILES: *C1CC2(CC2)C1 ; *C1CCC12CC2]

for example and without limitation examples of cycloheptyl groups include

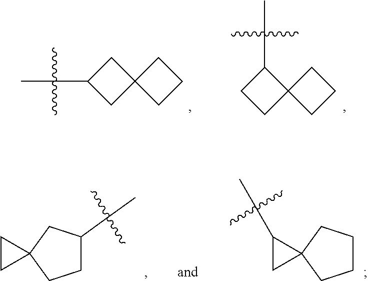 [SMILES: *C1CC2(CCC2)C1 ; *C1CCC12CCC2 ; *C1CCC2(CC2)C1 ; *C1CC12CCCC2]

and for example and without limitation examples of cyclooctyl groups include

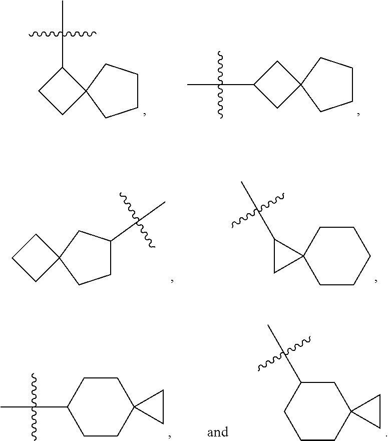 [SMILES: *C1CCC12CCCC2 ; *C1CC2(CCCC2)C1 ; *C1CCC2(CCC2)C1 ; *C1CC12CCCCC2 ; *C1CCC2(CC1)CC2 ; *C1CCCC2(CC2)C1]

Unless otherwise stated specifically in the specification, a siprocycloalkyl group may be unsubstituted or substituted. Bicyclic cycloalkyl ring systems also include

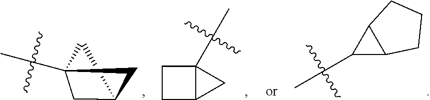

The term “heterocycle” or “heterocyclyl” refers to a saturated or partially unsaturated ring system that has at least one atom other than carbon in the ring system, wherein the atom is selected from the group consisting of oxygen, nitrogen and sulfur. The heterocyclyl group may, for example, consist of a single ring or multiple rings (e.g., in the form of a spirocyclic or bicyclic ring system). Exemplary heterocycles include, but are not limited to oxetanyl, aziridinyl, azetidinyl, pyrrolidinyl, piperidinyl, piperazinyl, morpholinyl, tetrahydropyranyl, tetrahydrofuranyl, and thiomorpholinyl.

The term “heteroaryl” refers to a single aromatic ring that has at least one atom other than carbon in the ring, wherein the atom is selected from the group consisting of oxygen, nitrogen and sulfur. The term “heteroaryl” includes single aromatic rings of from 1 to 6 carbon atoms and 1 to 4 heteroatoms selected from the group consisting of oxygen, nitrogen and sulfur. Exemplary heteroaryl ring systems include but are not limited to pyridinyl, pyrimidinyl, pyrazinyl, pyridazinyl, pyrimidinyl, pyrazolyl, oxazolyl, oxadiazolyl, isoxazolyl, triazolyl, imidazolyl, tetrazolyl, thienyl, thiazolyl, isothiazolyl, thiadiazolyl, or furyl.

Furthermore, it is intended that within the scope of the present invention, any element, in particular when mentioned in relation to a peptide of the disclosure, or pharmaceutically acceptable salt thereof, shall comprise all isotopes and isotopic mixtures of said element, either naturally occurring or synthetically produced, either with natural abundance or in an isotopically enriched form. For example, a reference to hydrogen includes within its scope ¹H, ²H (i.e., deuterium or D), and ³H (i.e., tritium or T). In some embodiments, the compounds described herein include a ²H (i.e., deuterium) isotope. By way of example, the group denoted —C₍₁₋₆₎alkyl includes not only —CH₃, but also CD3; not only CH₂CH₃, but also CD2CD3, etc. Similarly, references to carbon and oxygen include within their scope respectively ¹²C, ¹³C and ¹⁴C and ¹⁵O and ¹⁶O and ¹⁷O and ¹⁸O. The isotopes may be radioactive or non-radioactive. Radiolabelled compounds of the disclosure may include a radioactive isotope selected from the group comprising ³H, ¹¹C, ¹⁸F, ³⁵S ¹²²I, ¹²³I, ¹²⁵I, ¹³¹I, ⁷⁵Br, ⁷⁶Br, ⁷⁷Br and ⁸²Br. Preferably, the radioactive isotope is selected from the group of ³H, ¹¹C and ¹⁸F.

Abbreviation, “(V/V)” refers to the phrase “volume for volume”, i.e., the proportion of a particular substance within a mixture, as measured by volume or a volume amount of a component of the composition disclosed herein relative to the total volume amount of the composition. Accordingly, the quantity is unit less and represents a volume percentage amount of a component relative to the total volume of the composition. For example, a 2% (V/V) solvent mixture can indicate 2 mL of one solvent is present in 100 mL of the solvent mixture.

Systemic routes of administration as conventionally understood in the medicinal or pharmaceutical arts, refer to or are defined as a route of administration of drug, a pharmaceutical composition or formulation, or other substance into the circulatory system so that various body tissues and organs are exposed to the drug, formulation or other substance. As conventionally understood in the art, administration can take place orally (where drug or oral preparations are taken by mouth, and absorbed via the gastrointestinal tract), via enteral administration (absorption of the drug also occurs through the gastrointestinal tract) or parenteral administration (generally injection, infusion, or implantation, etc.

“Bioavailability” refers to the extent and rate at which the active moiety (drug or metabolite) enters systemic circulation, thereby accessing the site of action. Bioavailability of a drug could be impacted by the factors such as properties of the dosage form and properties of the drug.

“Digestive tract tissue” as used herein refers to all the tissues that comprise the organs of the alimentary canal. For example only, and without limitation, “digestive tract tissue” includes tissues of the mouth, esophagus, stomach, small intestine, large intestine, duodenum, and anus.

### Compounds

The present disclosure provides a peptide inhibitor of interleukin-23 receptor. In particular, the present disclosure provides a peptide of Formula (I), comprising the amino acid sequence:

X₃-X₄-X₅-X₆-X₇-X₈-X₉-X₁₀-X₁₁-X₁₂-X₁₃-X₁₄-X₁₅-
X₁₆-X₁₇ (I),

or a pharmaceutically acceptable salt thereof, wherein:
X₃ is any amino acid or absent;
X₄ is any amino acid;
X₅ is any amino acid;
X₆ is any amino acid;
X₇ is:

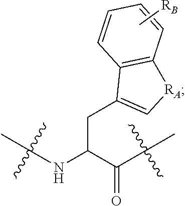

R_{A} is O, NH, N—C₍₁₋₅₎alkyl, or S;
R_{B} is —H, halo, C₍₁₋₃₎alkyl, or phenyl, wherein the phenyl is optionally substituted with one —N(H)C(O)C₍₁₋₃₎alkyl group;
X₈ is any amino acid;
X₉ is any amino acid;
X₁₀ is:

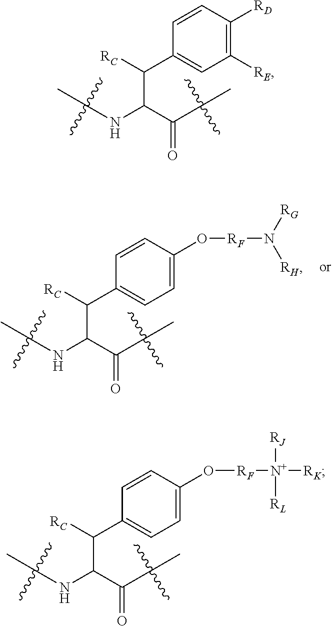

R_{C} is —H or —C₍₁₋₃₎alkyl;
R_{D} is —H, —OH, —CN, —C₍₁₋₃₎alkyl, —OC₍₁₋₃₎alkyl, —OC₍₁₋₃₎alkyl-(5-membered heteroaryl), —C(O)NH₂, or heterocyclyl, wherein the —OC₍₁₋₃₎alkyl-(5-membered heteroaryl) is optionally substituted with a polyethylene glycol chain, and wherein the heterocyclyl is optionally substituted with one-C(O)NH₂ group;
R_{E} is —H or halo;
R_{F} is —C₍₁₋₆₎alkylene or a bivalent polyethylene glycol chain;
R_{G} is —H, —C₍₁₋₃₎alkyl, or a bond to the amino acid at X₅ or X₁₃;
R_{H} is —H, —C₍₁₋₃₎alkyl, —C(NH)NH₂, —C(O)—R_{H1};
or R_{G} and R_{H} taken together with the nitrogen atom to which they are attached form a 5- to 8-membered heterocyclyl group;
R_{H1} is —C₍₁₋₅₎alkyl, —OC₍₁₋₅₎alkyl, —C₍₁₋₃₎alkyl-phenyl, -phenyl-C₍₁₋₃₎alkyl-N(H)—S(O)₂—C₍₁₋₃₎)alkyl, or a polyethylene glycol chain, wherein the —C₍₁₋₃₎alkyl-phenyl is optionally substituted with one to three groups selected from halo and —OH;
R_{J}, R_{K}, and R_{L} are each, independently, C₍₁₋₃₎alkyl;
or R_{J} and R_{K} taken together with the nitrogen atom to which they are attached form a 5- to 8-membered heterocyclyl group;
or X₁₀ is:

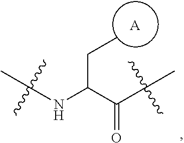

A is

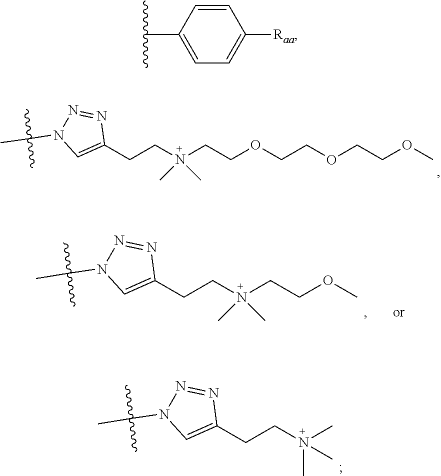

Rₐₐ is —OCHF₂, —O(CH₂)₉CO₂H,

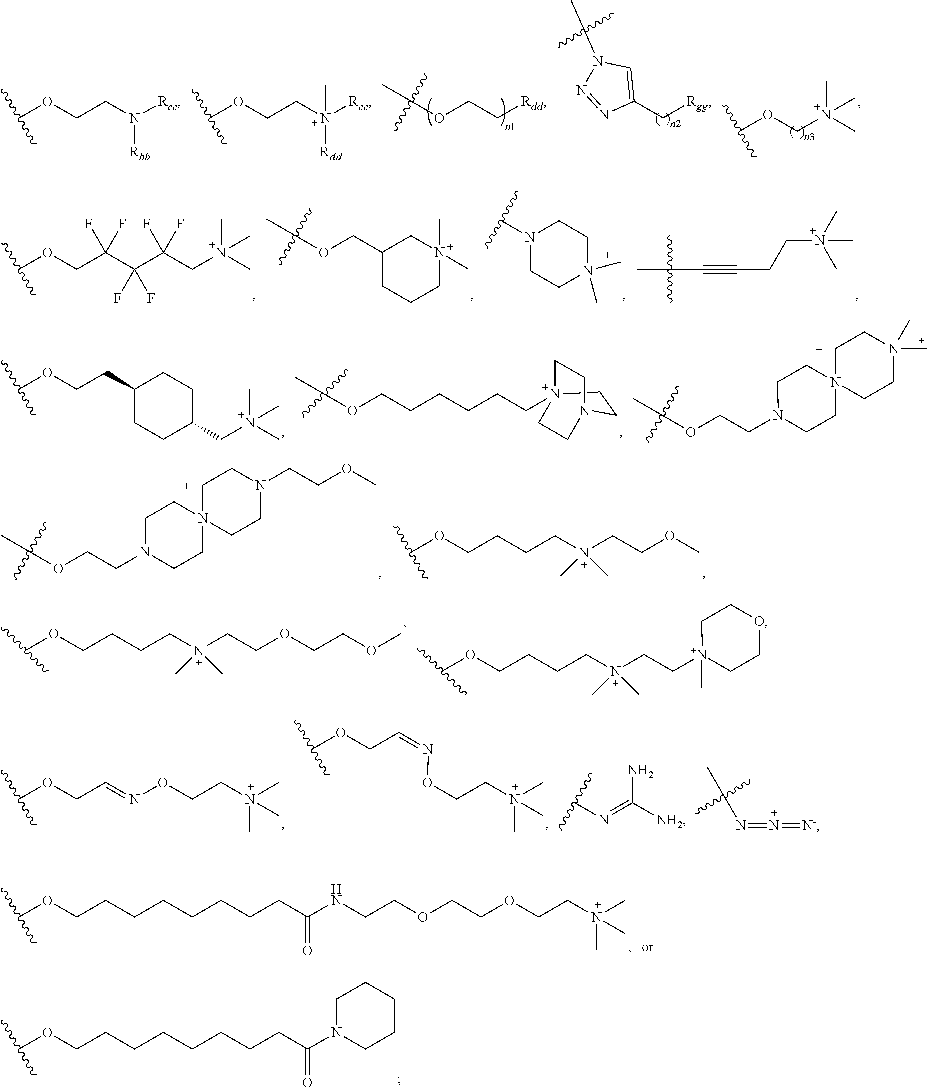

R_{bb} is —H, —CH₃, —C(O)CH₃, —C(NH)NH₂, —(CH₂)₃O(CH₂)₂OCH₃, —CH₂CH₂OCH₃, —(CH₂CH₂O)₃CH₃, —(CH₂CH₂O)₆CH₃,

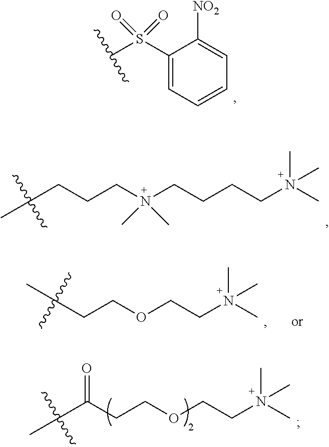

R_{cc} is —H, —CH₃, —(CH₂)₃O(CH₂)₂OCH₃,

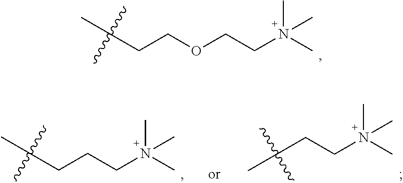

n1 is 1, 2, or 3;
R_{dd} is

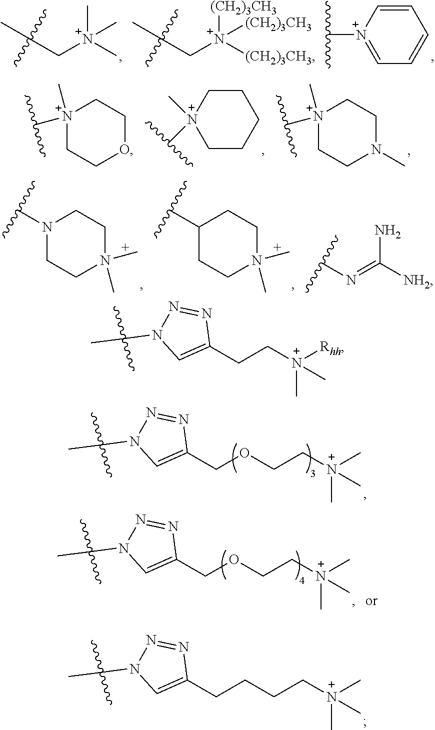

n2 is 1, 2, 3, 4, or 5;
R_{gg} is —OCH₃,

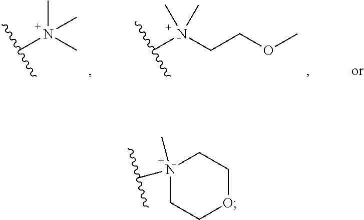

n3 is 3, 4, 5, 6, or 8;
Rₕₕ is —H, —(CH₂)₇CH₃, —(CH₂)₁₅CH₃, —(CH₂)₂OCH₃, or —(CH₂CH₂O)₃CH₃;
X₁₁ is:

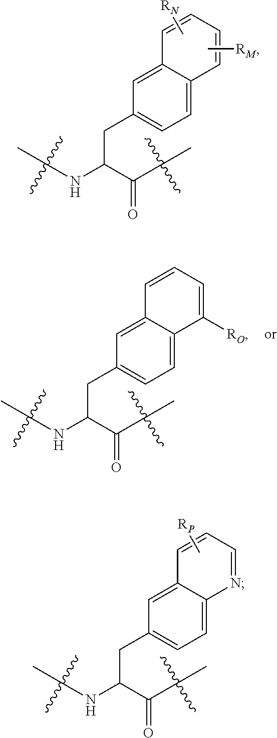

R_{M} is halo, —OH, —C₍₁₋₃₎alkyl, —OC(O)C₍₁₋₃₎haloalkyl, phenyl, or 5- to 6-membered heteroaryl, wherein the phenyl and 5- to 6-membered heteroaryl are each optionally substituted with one to three groups selected from —OH, —OC₍₁₋₃₎alkyl, —C₍₁₋₃₎haloalkyl, and 3- to 6-membered heterocyclyl;
R_{N} is —H or —OH;
R_{O} is —OC₍₁₋₃₎alkyl or —C(O)NH₂;
R_{P} is halo, —OH, —C₍₁₋₃₎alkyl, —OC₍₁₋₃₎alkyl, —C(O)NH₂, —OC(O)C₍₁₋₃₎haloalkyl, phenyl, or 5- to 6-membered heteroaryl, wherein the phenyl and 5- to 6-membered heteroaryl are each optionally substituted with one to three groups selected from —OH, —OC₍₁₋₃₎alkyl, —C₍₁₋₃₎haloalkyl, and 3- to 6-membered heterocyclyl;
X₁₂ is any amino acid;
X₁₃ is any amino acid;
X₁₄ is any amino acid;
X₁₅ is Ala, THP, or

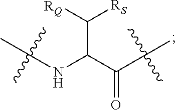

R_{Q} is —H or —C₍₁₋₃₎alkyl;
R_{S} is phenyl or 5- to 6-membered heteroaryl, each of which is optionally substituted with one —C(O)NH₂ group;
X₁₁ is any amino acid or absent; and
X₁₇ is any amino acid or absent;
wherein the peptide is cyclized to form a first ring, wherein the first ring comprises 4-11 or 14 amino acids.

In some embodiments, the present disclosure provides a peptide of Formula (I), comprising the amino acid sequence:

X₃-X₄-X₅-X₆-X₇-X₈-X₉-X₁₀-X₁₁-X₁₂-X₁₃-X₁₄-X₁₅-
X₁₆-X₁₇ (I),

or a pharmaceutically acceptable salt thereof, wherein:
X₃ is any amino acid or absent;
X₄ is an amino acid that is linked to the amino acid at X₉;
X₅ is any amino acid;
X₆ is any amino acid;
X₇ is:

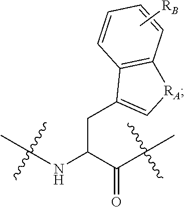

R_{A} is O, NH, N—C₍₁₋₅₎alkyl, or S;
R_{B} is —H, halo, C₍₁₋₃₎alkyl, or phenyl, wherein the phenyl is optionally substituted with one —N(H)C(O)C₍₁₋₃₎alkyl group;
X₈ is any amino acid;
X₉ is an amino acid that is linked to the amino acid at X₄;
X₁₀ is a substituted or unsubstituted aromatic amino acid;
X₁₁ is:

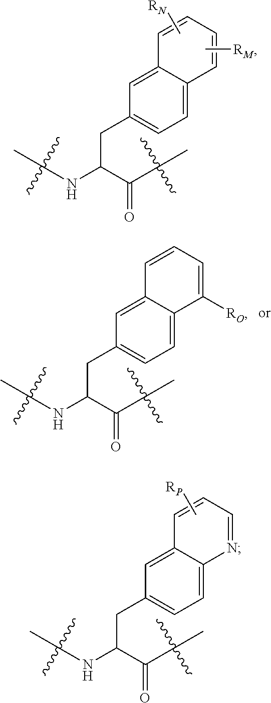

R_{M} is halo, —OH, —C₍₁₋₃₎alkyl, —OC(O)C₍₁₋₃₎haloalkyl, phenyl, or 5- to 6-membered heteroaryl, wherein the phenyl and 5- to 6-membered heteroaryl are each optionally substituted with one to three groups selected from —OH, —OC₍₁₋₃₎alkyl, —C₍₁₋₃₎haloalkyl, and 3- to 6-membered heterocyclyl;
R_{N} is —H or —OH;
R_{O} is —OC₍₁₋₃₎alkyl or —C(O)NH₂;
R_{P} is halo, —OH, —C₍₁₋₃₎alkyl, —OC₍₁₋₃₎alkyl, —C(O)NH₂, —OC(O)C₍₁₋₃₎haloalkyl, phenyl, or 5- to 6-membered heteroaryl, wherein the phenyl and 5- to 6-membered heteroaryl are each optionally substituted with one to three groups selected from —OH, —OC₍₁₋₃₎alkyl, —C₍₁₋₃₎haloalkyl, and 3- to 6-membered heterocyclyl;
X₁₂ is any amino acid;
X₁₃ is any amino acid;
X₁₄ is any amino acid;
X₁₅ is Ala, THP, or

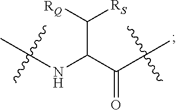

R_{Q} is —H or —C₍₁₋₃₎alkyl;
R_{S} is phenyl or 5- to 6-membered heteroaryl, each of which is optionally substituted with one —C(O)NH₂ group;
X₁₆ is any amino acid or absent; and
X₁₇ is any amino acid or absent.

In some embodiments, the present disclosure provides a peptide of Formula (I), comprising the amino acid sequence:

(I)
X₃-X₄-X₅-X₆-X₇-X₈-X₉-X₁₀-X₁₁-X₁₂-X₁₃-X₁₄-X₁₅-X₁₆-X₁₇,

or a pharmaceutically acceptable salt thereof, wherein:
X₃ is any amino acid or absent;
X₄ is an amino acid that is linked to the amino acid at X₉;
X₅ is any amino acid;
X₆ is any amino acid;
X₇ is:

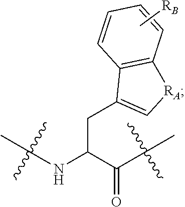

R_{A} is O, NH, N—C₍₁₋₅₎alkyl, or S;
R_{B} is —H, halo, C₍₁₋₃₎alkyl, or phenyl, wherein the phenyl is optionally substituted with one —N(H)C(O)C₍₁₋₃₎alkyl group;
X₈ is any amino acid;
X₉ is an amino acid that is linked to the amino acid at X₄;
X₁₀ is:

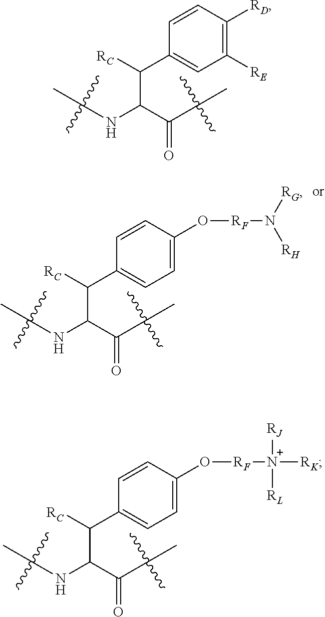

R_{C} is —H or —C₍₁₋₃₎alkyl;
R_{D} is —H, —OH, —CN, —C₍₁₋₃₎alkyl, —OC₍₁₋₃₎alkyl, —OC₍₁₋₃₎alkyl-(5-membered heteroaryl), —C(O)NH₂, or heterocyclyl, wherein the —OC₍₁₋₃₎alkyl-(5-membered heteroaryl) is optionally substituted with a polyethylene glycol chain, and wherein the heterocyclyl is optionally substituted with one —C(O)NH₂ group;
R_{E} is —H or halo;
R_{F} is —C₍₁₋₆₎alkylene or a bivalent polyethylene glycol chain;
R_{G} is —H, —C₍₁₋₃₎alkyl, or a bond to the amino acid at X₅ or X₁₃;
R_{H} is —H, —C₍₁₋₃₎alkyl, —C(NH)NH₂, —C(O)—R_{H1};
or R_{G} and R_{H} taken together with the nitrogen atom to which they are attached form a 5- to 8-membered heterocyclyl group;
R_{H1} is —C₍₁₋₅₎alkyl, —OC₍₁₋₅₎alkyl, —C₍₁₋₃₎alkyl-phenyl, -phenyl-C₍₁₋₃₎alkyl-N(H)—S(O)₂—C₍₁₋₃₎)alkyl, or a polyethylene glycol chain, wherein the —C₍₁₋₃₎alkyl-phenyl is optionally substituted with one to three groups selected from halo and —OH;
R_{J}, R_{K}, and R_{L} are each, independently, C₍₁₋₃₎alkyl;
or R_{J} and R_{K} taken together with the nitrogen atom to which they are attached form a 5- to 8-membered heterocyclyl group;
or X₁₀ is

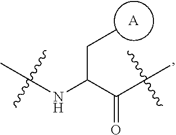

A is

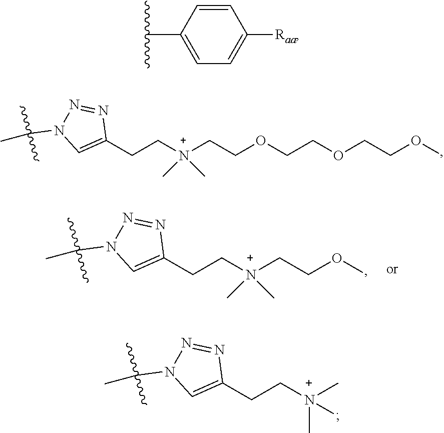

Rₐₐ is —OCHF₂, —O(CH₂)₉CO₂H,

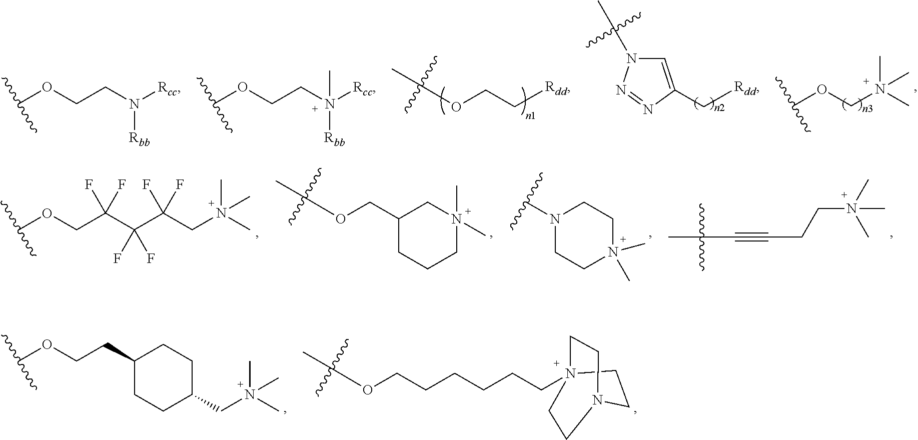

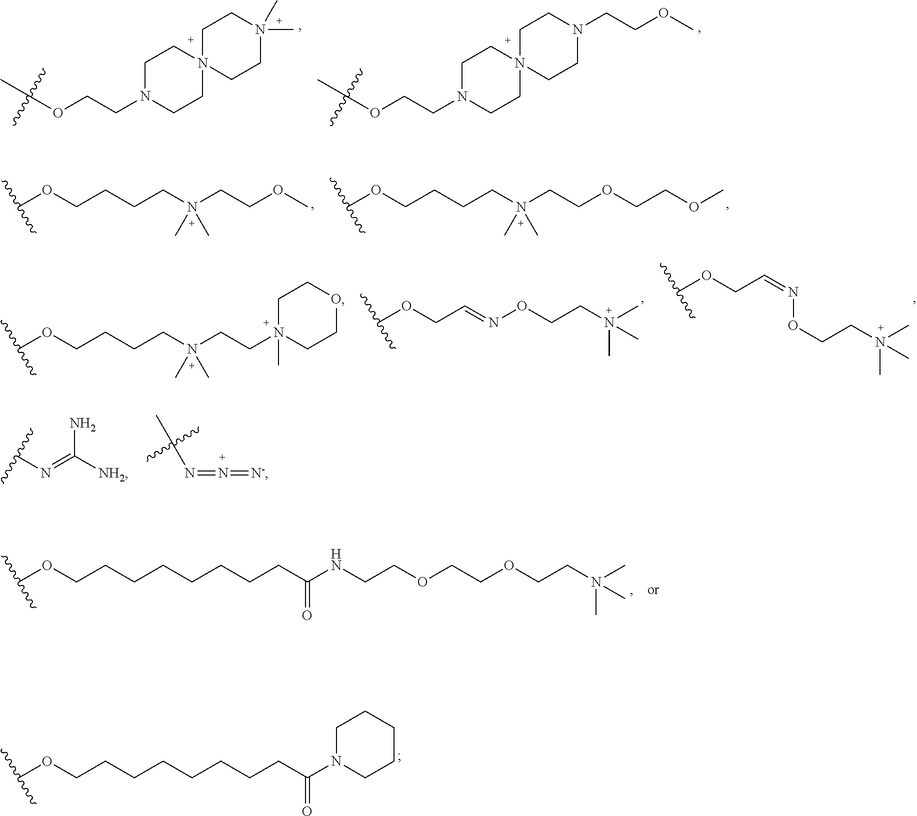

R_{bb} is —H, —CH₃, —C(O)CH₃, —C(NH)NH₂, —(CH₂)₃O(CH₂)₂OCH₃, —CH₂CH₂OCH₃, —(CH₂CH₂O)₃CH₃, —(CH₂CH₂O)₆CH₃,

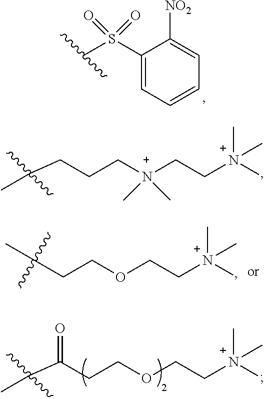

R_{cc} is —H, —CH₃, —(CH₂)₃O(CH₂)₂OCH₃,

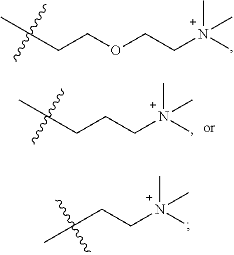

n1 is 1, 2, or 3;
R_{dd} is

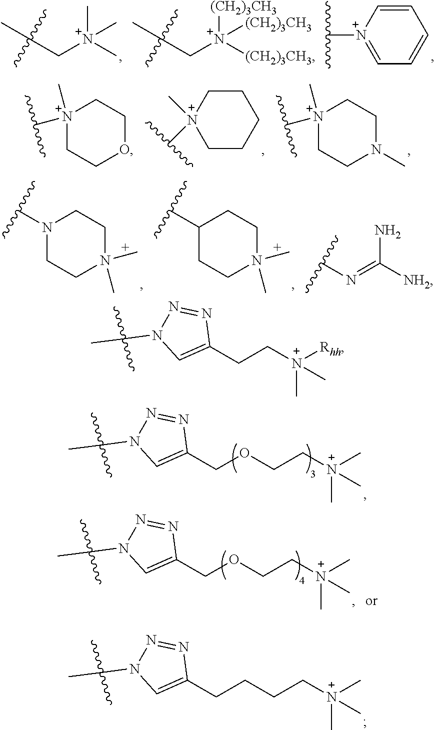

n2 is 1, 2, 3, 4, or 5;
R_{gg} is —OCH₃,

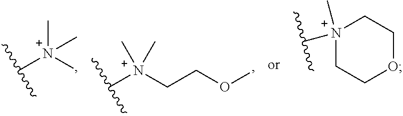

n3 is 3, 4, 5, 6, or 8;
Rₕₕ is —H, —(CH₂)₇CH₃, —(CH₂)₁₅CH₃, —(CH₂)₂OCH₃, or —(CH₂CH₂O)₃CH₃;
X₁₁ is:

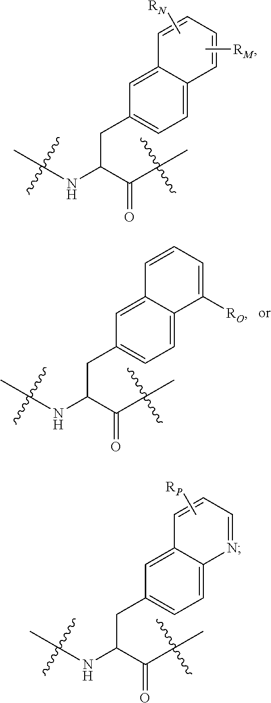

R_{M} is halo, —OH, —C₍₁₋₃₎alkyl, —OC(O)C₍₁₋₃₎haloalkyl, phenyl, or 5- to 6-membered heteroaryl, wherein the phenyl and 5- to 6-membered heteroaryl are each optionally substituted with one to three groups selected from —OH, —OC₍₁₋₃₎alkyl, —C₍₁₋₃₎haloalkyl, and 3- to 6-membered heterocyclyl;
R_{N} is —H or —OH;
R_{O} is —OC₍₁₋₃₎alkyl or —C(O)NH₂;
R_{P} is halo, —OH, —C₍₁₋₃₎alkyl, —OC₍₁₋₃₎alkyl, —C(O)NH₂, —OC(O)C₍₁₋₃₎haloalkyl, phenyl, or 5- to 6-membered heteroaryl, wherein the phenyl and 5- to 6-membered heteroaryl are each optionally substituted with one to three groups selected from —OH, —OC₍₁₋₃₎alkyl, —C₍₁₋₃₎haloalkyl, and 3- to 6-membered heterocyclyl;
X₁₂ is any amino acid;
X₁₃ is any amino acid;
X₁₄ is any amino acid;
X₁₅ is Ala, THP, or

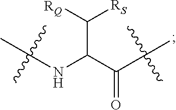

R_{Q} is —H or —C₍₁₋₃₎alkyl;
R_{S} is phenyl or 5- to 6-membered heteroaryl, each of which is optionally substituted with one —C(O)NH₂ group;
X₁₆ is any amino acid or absent; and
X₁₇ is any amino acid or absent.

In some embodiments, the present disclosure provides a peptide of Formula (I), comprising the amino acid sequence:

(I)
X₃-X₄-X₅-X₆-X₇-X₈-X₉-X₁₀-X₁₁-X₁₂-X₁₃-X₁₄-X₁₅-X₁₆-X₁₇,

or a pharmaceutically acceptable salt thereof, wherein:
X₃ is any amino acid or absent;
X₄ is any amino acid;
X₅ is any amino acid;
X₆ is any amino acid;
X₇ is:

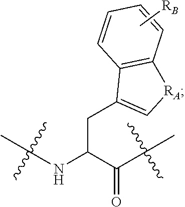

R_{A} is O, NH, N—C₍₁₋₅₎alkyl, or S;
R_{B} is —H, halo, C₍₁₋₃₎alkyl, or phenyl, wherein the phenyl is optionally substituted with one —N(H)C(O)C₍₁₋₃₎alkyl group;
X₈ is any amino acid;
X₉ is any amino acid;
X₁₀ is:

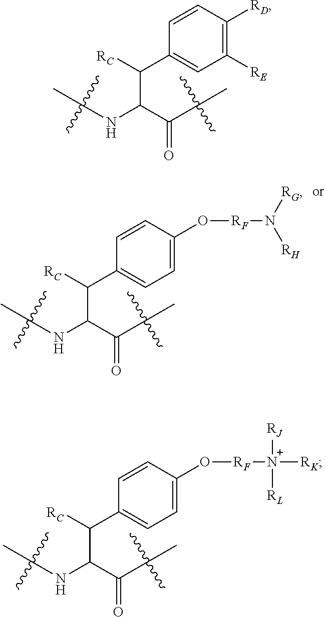

R_{C} is —H or —C₍₁₋₃₎alkyl;
R_{D} is —H, —OH, —CN, —C₍₁₋₃₎alkyl, —OC₍₁₋₃₎alkyl, —OC₍₁₋₃₎alkyl-(5-membered heteroaryl), —C(O)NH₂, or heterocyclyl, wherein the —OC₍₁₋₃₎alkyl-(5-membered heteroaryl) is optionally substituted with a polyethylene glycol chain, and wherein the heterocyclyl is optionally substituted with one —C(O)NH₂ group;
R_{E} is —H or halo;
R_{F} is —C₍₁₋₆₎alkylene or a bivalent polyethylene glycol chain;
R_{G} is —H, —C₍₁₋₃₎alkyl, or a bond to the amino acid at X₅ or X₁₃;
R_{H} is —H, —C₍₁₋₃₎alkyl, —C(NH)NH₂, —C(O)—R_{H1};
or R_{G} and R_{H} taken together with the nitrogen atom to which they are attached form a 5- to 8-membered heterocyclyl group;
R_{H1} is —C₍₁₋₅₎alkyl, —OC₍₁₋₅₎alkyl, —C₍₁₋₃₎alkyl-phenyl, -phenyl-C₍₁₋₃₎alkyl-N(H)—S(O)₂—C₍₁₋₃₎)alkyl, or a polyethylene glycol chain, wherein the —C₍₁₋₃₎alkyl-phenyl is optionally substituted with one to three groups selected from halo and —OH;
R_{J}, R_{K}, and R_{L} are each, independently, C₍₁₋₃₎alkyl;
or R_{J} and R_{K} taken together with the nitrogen atom to which they are attached form a 5- to 8-membered heterocyclyl group;
X₁₁ is:

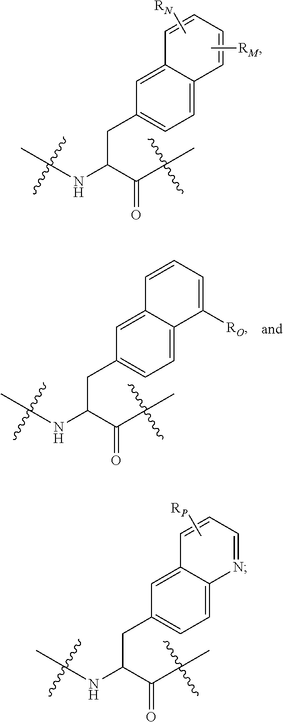

R_{M} is halo, —OH, —C₍₁₋₃₎alkyl, —OC(O)C₍₁₋₃₎haloalkyl, phenyl, or 5- to 6-membered heteroaryl, wherein the phenyl and 5- to 6-membered heteroaryl are each optionally substituted with one to three groups selected from —OH, —OC₍₁₋₃₎alkyl, —C₍₁₋₃₎haloalkyl, and 3- to 6-membered heterocyclyl;
R_{N} is —H or —OH;
R_{O} is —OC₍₁₋₃₎alkyl or —C(O)NH₂;
R_{P} is halo, —OH, —C₍₁₋₃₎alkyl, —OC₍₁₋₃₎alkyl, —C(O)NH₂, —OC(O)C₍₁₋₃₎haloalkyl, phenyl, or 5- to 6-membered heteroaryl, wherein the phenyl and 5- to 6-membered heteroaryl are each optionally substituted with one to three groups selected from —OH, —OC₍₁₋₃₎alkyl, —C₍₁₋₃₎haloalkyl, and 3- to 6-membered heterocyclyl;
X₁₂ is any amino acid;
X₁₃ is any amino acid;
X₁₄ is any amino acid;
X₁₅ is Ala, THP, or

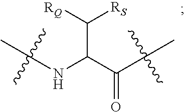

R_{Q} is —H or —C₍₁₋₃₎alkyl;
R_{S} is phenyl or 5- to 6-membered heteroaryl, each of which is optionally substituted with one —C(O)NH₂ group;
X₁₆ is any amino acid or absent; and
X₁₇ is any amino acid or absent;
X₁₇ is any amino acid or absent;
wherein the peptide is cyclized to form a first ring, wherein the first ring comprises 4-11 or 14 amino acids.

In some embodiments, the present disclosure provides a peptide of Formula (I), comprising the amino acid sequence:

(I)
X₃-X₄-X₅-X₆-X₇-X₈-X₉-X₁₀-X₁₁-X₁₂-X₁₃-X₁₄-X₁₅-X₁₆-X₁₇,

or a pharmaceutically acceptable salt thereof, wherein:
X₃ is any amino acid or absent;
X₄ is an amino acid that is linked to the amino acid at X₉;
X₅ is any amino acid;
X₆ is any amino acid;
X₇ is:

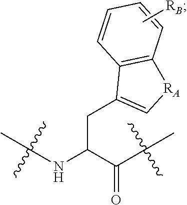

R_{A} is O, NH, N—C₍₁₋₅₎alkyl, or S;
R_{B} is —H, halo, C₍₁₋₃₎alkyl, or phenyl, wherein the phenyl is optionally substituted with one —N(H)C(O)C₍₁₋₃₎alkyl group;
X₈ is any amino acid;
X₉ is an amino acid that is linked to the amino acid at X₄;
X₁₀ is:

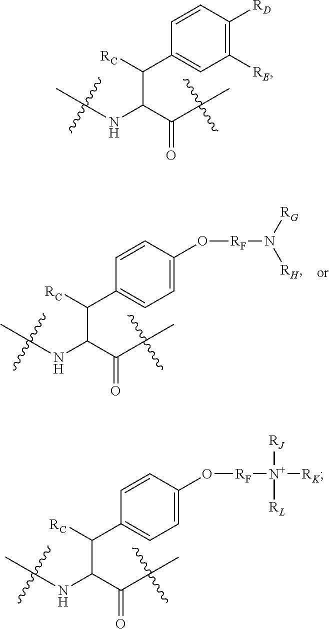

R_{C} is —H or —C₍₁₋₃₎alkyl;
R_{D} is —H, —OH, —CN, —C₍₁₋₃₎alkyl, —OC₍₁₋₃₎alkyl, —OC₍₁₋₃₎alkyl-(5-membered heteroaryl), —C(O)NH₂, or heterocyclyl, wherein the —OC₍₁₋₃₎alkyl-(5-membered heteroaryl) is optionally substituted with a polyethylene glycol chain, and wherein the heterocyclyl is optionally substituted with one —C(O)NH₂ group;
R_{E} is —H or halo;
R_{F} is —C₍₁₋₆₎alkylene or a bivalent polyethylene glycol chain;
R_{G} is —H, —C₍₁₋₃₎alkyl, or a bond to the amino acid at X₅ or X₁₃;
R_{H} is —H, —C₍₁₋₃₎alkyl, —C(NH)NH₂, —C(O)—R_{H1};
or R_{G} and R_{H} taken together with the nitrogen atom to which they are attached form a 5- to 8-membered heterocyclyl group;
R_{H1} is —C₍₁₋₅₎alkyl, —OC₍₁₋₅₎alkyl, —C₍₁₋₃₎alkyl-phenyl, -phenyl-C₍₁₋₃₎alkyl-N(H)—S(O)₂—C₍₁₋₃₎)alkyl, or a polyethylene glycol chain, wherein the —C₍₁₋₃₎alkyl-phenyl is optionally substituted with one to three groups selected from halo and —OH;
R_{J}, R_{K}, and R_{L} are each, independently, C₍₁₋₃₎alkyl;
or R_{J} and R_{K} taken together with the nitrogen atom to which they are attached form a 5- to 8-membered heterocyclyl group;
X₁₁ is:

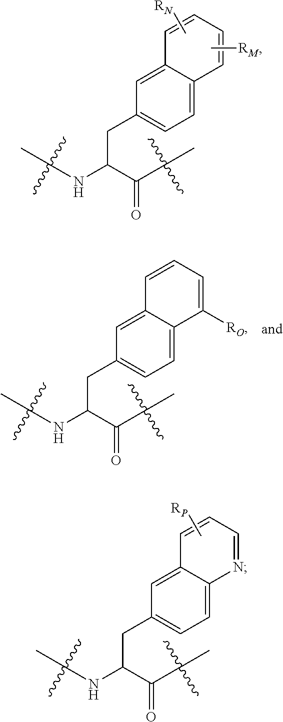

R_{M} is halo, —OH, —C₍₁₋₃₎alkyl, —OC(O)C₍₁₋₃₎haloalkyl, phenyl, or 5- to 6-membered heteroaryl, wherein the phenyl and 5- to 6-membered heteroaryl are each optionally substituted with one to three groups selected from —OH, —OC₍₁₋₃₎alkyl, —C₍₁₋₃₎haloalkyl, and 3- to 6-membered heterocyclyl;
R_{N} is —H or —OH;
R_{O} is —OC₍₁₋₃₎alkyl or —C(O)NH₂;
R_{P} is halo, —OH, —C₍₁₋₃₎alkyl, —OC₍₁₋₃₎alkyl, —C(O)NH₂, —OC(O)C₍₁₋₃₎haloalkyl, phenyl, or 5- to 6-membered heteroaryl, wherein the phenyl and 5- to 6-membered heteroaryl are each optionally substituted with one to three groups selected from —OH, —OC₍₁₋₃₎alkyl, —C₍₁₋₃₎haloalkyl, and 3- to 6-membered heterocyclyl;
X₁₂ is any amino acid;
X₁₃ is any amino acid;
X₁₄ is any amino acid;
X₁₅ is Ala, THP, or

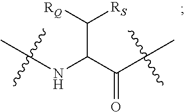

R_{Q} is —H or —C₍₁₋₃₎alkyl;
R_{S} is phenyl or 5- to 6-membered heteroaryl, each of which is optionally substituted with one —C(O)NH₂ group;
X₁₆ is any amino acid or absent; and
X₁₇ is any amino acid or absent.

In some embodiments, the peptide is not MeCO-Pen-N-T-7MeW-K(Ac)-Pen-AEF-6OHQui-THP-E-N-3Pya-Sar-CONH₂.

The linear form structure of Formula (I) is intended for exemplary and non-limiting purposes, which will be apparent from examples set forth and exemplified throughout the instant specification, e.g., where each such structure may be longer or shorter than the length of eighteen amino acids and/or other corresponding chemical moieties or functional group substituents as defined herein.

In some embodiments, the peptide comprises an amino acid sequence selected from the group consisting of

R₁-X₃-X₄-X₅-X₆-X₇-X₈-X₉-X₁₀-X₁₁-
X₁₂-X₁₃-X₁₄-X₁₅-X₁₆-X₁₇ (I-A1),
X₃-X₄-X₅-X₆-X₇-X₈-X₉-X₁₀-X₁₁-X₁₂-
X₁₃-X₁₄-X₁₅-X₁₆-X₁₇-R₂ (I-A2),
and
R₁-X₃-X₄-X₅-X₆-X₇-X₈-X₉-X₁₀-X₁₁-
X₁₂-X₁₃-X₁₄-X₁₅-X₁₆-X₁₇-R₂ (I-A3),

or a pharmaceutically acceptable salt thereof.

R₁ represents the N-terminal end of the peptide, which may be, for example, a hydrogen or a chemical moiety or functional group substituted on the amino group (e.g., an acetate group).

Similarly, R₂ represents the carboxyl end, which may be, for example the OH of the carboxyl or a chemical moiety or functional group attached thereto or substituted for the OH group (e.g., an amino group to give a terminal amide, e.g., —CONH₂); In some embodiments, R₁ is 5cpaCO, CF3CO, CF3Propylamide, EtCO, MeCO, a polyethylene glycol chain, or a lipophilic substituent, or R₁ is an alkyl or polyethylene glycol chain linked to the amino acid at X₁₃; and
R₂ is CONH2, CO(DiFPip), CON(Me)2, a polyethylene glycol chain, or a lipophilic substituent.

In some embodiments, the peptide comprises an amino acid sequence of Formula (I-A1):

R₁-X₃-X₄-X₅-X₆-X₇-X₈-X₉-X₁₀-X₁₁-
X₁₂-X₁₃-X₁₄-X₁₅-X₁₆-X₁₇ (I-A1),

or a pharmaceutically acceptable salt thereof, wherein:
R₁ is 5cpaCO, CF3CO, CF3Propylamide, EtCO, MeCO, a polyethylene glycol chain, or a lipophilic substituent, or R₁ is an alkyl or polyethylene glycol chain linked to the amino acid at X₁₃.

In some embodiments, the peptide comprises an amino acid sequence of Formula (I-A2):

X₃-X₄-X₅-X₆-X₇-X₈-X₉-X₁₀-X₁₁-X₁₂-
X₁₃-X₁₄-X₁₅-X₁₆-X₁₇-R₂ (I-A2),

or a pharmaceutically acceptable salt thereof, wherein:
R₂ is CONH2, CO(DiFPip), CON(Me)2, a polyethylene glycol chain, or a lipophilic substituent.

In some embodiments, the peptide comprises an amino acid sequence of Formula (I-A3):

R₁-X₃-X₄-X₅-X₆-X₇-X₈-X₉-X₁₀-X₁₁-X₁₂-
X₁₃-X₁₄-X₁₅-X₁₆-X₁₇-R₂ (I-A3),

or a pharmaceutically acceptable salt thereof, wherein:
R₁ is 5cpaCO, CF3CO, CF3Propylamide, EtCO, MeCO, a polyethylene glycol chain, or a lipophilic substituent, or R₁ is an alkyl or polyethylene glycol chain linked to the amino acid at X₁₃; and
R₂ is CONH2, CO(DiFPip), CON(Me)2, a polyethylene glycol chain, or a lipophilic substituent.

In some embodiments, the peptide further comprises a linkage between the amino acids at X₃ and X₁₃, between the amino acids at X₅ and X₁₀, between the amino acids at X₁₀ and X₁₃, or between R₁ and the amino acid at X₁₃. In some embodiments, the amino acid at X₃ is linked to the amino acid at X₁₃. In some embodiments, the amino acid at X₅ is linked to the amino acid at X₁₀. In some embodiments, the amino acid at X₁₀ is linked to the amino acid at X₁₃. In some embodiments, R₁ is linked to the amino acid at X₁₃.

In some embodiments, the amino acid at X₃, X₅, X₆, X₈, X₁₂, X₁₃, X₁₄, X₁₆, or X₁₇ is conjugated to a polyethylene glycol chain. In some embodiments, the amino acid at X₃, X₅, X₆, X₈, X₁₂, X₁₃, X₁₄, X₁₆, or X₁₇ is conjugated to a polyethylene glycol chain. In some embodiments, the amino acid at X₃, X₅, X₆, X₈, X₁₂, X₁₃, X₁₄, X₁₆, or X₁₇ is conjugated to a lipophilic substituent.

In some embodiments, the peptide comprises an amino acid sequence of Formula (I-B), comprising the amino acid sequence:

R₁-X₃-X₄-X₅-T-X₇-X₈-X₉-X₁₀-X₁₁-X₁₂-
X₁₃-N-X₁₅-X₁₆-X₁₇-R₂ (I-B),

or a pharmaceutically acceptable salt thereof, wherein:
R₁ is 5Ava, 5cpaCO, 6Ahx, 7Ahp, CF3CO, CF3Propylamide, EtCO, MeCO, PEG2, PEG2NMe, Z_{peg}, or Z_{lipid};
X₃ is Dab(COCH2), K(COCH2CH₂), hK(Me)3, K, K(5cpa), K-Z_{peg}, K-Z_{lipid}, K(d), K(Me)3, Ser(MePEG2), R, SP6, or absent;
X₄ is 4AminoPro, Abu, aG, aMeC, C, Dap, Pen, Pen(oXyl), Pen(mXyl), Pen(pXyl), or Pra;
X₅ is D, E, hE, K, K(a), K(Ac), K(d), K(G), K(NMe), K(NNs), K-Z_{peg}, K-Z_{lipid}, L, N, N(NMe), N(NMe2), Q, Q(NMe), or Q(NMe2);
X₇ is 7(3NacPh)W, 7BrW, 7MeW, BT, or W;
X₈ is Dab(NMeAc), Dab(NMecarn), Dab-Z_{peg}, hK(Me)3, K(Ac), K(Me)3, K(NMeAc), NMeK-Z_{peg}, K-Z_{peg}, K-Z_{lipid}, Lys(N+Me2)-Z_{peg}, Q, or Q(NMe2);
X₉ is aMeC, aG, C, D, E, hE, Pen, or Dap(N3);
X₁₀ is 4DMPzEF, 4OMeF, AEF, AEF(G), AEF(NMe), AEF(NMe2), AEF-Z_{peg}, AEF(NMe)-Z_{peg}, APEG3F, bMeAEF, F, MMoEF, TMAPF, or Y;
X₁₁ is:

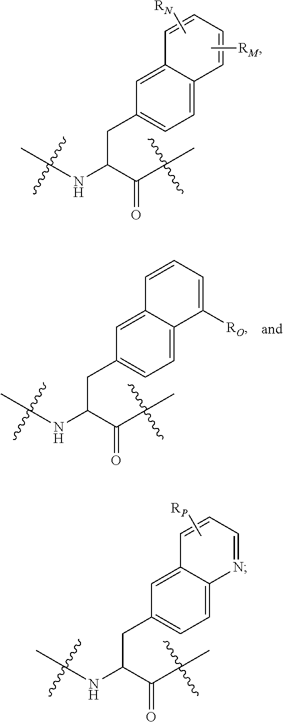

R_{M} is halo, —OH, —C₍₁₋₃₎alkyl, —OC(O)C₍₁₋₃₎haloalkyl, phenyl, or 5- to 6-membered heteroaryl, wherein the phenyl and 5- to 6-membered heteroaryl are each optionally substituted with one to three groups selected from —OH, —OC₍₁₋₃₎alkyl, —C₍₁₋₃₎haloalkyl, and 3- to 6-membered heterocyclyl;
R_{N} is —H or —OH;
R_{O} is —OC₍₁₋₃₎alkyl or —C(O)NH₂;
R_{P} is halo, —OH, —C₍₁₋₃₎alkyl, —OC₍₁₋₃₎alkyl, —C(O)NH₂, —OC(O)C₍₁₋₃₎haloalkyl, phenyl, or 5- to 6-membered heteroaryl, wherein the phenyl and 5- to 6-membered heteroaryl are each optionally substituted with one to three groups selected from —OH, —OC₍₁₋₃₎alkyl, —C₍₁₋₃₎haloalkyl, and 3- to 6-membered heterocyclyl;
X₁₂ is THP, aMeL, diFCpx, or Pip(NMe2);
X₁₃ is C, D, Dab(NMeAc), Dab(NMecarn), E, E(COcPEG3a), hE, K(Ac), K(Me)3, K(NMeAc), K-Z_{peg}, K-Z_{lipid}, L, or Q(NMe2);
X₁₅ is 3AmPyrazolAla, 3Pya, 5AmPyridinAla, 5MePyridinAla, Ala, ACIPA, aMePhe, H, or THP;
X₁₁ is Sar, NMeK-Z_{lipid}, or absent;
X₁₇ is K-Z_{lipid}, NMeK-Z_{lipid}, or absent;
R₂ is CONH2, CO(DiFPip), CON(Me)2, or Z_{peg};
Z_{peg}, independently for each occurrence, is a polyethylene glycol chain;
Z_{lipid}, independently for each occurrence, is a lipophilic substituent;
wherein the peptide is cyclized via a linkage between the residues at X₄ and X₉;
wherein:
   (a) when R₁ is 5Ava, 6Ahx, 7Ahp, PEG2, or PEG2NMe, then the peptide further comprises a linkage between the residue at R₁ and a residue selected from E or hE at X₁₃;
   (b) when X₃ is Dab(COCH2), k(COCH2CH₂), or Ser(MePEG2), and optionally when X₃ is k, then the peptide further comprises a linkage between the residue at X₃ and a residue selected from C, D, or hE at X₁₃;
   (c) when X₅ is D, E, hE, K, K(a), K(Ac), K(d), K(G), K(NMe), or K(NNs), and optionally when X₅ is K-Z_{peg} or K-Z_{lipid}, then the peptide further comprises a linkage between the residue at X₅ and a residue selected from AEF or AEF(NMe) at X₁₀;
   (d) when X₁₀ is AEF or AEF(NMe), the peptide optionally further comprises a linkage between the residue at X₁₀ and a residue selected from D, E, hE, K, K(a), K(Ac), K(d), K(G), K(NMe), K(NNs), K-Z_{peg}, and K-Z_{lipid} at X₅ or an E residue at X₁₃;
   (e) when X₁₃ is C, D, or hE, and optionally when X₁₃ is E, then the peptide further comprises a linkage between the residue at X₁₃ and a residue selected from 5Ava, 6Ahx, 7Ahp, PEG2, and PEG2NMe at R₁ or a residue selected from Dab(COCH2), k(COCH2CH₂), k, and Ser(MePEG2) at X₃ or an AEF residue at X₁₀;
provided that the peptide comprises no more than one linkage between any one of R₁ and X₁₃, X₃ and X₁₃, X₅ and X₁₀, and X₁₀ and X₁₃.

In some embodiments, the peptide comprises an amino acid sequence of Formula (I-C):

R₁-X₃-X₄-X₅-T-X₇-X₈-X₉-X₁₀-X₁₁-THP-
X₁₃-N-X₁₅-Sar-X₁₇-R₂ (I-C),

or a pharmaceutically acceptable salt thereof, wherein:
R₁ is 5cpaCO, CF3CO, MeCO, Z_{peg}, or Z_{lipid};
X₃ is hk(Me)3, k, k-Z_{peg}, k-Z_{lipid}, k(d), k(Me)3, K-Z_{peg}, K-Z_{lipid}, r, R, SP6, or absent
X₄ is 4AminoPro, Abu, aG, aMeC, C, Dap, Pen, Pen(oXyl), Pen(mXyl), Pen(pXyl), or Pra;
X₅ is D, E, hE, K, K(a), K(Ac), K(d), K(G), K(NMe), K(NNs), K-Z_{peg}, K-Z_{lipid}, N, N(NMe2), Q, or Q(NMe2);
X₇ is 7(3NAcPh)W, 7MeW, or W;
X₈ is Dab(NMeAc), Dab(NMecarn), Dab-Z_{peg}, K(Ac), K(NMeAc), NMeK-Z_{peg}, K-Z_{peg}, K-Z_{lipid}, Q, or Q(NMe2);
X₉ is aMeC, aG, C, D, E, hE, Pen, or Dap(N3);
X₁₀ is AEF, AEF(G), AEF(NMe), AEF(NMe2), AEF-Z_{peg}, AEF(NMe)-Z_{peg}, bMeAEF, MMoEF, or TMAPF;
X₁₁ is 2Nal6((5CF3)₃Pyrazole), 6OH2Nal, 2Nal6(Ph2OH), 2Nal6(Ph4(NMorph)), 2Nal6(3Pyrazole), 2Nal6(4OMePh), 5OMe2Nal, 5amido2Nal, 5Br2Nal, 5Me2Nal, 6MeQui, 6O(COCF3)₂Nal, 6F2Nal, 6Br2Nal, or 7OH2Nal;
X₁₃ is Dab(NMeAc), Dab(NMecam), E, K(Ac), K(NMeAc), K-Z_{peg}, or K-Z_{lipid};
X₁₅ is 3Pya, 5MePyridinAla, or THP;
X₁₇ is K-Z_{lipid}, NMeK-Z_{lipid}, or absent;
R₂ is CONH2, CON(Me)2, or Z_{peg};
Z_{peg}, independently for each occurrence, is a polyethylene glycol chain;
Z_{lipid}, independently for each occurrence, is a lipophilic substituent;
wherein the peptide is cyclized via a linkage between the residues at X₄ and X₉; and
wherein when X₅ is D, E, hE, K, K(a), K(Ac), K(d), K(G), K(NMe), or K(NNs), and optionally when X₅ is K-Z_{peg} or K-Z_{lipid}, then the peptide further comprises a linkage between the residue at X₅ and a residue selected from AEF or AEF(NMe) at X₁₀.

In some embodiments, the peptide comprises an amino acid sequence of Formula (I-D):

R₁-X₃-Pen-X₅-T-X₇-X₈-Pen-X₁₀-6OH2Nal-
THP-X₁₃-N-3Pya-Sar-X₁₇-R₂ (I-D),

or a pharmaceutically acceptable salt thereof, wherein:
R₁ is MeCO, Z_{peg}, or Z_{lipid};
X₃ is k-Z_{peg}, k-Z_{lipid}, k(Me)3, r, or absent
X₅ is D, E, hE, N, N(NMe2), Q, or Q(NMe2);
X₇ is 7(3NAcPh)W, 7MeW, or W;
X₈ is K(Ac), K(NMeAc), NMeK-Z_{peg}, K-Z_{peg}, or K-Z_{lipid};
X₁₀ is AEF or TMAPF;
X₁₃ is E, K(Ac), K(NMeAc), K-Z_{peg}, or K-Z_{lipid};
X₁₇ is K-Z_{lipid}, NMeK-Z_{lipid}, or absent;
R₂ is CONH2, CON(Me)2, or Z_{peg};
Z_{peg}, independently for each occurrence, is a polyethylene glycol chain;
Z_{lipid}, independently for each occurrence, is a lipophilic substituent;
wherein the peptide is cyclized via a linkage between the Pen residues at X₄ and X₉; and
wherein when X₅ is D, E, or hE, then the peptide further comprises a linkage between the residue at X₅ and the AEF residue at X₁₀.

In some embodiments, the peptide comprises an amino acid sequence of Formula (I-E):

R₁-X₃-Pen-X₅-T-7(3NAcPh)W-X₈-Pen-X₁₀-6OH2Nal-THP-X₁₃-N-3Pya-Sar-X₁₇-R₂ (I-E),

or a pharmaceutically acceptable salt thereof, wherein:
R₁ is MeCO or Z_{peg};
X₃ is r or absent
X₅ is E, N, or N(NMe2);
X₈ is K(Ac) or K(NMeAc);
X₁₀ is AEF or TMAPF;
X₁₃ is E, K(Ac), or K(NMeAc);
X₁₇ is K-Z_{lipid} or absent;
R₂ is CONH2 or CON(Me)2;
Z_{peg}, independently for each occurrence, is a polyethylene glycol chain;
Z_{lipid}, independently for each occurrence, is a lipophilic substituent;
wherein the peptide is cyclized via a linkage between the Pen residues at X₄ and X₉; and
wherein when X₅ is E then the peptide further comprises a linkage between the E residue at X₅ and the AEF residue at X₁₀.

In some embodiments, the peptide comprises an amino acid sequence of Formula (I-B):

R₁-X₃-X₄-X₅-T-X₇-X₈-X₉-X₁₀-X₁₁-X₁₂-
X₁₃-N-X₁₅-X₁₆-X₁₇-R₂ (I-B),

or a pharmaceutically acceptable salt thereof, wherein:
R₁ is 5cpaCO, CF3CO, CF3Propylamide, EtCO, MeCO, Z_{peg}, or Z_{lipid};
X₃ is hk(Me)3, k, k(5cpa), k-Z_{peg}, k-Z_{lipid}, k(d), k(Me)3, K-Z_{peg}, K-Z_{lipid}, r, R, SP6, or absent
X₄ is 4AminoPro, Abu, aG, aMeC, C, Dap, Pen, Pen(oXyl), Pen(mXyl), Pen(pXyl), or Pra;
X₅ is K-Z_{peg}, K-Z_{lipid}, L, N, N(NMe2), Q, or Q(NMe2);
X₇ is 7(3NAcPh)W, 7BrW, 7MeW, BT, or W;
X₈ is Dab(NMeAc), Dab(NMecarn), Dab-Z_{peg}, hK(Me)3, K(Ac), K(Me)3, K(NMeAc), NMeK-Z_{peg}, K-Z_{peg}, K-Z_{lipid}, Lys(N+Me2)-Z_{peg}, Q, or Q(NMe2);
X₉ is aMeC, aG, C, D, E, hE, Pen, or Dap(N3);
X₁₀ is 4DMPzEF, 4OMeF, AEF, AEF(G), AEF(NMe), AEF(NMe2), AEF-Z_{peg}, AEF(NMe)-Z_{peg}, APEG3F, bMeAEF, F, MMoEF, TMAPF, or Y;
X₁₁ is:

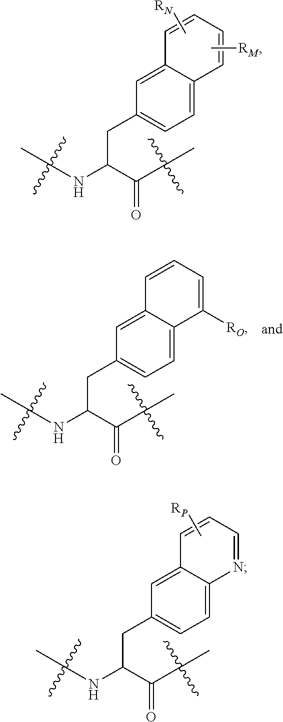

R_{M} is halo, —OH, —C₍₁₋₃₎alkyl, —OC(O)C₍₁₋₃₎haloalkyl, phenyl, or 5- to 6-membered heteroaryl, wherein the phenyl and 5- to 6-membered heteroaryl are each optionally substituted with one to three groups selected from —OH, —OC₍₁₋₃₎alkyl, —C₍₁₋₃₎haloalkyl, and 3- to 6-membered heterocycle;
R_{N} is —H or —OH;
R_{O} is —OC₍₁₋₃₎alkyl or —C(O)NH₂;
R_{P} is halo, —OH, —C₍₁₋₃₎alkyl, —OC₍₁₋₃₎alkyl, —C(O)NH₂, —OC(O)C₍₁₋₃₎haloalkyl, phenyl, or 5- to 6-membered heteroaryl, wherein the phenyl and 5- to 6-membered heteroaryl are each optionally substituted with one to three groups selected from —OH, —OC₍₁₋₃₎alkyl, —C₍₁₋₃₎haloalkyl, and 3- to 6-membered heterocycle;
X₁₂ is THP, aMeL, diFCpx, or Pip(NMe2);
X₁₃ is Dab(NMeAc), Dab(NMecarn), E, E(COcPEG3a), K(Ac), K(Me)3, K(NMeAc), K-Z_{peg}, K-Z_{lipid}, L, or Q(NMe2);
X₁₅ is 3AmPyrazolAla, 3Pya, 5AmPyridinAla, 5MePyridinAla, Ala, ACIPA, aMePhe, H, or THP;
X₁₆ is Sar, NMeK-Z_{lipid}, or absent;
X₁₇ is K-Z_{lipid}, NMeK-Z_{lipid}, or absent;
R₂ is CONH2, CO(DiFPip), CON(Me)2, or Z_{peg};
Z_{peg}, independently for each occurrence, is a polyethylene glycol chain;
Z_{lipid}, independently for each occurrence, is a lipophilic substituent;
wherein the peptide is cyclized via a linkage between the residues at X₄ and X₉.

In some embodiments, the peptide comprises an amino acid sequence of Formula (I-F):

R₁-X₃-X₄-X₅-T-X₇-X₈-X₉-X₁₀-X₁₁-X₁₂-
X₁₃-N-X₁₅-X₁₆-R₂ (I-F)

wherein:
R₁ is 5Ava, 5cpaCO, 6Ahx, 7Ahp, CF3CO, CF3Propylamide, EtCO, MeCO, PEG2, PEG2NMe, or Z_{peg};
X₃ is Dab(COCH2), k(COCH2CH₂), hk(Me)3, k, k(5cpa), k(d), k(Me)3, Ser(MePEG2), r, R, SP6, or absent;
X₄ is 4AminoPro, Abu, aG, aMeC, C, Dap, Pen, Pen(oXyl), Pen(mXyl), Pen(pXyl), or Pra;
X₅ is D, E, hE, K, K(a), K(Ac), K(d), K(G), K(NMe), K(NNs), K-Z_{peg}, L, N, N(NMe), N(NMe2), Q, Q(NMe), or Q(NMe2);
X₇ is 7(3NAcPh)W, 7BrW, 7MeW, BT, or W;
X₈ is Dab(NMeAc), Dab(NMecarn), Dab-Z_{peg}, hK(Me)3, K(Ac), K(Me)3, K(NMeAc), NMeK-Z_{peg}, K-Z_{peg}, Lys(N+Me2)-Z_{peg}, Q, or Q(NMe2);
X₉ is aMeC, aG, C, D, E, hE, Pen, or Dap(N3);
X₁₀ is 4DMPzEF, 4OMeF, AEF, AEF(G), AEF(NMe), AEF(NMe2), AEF-Z_{peg}, AEF(NMe)-Z_{peg}, APEG3F, bMeAEF, F, MMoEF, TMAPF, or Y;
X₁₁ is:

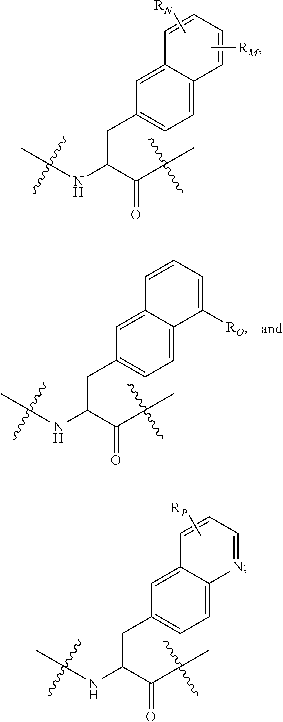

R_{M} is halo, —OH, —C₍₁₋₃₎alkyl, —OC(O)C₍₁₋₃₎haloalkyl, phenyl, or 5- to 6-membered heteroaryl, wherein the phenyl and 5- to 6-membered heteroaryl are each optionally substituted with one to three groups selected from —OH, —OC₍₁₋₃₎alkyl, —C₍₁₋₃₎haloalkyl, and 3- to 6-membered heterocyclyl;
R_{N} is —H or —OH;
R_{O} is —OC₍₁₋₃₎alkyl or —C(O)NH₂;
R_{P} is halo, —OH, —C₍₁₋₃₎alkyl, —OC₍₁₋₃₎alkyl, —C(O)NH₂, —OC(O)C₍₁₋₃₎haloalkyl, phenyl, or 5- to 6-membered heteroaryl, wherein the phenyl and 5- to 6-membered heteroaryl are each optionally substituted with one to three groups selected from —OH, —OC₍₁₋₃₎alkyl, —C₍₁₋₃₎haloalkyl, and 3- to 6-membered heterocyclyl;
X₁₂ is THP, aMeL, diFCpx, or Pip(NMe2);
X₁₃ is C, D, Dab(NMeAc), Dab(NMecarn), E, E(COcPEG3a), hE, K(Ac), K(Me)3, K(NMeAc), K-Z_{peg}, L, or Q(NMe2);
X₁₅ is 3AmPyrazolAla, 3Pya, 5AmPyridinAla, 5MePyridinAla, Ala, ACIPA, aMePhe, H, or THP;
X₁₆ is Sar or absent;
R₂ is CONH2, CO(DiFPip), CON(Me)2, or Z_{peg};
Z_{peg}, independently for each occurrence, is a polyethylene glycol;
wherein the peptide is cyclized via a linkage between the residues at X₄ and X₉;
wherein:
   (a) when R₁ is 5Ava, 6Ahx, 7Ahp, PEG2, or PEG2NMe, then the peptide further comprises a linkage between the residue at R₁ and a residue selected from E or hE at X₁₃;
   (b) when X₃ is Dab(COCH2), k(COCH2CH₂), or Ser(MePEG2), and optionally when X₃ is k, then the peptide further comprises a linkage between the residue at X₃ and a residue selected from C, D, or hE at X₁₃;
   (c) when X₅ is D, E, hE, K, K(a), K(Ac), K(d), K(G), K(NMe), or K(NNs), and optionally when X₅ is K-Z_{peg}, then the peptide further comprises a linkage between the residue at X₅ and a residue selected from AEF or AEF(NMe) at X₁₀;
   (d) when X₁₀ is AEF or AEF(NMe), the peptide optionally further comprises a linkage between the residue at X₁₀ and a residue selected from D, E, hE, K, K(a), K(Ac), K(d), K(G), K(NMe), K(NNs), and K-Z_{peg} at X₅ or an E residue at X₁₃;
   (e) when X₁₃ is C, D, or hE, and optionally when X₁₃ is E, then the peptide further comprises a linkage between the residue at X₁₃ and a residue selected from 5Ava, 6Ahx, 7Ahp, PEG2, and PEg2NMe at R₁ or a residue selected from Dab(COCH2), k(COCH2CH₂), k, and Ser(MePEG2) at X₃ or an AEF residue at X₁₀;
provided that the peptide comprises no more than one linkage between any one of R₁ and X₁₃, X₃ and X₁₃, X₅ and X₁₀, and X₁₀ and X₁₃.

In some embodiments, R₁ is 5Ava, 5cpaCO, 6Ahx, 7Ahp, CF3CO, CF3Propylamide, EtCO, MeCO, PEG2, PEG2NMe, Z_{peg} (i.e., a polyethylene glycol chain), or Z_{lipid} (i.e., a lipophilic substituent), wherein the 5Ava, 6Ahx, 7Ahp, PEG2, and PEG2NMe are linked to the amino acid at X₁₃. In some embodiments, R₁ is 5Ava, 5cpaCO, 6Ahx, 7Ahp, CF3CO, CF3Propylamide, EtCO, MeCO, PEG2, PEG2NMe, Z_{peg} (i.e., a polyethylene glycol chain), or Z_{lipid} (i.e., a lipophilic substituent), wherein the 5Ava, 6Ahx, 7Ahp, PEG2, and PEG2NMe are linked to a residue selected from E or hE at X₁₃ via an amide linkage.

In some embodiments, R₁ is 5Ava, 5cpaCO, 6Ahx, 7Ahp, CF3CO, CF3Propylamide, EtCO, MeCO, PEG2, PEG2NMe, a polyethylene glycol chain, or a lipophilic substituent, wherein the 5Ava, 6Ahx, 7Ahp, PEG2, and PEG2NMe are linked to the amino acid at X₁₃. In some embodiments, R₁ is 5Ava, 5cpaCO, 6Ahx, 7Ahp, CF3CO, CF3Propylamide, EtCO, MeCO, PEG2, PEG2NMe, a polyethylene glycol chain, or a lipophilic substituent, wherein the 5Ava, 6Ahx, 7Ahp, PEG2, and PEG2NMe are linked to a residue selected from E or hE at X₁₃ via an amide linkage.

In some embodiments, R₁ is 5cpaCO, CF3CO, MeCO, Z_{peg} (i.e., a polyethylene glycol chain), or Z_{lipid} (i.e., a lipophilic substituent). In some embodiments, R₁ is 5cpaCO, CF3CO, MeCO, cPEG3aCO, or Z_{lipid}. In some embodiments, R₁ is 5cpaCO, CF3CO, MeCO, or cPEG3aCO. In some embodiments, R₁ is MeCO, Z_{peg}, or Z_{lipid}. In some embodiments, R₁ is MeCO or Z_{peg}. In some embodiments, R₁ is MeCO or cPEG3aCO.

In some embodiments, R₁ is 5cpaCO, CF3CO, CF3Propylamide, EtCO, MeCO, Z_{peg}, or Z_{lipid}. In some embodiments, R₁ is 5Ava, 5cpaCO, 6Ahx, 7Ahp, CF3CO, CF3Propylamide, EtCO, MeCO, PEG2, PEG2NMe, or Z_{peg}, wherein the 5Ava, 6Ahx, 7Ahp, PEG2, and PEG2NMe are linked to a residue selected from E or hE at X₁₃ via an amide linkage.

In some embodiments, R₁ is an alkyl or polyethylene glycol chain linked to the amino acid at X₁₃. In some embodiments, R₁ is 5Ava linked to the amino acid at X₁₃. In some embodiments, X₁ is 5Ava linked to E at X₁₃. In some embodiments, R₁ is 5cpaCO. In some embodiments, R₁ is 6Ahx linked to the amino acid at X₁₃. In some embodiments, R₁ is 6Ahx linked to E at X₁₃. R₁ is 7Ahp linked to the amino acid at X₁₃. In some embodiments, R₁ is 7Ahp linked to E at X₁₃. In some embodiments, R₁ is CF3CO. In some embodiments, R₁ is CF3Propylamide. In some embodiments, R₁ is EtCO. In some embodiments, R₁ is MeCO. In some embodiments, R₁ is PEG2 linked to the amino acid at X₁₃. In some embodiments, R₁ is PEG2 linked to hE at X₁₃. In some embodiments, R₁ is PEG2NMe linked to the amino acid at X₁₃. In some embodiments, R₁ is PEG2NMe linked to hE at X₁₃. In some embodiments, R₁ is Z_{peg}, i.e., a polyethylene glycol chain. In some embodiments, R₁ is a polyethylene glycol chain terminating in an ammonium or methyl group. In some embodiments, R₁ is cPEG3aCO. In some embodiments, R₁ is Z_{lipid}, i.e., a lipophilic substituent.

In some embodiments, X₃ is Dab(COCH2), Dab(NMeAc), Dab(NMecam), Dab-Z_{peg}, Dab-Z_{lipid}, K(COCH2CH₂), hK(Me)3), K, K(5cpa), K(Ac), K(d), K(G), K(Me)3, K(NMe), K(NMeAc), K(NNs), K-Z_{peg}, K-Z_{lipid}, NMeK-Z_{peg}, NMeK-Z_{lipid}, Ser(MePEG2), R, SP6, or absent; wherein the Dab(COCH2), K(COCH2CH₂), and Ser(MePEG2) are linked to the amino acid at X₁₃.

In some embodiments, X₃ is Dab(COCH2), Dab(NMeAc), Dab(NMecam), Dab-Z_{peg}, Dab-Z_{lipid}, K(COCH2CH₂), hK(Me)3), K, K(5cpa), K(Ac), K(d), K(G), K(Me)3, K(NMe), K(NMeAc), K(NNs), K-Z_{peg}, K-Z_{lipid}, NMeK-Z_{peg}, NMeK-Z_{lipid}, Ser(MePEG2), R, SP6, or absent; wherein the Dab(COCH2), K(COCH2CH₂), and Ser(MePEG2) are linked to the amino acid at X₁₃; and wherein the Dab(COCH2), Dab(NMeAc), Dab(NMecarn), Dab-Z_{peg}, Dab-Z_{lipid}, K(COCH2CH₂), hK(Me)3), K, K(5cpa), K(Ac), K(d), K(G), K(Me)3, K(NMe), K(NMeAc), K(NNs), K-Z_{peg}, K-Z_{lipid}, NMeK-Z_{peg}, NMeK-Z_{lipid}, Ser(MePEG2), and R are L amino acids.

In some embodiments, X₃ is dDab(COCH2), dDab(NMeAc), dDab(NMecam), dDab-Z_{peg}, dDab-Z_{lipid}, k(COCH2CH₂), hk(Me)3), k, k(5cpa), k(Ac), k(d), k(G), k(Me)3, k(NMe), k(NMeAc), k(NNs), k-Z_{peg}, k-Z_{lipid}, NMek-Z_{peg}, NMek-Z_{lipid}, dSer(MePEG2), r, SP6, or absent; wherein the dDab(COCH2), k(COCH2CH₂), and dSer(MePEG2) are linked to the amino acid at X₁₃.

In some embodiments, X₃ is Dab(COCH2), k(COCH2CH₂), hk(Me)3, k, k(5cpa), k-Z_{peg}, k-Z_{lipid}, k(d), k(Me)3, K-Z_{peg}, K-Z_{lipid}, Ser(MePEG2), r, R, SP6, or absent; wherein the Dab(COCH2), k(COCH2CH₂), and Ser(MePEG2) are linked to the amino acid at X₁₃. In some embodiments, X₃ is hk(Me)3, k, k-Z_{peg}, k-Z_{lipid}, k(d), k(Me)3, K-Z_{peg}, K-Z_{lipid}, r, R, SP6, or absent. In some embodiments, X₃ is k-Z_{peg}, k-Z_{lipid}, k(Me)3, r, or absent. In some embodiments, X₃ is r or absent.

In some embodiments, X₃ is hk(Me)3, k, k(5cpa), k-Z_{peg}, k-Z_{lipid}, k(d), k(Me)3, K-Z_{peg}, K-Z_{lipid}, r, R, SP6, or absent. In some embodiments, X₃ is Dab(COCH2), k(COCH2CH₂), hk(Me)3, k, k(5cpa), k(d), k(Me)3, Ser(MePEG2), r, R, SP6, or absent, wherein the Dab(COCH2), k(COCH2CH₂), and Ser(MePEG2) are linked to the amino acid at X₁₃.

In some embodiments, X₃ is Dab(COCH2) linked to the amino acid at X₁₃. In some embodiments, X₃ is Dab(COCH2) linked to C at X₁₃. In some embodiments, X₃ is Dab(NMeAc). In some embodiments, X₃ is Dab(NMecarn). In some embodiments, X₃ is Dab-Z_{peg}. In some embodiments, X₃ is Dab-Z_{lipid}. In some embodiments, X₃ is K(COCH2CH₂) linked to the amino acid at X₁₃. In some embodiments, X₃ is K(COCH2CH₂) linked to C at X₁₃. In some embodiments, X₃ is hK(Me)3). In some embodiments, X₃ is K. In some embodiments, X₃ is K(5cpa). In some embodiments, X₃ is K(Ac). In some embodiments, X₃ is K(d). In some embodiments, X₃ is K(G). In some embodiments, X₃ is K(Me)3. In some embodiments, X₃ is K(NMe). In some embodiments, X₃ is K(NMeAc). In some embodiments, X₃ is K(NNs). In some embodiments, X₃ is K-Z_{peg}. In some embodiments, X₃ is K-Z_{lipid}. In some embodiments, X₃ is NMeK-Z_{peg}. In some embodiments, X₃ is NMeK-Z_{lipid}. In some embodiments, X₃ is Ser(MePEG2) linked to the amino acid at X₁₃. In some embodiments, X₃ is Ser(MePEG2) linked to D at X₁₃. In some embodiments, X₃ is R. In some embodiments, X₃ is SP6. In some embodiments, X₃ is absent.

In some embodiments, X₃ is dDab(COCH2) linked to the amino acid at X₁₃. In some embodiments, X₃ is dDab(COCH2) linked to C at X₁₃. In some embodiments, X₃ is dDab(NMeAc). In some embodiments, X₃ is dDab(NMecarn). In some embodiments, X₃ is dDab-Z_{peg}. In some embodiments, X₃ is dDab-Z_{lipid}. In some embodiments, X₃ is k(COCH2CH₂) linked to the amino acid at X₁₃. In some embodiments, X₃ is k(COCH2CH₂) linked to C at X₁₃.

In some embodiments, X₃ is hk(Me)3). In some embodiments, X₃ is k. In some embodiments, X₃ is k(5cpa). In some embodiments, X₃ is k(Ac). In some embodiments, X₃ is k(d). In some embodiments, X₃ is k(G). In some embodiments, X₃ is k(Me)3. In some embodiments, X₃ is k(NMe). In some embodiments, X₃ is k(NMeAc). In some embodiments, X₃ is k(NNs). In some embodiments, X₃ is k-Z_{peg}. In some embodiments, X₃ is k-Z_{lipid}. In some embodiments, X₃ is NMek-Z_{peg}. In some embodiments, X₃ is NMek-Z_{lipid}. In some embodiments, X₃ is dSer(MePEG2) linked to the amino acid at X₁₃. In some embodiments, X₃ is dSer(MePEG2) linked to D at X₁₃. In some embodiments, X₃ is r.

In some embodiments, X₄ is 4AminoPro, Abu, aG, aMeC, C, Dap, Pen, Pen(oXyl), Pen(mXyl), Pen(pXyl), or Pra. In some embodiments, X₄ is 4AminoPro, Abu, aG, aMeC, C, Dap, Pen, Pen(oXyl), Pen(mXyl), Pen(pXyl), or Pra, each of which is an L amino acid. In some embodiments, X₄ is 4Amino-d-Pro, dAbu, d-aG, aMe-d-C, c, dDap, dPen, dPen(oXyl), dPen(mXyl), dPen(pXyl), or dPra.

In some embodiments, X₄ is 4AminoPro, aG, Dap, Pen(oXyl), Pen(mXyl), Pen(pXyl), or Pra. In some embodiments, X₄ is Abu, aMeC, C, Pen, Pen(oXyl), Pen(mXyl), or Pen(pXyl). In some embodiments, X₄ is Abu, aMeC, C, or Pen. In some embodiments, X₄ is Abu, C, or Pen. In some embodiments, X₄ is Abu or Pen.

In some embodiments, X₄ is 4AminoPro. In some embodiments, X₄ is 4RAminoPro. In some embodiments, X₄ is 4SAminoPro. In some embodiments, X₄ is Abu. In some embodiments, X₄ is aG. In some embodiments, X₄ is aMeC. In some embodiments, X₄ is C. In some embodiments, X₄ is Dap. In some embodiments, X₄ is Pen. In some embodiments, X₄ is Pen(oXyl). In some embodiments, X₄ is Pen(mXyl). In some embodiments, X₄ is Pen(pXyl). In some embodiments, X₄ is Pra.

In some embodiments, X₄ is 4Amino-d-Pro. In some embodiments, X₄ is 4RAmino-d-Pro. In some embodiments, X₄ is 4SAmino-d-Pro. In some embodiments, X₄ is dAbu. In some embodiments, X₄ is d-aG. In some embodiments, X₄ is aMe-d-C. In some embodiments, X₄ is c.

In some embodiments, X₄ is dDap. In some embodiments, X₄ is dPen. In some embodiments, X₄ is dPen(oXyl). In some embodiments, X₄ is dPen(mXyl). In some embodiments, X₄ is dPen(pXyl). In some embodiments, X₄ is dPra.

In some embodiments, X₅ is D, E, hE, K, K(5cpa), K(a), K(Ac), K(d), K(G), K(Me)3, K(NMe), K(NMeAc), K(NNs), K-Z_{peg}, K-Z_{lipid}, NMeK-Z_{peg}, NMeK-Z_{lipid}, L, N, N(NMe), N(NMe2), Q, Q(NMe), or Q(NMe2); wherein the D, E, hE, K, K(a), K(Ac), K(d), K(G), K(NMe), and K(NNs) are linked to the amino acid at X₁₀, and optionally wherein the K-Z_{peg} or K-Z_{lipid} are linked to the amino acid at X₁₀. In some embodiments, X₅ is D, E, hE, K, K(5cpa), K(a), K(Ac), K(d), K(G), K(Me)3, K(NMe), K(NMeAc), K(NNs), K-Z_{peg}, K-Z_{lipid}, NMeK-Z_{peg}, NMeK-Z_{lipid}, L, N, N(NMe), N(NMe2), Q, Q(NMe), or Q(NMe2), each of which is an L amino acid; wherein the D, E, hE, K, K(a), K(Ac), K(d), K(G), K(NMe), and K(NNs) are linked to the amino acid at X₁₀, and optionally wherein the K-Z_{peg} or K-Z_{lipid} are linked to the amino acid at X₁₀. In some embodiments, X₅ is d, e, he, k, k(5cpa), k(a), k(Ac), k(d), k(G), k(Me)3, k(NMe), k(NMeAc), k(NNs), k-Z_{peg}, k-Z_{lipid}, NMek-Z_{peg}, NMek-Z_{lipid}, 1, n, n(NMe), n(NMe2), q, q(NMe), or q(NMe2); wherein the d, e, he, k, k(a), k(Ac), k(d), k(G), k(NMe), and k(NNs) are linked to the amino acid at X₁₀, and optionally wherein the k-Z_{peg} or k-Z_{lipid} are linked to the amino acid at X₁₀.

In some embodiments, X₅ is E, K, K(5cpa), K(a), K(Ac), K(d), K(G), K(Me)3, K(NMe), K(NMeAc), K(NNs), K-Z_{peg}, K-Z_{lipid}, NMeK-Z_{peg}, NMeK-Z_{lipid}, L, N, N(NMe2), or Q; wherein the E, K, K(a), K(Ac), K(d), K(G), K(NMe), and K(NNs) are linked to the amino acid at X₁₀, and optionally wherein the K-Z_{peg} or K-Z_{lipid} are linked to the amino acid at X₁₀. In some embodiments, X₅ is D, E, hE, K, K(a), K(Ac), K(d), K(G), K(Nme), K(NNs), K-Z_{peg}, K-Z_{lipid}, L, N, N(NMe2), Q, or Q(NMe2); wherein the D, E, hE, K, K(a), K(Ac), K(d), K(G), K(NMe), and K(NNs) are linked to the amino acid at X₁₀, and optionally wherein the K-Z_{peg} or K-Z_{lipid} are linked to the amino acid at X₁₀. In some embodiments, X₅ is E, K, K(a), K(Ac), K(d), K(G), K(Nme), K(NNs), K-Z_{peg}, K-Z_{lipid}, L, N, N(NMe2), Q, or Q(NMe2); wherein the E, K, K(a), K(Ac), K(d), K(G), K(NMe), and K(NNs) are linked to the amino acid at X₁₀, and optionally wherein the K-Z_{peg} or K-Z_{lipid} are linked to the amino acid at X₁₀. In some embodiments, X₅ is D, E, hE, N, N(NMe2), Q, or Q(NMe2), wherein the D, E, and hE are linked to the amino acid at X₁₀. In some embodiments, X₅ is E, N, or N(NMe2), wherein the E is linked to the amino acid at X₁₀.

In some embodiments, X₅ is K-Z_{peg}, K-Z_{lipid}, L, N, N(NMe2), Q, or Q(NMe2). In some embodiments, X₅ is D, E, hE, K, K(a), K(Ac), K(d), K(G), K(NMe), K(NNs), L, N, N(NMe), N(NMe2), Q, Q(NMe), or Q(NMe2); wherein the D, E, hE, K, K(a), K(Ac), K(d), K(G), K(NMe), and K(NNs) are linked to the amino acid at X₁₀.

In some embodiments, X₅ is D linked to the amino acid at X₁₀. In some embodiments, X₅ is D linked to AEF at X₁₀. In some embodiments, X₅ is D linked to AEF(NMe) at X₁₀. In some embodiments, X₅ is E linked to the amino acid at X₁₀. In some embodiments, X₅ is E linked to AEF at X₁₀. In some embodiments, X₅ is E linked to AEF(NMe) at X₁₀. In some embodiments, X₅ is hE linked to the amino acid at X₁₀. In some embodiments, X₅ is hE linked to AEF at X₁₀. In some embodiments, X₅ is hE linked to AEF(NMe) at X₁₀.

In some embodiments, X₅ is K linked to the amino acid at X₁₀. In some embodiments, X₅ is K linked to AEF at X₁₀. In some embodiments, X₅ is K linked to AEF(NMe) at X₁₀. In some embodiments, X₅ is K(a) linked to the amino acid at X₁₀. In some embodiments, X₅ is K(a) linked to AEF at X₁₀. In some embodiments, X₅ is K(a) linked to AEF(NMe) at X₁₀. In some embodiments, X₅ is K(Ac) linked to the amino acid at X₁₀. In some embodiments, X₅ is K(Ac) linked to AEF at X₁₀. In some embodiments, X₅ is K(Ac) linked to AEF(NMe) at X₁₀. In some embodiments, X₅ is K(d) linked to the amino acid at X₁₀. In some embodiments, X₅ is K(d) linked to AEF at X₁₀. In some embodiments, X₅ is K(d) linked to AEF(NMe) at X₁₀. In some embodiments, X₅ is K(G) linked to the amino acid at X₁₀. In some embodiments, X₅ is K(G) linked to AEF at X₁₀. In some embodiments, X₅ is K(G) linked to AEF(NMe) at X₁₀. In some embodiments, X₅ is K(NMe) linked to the amino acid at X₁₀. In some embodiments, X₅ is K(NMe) linked to AEF at X₁₀. In some embodiments, X₅ is K(NMe) linked to AEF(NMe) at X₁₀. In some embodiments, X₅ is K(NNs) linked to the amino acid at X₁₀. In some embodiments, X₅ is K(NNs) linked to AEF at X₁₀. In some embodiments, X₅ is K(NNs) linked to AEF(NMe) at X₁₀.

In some embodiments, X₅ is K-Z_{peg}. In some embodiments, X₅ is K-Z_{peg} linked to the amino acid at X₁₀. In some embodiments, X₅ is K-Z_{peg} linked to AEF at X₁₀. In some embodiments, X₅ is K-Z_{peg} linked to AEF(NMe) at X₁₀. In some embodiments, X₅ is K-Z_{lipid}. In some embodiments, X₅ is K-Z_{lipid} linked to the amino acid at X₁₀. In some embodiments, X₅ is K-Z_{lipid} linked to AEF at X₁₀. In some embodiments, X₅ is K-Z_{lipid} linked to AEF(NMe) at X₁₀. In some embodiments, X₅ is L. In some embodiments, X₅ is N. In some embodiments, X₅ is N(NMe). In some embodiments, X₅ is N(NMe2). In some embodiments, X₅ is Q. In some embodiments, X₅ is Q(NMe). In some embodiments, X₅ is Q(NMe2).

In some embodiments, X₅ is d linked to the amino acid at X₁₀. In some embodiments, X₅ is d linked to AEF at X₁₀. In some embodiments, X₅ is d linked to AEF(NMe) at X₁₀. In some embodiments, X₅ is e linked to the amino acid at X₁₀. In some embodiments, X₅ is e linked to AEF at X₁₀. In some embodiments, X₅ is e linked to AEF(NMe) at X₁₀. In some embodiments, X₅ is he linked to the amino acid at X₁₀. In some embodiments, X₅ is he linked to AEF at X₁₀. In some embodiments, X₅ is he linked to AEF(NMe) at X₁₀.

In some embodiments, X₅ is k linked to the amino acid at X₁₀. In some embodiments, X₅ is k linked to AEF at X₁₀. In some embodiments, X₅ is k linked to AEF(NMe) at X₁₀. In some embodiments, X₅ is k(a) linked to the amino acid at X₁₀. In some embodiments, X₅ is k(a) linked to AEF at X₁₀. In some embodiments, X₅ is k(a) linked to AEF(NMe) at X₁₀. In some embodiments, X₅ is k(Ac) linked to the amino acid at X₁₀. In some embodiments, X₅ is k(Ac) linked to AEF at X₁₀. In some embodiments, X₅ is k(Ac) linked to AEF(NMe) at X₁₀. In some embodiments, X₅ is k(d) linked to the amino acid at X₁₀. In some embodiments, X₅ is k(d) linked to AEF at X₁₀. In some embodiments, X₅ is k(d) linked to AEF(NMe) at X₁₀. In some embodiments, X₅ is k(G) linked to the amino acid at X₁₀. In some embodiments, X₅ is k(G) linked to AEF at X₁₀. In some embodiments, X₅ is k(G) linked to AEF(NMe) at X₁₀. In some embodiments, X₅ is k(NMe) linked to the amino acid at X₁₀. In some embodiments, X₅ is k(NMe) linked to AEF at X₁₀. In some embodiments, X₅ is k(NMe) linked to AEF(NMe) at X₁₀. In some embodiments, X₅ is k(NNs) linked to the amino acid at X₁₀. In some embodiments, X₅ is k(NNs) linked to AEF at X₁₀. In some embodiments, X₅ is k(NNs) linked to AEF(NMe) at X₁₀.

In some embodiments, X₅ is k-Z_{peg}. In some embodiments, X₅ is k-Z_{peg} linked to the amino acid at X₁₀. In some embodiments, X₅ is k-Z_{peg} linked to AEF at X₁₀. In some embodiments, X₅ is k-Z_{peg} linked to AEF(NMe) at X₁₀. In some embodiments, X₅ is k-Z_{lipid}. In some embodiments, X₅ is k-Z_{lipid} linked to the amino acid at X₁₀. In some embodiments, X₅ is k-Z_{lipid} linked to AEF at X₁₀. In some embodiments, X₅ is k-Z_{lipid} linked to AEF(NMe) at X₁₀. In some embodiments, X₅ is 1. In some embodiments, X₅ is n. In some embodiments, X₅ is n(NMe). In some embodiments, X₅ is n(NMe2). In some embodiments, X₅ is q. In some embodiments, X₅ is q(NMe). In some embodiments, X₅ is q(NMe2).

In some embodiments, X₆ is T. In some embodiments, X₆ is T, wherein the T is an L-amino acid. In some embodiments, X₆ is t.

In some embodiments, X₇ is:

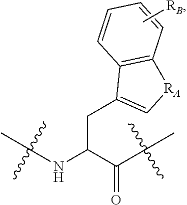

wherein:
R_{A} is NH or S; and
R_{B} is —H, halo, C₍₁₋₃₎alkyl, or phenyl, wherein the phenyl is optionally substituted with one —N(H)C(O)C₍₁₋₃₎alkyl group.

In some embodiments, X₇ is:

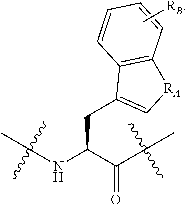

In some embodiments, X₇ is:

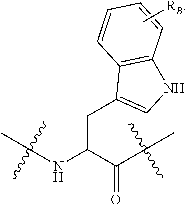

In some embodiments, X₇ is:

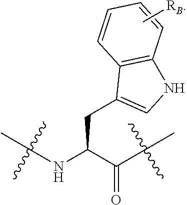

In some embodiments, R_{B} is C₍₁₋₃₎alkyl or phenyl, wherein the phenyl is optionally substituted with one —N(H)C(O)C₍₁₋₃₎alkyl group. In some embodiments, R_{B} is C₍₁₋₃₎alkyl. In some embodiments, R_{B} is phenyl substituted with one —N(H)C(O)C₍₁₋₃₎alkyl group.

In some embodiments, X₇ is 7(3NacPh)W, 7BrW, 7MeW, BT, or W. In some embodiments, X₇ is 7(3NacPh)W, 7BrW, 7MeW, BT, or W, each of which is an L-amino acid.

In some embodiments, X₇ is 7(3NacPh)w, 7Brw, 7Mew, dBT, or w.

In some embodiments, X₇ is 7(3NAcPh)W or 7MeW.

In some embodiments, X₇ is 7(3NacPh)W. In some embodiments, X₇ is 7BrW. In some embodiments, X₇ is 7MeW. In some embodiments, X₇ is BT. In some embodiments, X₇ is W.

In some embodiments, X₇ is 7(3NacPh)w. In some embodiments, X₇ is 7Brw. In some embodiments, X₇ is 7Mew. In some embodiments, X₇ is dBT. In some embodiments, X₇ is w.

In some embodiments, X₈ is Dab(NMeAc), Dab(NMecarn), Dab-Z_{peg}, Dab-Z_{lipid}, hK(Me)3, K, K(5cpa), K(Ac), K(d), K(G), K(Me)3, K(NMe), K(NMeAc), K(NNs), K-Z_{peg}, K-Z_{lipid}, NMeK-Z_{peg}, NMeK-Z_{lipid}, Lys(N+Me2)-Z_{peg}, Lys(N+Me2)-Z_{lipid}, Q, or Q(NMe2). In some embodiments, X₈ is Dab(NMeAc), Dab(NMecarn), Dab-Z_{peg}, Dab-Z_{lipid}, hK(Me)3, K, K(5cpa), K(Ac), K(d), K(G), K(Me)3, K(NMe), K(NMeAc), K(NNs), K-Z_{peg}, K-Z_{lipid}, NMeK-Z_{peg}, NMeK-Z_{lipid}, Lys(N+Me2)-Z_{peg}, Lys(N+Me2)-Z_{lipid}, Q, or Q(NMe2), each of which is an L-amino acid. In some embodiments, X₈ is dDab(NMeAc), dDab(NMecarn), dDab-Z_{peg}, dDab-Z_{lipid}, hk(Me)3, k, k(5cpa), k(Ac), k(d), k(G), k(Me)3, k(NMe), k(NMeAc), k(NNs), k-Z_{peg}, k-Z_{lipid}, NMek-Z_{peg}, NMek-Z_{lipid}, dLys(N+Me2)-Z_{peg}, dLys(N+Me2)-Z_{lipid}, q, or q(NMe2).

In some embodiments, X₈ is Dab(NMeAc), Dab(NMecarn), Dab-Z_{peg}, hK(Me)3, K(Ac), K(Me)3, K(NMeAc), NMeK-Z_{peg}, K-Z_{peg}, K-Z_{lipid}, Lys(N+Me2)-Z_{peg}, Q, or Q(NMe2).

In some embodiments, X₈ is Dab(NMeAc), Dab(NMecarn), Dab-Z_{peg}, hK(Me)3, K(Ac), K(Me)3, K(NMeAc), NMeK-Z_{peg}, K-Z_{peg}, Lys(N+Me2)-Z_{peg}, Q, or Q(NMe2). In some embodiments, X₈ is Dab(NMeAc), Dab(NMecarn), Dab-Z_{peg}, K(Ac), K(NMeAc), NMeK-Z_{peg}, K-Z_{peg}, K-Z_{lipid}, Q, or Q(NMe2). In some embodiments, X₈ is K(Ac), K(NMeAc), NMeK-Z_{peg}, K-Z_{peg}, or K-Z_{lipid}. In some embodiments, X₈ is K(Ac) or K(NMeAc).

In some embodiments, X₈ is Dab(NMeAc). In some embodiments, X₈ is Dab(NMecarn). In some embodiments, X₈ is Dab-Z_{peg}. In some embodiments, X₈ is Dab-Z_{lipid}. In some embodiments, X₈ is hK(Me)3. In some embodiments, X₈ is K. In some embodiments, X₈ is K(5cpa). In some embodiments, X₈ is K(Ac). In some embodiments, X₈ is K(d). In some embodiments, X₈ is K(G). In some embodiments, X₈ is K(Me)3. In some embodiments, X₈ is K(NMe). In some embodiments, X₈ is K(NMeAc). In some embodiments, X₈ is K(NNs). In some embodiments, X₈ is K-Z_{peg}. In some embodiments, X₈ is K-Z_{lipid}. In some embodiments, X₈ is NMeK-Z_{peg}. In some embodiments, X₈ is NMeK-Z_{lipid}. In some embodiments, X₈ is Lys(N+Me2)-Z_{peg}. In some embodiments, X₈ is Lys(N+Me2)-Z_{lipid}. In some embodiments, X₈ is Q. In some embodiments, X₈ is Q(NMe2).

In some embodiments, X₈ is dDab(NMeAc). In some embodiments, X₈ is dDab(NMecarn). In some embodiments, X₈ is dDab-Z_{peg}. In some embodiments, X₈ is dDab-Z_{lipid}. In some embodiments, X₈ is hk(Me)3. In some embodiments, X₈ is k. In some embodiments, X₈ is k(5cpa). In some embodiments, X₈ is k(Ac). In some embodiments, X₈ is k(d). In some embodiments, X₈ is k(G). In some embodiments, X₈ is k(Me)3. In some embodiments, X₈ is k(NMe). In some embodiments, X₈ is k(NMeAc). In some embodiments, X₈ is k(NNs). In some embodiments, X₈ is k-Z_{peg}. In some embodiments, X₈ is k-Z_{lipid}. In some embodiments, X₈ is NMek-Z_{peg}. In some embodiments, X₈ is NMek-Z_{lipid}. In some embodiments, X₈ is dLys(N+Me2)-Z_{peg}. In some embodiments, X₈ is dLys(N+Me2)-Z_{lipid}. In some embodiments, X₈ is q. In some embodiments, X₈ is q(NMe2).

In some embodiments, X₉ is aMeC, aG, C, D, E, hE, Pen, or Dap(N3). In some embodiments, X₉ is aMeC, aG, C, D, E, hE, Pen, or Dap(N3), each of which is an L-amino acid. In some embodiments, X₉ is aMe-d-C, d-aG, c, d, e, he, dPen, or dDap(N3).

In some embodiments, X₉ is aG, D, E, hE, or Dap(N3). In some embodiments, X₉ is aMeC, C, or Pen. In some embodiments, X₉ is aMeC or Pen.

In some embodiments, X₉ is aMeC. In some embodiments, X₉ is aG. In some embodiments, X₉ is C. In some embodiments, X₉ is D. In some embodiments, X₉ is E. In some embodiments, X₉ is hE. In some embodiments, X₉ is Pen. In some embodiments, X₉ is Dap(N3).

In some embodiments, X₉ is aMe-d-C. In some embodiments, X₉ is d-aG. In some embodiments, X₉ is c. In some embodiments, X₉ is d. In some embodiments, X₉ is e. In some embodiments, X₉ is he. In some embodiments, X₉ is dPen. In some embodiments, X₉ is dDap(N3).

In some embodiments, X₁₀ is:

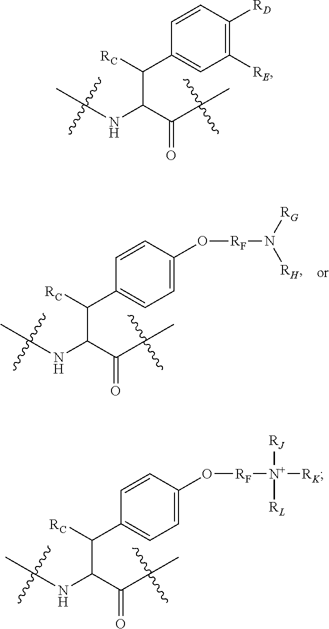

R_{C} is —H or —C₍₁₋₃₎alkyl;
R_{D} is —H, —OH, —CN, —C₍₁₋₃₎alkyl, —OC₍₁₋₃₎alkyl, —OC(1-3alkyl-(5-member heteroaryl), —C(O)NH₂, or heterocyclyl, wherein the —OC₍₁₋₃₎alkyl-(5-membered heteroaryl) is optionally substituted with a polyethylene glycol chain, and wherein the heterocyclyl is optionally substituted with one —C(O)NH₂ group;
R_{E} is —H or halo;
R_{F} is —C₍₁₋₆₎alkylene or a bivalent polyethylene glycol chain;
R_{G} is —H, —C₍₁₋₃₎alkyl, or a bond to the amino acid at X₅ or X₁₃;
R_{H} is —H, —C₍₁₋₃₎alkyl, —C(NH)NH₂, —C(O)—R_{H1};
or R_{G} and R_{H} taken together with the nitrogen atom to which they are attached form a 5- to 8-membered heterocyclyl group;
R_{H1} is —C₍₁₋₅₎alkyl, —OC₍₁₋₅₎alkyl, —C₍₁₋₃₎alkyl-phenyl, -phenyl-C₍₁₋₃₎alkyl-N(H)—S(O)₂—C₍₁₋₃₎)alkyl, or a polyethylene glycol chain, wherein the —C₍₁₋₃₎alkyl-phenyl is optionally substituted with one to three groups selected from halo and —OH;
R_{J}, R_{K}, and R_{L} are each, independently, C₍₁₋₃₎alkyl; and
or R_{J} and R_{K} taken together with the nitrogen atom to which they are attached form a 5- to 8-membered heterocyclyl group.

In some embodiments, X₁₀ is:

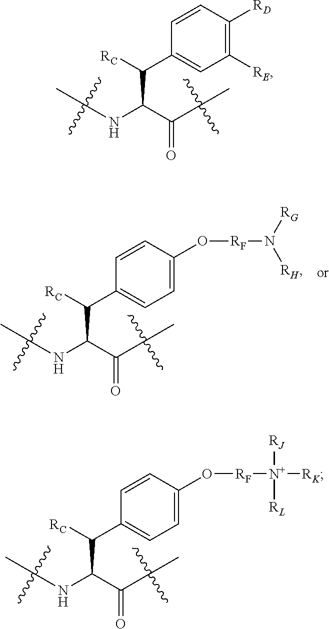

In some embodiments, X₁₀ is

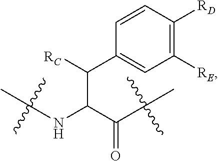

wherein:
R_{C} is —H or —C₍₁₋₃₎alkyl;
R_{D} is —H, —OH, —CN, —C₍₁₋₃₎alkyl, —OC₍₁₋₃₎alkyl, —OC₍₁₋₃₎alkyl-(5-membered heteroaryl), —C(O)NH₂, or heterocyclyl, wherein the —OC₍₁₋₃₎alkyl-(5-membered heteroaryl) is optionally substituted with a polyethylene glycol chain, and wherein the heterocyclyl is optionally substituted with one —C(O)NH₂ group; and
R_{E} is —H or halo.

In some embodiments, X₁₀ is

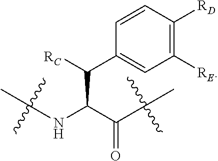

In some embodiments, X₁₀ is

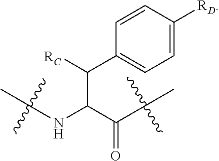

In some embodiments, X₁₀ is

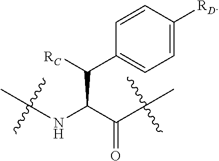

In some embodiments, X₁₀ is

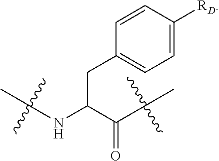

In some embodiments, X₁₀ is

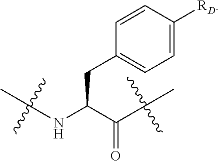

In some embodiments, R_{C} is —H. In some embodiments, R_{C} is —CH₃.

In some embodiments, R_{D} is —H, —OH, or —OC₍₁₋₃₎alkyl.

In some embodiments, R_{E} is —H. In some embodiments, R_{E} is —F.

In some embodiments, X₁₀ is

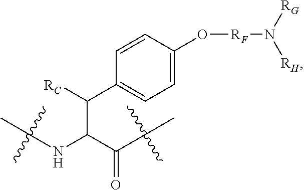

wherein:
R_{C} is —H or —C₍₁₋₃₎alkyl;
R_{F} is —C₍₁₋₆₎alkylene or a bivalent polyethylene glycol chain;
R_{G} is —H, —C₍₁₋₃₎alkyl, or a bond to the amino acid at X₅ or X₁₃;
R_{H} is —H, —C₍₁₋₃₎alkyl, —C(NH)NH₂, —C(O)—R_{H1};
or R_{G} and R_{H} taken together with the nitrogen atom to which they are attached form a 5- to 8-membered heterocyclyl group; and
R_{H1} is —C₍₁₋₅₎alkyl, —OC₍₁₋₅₎alkyl, —C₍₁₋₃₎alkyl-phenyl, -phenyl-C₍₁₋₃₎alkyl-N(H)—S(O)₂—C₍₁₋₃₎)alkyl, or a polyethylene glycol chain, wherein the —C₍₁₋₃₎alkyl-phenyl is optionally substituted with one to three groups selected from halo and —OH.

In some embodiments, X₁₀ is

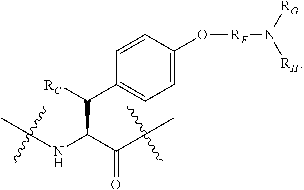

In some embodiments, X₁₀

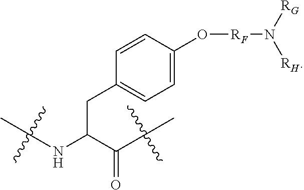

In some embodiments, X₁₀

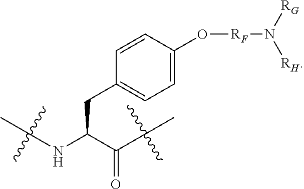

In some embodiments, R_{C} is —H. In some embodiments, R_{C} is —CH₃.

In some embodiments, R_{F} is —C₍₁₋₆₎alkylene. In some embodiments, R_{F} is a bivalent polyethylene glycol chain having between 1 and 12 polyethylene glycol units. In some embodiments, R_{F} is a bivalent polyethylene glycol chain having between 1 and 8 polyethylene glycol units. In some embodiments, R_{F} is a bivalent polyethylene glycol chain having between 1 and 4 polyethylene glycol units.

In some embodiments, R_{G} is —H. In some embodiments, R_{G} is —CH₃. In some embodiments, R_{G} is a bond to the amino acid at X₅ or X₁₃. In some embodiments, R_{G} is a bond to the amino acid at X₅. In some embodiments, R_{G} is a bond to the amino acid at X₁₃.

In some embodiments, R_{H} is —H. In some embodiments, R_{H} is —CH₃. In some embodiments, R_{H} is —C(O)—R_{H1}.

In some embodiments, R_{H1} is a polyethylene glycol chain. In some embodiments, R_{H1} is a polyethylene glycol chain terminating in an ammonium or methyl group.

In some embodiments, X₁₀ is

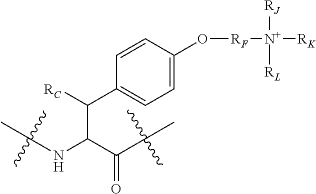

wherein:
R_{C} is —H or —C₍₁₋₃₎alkyl;
R_{F} is —C₍₁₋₆₎alkylene or a bivalent polyethylene glycol chain;
R_{J}, R_{K}, and R_{L} are each, independently, C₍₁₋₃₎alkyl;
or R_{J} and R_{K} taken together with the nitrogen atom to which they are attached form a 5- to 8-membered heterocyclyl group.

In some embodiments, X₁₀ is

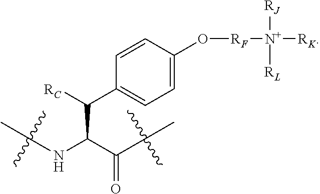

In some embodiments, X₁₀ is

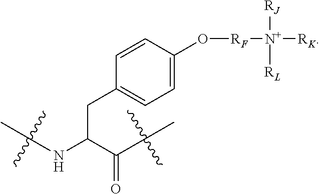

In some embodiments, X₁₀ is

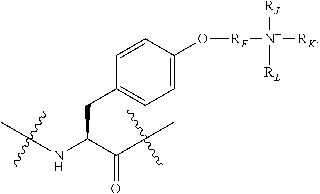

In some embodiments, R_{C} is —H. In some embodiments, R_{C} is —CH₃.

In some embodiments, R_{F} is —C₍₁₋₆₎alkylene. In some embodiments, R_{F} is a bivalent polyethylene glycol chain having between 1 and 12 polyethylene glycol units. In some embodiments, R_{F} is a bivalent polyethylene glycol chain having between 1 and 8 polyethylene glycol units. In some embodiments, R_{F} is a bivalent polyethylene glycol chain having between 1 and 4 polyethylene glycol units.

In some embodiments, R_{J}, R_{K}, and R_{L} are each, independently, methyl.

In some embodiments, X₁₀ is 3FTyr, 4AmF, 4CNF, 4DMPzEF, 4OMeF, 4MeF, 4PipPhe, AEF, AEF(Ac), AEF(BH), AEF(Boc), AEF(EtCO), AEF(G), AEF(NMe), AEF(NMe2), AEF(NMe3), AEF(SMSB), AEF-Z_{peg}, AEF(NMe)-Z_{peg}, APEG3F, bMeAEF, F, MMoEF, TMAPF, Y, Y(OTzl), or Y(OTzl(mPEG3)), optionally wherein the AEF or AEF(NMe) is linked to the amino acid at X₅ or the amino acid at X₁₃. In some embodiments, X₁₀ is 3FTyr, 4AmF, 4CNF, 4DMPzEF, 4OMeF, 4MeF, 4PipPhe, AEF, AEF(Ac), AEF(BH), AEF(Boc), AEF(EtCO), AEF(G), AEF(NMe), AEF(NMe2), AEF(NMe3), AEF(SMSB), AEF-Z_{peg}, AEF(NMe)-Z_{peg}, APEG3F, bMeAEF, F, MMoEF, TMAPF, Y, Y(OTzl), or Y(OTzl(mPEG3)), each of which is an L-amino acid, optionally wherein the AEF or AEF(NMe) is linked to the amino acid at X₅ or the amino acid at X₁₃. In some embodiments, X₁₀ is 3F-d-Tyr, d-4AmF, d-4CNF, d-4DMPzEF, d-4OMeF, d-4MeF, 4Pip-d-Phe, dAEF, dAEF(Ac), dAEF(BH), dAEF(Boc), dAEF(EtCO), dAEF(G), dAEF(NMe), dAEF(NMe2), dAEF(NMe3), dAEF(SMSB), dAEF-Z_{peg}, dAEF(NMe)-Z_{peg}, dAPEG3F, d-bMeAEF, f, dMMoEF, dTMAPF, y, y(OTzl), or y(OTzl(mPEG3)), optionally wherein the dAEF or dAEF(NMe) is linked to the amino acid at X₅ or the amino acid at X₁₃.

In some embodiments, X₁₀ is 3FTyr, 4AmF, 4CNF, 4DMPzEF, 4OMeF, 4MeF, 4PipPhe, AEF, AEF(Ac), AEF(BH), AEF(Boc), AEF(EtCO), AEF(G), AEF(NMe), AEF(NMe2), AEF(NMe3), AEF(SMSB), AEF-Z_{peg}, AEF(NMe)-Z_{peg}, APEG3F, bMeAEF, F, MMoEF, TMAPF, Y, Y(OTzl), or Y(OTzl(mPEG3)).

In some embodiments, X₁₀ is 4DMPzEF, 4OMeF, AEF, AEF(G), AEF(NMe), AEF(NMe2), AEF-Z_{peg}, AEF(NMe)-Z_{peg}, APEG3F, bMeAEF, F, MMoEF, TMAPF, or Y, optionally wherein the AEF or AEF(NMe) is linked to the amino acid at X₅ or the amino acid at X₁₃. In some embodiments, X₁₀ is 4DMPzEF, 4OMeF, AEF, AEF(G), AEF(NMe), AEF(NMe2), AEF-Z_{peg}, AEF(NMe)-Z_{peg}, APEG3F, bMeAEF, F, MMoEF, TMAPF, or Y.

In some embodiments, X₁₀ is AEF, AEF(G), AEF(NMe), AEF(NMe2), AEF-Z_{peg}, AEF(NMe)-Z_{peg}, bMeAEF, MMoEF, or TMAPF, optionally wherein the AEF or AEF(NMe) is linked to the amino acid at X₅ or the amino acid at X₁₃. In some embodiments, X₁₀ is AEF or TMAPF, optionally wherein the AEF is linked to the amino acid at X₅ or the amino acid at X₁₃.

In some embodiments, X₁₀ is AEF or TMAPF.

In some embodiments, X₁₀ is 3FTyr. In some embodiments, X₁₀ is 4AmF. In some embodiments, X₁₀ is 4CNF. In some embodiments, X₁₀ is 4DMPzEF. In some embodiments, X₁₀ is 4OMeF. In some embodiments, X₁₀ is 4MeF. In some embodiments, X₁₀ is 4PipPhe. In some embodiments, X₁₀ is AEF. In some embodiments, X₁₀ is AEF linked to the amino acid at X₅. In some embodiments, X₁₀ is AEF linked to E at X₅. In some embodiments, X₁₀ is AEF linked to K at X₅. In some embodiments, X₁₀ is AEF linked to K(a) at X₅. In some embodiments, X₁₀ is AEF linked to K(Ac) at X₅. In some embodiments, X₁₀ is AEF linked to K(d) at X₅. In some embodiments, X₁₀ is AEF linked to K(G) at X₅. In some embodiments, X₁₀ is AEF linked to K(NMe) at X₅. In some embodiments, X₁₀ is AEF linked to K(NNs) at X₅. In some embodiments, X₁₀ is AEF linked to K-Z_{peg} at X₅. In some embodiments, X₁₀ is AEF linked to K-Z_{lipid} at X₅. In some embodiments, X₁₀ is AEF linked to the amino acid at X₁₃. In some embodiments, X₁₀ is AEF linked to E at X₁₃. In some embodiments, X₁₀ is AEF(Ac). In some embodiments, X₁₀ is AEF(BH). In some embodiments, X₁₀ is AEF(Boc). In some embodiments, X₁₀ is AEF(EtCO). In some embodiments, X₁₀ is AEF(G). In some embodiments, X₁₀ is AEF(NMe). In some embodiments, X₁₀ is AEF(NMe) linked to the amino acid at X₅. In some embodiments, X₁₀ is AEF(NMe) linked to the amino acid at X₁₃. In some embodiments, X₁₀ is AEF(NMe2). In some embodiments, X₁₀ is AEF(NMe3). In some embodiments, X₁₀ is AEF(SMSB). In some embodiments, X₁₀ is AEF-Z_{peg}. In some embodiments, X₁₀ is AEF(NMe)-Z_{peg}. In some embodiments, X₁₀ is APEG3F. In some embodiments, X₁₀ is bMeAEF. In some embodiments, X₁₀ is F. In some embodiments, X₁₀ is MMoEF. In some embodiments, X₁₀ is TMAPF. In some embodiments, X₁₀ is Y. In some embodiments, X₁₀ is Y(OTzl). In some embodiments, X₁₀ is Y(OTzl(mPEG3)).

In some embodiments, X₁₀ is 3F-d-Tyr. In some embodiments, X₁₀ is d-4AmF. In some embodiments, X₁₀ is d-4CNF. In some embodiments, X₁₀ is d-4DMPzEF. In some embodiments, X₁₀ is d-4OMeF. In some embodiments, X₁₀ is d-4MeF. In some embodiments, X₁₀ is 4Pip-d-Phe. In some embodiments, X₁₀ is dAEF. In some embodiments, X₁₀ is dAEF linked to the amino acid at X₅. In some embodiments, X₁₀ is dAEF linked to E at X₅. In some embodiments, X₁₀ is dAEF linked to K at X₅. In some embodiments, X₁₀ is dAEF linked to K(a) at X₅. In some embodiments, X₁₀ is dAEF linked to K(Ac) at X₅. In some embodiments, X₁₀ is dAEF linked to K(d) at X₅. In some embodiments, X₁₀ is dAEF linked to K(G) at X₅. In some embodiments, X₁₀ is dAEF linked to K(NMe) at X₅. In some embodiments, X₁₀ is dAEF linked to K(NNs) at X₅. In some embodiments, X₁₀ is dAEF linked to K-Z_{peg} at X₅. In some embodiments, X₁₀ is dAEF linked to K-Z_{lipid} at X₅. In some embodiments, X₁₀ is dAEF linked to the amino acid at X₁₃. In some embodiments, X₁₀ is dAEF linked to E at X₁₃. In some embodiments, X₁₀ is dAEF(Ac). In some embodiments, X₁₀ is dAEF(BH). In some embodiments, X₁₀ is dAEF(Boc). In some embodiments, X₁₀ is dAEF(EtCO). In some embodiments, X₁₀ is dAEF(G). In some embodiments, X₁₀ is dAEF(NMe). In some embodiments, X₁₀ is dAEF(NMe) linked to the amino acid at X₅. In some embodiments, X₁₀ is dAEF(NMe) linked to the amino acid at X₁₃. In some embodiments, X₁₀ is dAEF(NMe2). In some embodiments, X₁₀ is dAEF(NMe3). In some embodiments, X₁₀ is dAEF(SMSB). In some embodiments, X₁₀ is dAEF-Z_{peg}. In some embodiments, X₁₀ is dAEF(NMe)-Z_{peg}. In some embodiments, X₁₀ is dAPEG3F. In some embodiments, X₁₀ is d-bMeAEF. In some embodiments, X₁₀ is f. In some embodiments, X₁₀ is dMMoEF. In some embodiments, X₁₀ is dTMAPF. In some embodiments, X₁₀ is y. In some embodiments, X₁₀ is y(OTzl). In some embodiments, X₁₀ is y(OTzl(mPEG3)).

In some embodiments, X₁₀ is:

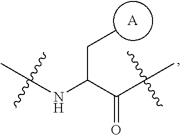

wherein:
A is

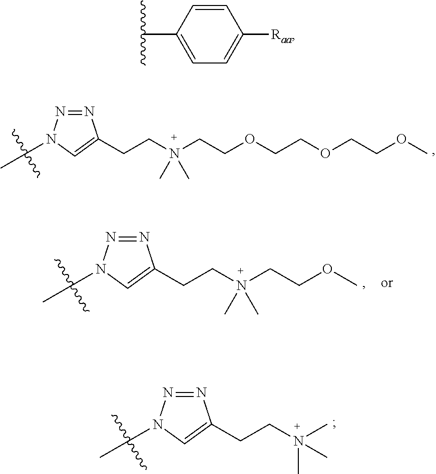

Rₐₐ is —OCHF₂, —O(CH₂)₉CO₂H,

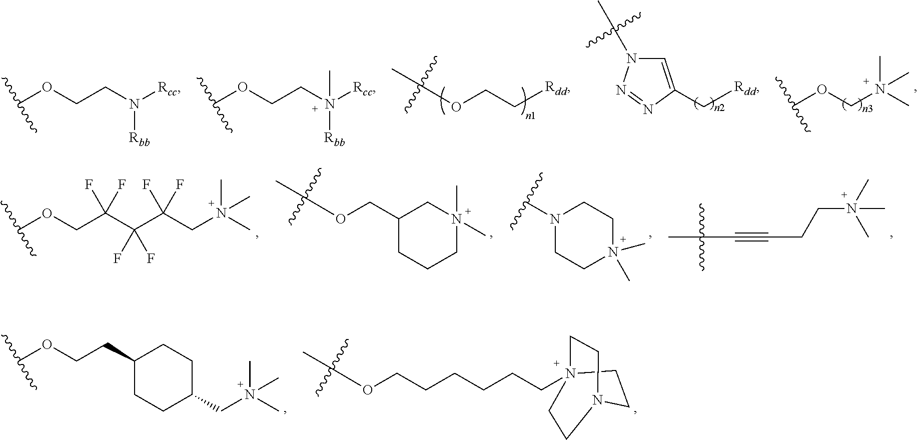

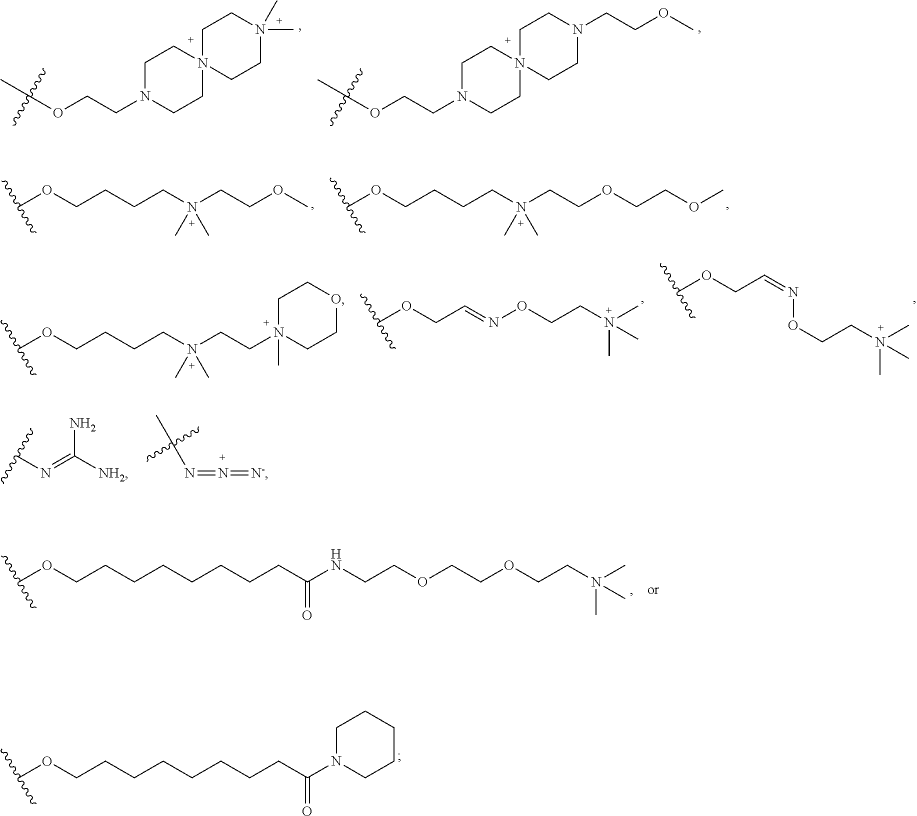

R_{bb} is —H, —CH₃, —C(O)CH₃, —C(NH—)NH₂, —(CH₂)₃O(CH₂)₂OCH₃, —CH₂CH₂OCH₃, —(CH₂CH₂O)₃CH₃, —(CH₂CH₂O)₆CH₃,

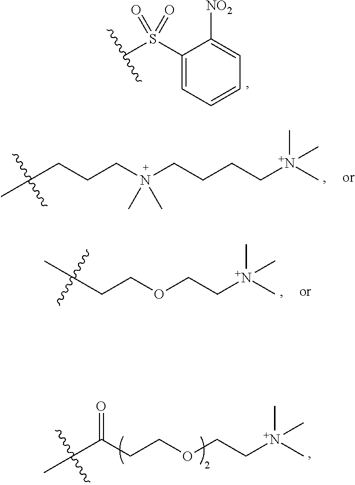

R_{cc} is —H, —CH₃, —(CH₂)₃O(CH₂)₂OCH₃,

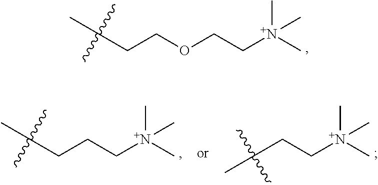

n1 is 1, 2, or 3;
R_{dd} is

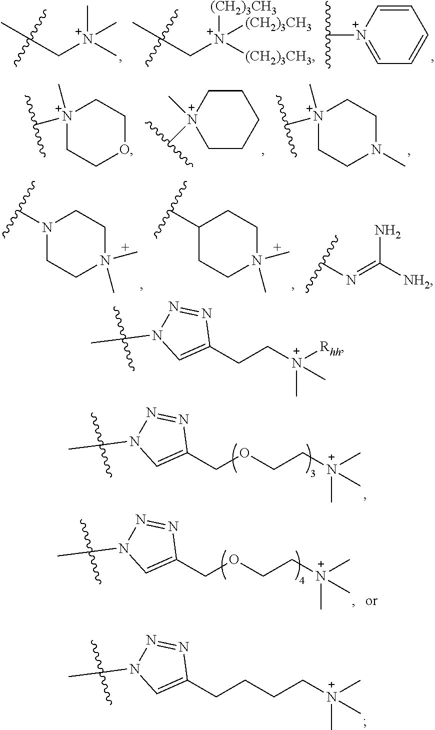

n2 is 1, 2, 3, 4, or 5;
R_{gg} is OCH₃

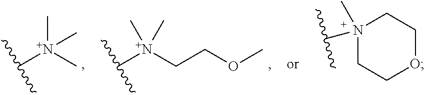

n3 is 3, 4, 5, 6, or 8; and
Rₕₕ is —H, —(CH₂)₇CH₃, —(CH₂)₁₅CH₃, —(CH₂)₂OCH₃, or —(CH₂CH₂O)₃CH₃.

In some embodiments, X₁₀ is:

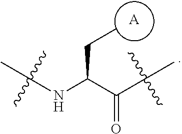

In some embodiments, X₁₁ is

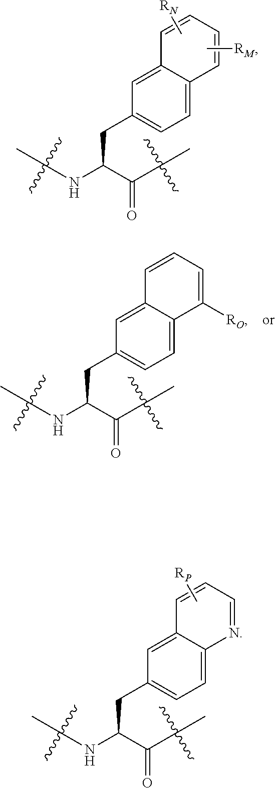

In some embodiments, X₁₁ is

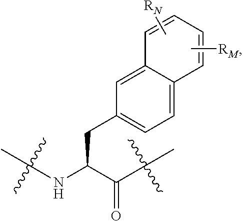

wherein:
R_{M} is halo, —OH, —C₍₁₋₃₎alkyl, —OC(O)C₍₁₋₃₎haloalkyl, phenyl, or 5- to 6-membered heteroaryl, wherein the phenyl and 5- to 6-membered heteroaryl are each optionally substituted with one to three groups selected from —OH, —OC₍₁₋₃₎alkyl, —C₍₁₋₃₎haloalkyl, and 3- to 6-membered heterocyclyl; and
R_{N} is —H or —OH.

In some embodiments, X₁₁ is

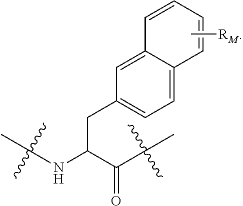

In some embodiments, X₁₁ is

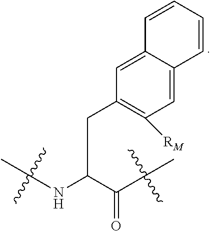

In some embodiments, X₁₁ is

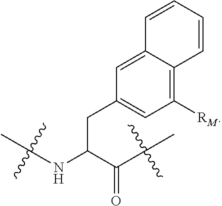

In some embodiments, X₁₁ is

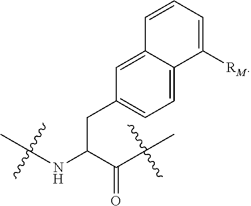

In some embodiments, X₁₁ is

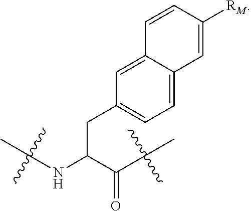

In some embodiments, X₁₁ is

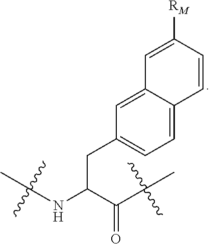

In some embodiments, X₁₁ is

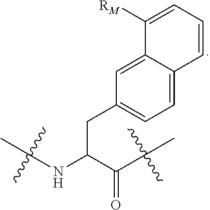

In some embodiments, X₁₁ is

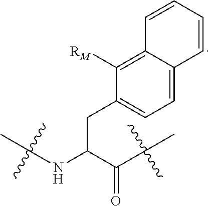

In some embodiments, X₁₁ is

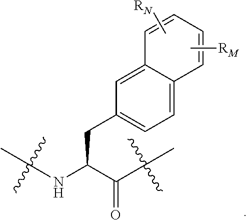

In some embodiments, X₁₁ is

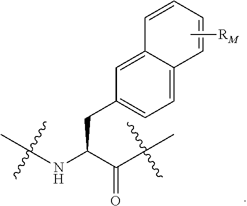

In some embodiments, X₁₁ is

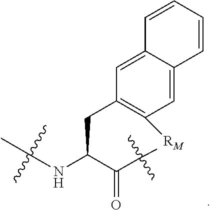

In some embodiments, X₁₁ is

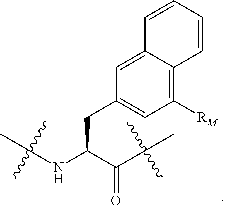

In some embodiments, X₁₁ is

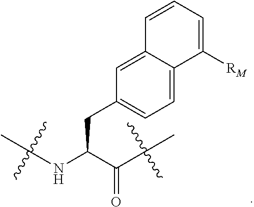

In some embodiments, X₁₁ is

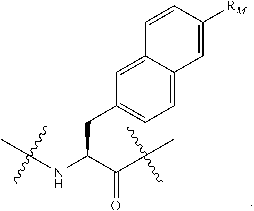

In some embodiments, X₁₁ is

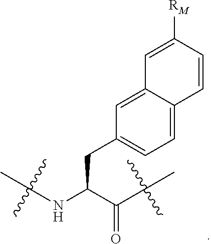

In some embodiments, X₁₁ is

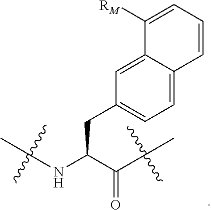

In some embodiments, X₁₁ is

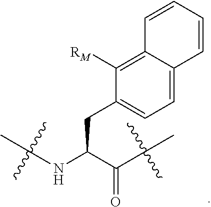

In some embodiments, R_{M} is —OH, —CH₃, —OC(O)CF3, phenyl, or 5-membered heteroaryl, wherein the phenyl and 5- to 6-membered heteroaryl are each optionally substituted with one to three groups selected from —OH, —OCH₃, —CF₃, and 6-membered heterocyclyl. In some embodiments, R_{M} is —OH.

In some embodiments, R_{N} is —H. In some embodiments, R_{N} is —OH.

In some embodiments, X₁ is

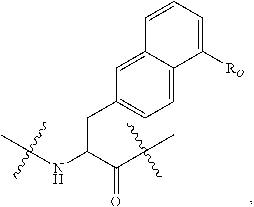

wherein R_{O} is —OC₍₁₋₃₎alkyl or —C(O)NH₂.

In some embodiments, R_{O} is —OC₍₁₋₃₎alkyl. In some embodiments, R_{O} is —C(O)NH₂.

In some embodiments, X₁ is

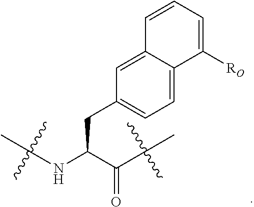

In some embodiments, X₁ is

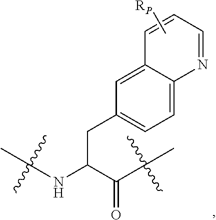

wherein R_{P} is halo, —OH, —C₍₁₋₃₎alkyl, —OC₍₁₋₃₎alkyl, —C(O)NH₂, —OC(O)C₍₁₋₃₎haloalkyl, phenyl, or 5- to 6-membered heteroaryl, wherein the phenyl and 5- to 6-membered heteroaryl are each optionally substituted with one to three groups selected from —OH, —OC₍₁₋₃₎alkyl, —C₍₁₋₃₎haloalkyl, and 3- to 6-membered heterocyclyl.

In some embodiments, X₁₁ is

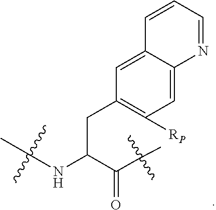

In some embodiments, X₁₁ is

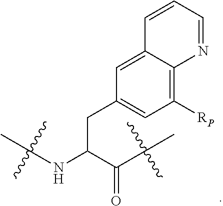

In some embodiments, X₁₁ is

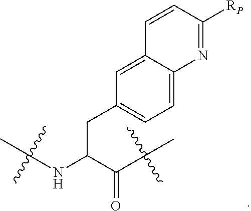

In some embodiments, X₁₁ is

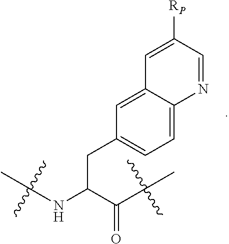

In some embodiments, X₁₁ is

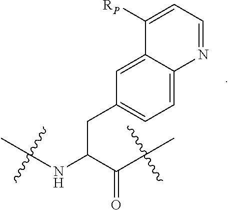

In some embodiments, X₁₁ is

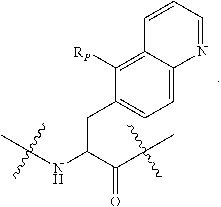

In some embodiments, X₁₁ is

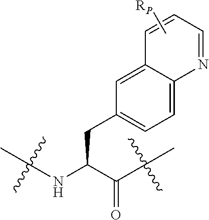

In some embodiments, X₁₁ is

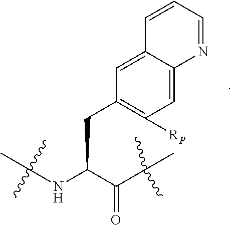

In some embodiments, X₁₁ is

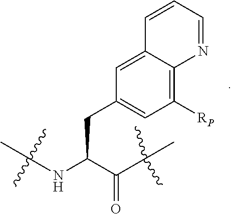

In some embodiments, X₁₁ is

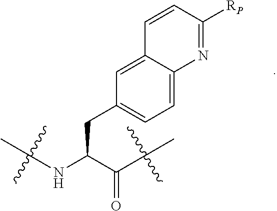

In some embodiments, X₁₁ is

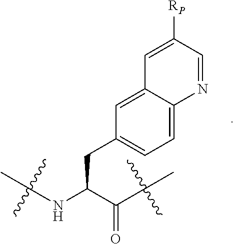

In some embodiments, X₁₁ is

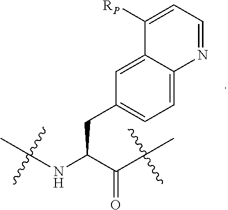

In some embodiments, X₁₁ is

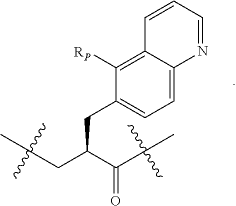

In some embodiments, R_{P} is halo, —C₍₁₋₃₎alkyl, —OC₍₁₋₃₎alkyl, —C(O)NH₂, —OC(O)C₍₁₋₃₎haloalkyl, phenyl, or 5- to 6-membered heteroaryl, wherein the phenyl and 5- to 6-membered heteroaryl are each optionally substituted with one to three groups selected from —OH, —OC₍₁₋₃₎alkyl, —C₍₁₋₃₎haloalkyl, and 3- to 6-membered heterocyclyl. In some embodiments, R_{P} is —OH or —OC₍₁₋₃₎alkyl. In some embodiments, R_{P} is —OH. In some embodiments, R_{P} is —OC₍₁₋₃₎alkyl.

In some embodiments, X₁₁ is 2Nal6((5CF₃)₃Pyrazole), 6OH2Nal, 6OHQui, 2Nal6(Ph2OH), 2Nal6(Ph4(NMorph)), 2Nal6(3Pyrazole), 2Nal6(4OMePh), 5OMe2Nal, 5amido2Nal, 5Br2Nal, 5Me2Nal, 6MeQui, 6O(COCF3)₂Nal, 6F2Nal, 6Br2Nal, or 7OH2Nal. In some embodiments, X₁₁ is 2Nal6((5CF₃)₃Pyrazole), 6OH2Nal, 6OHQui, 2Nal6(Ph2OH), 2Nal6(Ph4(NMorph)), 2Nal6(3Pyrazole), 2Nal6(4OMePh), 5OMe2Nal, 5amido2Nal, 5Br2Nal, 5Me2Nal, 6MeQui, 6O(COCF3)₂Nal, 6F2Nal, 6Br2Nal, or 7OH2Nal, each of which is an L-amino acid. In some embodiments, X₁₁ is d-2Nal6((5CF₃)₃Pyrazole), d-6OH2Nal, d-6OHQui, d-2Nal6(Ph2OH), d-2Nal6(Ph4(NMorph)), d-2Nal6(3Pyrazole), d-2Nal6(4OMePh), d-5OMe2Nal, d-5amido2Nal, d-5Br2Nal, d-5Me2Nal, d-6MeQui, d-6O(COCF3)₂Nal, d-6F2Nal, d-6Br2Nal, or d-7OH2Nal.

In some embodiments, X₁₁ is 2Nal6((5CF₃)₃Pyrazole), 6OH2Nal, 2Nal6(Ph2OH), 2Nal6(Ph4(NMorph)), 2Nal6(3Pyrazole), 2Nal6(4OMePh), 5OMe2Nal, 5amido2Nal, 5Br2Nal, 5Me2Nal, 6MeQui, 6O(COCF3)₂Nal, 6F2Nal, 6Br2Nal, or 7OH2Nal. In some embodiments, X₁₁ is 2Nal6((5CF₃)₃Pyrazole), 6OH2Nal, 2Nal6(Ph2OH), 2Nal6(Ph4(NMorph)), 2Nal6(3Pyrazole), 2Nal6(4OMePh), 5Br2Nal, 5Me2Nal, 6O(COCF3)₂Nal, 6F2Nal, 6Br2Nal, or 7OH2Nal.

In some embodiments, X₁₁ is 2Nal6((5CF₃)₃Pyrazole). In some embodiments, X₁₁ is 6OH2Nal. In some embodiments, X₁₁ is 6OHQui. In some embodiments, X₁₁ is 2Nal6(Ph2OH).

In some embodiments, X₁₁ is 2Nal6(Ph4(NMorph)). In some embodiments, X₁₁ is 2Nal6(3Pyrazole). In some embodiments, X₁₁ is 2Nal6(4OMePh). In some embodiments, X₁₁ is 5OMe2Nal. In some embodiments, X₁₁ is 5amido2Nal. In some embodiments, X₁₁ is 5Br2Nal.

In some embodiments, X₁₁ is 5Me2Nal. In some embodiments, X₁₁ is 6MeQui. In some embodiments, X₁₁ is 6O(COCF3)₂Nal. In some embodiments, X₁₁ is 6F2Nal. In some embodiments, X₁₁ is 6Br2Nal. In some embodiments, X₁₁ is 7OH2Nal.

In some embodiments, X₁₁ is d-2Nal6((5CF₃)₃Pyrazole). In some embodiments, X₁₁ is d-6OH2Nal. In some embodiments, X₁₁ is d-6OHQui. In some embodiments, X₁₁ is d-2Nal6(Ph2OH). In some embodiments, X₁₁ is d-2Nal6(Ph4(NMorph)). In some embodiments, X₁₁ is d-2Nal6(3Pyrazole). In some embodiments, X₁₁ is d-2Nal6(4OMePh). In some embodiments, X₁₁ is d-5OMe2Nal. In some embodiments, X₁₁ is d-5amido2Nal. In some embodiments, X₁₁ is d-5Br2Nal. In some embodiments, X₁₁ is d-5Me2Nal. In some embodiments, X₁₁ is d-6MeQui. In some embodiments, X₁₁ is d-6O(COCF3)₂Nal. In some embodiments, X₁₁ is d-6F2Nal. In some embodiments, X₁₁ is d-6Br2Nal. In some embodiments, X₁₁ is d-7OH2Nal.

In some embodiments, X₁₂ is THP, aMeL, diFCpx, or Pip(Nme2). In some embodiments, X₁₂ is THP, aMeL, diFCpx, or Pip(Nme2), wherein the aMeL is an L-amino acid. In some embodiments, X₁₂ is THP, aMel, diFCpx, or Pip(Nme2).

In some embodiments, X₁₂ is THP. In some embodiments, X₁₂ is aMeL. In some embodiments, X₁₂ is aMel. In some embodiments, X₁₂ is diFCpx. In some embodiments, X₁₂ is Pip(Nme2).

In some embodiments, X₁₃ is C, D, Dab(NMeAc), Dab(NMecam), Dab-Z_{peg}, Dab-Z_{lipid}, E, E(COcPEG3a), hE, K, K(5cpa), K(Ac), K(d), K(G), K(Me)3, K(NMe), K(NMeAc), K(NNs), K-Z_{peg}, K-Z_{lipid}, NMeK-Z_{peg}, NMeK-Z_{lipid}, L, or Q(NMe2); wherein the C, D, and hE are linked to R₁, the amino acid at X₃, or the amino acid at X₁₀, and optionally wherein the E is linked to R₁, the amino acid at X₃, or the amino acid at X₁₀. In some embodiments, X₁₃ is C, D, Dab(NMeAc), Dab(NMecarn), Dab-Z_{peg}, Dab-Z_{lipid}, E, E(COcPEG3a), hE, K, K(5cpa), K(Ac), K(d), K(G), K(Me)3, K(NMe), K(NMeAc), K(NNs), K-Z_{peg}, K-Z_{lipid}, NMeK-Z_{peg}, NMeK-Z_{lipid}, L, or Q(NMe2), each of which is an L-amino acid; wherein the C, D, and hE are linked to R₁, the amino acid at X₃, or the amino acid at X₁₀, and optionally wherein the E is linked to R₁, the amino acid at X₃, or the amino acid at X₁₀. In some embodiments, X₁₃ is c, d, dDab(NMeAc), dDab(NMecarn), dDab-Z_{peg}, dDab-Z_{lipid}, e, e(COcPEG3a), he, k, k(5cpa), k(Ac), k(d), k(G), k(Me)3, k(NMe), k(NMeAc), k(NNs), k-Z_{peg}, k-Z_{lipid}, NMek-Z_{peg}, NMek-Z_{lipid}, 1, or q(NMe2); wherein the c, d, and he are linked to R₁, the amino acid at X₃, or the amino acid at X₁₀, and optionally wherein the e is linked to R₁, the amino acid at X₃, or the amino acid at X₁₀.

In some embodiments, X₁₃ is C, D, Dab(NMeAc), Dab(NMecam), E, E(COcPEG3a), hE, K(Ac), K(Me)3, K(NMeAc), K-Z_{peg}, K-Z_{lipid}, L, or Q(NMe2); wherein the C, D, and hE are linked to R₁, the amino acid at X₃, or the amino acid at X₁₀, and optionally wherein the E is linked to R₁, the amino acid at X₃, or the amino acid at X₁₀. In some embodiments, X₁₃ is Dab(NMeAc), Dab(NMecarn), E, K(Ac), K(NMeAc), K-Z_{peg}, or K-Z_{lipid}. In some embodiments, X₁₃ is E, K(Ac), K(NMeAc), K-Z_{peg}, or K-Z_{lipid}. In some embodiments, X₁₃ is E, K(Ac), or K(NMeAc). In some embodiments, X₁₃ is E or K(Ac).

In some embodiments, X₁₃ is Dab(NMeAc), Dab(NMecam), Dab-Z_{peg}, Dab-Z_{lipid}, E, E(COcPEG3a), K, K(5cpa), K(Ac), K(d), K(G), K(Me)3, K(NMe), K(NMeAc), K(NNs), K-Z_{peg}, K-Z_{lipid}, NMeK-Z_{peg}, NMeK-Z_{lipid}, L, or Q(NMe2). In some embodiments, X₁₃ is Dab(NMeAc), Dab(NMecarn), E, E(COcPEG3a), K(Ac), K(Me)3, K(NMeAc), K-Z_{peg}, K-Z_{lipid}, L, or Q(NMe2).

In some embodiments, X₁₃ is C, D, Dab(NMeAc), Dab(NMecam), Dab-Z_{peg}, E, E(COcPEG3a), hE, K, K(5cpa), K(Ac), K(d), K(G), K(Me)3, K(NMe), K(NMeAc), K(NNs), K-Z_{peg}, NMeK-Z_{peg}, L, or Q(NMe2); wherein the C, D, and hE are linked to R₁, the amino acid at X₃, or the amino acid at X₁₀, and optionally wherein the E is linked to R₁, the amino acid at X₃, or the amino acid at X₁₀. In some embodiments, X₁₃ is C, D, Dab(NMeAc), Dab(NMecarn), E, E(COcPEG3a), hE, K(Ac), K(Me)3, K(NMeAc), K-Z_{peg}, L, or Q(NMe2); wherein the C, D, and hE are linked to R₁, the amino acid at X₃, or the amino acid at X₁₀, and optionally wherein the E is linked to R₁, the amino acid at X₃, or the amino acid at X₁₀.

In some embodiments, X₁₃ is C linked to the amino acid at X₃. In some embodiments, X₁₃ is C linked to dK(COCH2CH₂) at X₃. In some embodiments, X₁₃ is C linked to dab(COCH2) at X₃. In some embodiments, X₁₃ is D linked to the amino acid at X₃. In some embodiments, X₁₃ is D linked to ser(MePEG2) at X₃. In some embodiments, X₁₃ is Dab(NMeAc). In some embodiments, X₁₃ is Dab(NMecam). In some embodiments, X₁₃ is Dab-Z_{peg}. In some embodiments, X₁₃ is Dab-Z_{lipid}. In some embodiments, X₁₃ is E. In some embodiments, X₁₃ is E linked to R₁. In some embodiments, X₁₃ is E linked to 5Ava at R₁. In some embodiments, X₁₃ is E linked to 6Ahx at R₁. In some embodiments, X₁₃ is E linked to 7Ahp at R₁. In some embodiments, X₁₃ is E linked to the amino acid at X₃. In some embodiments, X₁₃ is E linked to the amino acid at X₁₀. In some embodiments, X₁₃ is E linked to AEF at X₁₀. In some embodiments, X₁₃ is E(COcPEG3a). In some embodiments, X₁₃ is hE. In some embodiments, X₁₃ is hE linked to R₁. In some embodiments, X₁₃ is hE linked to PEG2 at R₁. In some embodiments, X₁₃ is hE linked to PEG2NMe at R₁. In some embodiments, X₁₃ is hE linked to the amino acid at X₃. In some embodiments, X₁₃ is hE linked to K at X₃. In some embodiments, X₁₃ is K. In some embodiments, X₁₃ is K(5cpa). In some embodiments, X₁₃ is K(Ac). In some embodiments, X₁₃ is K(d). In some embodiments, X₁₃ is K(G). In some embodiments, X₁₃ is K(Me)3. In some embodiments, X₁₃ is K(NMe). In some embodiments, X₁₃ is K(NMeAc). In some embodiments, X₁₃ is K(NNs). In some embodiments, X₁₃ is K-Z_{peg}. In some embodiments, X₁₃ is K-Z_{lipid}. In some embodiments, X₁₃ is NMeK-Z_{peg}. In some embodiments, X₁₃ is NMeK-Z_{lipid}. In some embodiments, X₁₃ is L. In some embodiments, X₁₃ is Q(NMe2).

In some embodiments, X₁₃ is c linked to the amino acid at X₃. In some embodiments, X₁₃ is c linked to dK(COCH2CH₂) at X₃. In some embodiments, X₁₃ is c linked to dab(COCH2) at X₃. In some embodiments, X₁₃ is d linked to the amino acid at X₃. In some embodiments, X₁₃ is d linked to ser(MePEG2) at X₃. In some embodiments, X₁₃ is dDab(NMeAc). In some embodiments, X₁₃ is dDab(NMecam). In some embodiments, X₁₃ is dDab-Z_{peg}. In some embodiments, X₁₃ is dDab-Z_{lipid}. In some embodiments, X₁₃ is e. In some embodiments, X₁₃ is e linked to R₁. In some embodiments, X₁₃ is e linked to 5Ava at R₁. In some embodiments, X₁₃ is e linked to 6Ahx at R₁. In some embodiments, X₁₃ is e linked to 7Ahp at R₁. In some embodiments, X₁₃ is e linked to the amino acid at X₃. In some embodiments, X₁₃ is e linked to the amino acid at X₁₀. In some embodiments, X₁₃ is e linked to AEF at X₁₀. In some embodiments, X₁₃ is e(COcPEG3a). In some embodiments, X₁₃ is he. In some embodiments, X₁₃ is he linked to R₁. In some embodiments, X₁₃ is he linked to PEG2 at R₁. In some embodiments, X₁₃ is he linked to PEG2NMe at R₁. In some embodiments, X₁₃ is he linked to the amino acid at X₃. In some embodiments, X₁₃ is he linked to K at X₃. In some embodiments, X₁₃ is k. In some embodiments, X₁₃ is k(5cpa). In some embodiments, X₁₃ is k(Ac). In some embodiments, X₁₃ is k(d). In some embodiments, X₁₃ is k(G). In some embodiments, X₁₃ is k(Me)3. In some embodiments, X₁₃ is k(NMe). In some embodiments, X₁₃ is k(NMeAc). In some embodiments, X₁₃ is k(NNs). In some embodiments, X₁₃ is k-Z_{peg}. In some embodiments, X₁₃ is k-Z_{lipid}. In some embodiments, X₁₃ is NMek-Z_{peg}. In some embodiments, X₁₃ is NMek-Z_{lipid}. In some embodiments, X₁₃ is 1. In some embodiments, X₁₃ is q(NMe2).

In some embodiments, X₁₄ is N. In some embodiments, X₁₄ is N, wherein the N is an L-amino acid. In some embodiments, X₁₄ is n.

In some embodiments, X₁₅ is

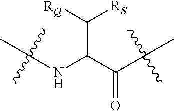

wherein:
R_{Q} is —H or —C₍₁₋₃₎alkyl; and
R_{S} is phenyl or 5- to 6-membered heteroaryl, each of which is optionally substituted with one —C(O)NH₂ group.

In some embodiments, X₁₅ is

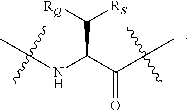

In some embodiments, X₁₅ is

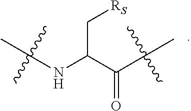

In some embodiments, X₁₅ is

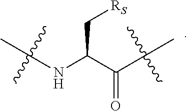

In some embodiments, R_{Q} is —H. In some embodiments, R_{Q} is —CH₃.

In some embodiments, R_{S} is phenyl which is optionally substituted with one —C(O)NH₂ group. In some embodiments, R_{S} is 5- to 6-membered heteroaryl which is optionally substituted with one —C(O)NH₂ group.

In some embodiments, X₁₅ is 3AmPyrazolAla, 3Pya, 5AmPyridinAla, 5MePyridinAla, Ala, ACIPA, aMePhe, H, or THP. In some embodiments, X₁₅ is 3AmPyrazolAla, 3Pya, 5AmPyridinAla, 5MePyridinAla, Ala, ACIPA, aMePhe, H, or THP, wherein the 3AmPyrazolAla, 3Pya, 5AmPyridinAla, 5MePyridinAla, Ala, ACIPA, aMePhe, and H are L-amino acids. In some embodiments, X₁₅ is 3AmPyrazol-d-Ala, d-3Pya, d-5AmPyridinAla, d-5MePyridinAla, dAla, dACIPA, aMe-d-Phe, h, or THP.

In some embodiments, X₁₅ is 3AmPyrazolAla. In some embodiments, X₁₅ is 3Pya. In some embodiments, X₁₅ is 5AmPyridinAla. In some embodiments, X₁₅ is 5MePyridinAla. In some embodiments, X₁₅ is Ala. In some embodiments, X₁₅ is ACIPA. In some embodiments, X₁₅ is aMePhe. In some embodiments, X₁₅ is H. In some embodiments, X₁₅ is THP.

In some embodiments, X₁₅ is 3AmPyrazol-d-Ala. In some embodiments, X₁₅ is d-3Pya.

In some embodiments, X₁₅ is d-5AmPyridinAla. In some embodiments, X₁₅ is d-5MePyridinAla. In some embodiments, X₁₅ is dAla. In some embodiments, X₁₅ is dACIPA. In some embodiments, X₁₅ is aMe-d-Phe. In some embodiments, X₁₅ is h.

In some embodiments, X₁₁ is Sar, Dab-Z_{peg}, Dab-Z_{lipid}, K-Z_{peg}, K-Z_{lipid}, NMeK-Z_{peg}, NMeK-Z_{lipid}, or absent. In some embodiments, X₁₁ is Sar, Dab-Z_{peg}, Dab-Z_{lipid}, K-Z_{peg}, K-Z_{lipid}, NMeK-Z_{peg}, NMeK-Z_{lipid}, or absent, wherein the Dab-Z_{peg}, Dab-Z_{lipid}, K-Z_{peg}, K-Z_{lipid}, NMeK-Z_{peg}, and NMeK-Z_{lipid} are L-amino acids. In some embodiments, X₁₆ is Sar, dDab-Z_{peg}, dDab-Z_{lipid}, k-Z_{peg}, k-Z_{lipid}, NMek-Z_{peg}, NMek-Z_{lipid}, or absent.

In some embodiments, X₁₁ is Sar, NMeK-Z_{lipid}, or absent. In some embodiments, X₁₇ is Sar or absent. In some embodiments, X₁₆ is Sar or NMeK-Z_{lipid}.

In some embodiments, X₁₁ is Sar. In some embodiments, X₁₁ is Dab-Z_{peg}. In some embodiments, X₁₁ is Dab-Z_{lipid}. In some embodiments, X₁₁ is K-Z_{peg}. In some embodiments, X₁₁ is K-Z_{lipid}. In some embodiments, X₁₆ is NMeK-Z_{peg}. In some embodiments, X₁₁ is NMeK-Z_{lipid}.

In some embodiments, X₁₁ is absent.

In some embodiments, X₁₁ is dDab-Z_{peg}. In some embodiments, X₁₆ is dDab-Z_{lipid}. In some embodiments, X₁₁ is k-Z_{peg}. In some embodiments, X₁₁ is k-Z_{lipid}. In some embodiments, X₁₆ is NMek-Z_{peg}. In some embodiments, X₁₆ is NMek-Z_{lipid}.

In some embodiments, X₁₇ is Dab-Z_{peg}, Dab-Z_{lipid}, K-Z_{peg}, K-Z_{lipid}, NMeK-Z_{peg}, NMeK-Z_{lipid}, or absent. In some embodiments, X₁₇ is Dab-Z_{peg}, Dab-Z_{lipid}, K-Z_{peg}, K-Z_{lipid}, NMeK-Z_{peg}, NMeK-Z_{lipid}, or absent, wherein the Dab-Z_{peg}, Dab-Z_{lipid}, K-Z_{peg}, K-Z_{lipid}, NMeK-Z_{peg}, and NMeK-Z_{lipid} are L-amino acids. In some embodiments, X₁₇ is dDab-Z_{peg}, dDab-Z_{lipid}, k-Z_{peg}, k-Z_{lipid}, NMek-Z_{peg}, NMek-Z_{lipid}, or absent.

In some embodiments, X₁₇ is Dab-Z_{peg}, Dab-Z_{lipid}, K-Z_{peg}, K-Z_{lipid}, NMeK-Z_{peg}, or NMeK-Z_{lipid}. In some embodiments, X₁₇ is K-Z_{lipid}, NMeK-Z_{lipid}, or absent.

In some embodiments, X₁₇ is Dab-Z_{peg}. In some embodiments, X₁₇ is Dab-Z_{lipid}. In some embodiments, X₁₇ is K-Z_{peg}. In some embodiments, X₁₇ is K-Z_{lipid}. In some embodiments, X₁₇ is NMeK-Z_{peg}. In some embodiments, X₁₇ is NMeK-Z_{lipid}. In some embodiments, X₁₇ is absent.

In some embodiments, X₁₇ is dDab-Z_{peg}. In some embodiments, X₁₇ is dDab-Z_{lipid}. In some embodiments, X₁₇ is k-Z_{peg}. In some embodiments, X₁₇ is k-Z_{lipid}. In some embodiments, X₁₇ is NMek-Z_{peg}. In some embodiments, X₁₇ is NMek-Z_{lipid}.

In some embodiments, R₂ is CONH2, CO(DiFPip), CON(Me)2, a polyethylene glycol chain terminating in an ammonium or methyl group, or a lipophilic substituent. In some embodiments, R₂ is CONH2, CO(DiFPip), CON(Me)2, a polyethylene glycol chain terminating in an ammonium or methyl group, or a lipophilic substituent.

In some embodiments, R₂ is CONH2, CO(DiFPip), CON(Me)2, or Z_{peg}. In some embodiments, R₂ is CONH2, CON(Me)2, or Z_{peg}. In some embodiments, R₂ is CONH2 or CON(Me)2.

In some embodiments, R₂ is CONH2. In some embodiments, R₂ is CO(DiFPip). In some embodiments, R₂ is CON(Me)2. In some embodiments, R₂ is Z_{peg} (i.e., a polyethylene glycol chain). In some embodiments, R₂ is Z_{lipid} (i.e., a lipophilic substituent).

When two amino acid positions in the peptide molecule are linked to form a ring, the linker between the α-carbon of one amino acid position and the α-carbon of the other amino acid position can be less than 30, 28, 26, 24, 22, 20, 18, 16, 14, 12, 10, 8, 6, or 4 Angstroms (Å). In some embodiments, the linker is between 2 and 30 Å in length. In some embodiments, the linker is between 4 Å and 24 Å in length. In some embodiments, the linker is between 4 Å and 20 Å in length. In some embodiments, the linker is between 10 Å and 24 Å in length. In some embodiments, the linker can comprises between 4 and 18 atoms selected from C, N, S, and O. In some embodiments, the linker can comprise one or more cycloalkyl, heterocyclyl, aryl, or heteroaryl groups. In some embodiments, the linker can consists of C, N, S, O, and H atoms, and comprises between 2 and 16 carbon atoms. In some embodiments, the linker consists of C, N, S, O, and H atoms, and comprises between 4 and 16 atoms selected from C, N, S, and O. In some embodiments, the linker consists of C, S, and H atoms, and comprises between 4 and 16 atoms selected from C and S. In some embodiments, the linker consists of C, N, S, O, and H atoms, and comprises between 2 and 14 carbon atoms. In some embodiments, the linker consists of C, S, and H atoms, and comprises between 2 and 14 carbon atoms. In some embodiments, the linker consists of C, N, S, O, and H atoms, and comprises between 4 and 14 atoms selected from C, N, S, and O. In some embodiments, the linker consists of C, N, S, O, and H atoms, and comprises between 4 and 12 atoms selected from C, N, S, and O. In some embodiments, the linker consists of C, N, S, O, and H atoms, and comprises between 2 and 12 carbon atoms.

In some embodiments, the peptide comprises a linker between the α-carbon of X₄ and the α-carbon of X₉. In some embodiments, the linker is less than 24 Angstroms (Å) in length. In some embodiments, the linker is less than 20 Å in length. In some embodiments, the linker is between 4 Å and 24 Å in length. In some embodiments, the linker is between 4 Å and 20 Å in length. In some embodiments, the linker is between 10 Å and 24 Å in length. In some embodiments, the linker can comprise one or more cycloalkyl, heterocyclyl, aryl, or heteroaryl groups. In some embodiments, the linker can comprise one or more heterocyclyl, aryl, or heteroaryl groups. In some embodiments, the linker consists of C, N, S, O, and H atoms, and comprises between 4 and 18 atoms selected from C, N, S, and O. In some embodiments, the linker consists of C, N, S, O, and H atoms, and comprises between 2 and 16 carbon atoms. In some embodiments, the linker consists of C, N, S, O, and H atoms, and comprises between 4 and 16 atoms selected from C, N, S, and O. In some embodiments, the linker consists of C, S, and H atoms, and comprises between 4 and 16 atoms selected from C and S. In some embodiments, the linker consists of C, N, S, O, and H atoms, and comprises between 2 and 14 carbon atoms. In some embodiments, the linker consists of C, S, and H atoms, and comprises between 2 and 14 carbon atoms. In some embodiments, the linker consists of C, N, S, O, and H atoms, and comprises between 4 and 14 atoms selected from C, N, S, and O. In some embodiments, the linker consists of C, N, S, O, and H atoms, and comprises between 4 and 12 atoms selected from C, N, S, and O. In some embodiments, the linker consists of C, N, S, O, and H atoms, and comprises between 2 and 12 carbon atoms.

In some embodiments, X₄ is Abu and X₉ is C. In some embodiments, X₄ is Abu and X₉ is aMeC. In some embodiments, X₄ is Abu and X₉ is Pen.

In some embodiments, X₄ is C and X₉ is C. In some embodiments, X₄ is C and X₉ is aMeC. In some embodiments, X₄ is C and X₉ is Pen.

In some embodiments, X₄ is Pen and X₉ is C. In some embodiments, X₄ is Pen and X₉ is aMeC. In some embodiments, X₄ is Pen and X₉ is Pen.

In some embodiments, X₄ is aMeC and X₉ is C. In some embodiments, X₄ is aMeC and X₉ is aMeC. In some embodiments, X₄ is aMeC and X₉ is Pen.

In some embodiments, X₄ is Pen(oXyl) and X₉ is C. In some embodiments, X₄ is Pen(oXyl) and X₉ is aMeC. In some embodiments, X₄ is Pen(oXyl) and X₉ is Pen.

In some embodiments, X₄ is Pen(mXyl) and X₉ is C. In some embodiments, X₄ is Pen(mXyl) and X₉ is aMeC. In some embodiments, X₄ is Pen(mXyl) and X₉ is Pen.

In some embodiments, X₄ is Pen(pXyl) and X₉ is C. In some embodiments, X₄ is Pen(pXyl) and X₉ is aMeC. In some embodiments, X₄ is Pen(pXyl) and X₉ is Pen.

In some embodiments, X₄ is 4AminoPro and X₉ is D. In some embodiments, X₄ is 4AminoPro and X₉ is E. In some embodiments, X₄ is 4AminoPro and X₉ is hE.

In some embodiments, X₄ is Dap and X₉ is D. In some embodiments, X₄ is Dap and X₉ is E. In some embodiments, X₄ is Dap and X₉ is hE.

In some embodiments, X₄ is Pra and X₉ is Dap(N3). In some embodiments, X₄ is aG and X₉ is aG.

In some embodiments, the peptide is cyclized via a linkage between the residues at X₄ and X₉ having a structure selected from the following:

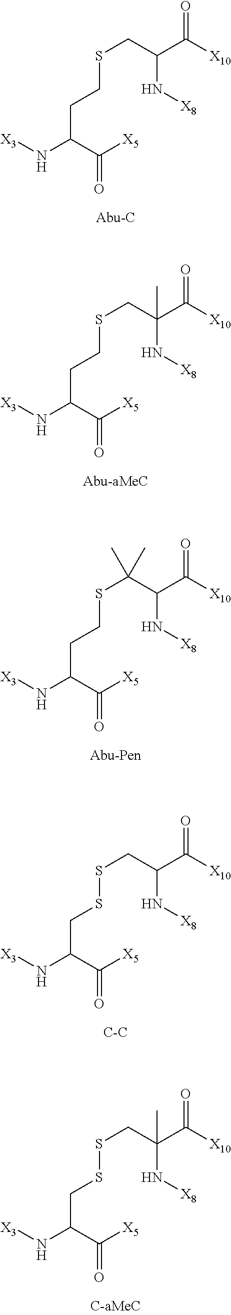

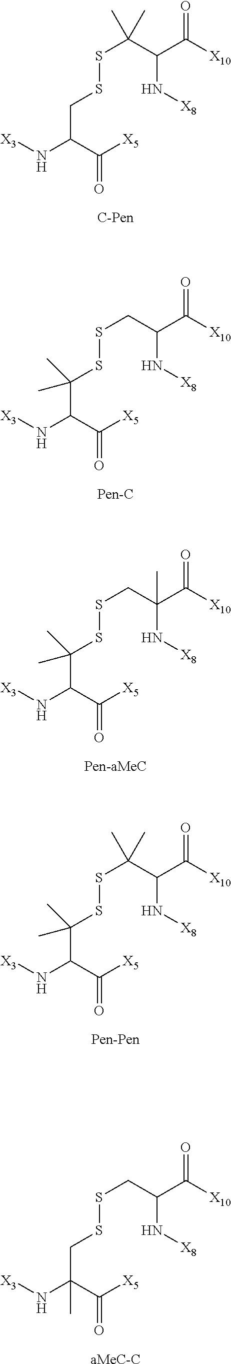

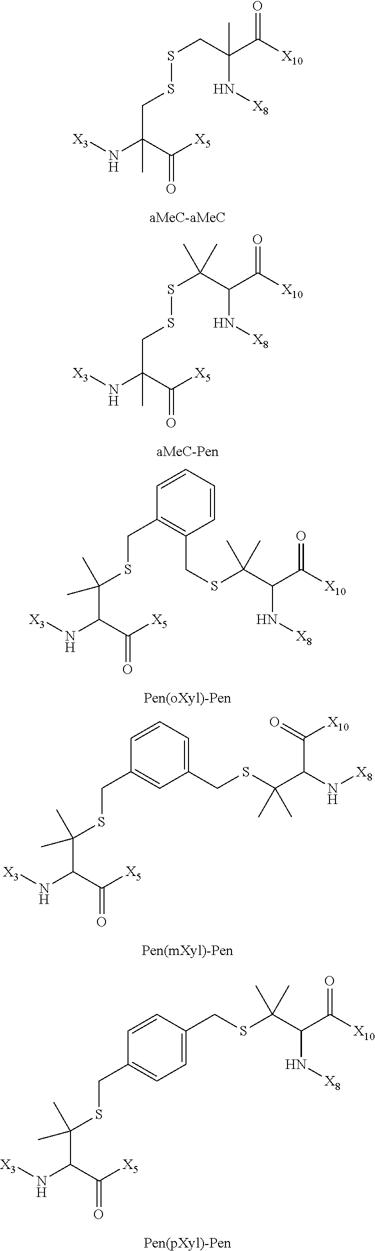

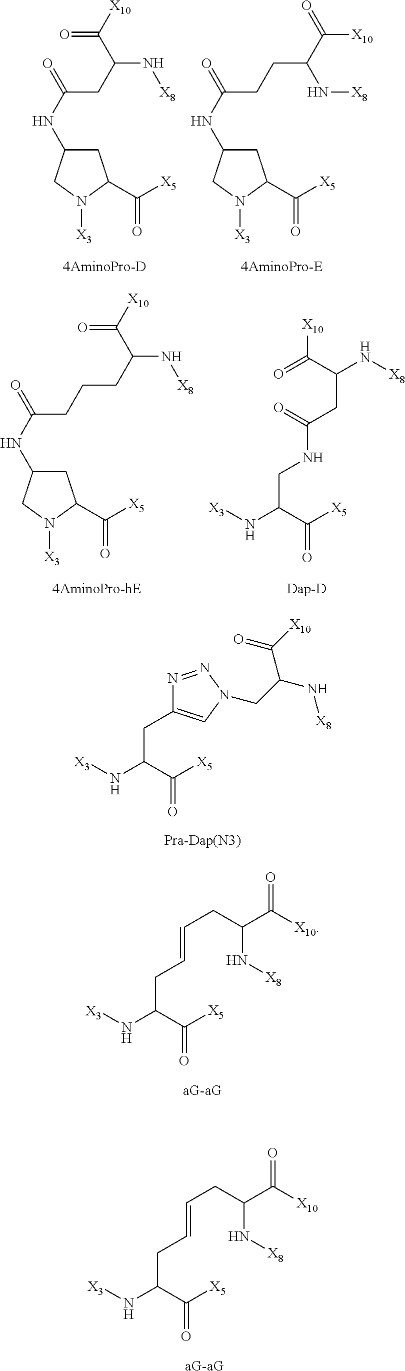

In some embodiments, the peptide is cyclized via a linkage between the residues at X₄ and X₉ having a structure selected from the following:

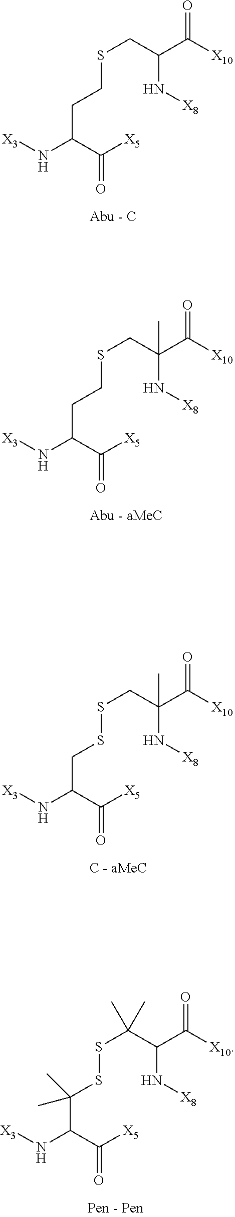

In some embodiments, the peptide is cyclized via a linkage between the residues at X₄ and X₉ having the following structure:

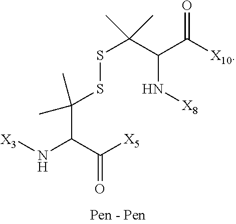

In some embodiments, the peptide comprises one linkage between any one of R₁ and X₁₃, X₃ and X₁₃, X₅ and X₁₀, and X₁₀ and X₁₃. In some embodiments, the peptide comprises no more than one linkage between any one of R₁ and X₁₃, X₃ and X₁₃, X₅ and X₁₀, and X₁₀ and X₁₃.

In some embodiments, the peptide comprises a linkage between R₁ and X₁₃ having a structure selected from the following:

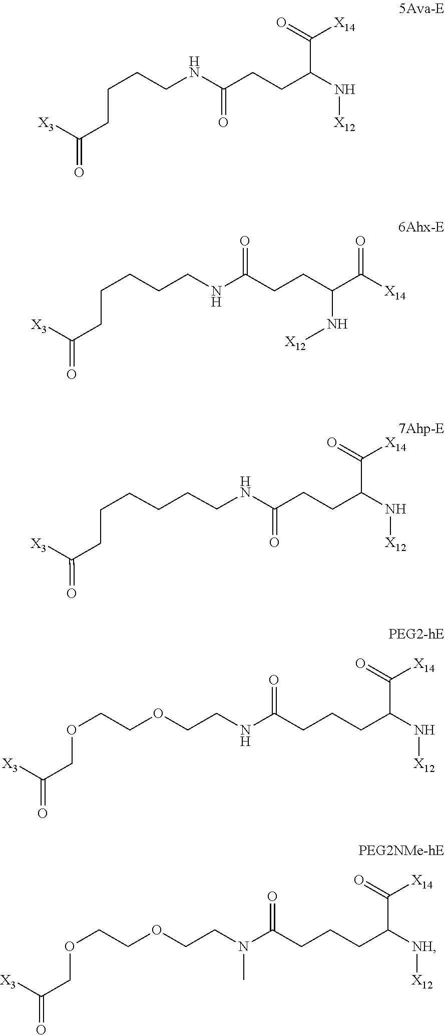

In some embodiments, the peptide comprises a linkage between X₃ and X₁₃ having a structure selected from the following:

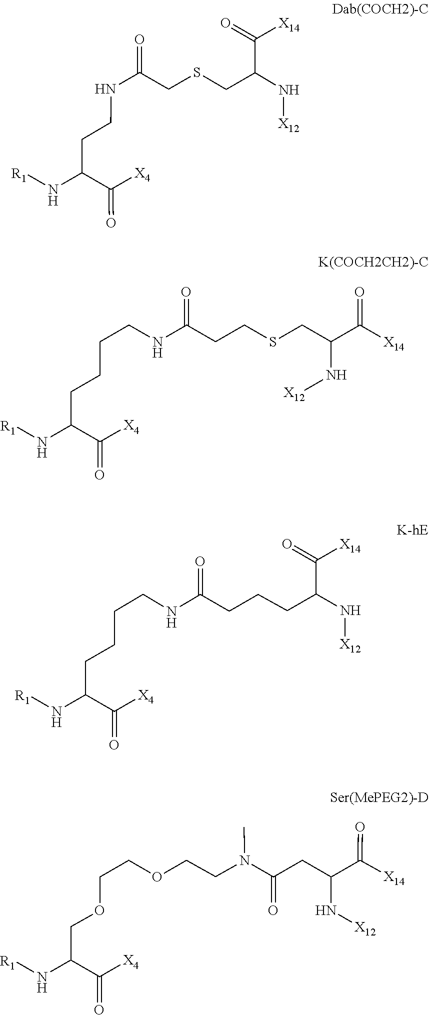

In some embodiments, the peptide comprises a linkage between X₅ and X₁₀ having a structure selected from the following:

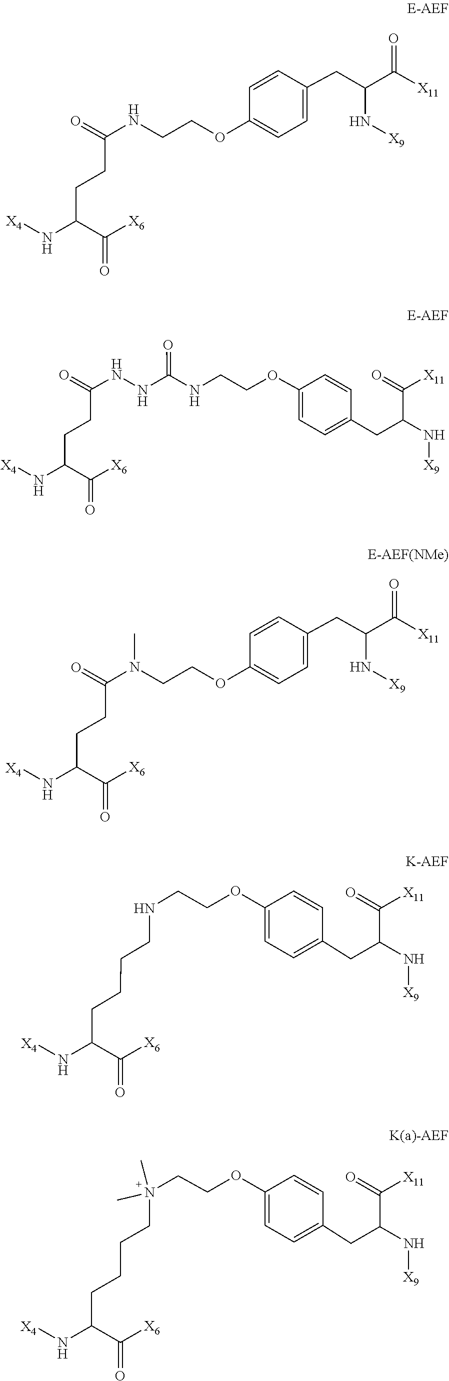

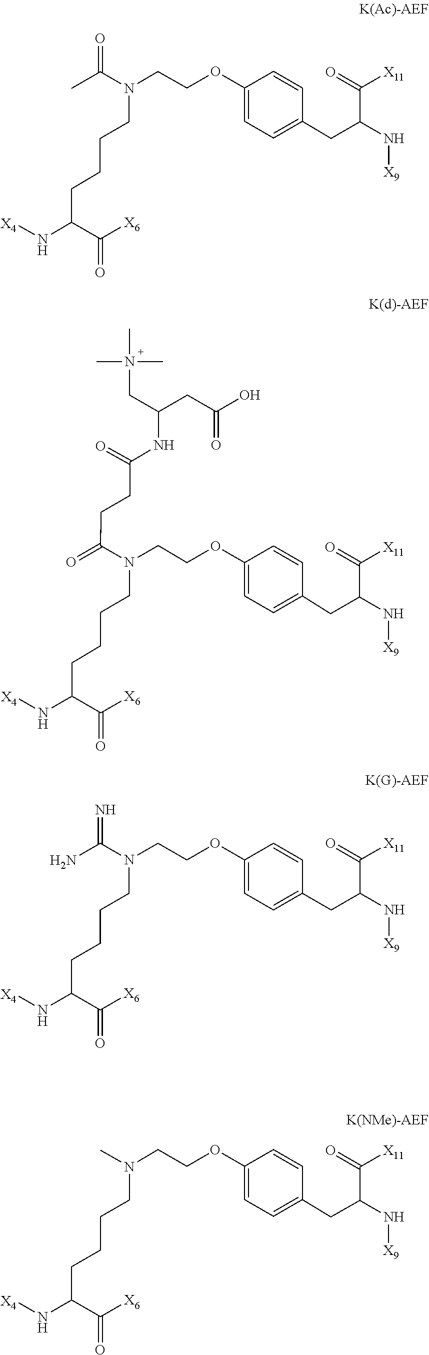

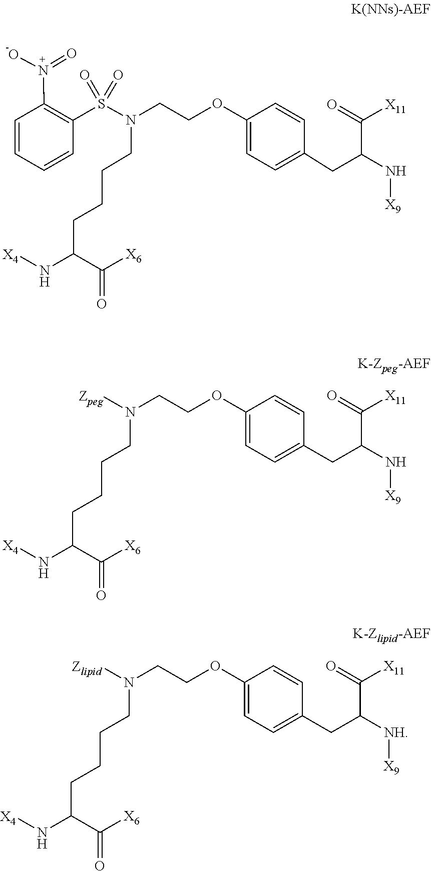

In some embodiments, the peptide comprises a linkage between X₁₀ and X₁₃ having the following structure:

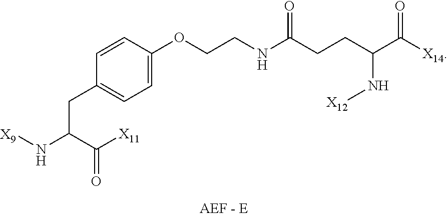

In some embodiments, the peptide is cyclized to form a first ring, wherein the first ring comprises 3 to 14 amino acids. In some embodiments, the peptide is cyclized to form a first ring, wherein the first ring comprises 4-11 or 14 amino acids. In some embodiments, the first ring comprises 4-9 or 11 amino acids. In some embodiments, the first ring comprises 4, 6, or 10 amino acids. In some embodiments, the first ring comprises 4 amino acids. In some embodiments, the first ring comprises 5 amino acids. In some embodiments, the first ring comprises 6 amino acids. In some embodiments, the first ring comprises 7 amino acids. In some embodiments, the first ring comprises 8 amino acids. In some embodiments, the first ring comprises 9 amino acids. In some embodiments, the first ring comprises 10 amino acids. In some embodiments, the first ring comprises 11 amino acids. In some embodiments, the first ring comprises 14 amino acids.

In some embodiments, the first ring comprises a linkage between two amino acids having a structure selected from the following:

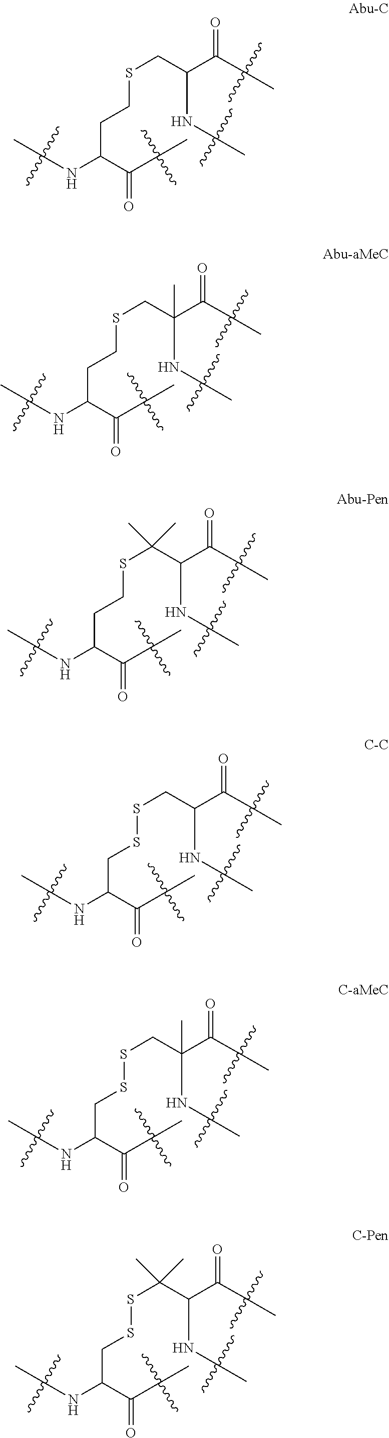 [SMILES: *NC(CCSCC(N*)C(*)=O)C(*)=O ; *NC(CCSCC(C)(N*)C(*)=O)C(*)=O ; *NC(CCSC(C)(C)C(N*)C(*)=O)C(*)=O ; *NC(CSSCC(N*)C(*)=O)C(*)=O ; *NC(CSSCC(C)(N*)C(*)=O)C(*)=O ; *NC(CSSC(C)(C)C(N*)C(*)=O)C(*)=O]

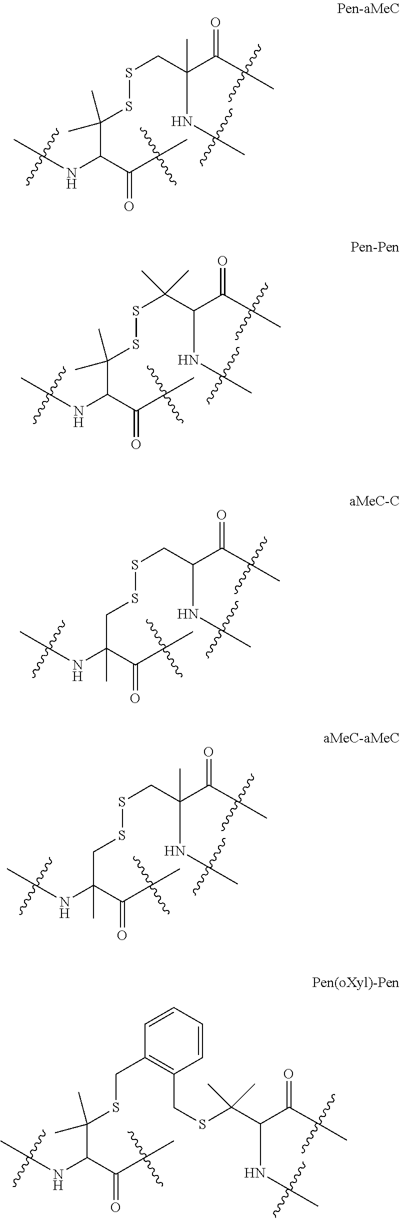 [SMILES: *NC(C(*)=O)C(C)(C)SSCC(C)(N*)C(*)=O ; *NC(C(*)=O)C(C)(C)SSC(C)(C)C(N*)C(*)=O ; *NC(CSSCC(C)(N*)C(*)=O)C(*)=O ; *NC(C)(CSSCC(C)(N*)C(*)=O)C(*)=O ; *NC(C(*)=O)C(C)(C)SCc1ccccc1CSC(C)(C)C(N*)C(*)=O]

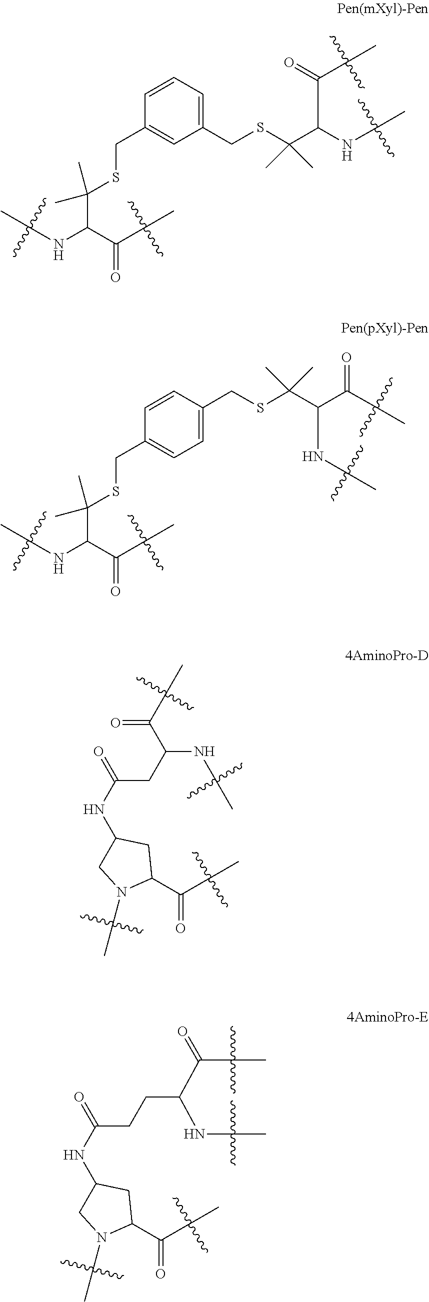 [SMILES: *NC(C(*)=O)C(C)(C)SCc1cccc(CSC(C)(C)C(N*)C(*)=O)c1 ; *NC(C(*)=O)C(C)(C)SCc1ccc(CSC(C)(C)C(N*)C(*)=O)cc1 ; *NC(CC(=O)NC1CC(C(*)=O)N(*)C1)C(*)=O ; *NC(CCC(=O)NC1CC(C(*)=O)N(*)C1)C(*)=O]

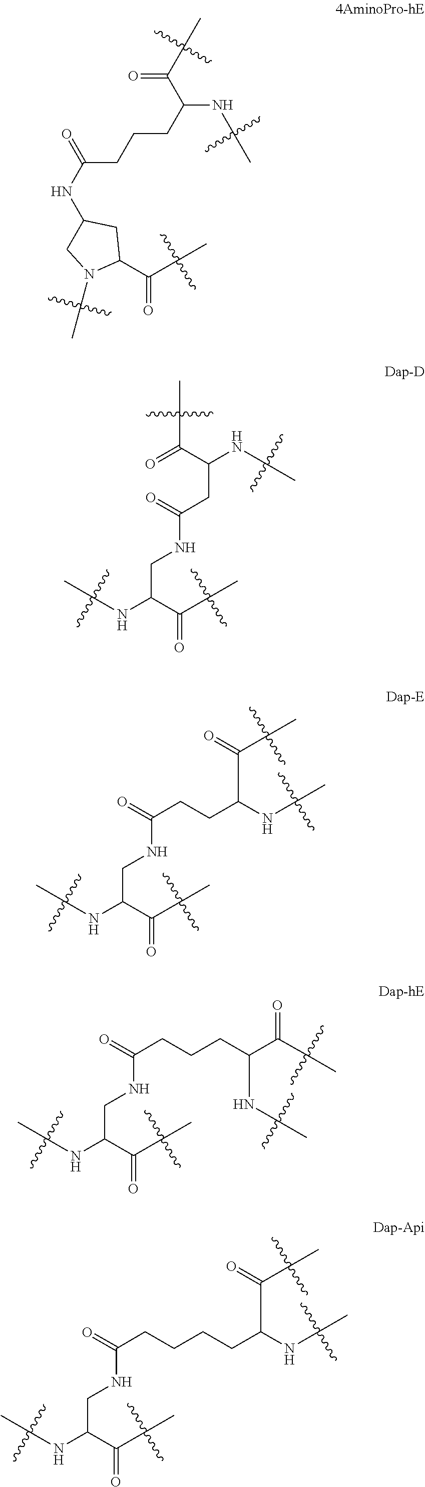 [SMILES: *NC(CCCC(=O)NC1CC(C(*)=O)N(*)C1)C(*)=O ; *NC(CNC(=O)CC(N*)C(*)=O)C(*)=O ; *NC(CCC(=O)NCC(N*)C(*)=O)C(*)=O ; *NC(CCCC(=O)NCC(N*)C(*)=O)C(*)=O ; *NC(CCCCC(=O)NCC(N*)C(*)=O)C(*)=O]

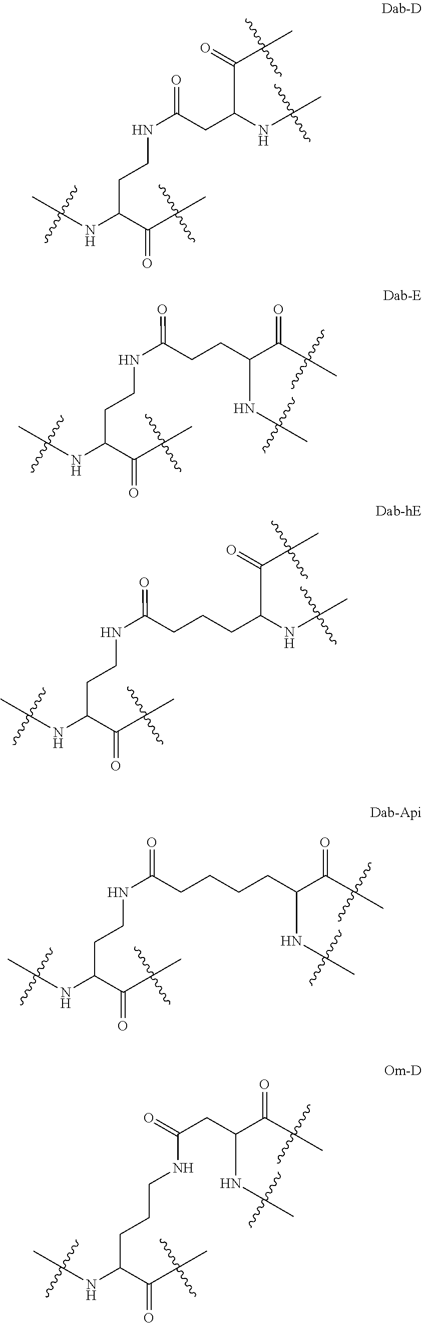 [SMILES: *NC(CCNC(=O)CC(N*)C(*)=O)C(*)=O ; *NC(CCNC(=O)CCC(N*)C(*)=O)C(*)=O ; *NC(CCCC(=O)NCCC(N*)C(*)=O)C(*)=O ; *NC(CCCCC(=O)NCCC(N*)C(*)=O)C(*)=O ; *NC(CCCNC(=O)CC(N*)C(*)=O)C(*)=O]

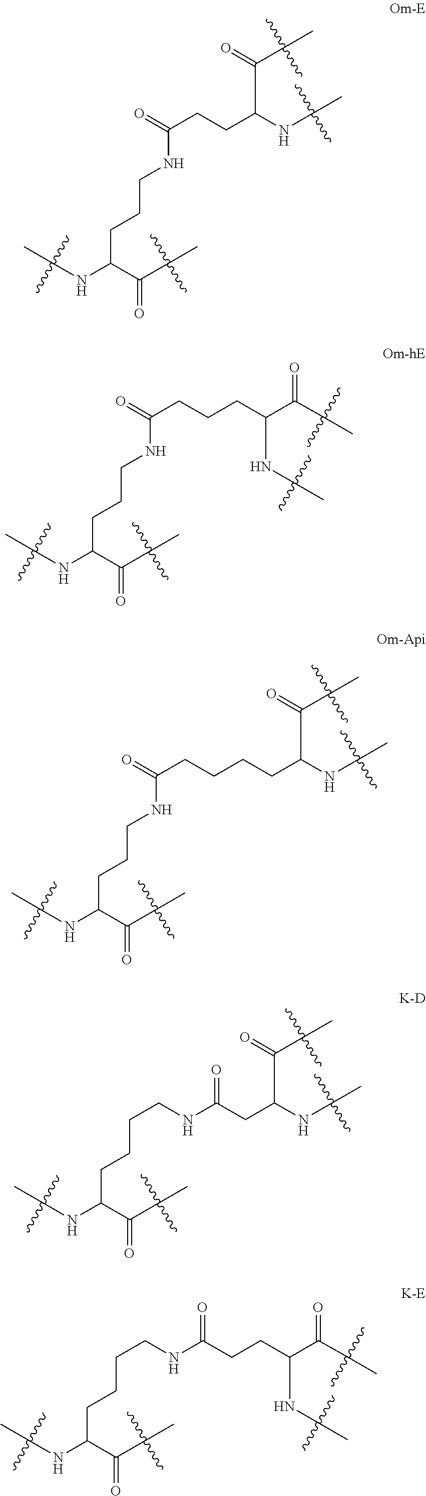 [SMILES: *NC(CCCNC(=O)CCC(N*)C(*)=O)C(*)=O ; *NC(CCCNC(=O)CCCC(N*)C(*)=O)C(*)=O ; *NC(CCCCC(=O)NCCCC(N*)C(*)=O)C(*)=O ; *NC(CCCCNC(=O)CC(N*)C(*)=O)C(*)=O ; *NC(CCCCNC(=O)CCC(N*)C(*)=O)C(*)=O]

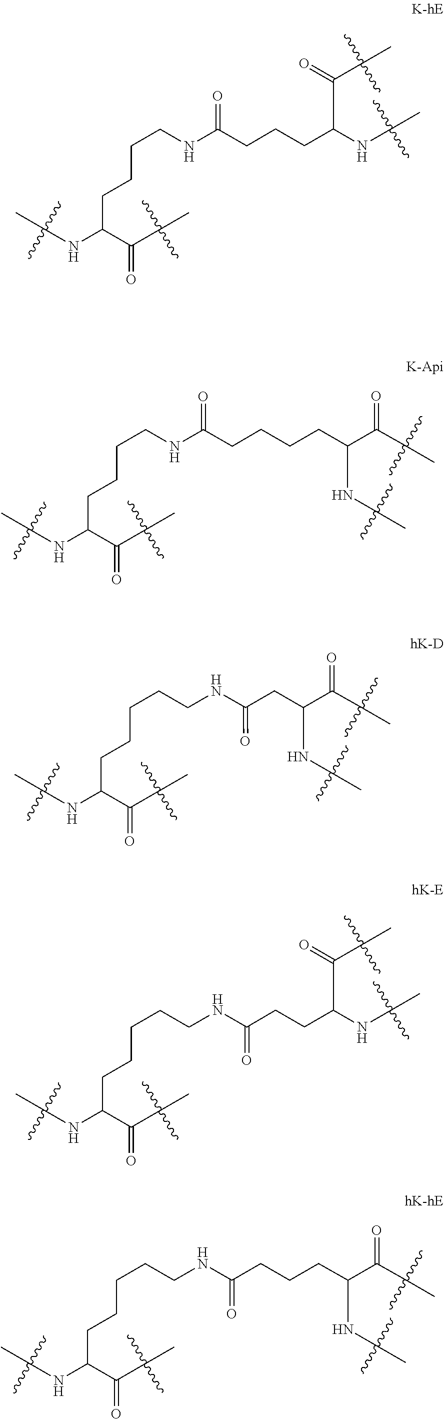 [SMILES: *NC(CCCCNC(=O)CCCC(N*)C(*)=O)C(*)=O ; *NC(CCCCNC(=O)CCCCC(N*)C(*)=O)C(*)=O ; *NC(CCCCCNC(=O)CC(N*)C(*)=O)C(*)=O ; *NC(CCCCCNC(=O)CCC(N*)C(*)=O)C(*)=O ; *NC(CCCCCNC(=O)CCCC(N*)C(*)=O)C(*)=O]

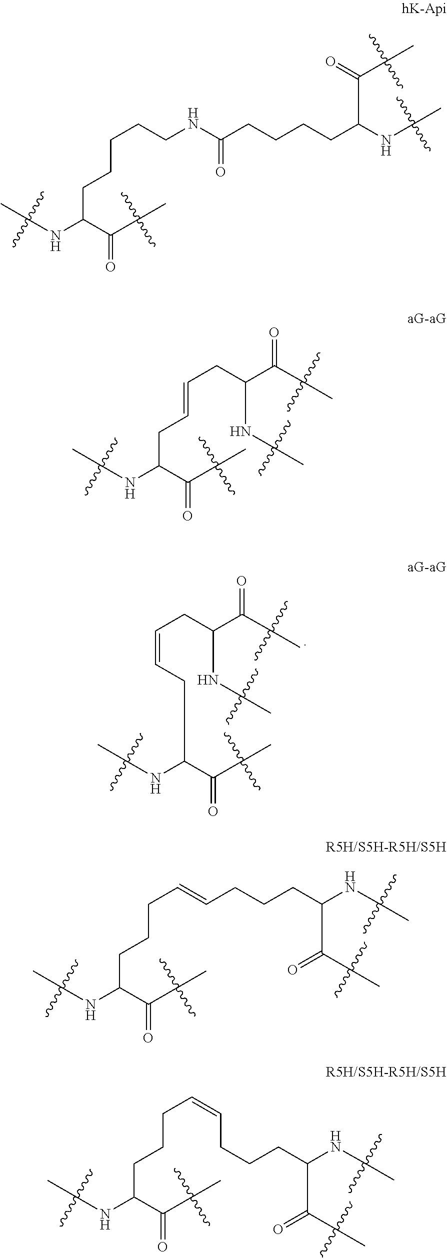 [SMILES: *NC(CCCCCNC(=O)CCCCC(N*)C(*)=O)C(*)=O ; *NC(C/C=C/CC(N*)C(*)=O)C(*)=O ; *NC(C/C=C\CC(N*)C(*)=O)C(*)=O ; *NC(CCC/C=C/CCCC(N*)C(*)=O)C(*)=O ; *NC(CCC/C=C\CCCC(N*)C(*)=O)C(*)=O]

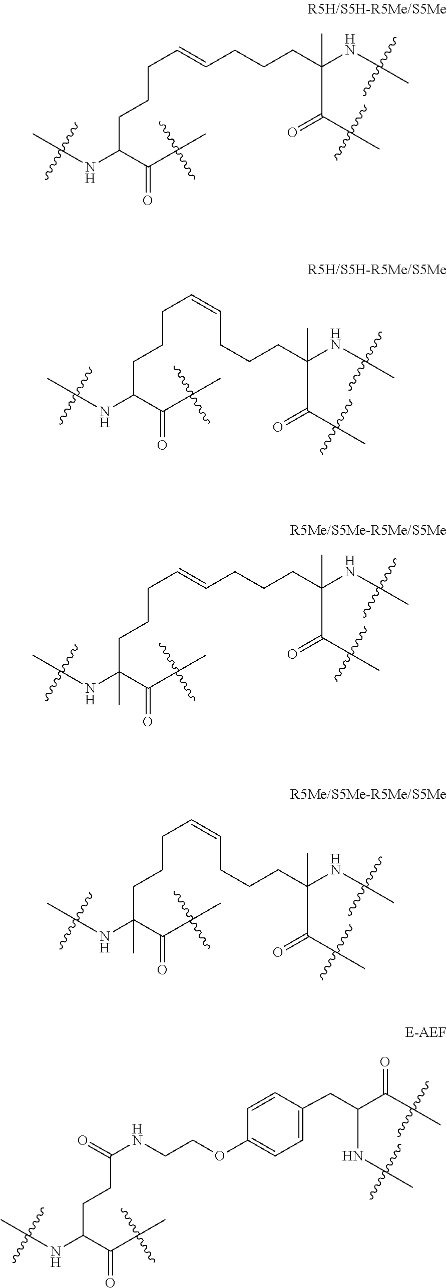 [SMILES: *NC(CCC/C=C/CCCC(C)(N*)C(*)=O)C(*)=O ; *NC(CCC/C=C\CCCC(C)(N*)C(*)=O)C(*)=O ; *NC(C)(CCC/C=C/CCCC(C)(N*)C(*)=O)C(*)=O ; *NC(C)(CCC/C=C\CCCC(C)(N*)C(*)=O)C(*)=O ; *NC(CCC(=O)NCCOc1ccc(CC(N*)C(*)=O)cc1)C(*)=O]

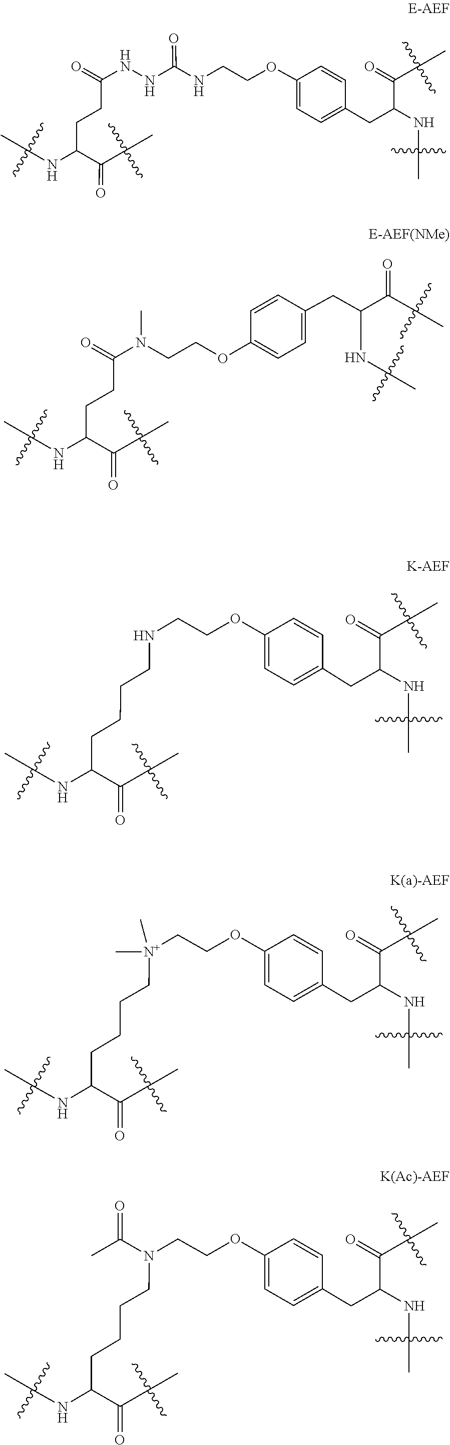 [SMILES: *NC(CCC(=O)NNC(=O)NCCOc1ccc(CC(N*)C(*)=O)cc1)C(*)=O ; *NC(CCC(=O)N(C)CCOc1ccc(CC(N*)C(*)=O)cc1)C(*)=O ; *NC(CCCCNCCOc1ccc(CC(N*)C(*)=O)cc1)C(*)=O ; *NC(CCCC[N+](C)(C)CCOc1ccc(CC(N*)C(*)=O)cc1)C(*)=O ; *NC(CCCCN(CCOc1ccc(CC(N*)C(*)=O)cc1)C(C)=O)C(*)=O]

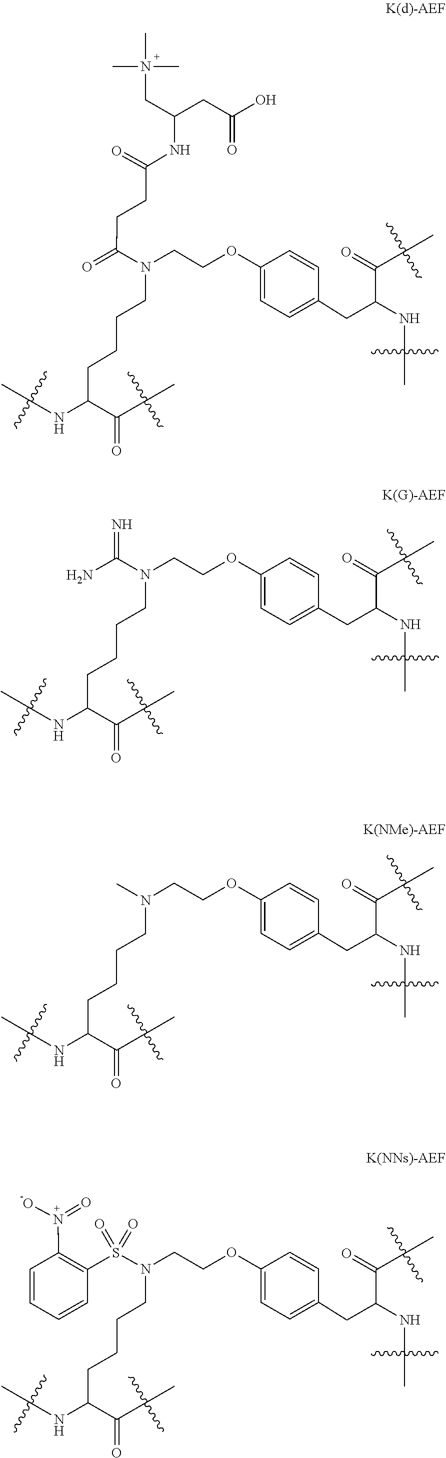

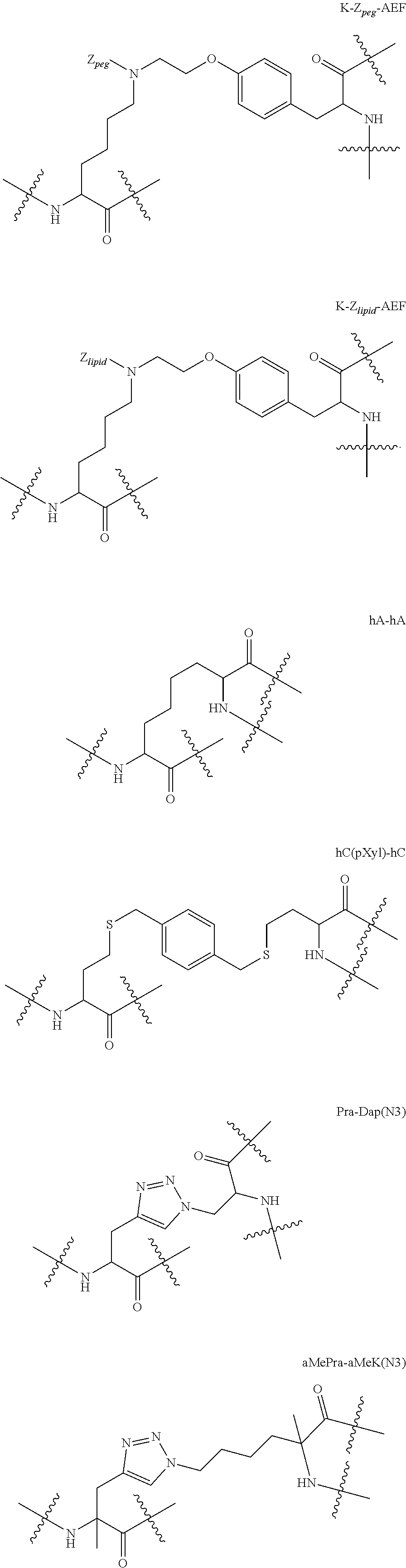

In some embodiments, the first ring comprises a linkage between the N-terminus of the peptide and an amino acid and has a structure selected from the following:

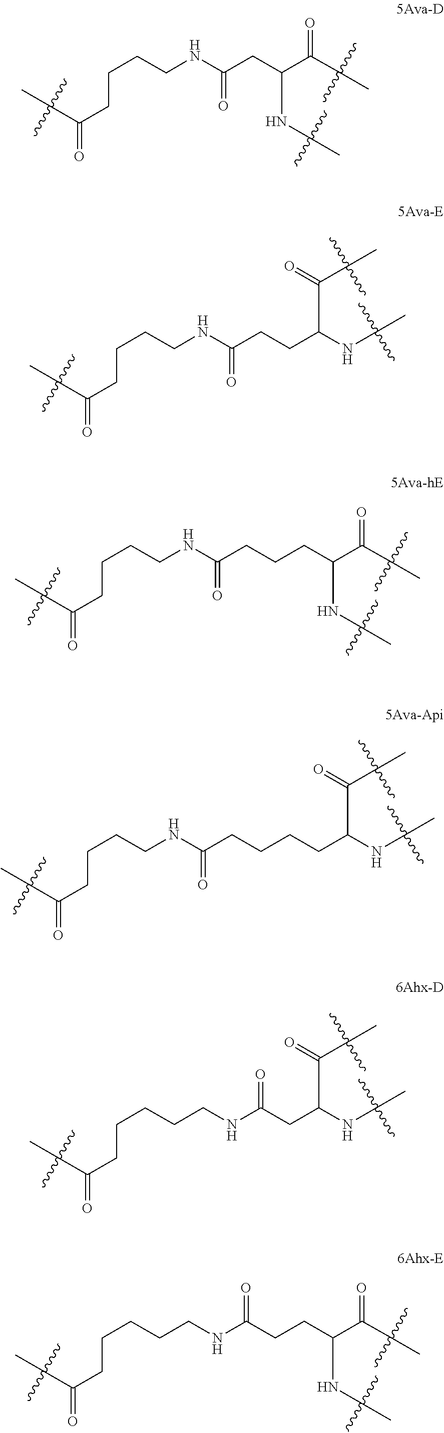 [SMILES: *NC(CC(=O)NCCCCC(*)=O)C(*)=O ; *NC(CCC(=O)NCCCCC(*)=O)C(*)=O ; *NC(CCCC(=O)NCCCCC(*)=O)C(*)=O ; *NC(CCCCC(=O)NCCCCC(*)=O)C(*)=O ; *NC(CC(=O)NCCCCCC(*)=O)C(*)=O ; *NC(CCC(=O)NCCCCCC(*)=O)C(*)=O]

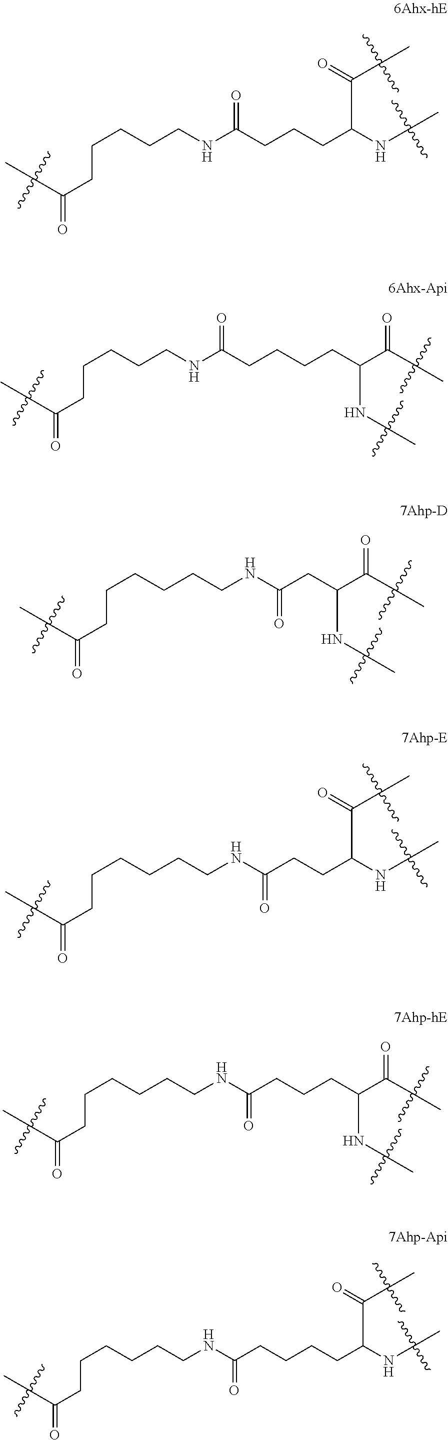 [SMILES: *NC(CCCC(=O)NCCCCCC(*)=O)C(*)=O ; *NC(CCCCC(=O)NCCCCCC(*)=O)C(*)=O ; *NC(CC(=O)NCCCCCCC(*)=O)C(*)=O ; *NC(CCC(=O)NCCCCCCC(*)=O)C(*)=O ; *NC(CCCC(=O)NCCCCCCC(*)=O)C(*)=O ; *NC(CCCCC(=O)NCCCCCCC(*)=O)C(*)=O]

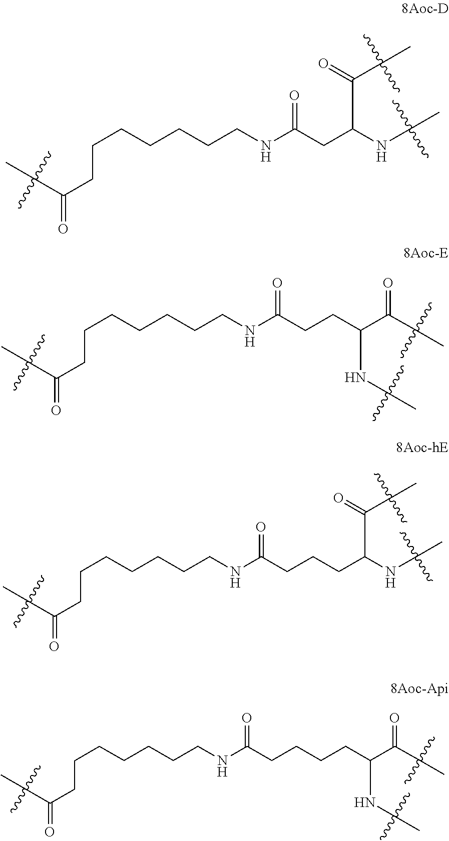 [SMILES: *NC(CC(=O)NCCCCCCCC(*)=O)C(*)=O ; *NC(CCC(=O)NCCCCCCCC(*)=O)C(*)=O ; *NC(CCCC(=O)NCCCCCCCC(*)=O)C(*)=O ; *NC(CCCCC(=O)NCCCCCCCC(*)=O)C(*)=O]

In some embodiments, the first ring is formed between X₄ and X₉, X₄ and X₁₃, X₅ and X₁₀, X₃ and X₁₃, or X₆ and X₉. In some embodiments, the first ring is formed between X₄ and X₉, X₄ and X₁₃, X₅ and X₁₀, X₃ and X₁₃, or X₆ and X₉ via a linker having one or more groups selected from the group consisting of a disulfide, thioether, amide, olefin, ether, alkylene, and triazole. In some embodiments, the first ring is formed between X₄ and X₉, X₄ and X₁₃, X₅ and X₁₀, X₃ and X₁₃, or X₆ and X₉ via a linker selected from the group consisting of a disulfide, thioether, amide, olefin, and triazole.

In some embodiments, the first ring is formed between X₄ and X₉, X₄ and X₁₃, or X₆ and X₉. In some embodiments, the first ring is formed between X₄ and X₉, X₄ and X₁₃, or X₆ and X₉ via a linker having one or more groups selected from the group consisting of a disulfide, thioether, amide, olefin, ether, alkylene, and triazole. In some embodiments, the first ring is formed between X₄ and X₉, X₄ and X₁₃, or X₆ and X₉ via a linker selected from the group consisting of a disulfide, thioether, amide, olefin, and triazole.

In some embodiments, the first ring is formed between X₄ and X₉. In some embodiments, the first ring is formed between X₄ and X₉ via a linker having one or more groups selected from the group consisting of a disulfide, thioether, amide, olefin, ether, alkylene, and triazole. In some embodiments, the first ring is formed between X₄ and X₉ via a linker selected from the group consisting of a disulfide, thioether, amide, olefin, and triazole.

In some embodiments, the first ring is formed between X₄ and X₁₃. In some embodiments, the first ring is formed between X₄ and X₁₃ via a linker having one or more groups selected from the group consisting of a disulfide, thioether, amide, olefin, ether, alkylene, and triazole. In some embodiments, the first ring is formed between X₄ and X₁₃ via a linker selected from the group consisting of a disulfide, thioether, amide, olefin, and triazole.

In some embodiments, the first ring is formed between X₅ and X₁₀. In some embodiments, the first ring is formed between X₅ and X₁₀ via a linker having one or more groups selected from the group consisting of a disulfide, thioether, amide, olefin, ether, alkylene, and triazole. In some embodiments, the first ring is formed between X₅ and X₁₀ via a linker selected from the group consisting of a disulfide, thioether, amide, olefin, and triazole.

In some embodiments, the first ring is formed between X₃ and X₁₃. In some embodiments, the first ring is formed between X₃ and X₁₃ via a linker having one or more groups selected from the group consisting of a disulfide, thioether, amide, olefin, ether, alkylene, and triazole. In some embodiments, the first ring is formed between X₃ and X₁₃ via a linker selected from the group consisting of a disulfide, thioether, amide, olefin, and triazole.

In some embodiments, the first ring is formed between X₆ and X₉. In some embodiments, the first ring is formed between X₆ and X₉ via a linker having one or more groups selected from the group consisting of a disulfide, thioether, amide, olefin, ether, alkylene, and triazole. In some embodiments, the first ring is formed between X₆ and X₉ via a linker selected from the group consisting of a disulfide, thioether, amide, olefin, and triazole.

In some embodiments, the peptide is further cyclized to form a second ring, wherein the second ring comprises 3 to 14 amino acids. In some embodiments, the peptide is further cyclized to form a second ring, wherein the second ring comprises 4-11 or 14 amino acids. In some embodiments, the second ring comprises 4-9 or 11 amino acids. In some embodiments, the second ring comprises 4, 6, 10 or 11 amino acids. In some embodiments, the second ring comprises 4 amino acids. In some embodiments, the second ring comprises 5 amino acids. In some embodiments, the second ring comprises 6 amino acids. In some embodiments, the second ring comprises 7 amino acids. In some embodiments, the second ring comprises 8 amino acids. In some embodiments, the second ring comprises 9 amino acids. In some embodiments, the second ring comprises 10 amino acids. In some embodiments, the second ring comprises 11 amino acids. In some embodiments, the second ring comprises 14 amino acids.

In some embodiments, the second ring comprises a linkage between two amino acids having a structure selected from the following:

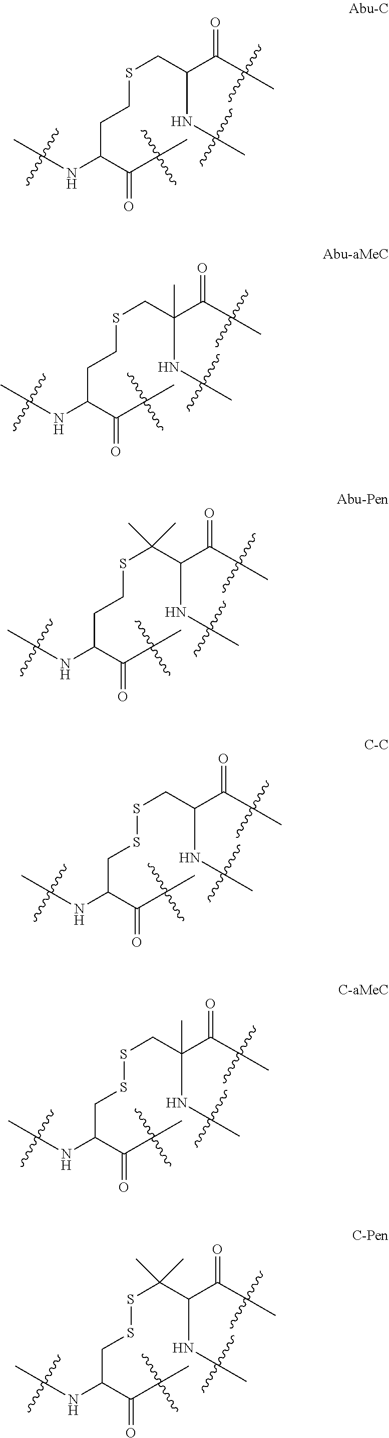 [SMILES: *NC(CCSCC(N*)C(*)=O)C(*)=O ; *NC(CCSCC(C)(N*)C(*)=O)C(*)=O ; *NC(CCSC(C)(C)C(N*)C(*)=O)C(*)=O ; *NC(CSSCC(N*)C(*)=O)C(*)=O ; *NC(CSSCC(C)(N*)C(*)=O)C(*)=O ; *NC(CSSC(C)(C)C(N*)C(*)=O)C(*)=O]

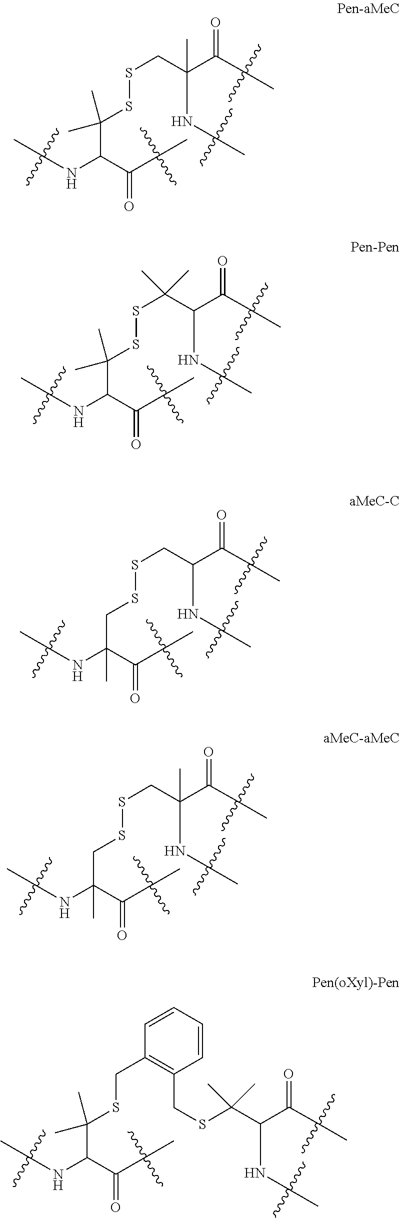 [SMILES: *NC(C(*)=O)C(C)(C)SSCC(C)(N*)C(*)=O ; *NC(C(*)=O)C(C)(C)SSC(C)(C)C(N*)C(*)=O ; *NC(CSSCC(C)(N*)C(*)=O)C(*)=O ; *NC(C)(CSSCC(C)(N*)C(*)=O)C(*)=O ; *NC(C(*)=O)C(C)(C)SCc1ccccc1CSC(C)(C)C(N*)C(*)=O]

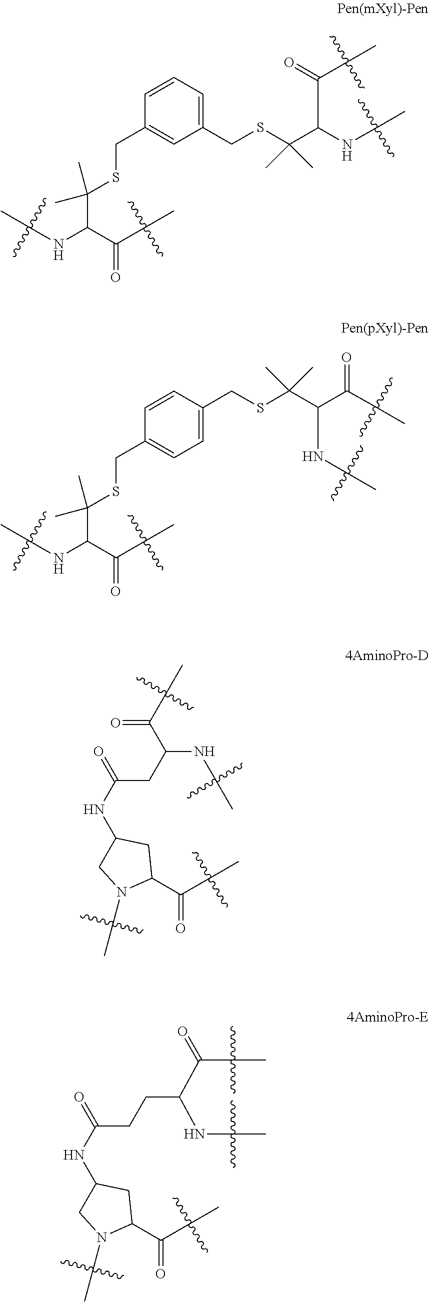 [SMILES: *NC(C(*)=O)C(C)(C)SCc1cccc(CSC(C)(C)C(N*)C(*)=O)c1 ; *NC(C(*)=O)C(C)(C)SCc1ccc(CSC(C)(C)C(N*)C(*)=O)cc1 ; *NC(CC(=O)NC1CC(C(*)=O)N(*)C1)C(*)=O ; *NC(CCC(=O)NC1CC(C(*)=O)N(*)C1)C(*)=O]

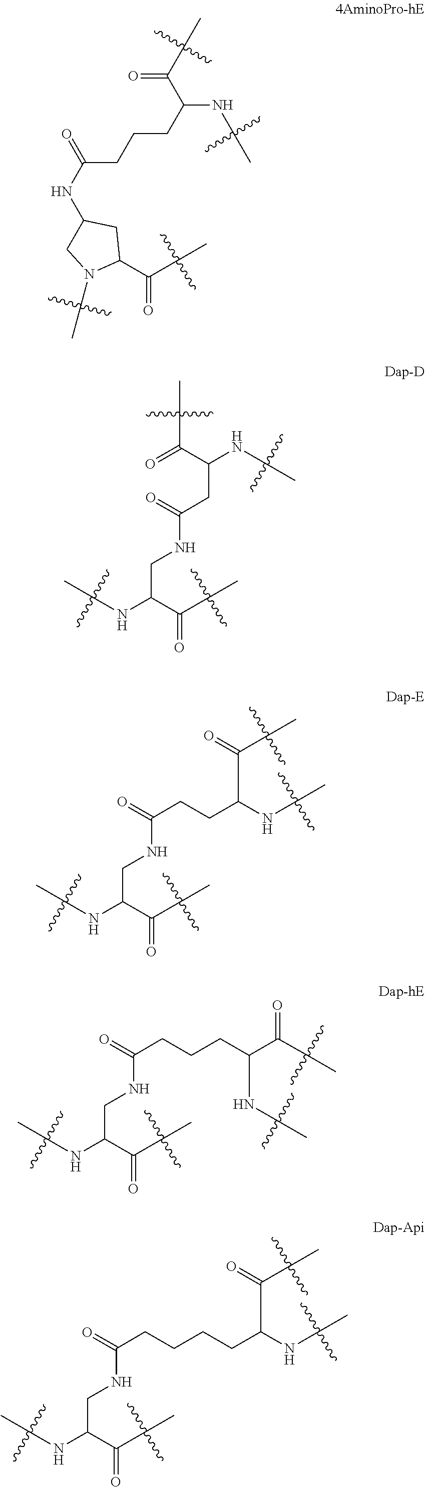 [SMILES: *NC(CCCC(=O)NC1CC(C(*)=O)N(*)C1)C(*)=O ; *NC(CNC(=O)CC(N*)C(*)=O)C(*)=O ; *NC(CCC(=O)NCC(N*)C(*)=O)C(*)=O ; *NC(CCCC(=O)NCC(N*)C(*)=O)C(*)=O ; *NC(CCCCC(=O)NCC(N*)C(*)=O)C(*)=O]

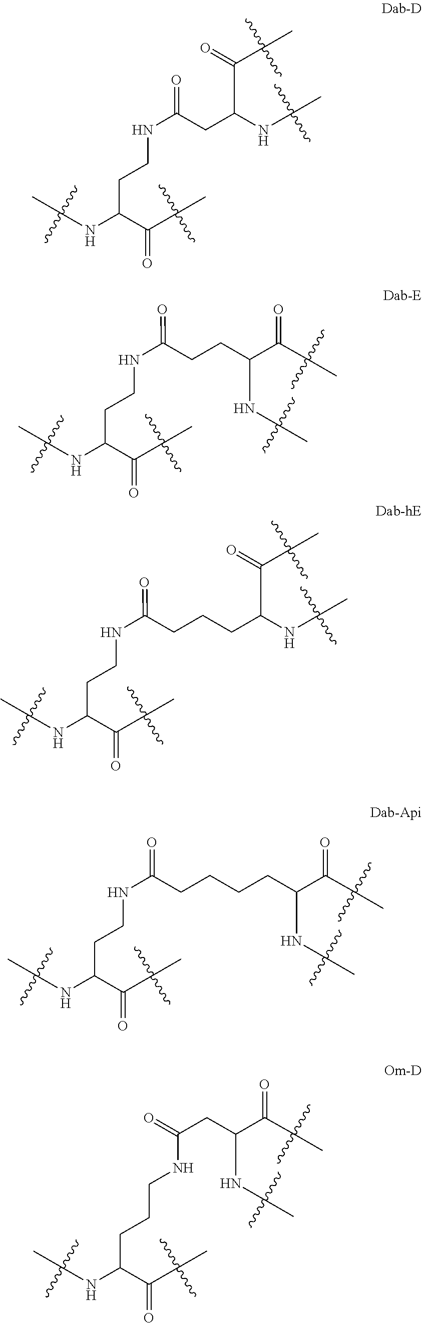 [SMILES: *NC(CCNC(=O)CC(N*)C(*)=O)C(*)=O ; *NC(CCNC(=O)CCC(N*)C(*)=O)C(*)=O ; *NC(CCCC(=O)NCCC(N*)C(*)=O)C(*)=O ; *NC(CCCCC(=O)NCCC(N*)C(*)=O)C(*)=O ; *NC(CCCNC(=O)CC(N*)C(*)=O)C(*)=O]

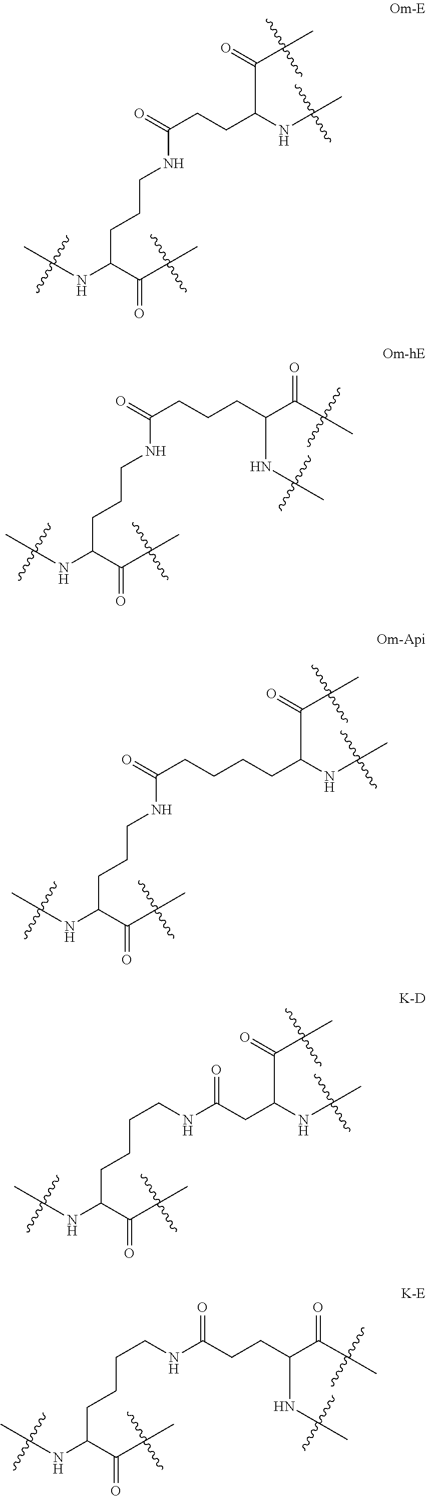 [SMILES: *NC(CCCNC(=O)CCC(N*)C(*)=O)C(*)=O ; *NC(CCCNC(=O)CCCC(N*)C(*)=O)C(*)=O ; *NC(CCCCC(=O)NCCCC(N*)C(*)=O)C(*)=O ; *NC(CCCCNC(=O)CC(N*)C(*)=O)C(*)=O ; *NC(CCCCNC(=O)CCC(N*)C(*)=O)C(*)=O]

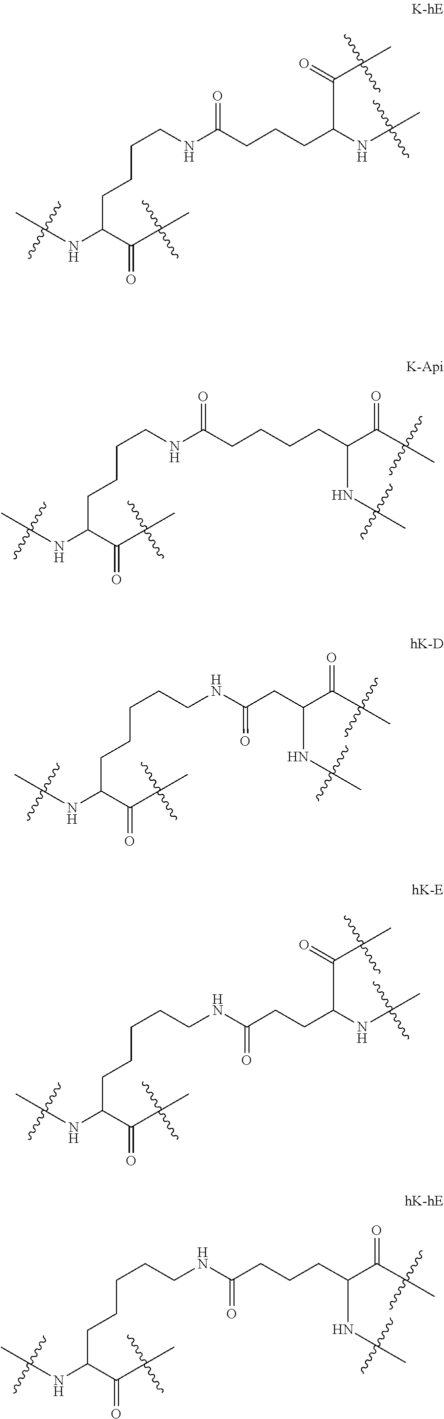 [SMILES: *NC(CCCCNC(=O)CCCC(N*)C(*)=O)C(*)=O ; *NC(CCCCNC(=O)CCCCC(N*)C(*)=O)C(*)=O ; *NC(CCCCCNC(=O)CC(N*)C(*)=O)C(*)=O ; *NC(CCCCCNC(=O)CCC(N*)C(*)=O)C(*)=O ; *NC(CCCCCNC(=O)CCCC(N*)C(*)=O)C(*)=O]

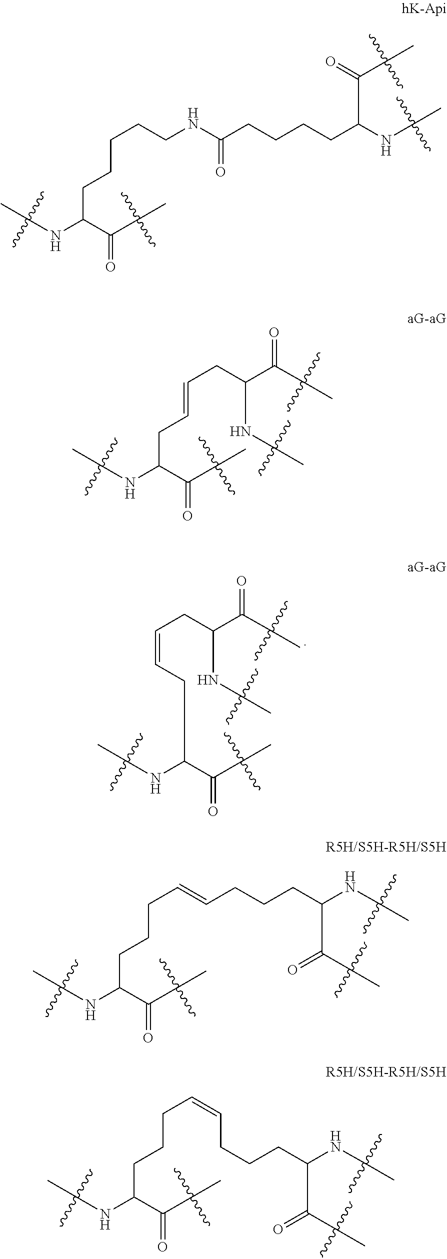 [SMILES: *NC(CCCCCNC(=O)CCCCC(N*)C(*)=O)C(*)=O ; *NC(C/C=C/CC(N*)C(*)=O)C(*)=O ; *NC(C/C=C\CC(N*)C(*)=O)C(*)=O ; *NC(CCC/C=C/CCCC(N*)C(*)=O)C(*)=O ; *NC(CCC/C=C\CCCC(N*)C(*)=O)C(*)=O]

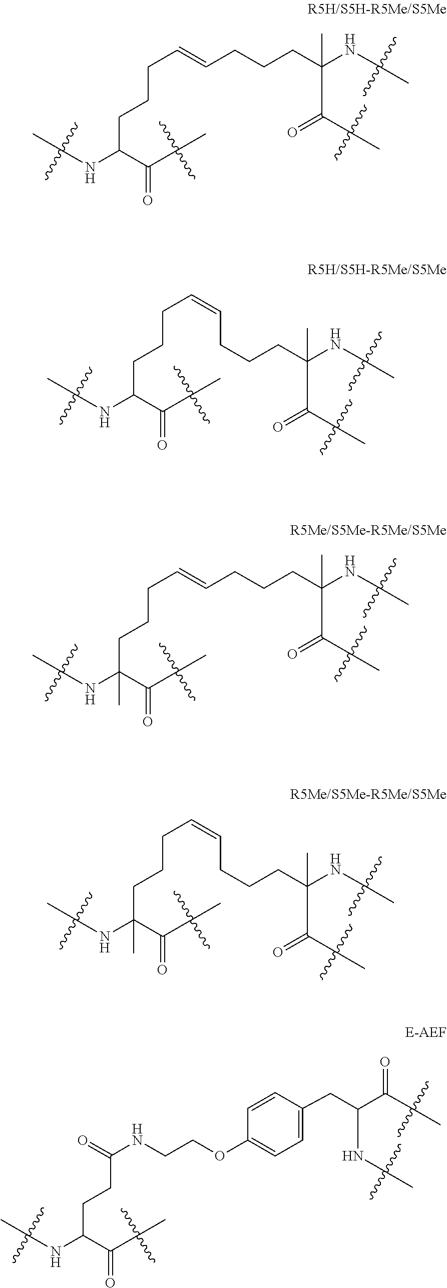 [SMILES: *NC(CCC/C=C/CCCC(C)(N*)C(*)=O)C(*)=O ; *NC(CCC/C=C\CCCC(C)(N*)C(*)=O)C(*)=O ; *NC(C)(CCC/C=C/CCCC(C)(N*)C(*)=O)C(*)=O ; *NC(C)(CCC/C=C\CCCC(C)(N*)C(*)=O)C(*)=O ; *NC(CCC(=O)NCCOc1ccc(CC(N*)C(*)=O)cc1)C(*)=O]

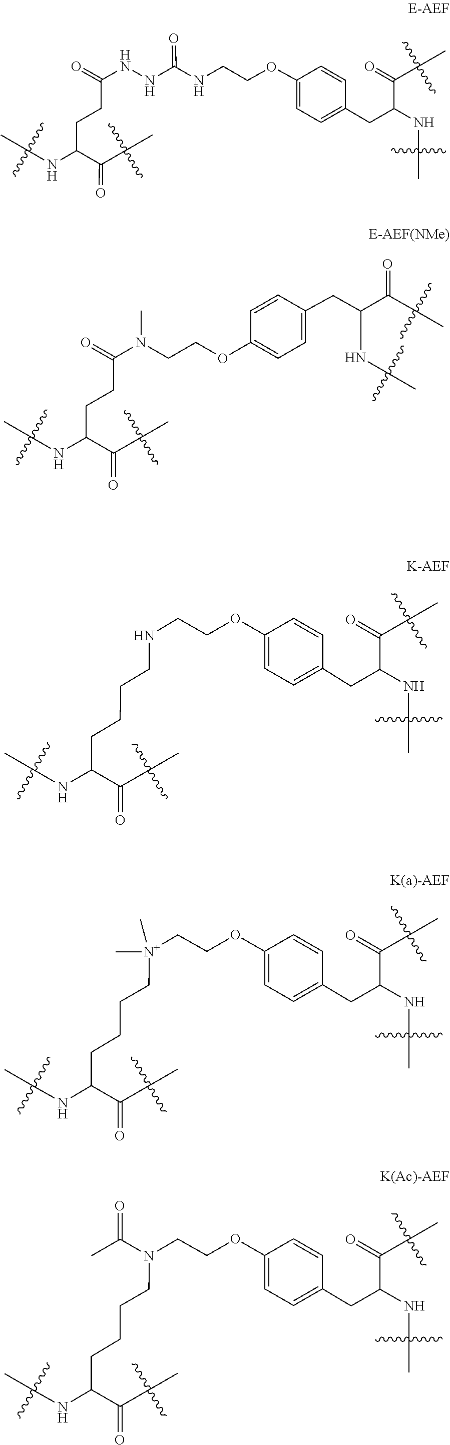 [SMILES: *NC(CCC(=O)NNC(=O)NCCOc1ccc(CC(N*)C(*)=O)cc1)C(*)=O ; *NC(CCC(=O)N(C)CCOc1ccc(CC(N*)C(*)=O)cc1)C(*)=O ; *NC(CCCCNCCOc1ccc(CC(N*)C(*)=O)cc1)C(*)=O ; *NC(CCCC[N+](C)(C)CCOc1ccc(CC(N*)C(*)=O)cc1)C(*)=O ; *NC(CCCCN(CCOc1ccc(CC(N*)C(*)=O)cc1)C(C)=O)C(*)=O]

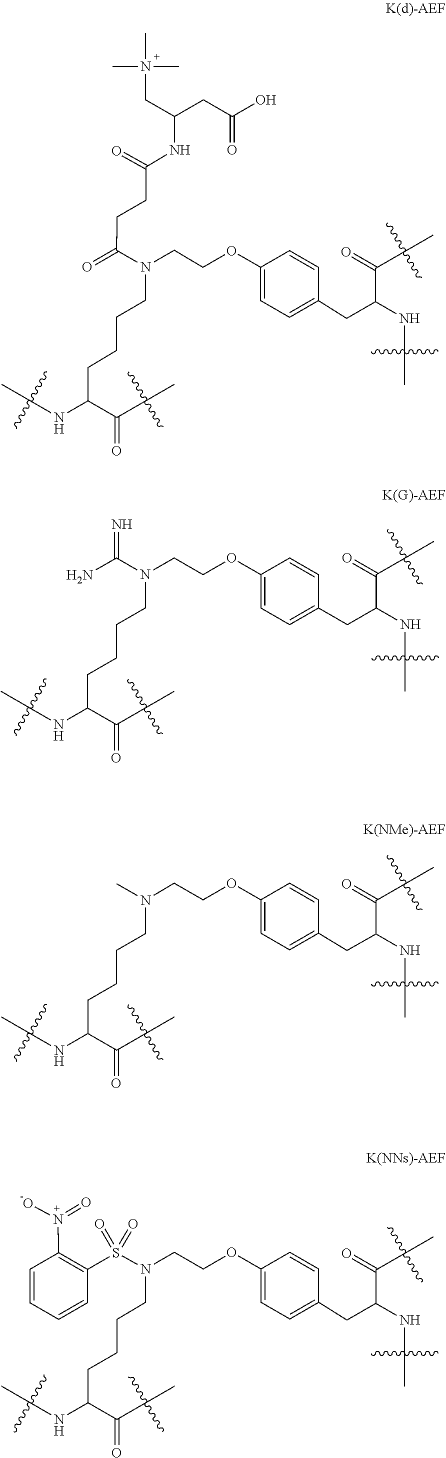

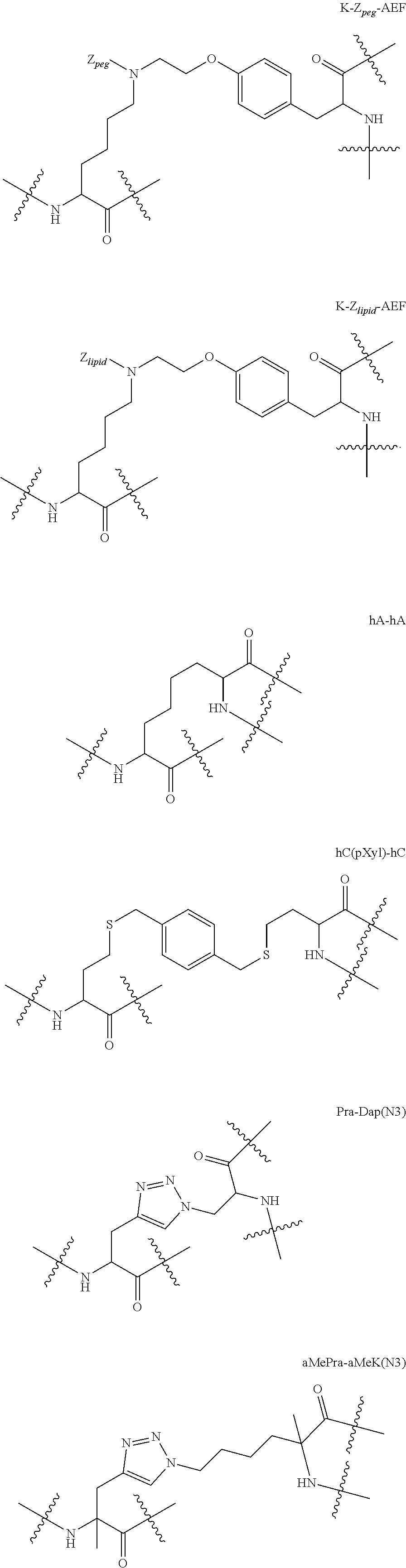

In some embodiments, the second ring comprises a linkage between the N-terminus of the peptide and an amino acid and has a structure selected from the following:

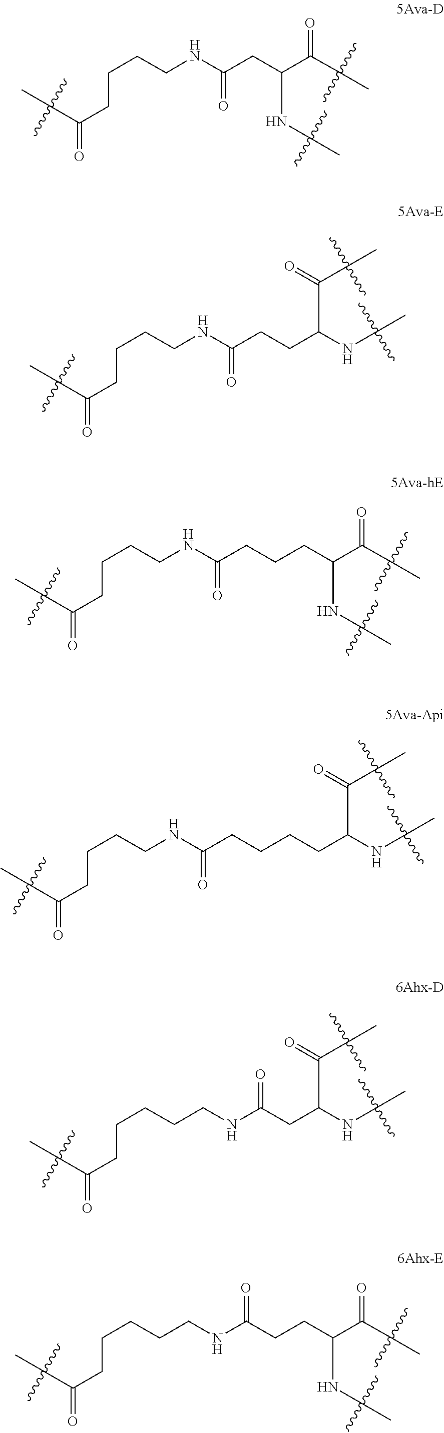 [SMILES: *NC(CC(=O)NCCCCC(*)=O)C(*)=O ; *NC(CCC(=O)NCCCCC(*)=O)C(*)=O ; *NC(CCCC(=O)NCCCCC(*)=O)C(*)=O ; *NC(CCCCC(=O)NCCCCC(*)=O)C(*)=O ; *NC(CC(=O)NCCCCCC(*)=O)C(*)=O ; *NC(CCC(=O)NCCCCCC(*)=O)C(*)=O]

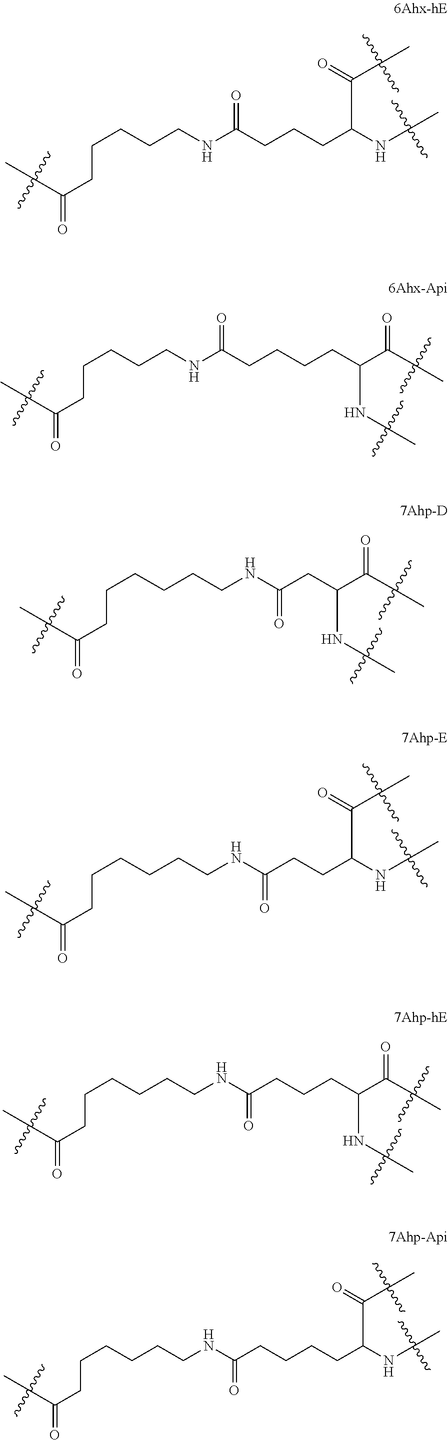 [SMILES: *NC(CCCC(=O)NCCCCCC(*)=O)C(*)=O ; *NC(CCCCC(=O)NCCCCCC(*)=O)C(*)=O ; *NC(CC(=O)NCCCCCCC(*)=O)C(*)=O ; *NC(CCC(=O)NCCCCCCC(*)=O)C(*)=O ; *NC(CCCC(=O)NCCCCCCC(*)=O)C(*)=O ; *NC(CCCCC(=O)NCCCCCCC(*)=O)C(*)=O]

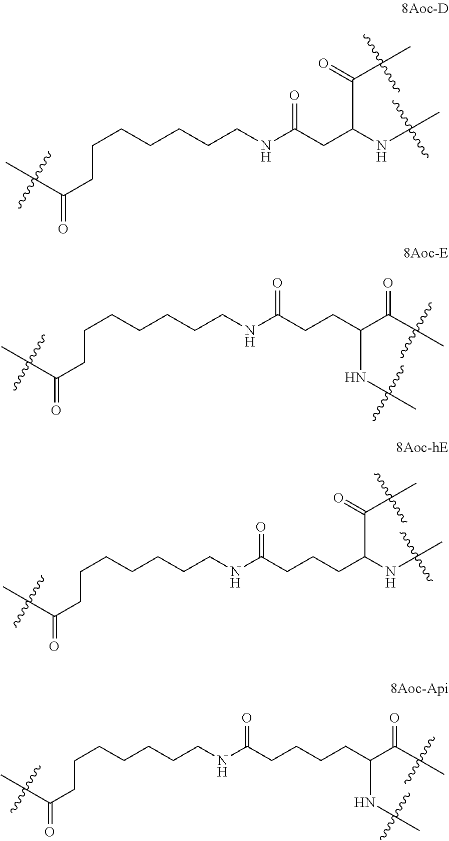 [SMILES: *NC(CC(=O)NCCCCCCCC(*)=O)C(*)=O ; *NC(CCC(=O)NCCCCCCCC(*)=O)C(*)=O ; *NC(CCCC(=O)NCCCCCCCC(*)=O)C(*)=O ; *NC(CCCCC(=O)NCCCCCCCC(*)=O)C(*)=O]

In some embodiments, the second ring is formed between X₄ and X₉, X₄ and X₁₃, X₅ and X₁₀, X₃ and X₁₃, or X₆ and X₉. In some embodiments, the second ring is formed between X₄ and X₉, X₄ and X₁₃, X₅ and X₁₀, X₃ and X₁₃, or X₆ and X₉ via a linker having one or more groups selected from the group consisting of a disulfide, thioether, amide, olefin, ether, alkylene, and triazole. In some embodiments, the second ring is formed between X₄ and X₉, X₄ and X₁₃, X₅ and X₁₀, X₃ and X₁₃, or X₆ and X₉ via a linker selected from the group consisting of a disulfide, thioether, amide, olefin, and triazole.

In some embodiments, the second ring is formed between X₅ and X₁₀ or X₃ and X₁₃. In some embodiments, the second ring is formed between X₅ and X₁₀ or X₃ and X₁₃ via a linker having one or more groups selected from the group consisting of a disulfide, thioether, amide, olefin, ether, alkylene, and triazole. In some embodiments, the second ring is formed between X₅ and X₁₀ or X₃ and X₁₃ via a linker having one or more groups selected from the group consisting of a disulfide, thioether, amide, olefin, and triazole. In some embodiments, the second ring is formed between X₅ and X₁₀ or X₃ and X₁₃ via a linker selected from the group consisting of a disulfide, thioether, amide, olefin, and triazole.

In some embodiments, the second ring is formed between X₄ and X₉. In some embodiments, the second ring is formed between X₄ and X₉ via a linker having one or more groups selected from the group consisting of a disulfide, thioether, amide, olefin, ether, alkylene, and triazole. In some embodiments, the second ring is formed between X₄ and X₉ via a linker selected from the group consisting of a disulfide, thioether, amide, olefin, and triazole.

In some embodiments, the second ring is formed between X₄ and X₁₃. In some embodiments, the second ring is formed between X₄ and X₁₃ via a linker having one or more groups selected from the group consisting of a disulfide, thioether, amide, olefin, ether, alkylene, and triazole. In some embodiments, the second ring is formed between X₄ and X₁₃ via a linker selected from the group consisting of a disulfide, thioether, amide, olefin, and triazole.

In some embodiments, the second ring is formed between X₅ and X₁₀. In some embodiments, the second ring is formed between X₅ and X₁₀ via a linker having one or more groups selected from the group consisting of a disulfide, thioether, amide, olefin, ether, alkylene, and triazole. In some embodiments, the second ring is formed between X₅ and X₁₀ via a linker selected from the group consisting of a disulfide, thioether, amide, olefin, and triazole.

In some embodiments, the second ring is formed between X₃ and X₁₃. In some embodiments, the second ring is formed between X₃ and X₁₃ via a linker having one or more groups selected from the group consisting of a disulfide, thioether, amide, olefin, ether, alkylene, and triazole. In some embodiments, the second ring is formed between X₃ and X₁₃ via a linker selected from the group consisting of a disulfide, thioether, amide, olefin, and triazole.

In some embodiments, the second ring is formed between X₆ and X₉. In some embodiments, the second ring is formed between X₆ and X₉ via a linker having one or more groups selected from the group consisting of a disulfide, thioether, amide, olefin, ether, alkylene, and triazole. In some embodiments, the second ring is formed between X₆ and X₉ via a linker selected from the group consisting of a disulfide, thioether, amide, olefin, and triazole.

In some embodiments, the second ring is formed between X₁₃ and the N-terminus of the peptide. In some embodiments, the second ring is formed between X₁₃ and the N-terminus of the peptide via a linker having one or more groups selected from the group consisting of a disulfide, thioether, amide, olefin, ether, alkylene, and triazole. In some embodiments, the second ring is formed between X₁₃ and the N-terminus of the peptide via a linker selected from the group consisting of a disulfide, thioether, amide, olefin, and triazole.

In some embodiments, the first ring is formed between X₄ and X₉ via a linker having one or more groups selected from the group consisting of a disulfide, thioether, amide, olefin, ether, alkylene, and triazole; and the second ring is formed between X₃ and X₁₃, between X₅ and X₁₀, between X₁₀ and X₁₃, or between X₁₃ and the N-terminus of the peptide via a linker having one or more groups selected from the group consisting of a disulfide, thioether, amide, olefin, ether, alkylene, and triazole.

In some embodiments, the first ring is formed between X₄ and X₉ via a linker selected from the group consisting of a disulfide, thioether, amide, olefin, and triazole; and the second ring is formed between X₃ and X₁₃, between X₅ and X₁₀, between X₁₀ and X₁₃, or between X₁₃ and the N-terminus of the peptide via a linker selected from the group consisting of a disulfide, thioether, amide, olefin, and triazole.

In some embodiments, the first ring is formed between X₄ and X₁₃ via a linker having one or more groups selected from the group consisting of a disulfide, thioether, amide, olefin, ether, alkylene, and triazole; and the second ring is formed between X₅ and X₁₀ or between X₆ and X₉ via a linker having one or more groups selected from the group consisting of a disulfide, thioether, amide, olefin, ether, alkylene, and triazole.

In some embodiments, the first ring is formed between X₄ and X₁₃ via a linker selected from the group consisting of a disulfide, thioether, amide, olefin, and triazole; and the second ring is formed between X₅ and X₁₀ or between X₆ and X₉ via a linker selected from the group consisting of a disulfide, thioether, amide, olefin, and triazole.

In some embodiments, the first ring is formed between X₆ and X₉ via a linker having one or more groups selected from the group consisting of a disulfide, thioether, amide, olefin, ether, alkylene, and triazole; and the second ring is formed between X₃ and X₁₃, between X₄ and X₁₃, between X₅ and X₁₀, between X₁₀ and X₁₃, or between X₁₃ and the N-terminus of the peptide via a linker having one or more groups selected from the group consisting of a disulfide, thioether, amide, olefin, ether, alkylene, and triazole.

In some embodiments, the first ring is formed between X₆ and X₉ via a linker selected from the group consisting of a disulfide, thioether, amide, olefin, and triazole; and the second ring is formed between X₃ and X₁₃, between X₄ and X₁₃, between X₅ and X₁₀, between X₁₀ and X₁₃, or between X₁₃ and the N-terminus of the peptide via a linker selected from the group consisting of a disulfide, thioether, amide, olefin, and triazole.

In some embodiments, the peptide comprises at least one polyethylene glycol chain. In some embodiments, the peptide comprises no more than five, no more than four, no more than three, no more than two, or no more than one polyethylene glycol chain.

In some embodiments, each polyethylene glycol chain, independently, terminates in an ammonium group of a methyl group. In some embodiments, the polyethylene glycol chain terminates in an ammonium group. In some embodiments, the polyethylene glycol chain terminates in a methyl group.

In some embodiments, the peptide comprises a polyethylene glycol chain having the following structure:

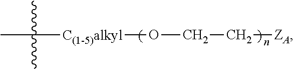

wherein:
Z_{A} is —OCH₃ or —N⁺(CH₃)₃; and
n is an integer from 2 to 24.

In some embodiments, the peptide comprises a polyethylene glycol chain having the following structure:

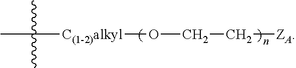

In some embodiments, the peptide comprises a polyethylene glycol chain having the following structure:

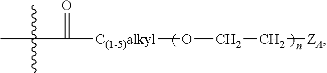

wherein:
Z_{A} is —OCH₃ or —N⁺(CH₃)₃; and
n is an integer from 2 to 24.

In some embodiments, the peptide comprises a polyethylene glycol chain having the following structure:

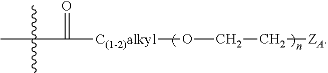

In some embodiments, Z_{A} is —OCH₃. In some embodiments, Z_{A} is —N⁺(CH₃)₃.

In some embodiments, n is an integer from 2 to 15. In some embodiments, n is an integer from 2 to 5. 2. In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 9. In some embodiments, n is 10. In some embodiments, n is 11. In some embodiments, n is 12. In some embodiments, n is 14. In some embodiments, n is 14. In some embodiments, n is 15.

In some embodiments, R₁, R₂, or any amino acid of the peptide is conjugated to a polyethylene glycol chain.

Non-limiting examples of polyethylene glycol chains are provided in Table 2.

TABLE 2
Exemplary polyethylene glycol chains

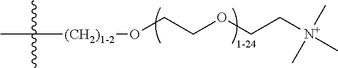

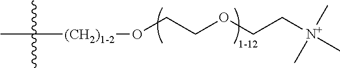

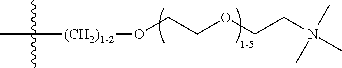

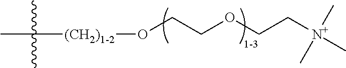

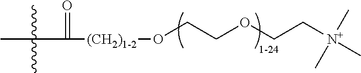

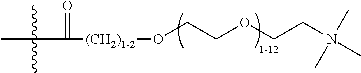

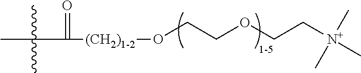

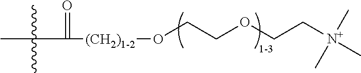

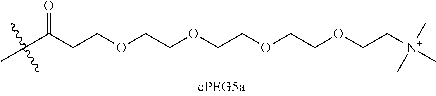 [SMILES: *C(=O)CCOCCOCCOCCOCC[N+](C)(C)C]

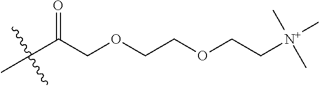 [SMILES: *C(=O)COCCOCC[N+](C)(C)C]

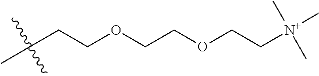 [SMILES: *CCOCCOCC[N+](C)(C)C]

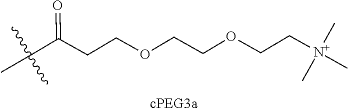 [SMILES: *C(=O)CCOCCOCC[N+](C)(C)C]

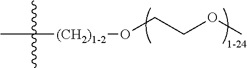

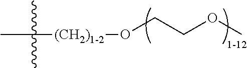

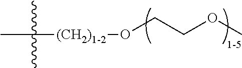

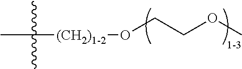

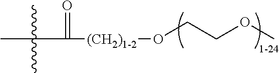

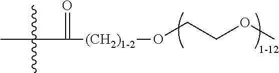

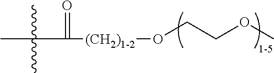

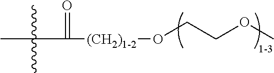

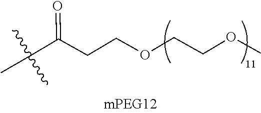

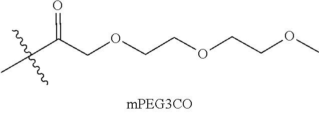 [SMILES: *C(=O)COCCOCCOC]

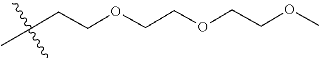 [SMILES: *CCOCCOCCOC]

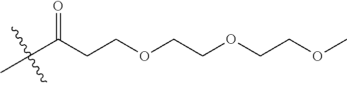 [SMILES: *C(=O)CCOCCOCCOC]

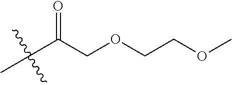 [SMILES: *C(=O)COCCOC]

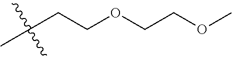 [SMILES: *CCOCCOC]

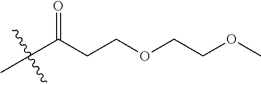 [SMILES: *C(=O)CCOCCOC]

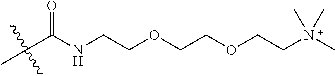

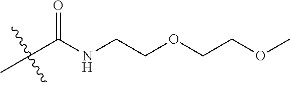 [SMILES: *C(=O)NCCOCCOC]

In some embodiments, the peptide comprises at least one lipophilic substituent. In some embodiments, the peptide comprises one lipophilic substituent. In some embodiments, the peptide comprises no more than three, no more than two, or no more than one lipophilic substituent. In some embodiments, the peptide comprises no more than one lipophilic substituent.

In some embodiments, the peptide comprises a lipophilic substituent having the following structure:

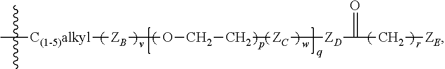

wherein:
Z_{B} is

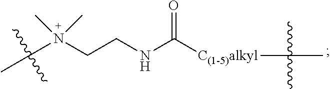

Z_{C} is Z_{C1}, Z_{C2}, or Z_{C3};
Z_{C1} is

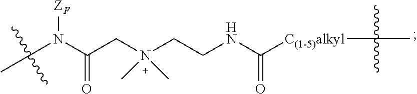

Z_{C2} is

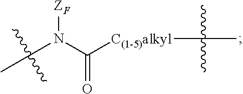

Z_{C3} is

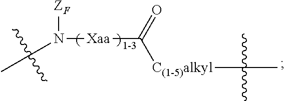

Z_{D} is

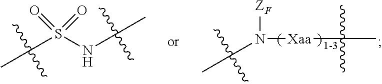

Z_{E} is —H, —COOH, or tetrazolyl;
Z_{F} is —H or —CH₃;
Xaa is independently for each occurrence,

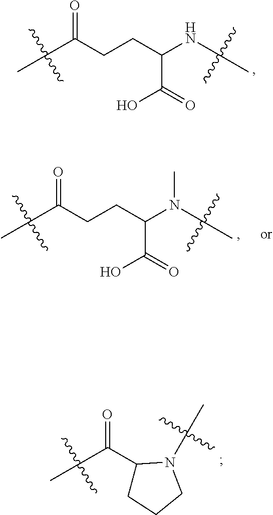 [SMILES: *NC(CCC(*)=O)C(=O)O ; *C(=O)CCC(C(=O)O)N(*)C ; *C(=O)C1CCCN1*]

p, independently for each occurrence, is 1, 2, 3, 4, 5, or 6;
q is 1, 2, 3, 4, 5, or 6;
r is an integer from 6 to 24;
v is 0 or 1; and
w, independently for each occurrence, is 0 or 1.

In some embodiments, the peptide comprises a lipophilic substituent having the following structure:

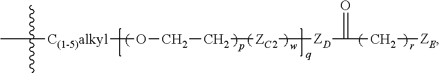

wherein:
Z_{C2} is

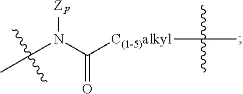

Z_{D} is

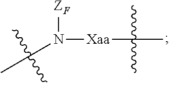

Z_{E} is —H, —COOH, or tetrazolyl;
Z_{F} is —H or —CH₃;
Xaa is

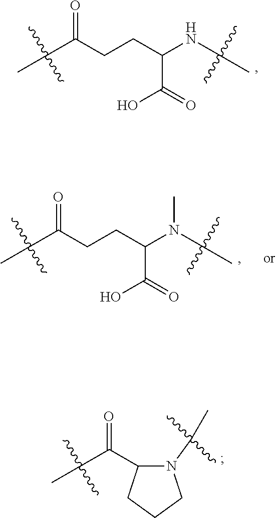 [SMILES: *NC(CCC(*)=O)C(=O)O ; *C(=O)CCC(C(=O)O)N(*)C ; *C(=O)C1CCCN1*]

p, independently for each occurrence, is 1, 2, 3, 4, 5, or 6;
q is 1, 2, 3, 4, 5, or 6;
r is an integer from 6 to 24; and
w, independently for each occurrence, is 0 or 1.

In some embodiments, the peptide comprises a lipophilic substituent having the following structure:

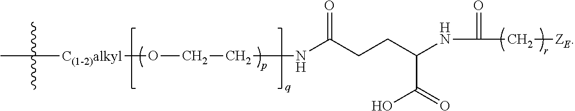

In some embodiments, the peptide comprises a lipophilic substituent having the following structure:

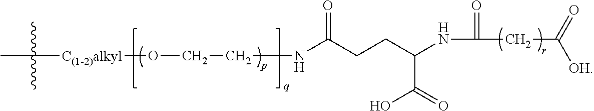

In some embodiments, the peptide comprises a lipophilic substituent having the following structure:

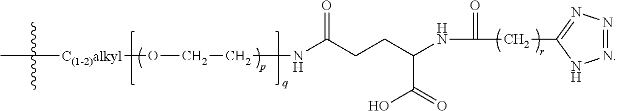

In some embodiments, the peptide comprises a lipophilic substituent having the following structure:

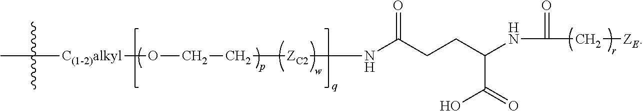

In some embodiments, the peptide comprises a lipophilic substituent having the following structure:

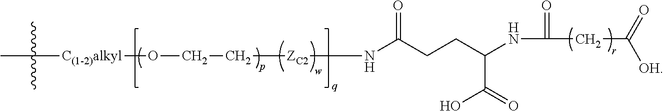

In some embodiments, the peptide comprises a lipophilic substituent having the following structure:

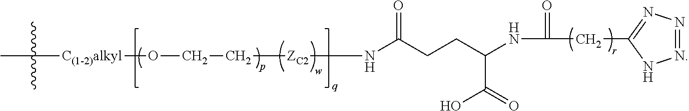

In some embodiments, the peptide comprises a lipophilic substituent having the following structure:

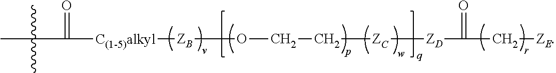

wherein:

Z_{B} is

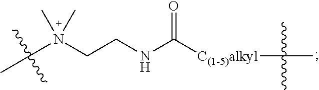

Z_{C} is Z_{C1}, Z_{C2}, or Z_{C3};

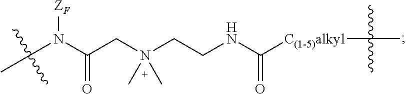

Z_{C2} is

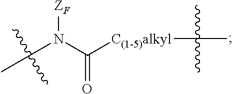

Z_{C3} is

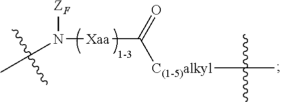

Z_{D} is

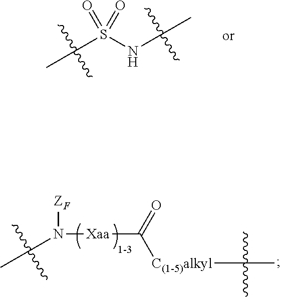

Z_{E} is —H, —COOH, or tetrazolyl;
Z_{F} is —H or —CH₃;
Xaa is, independently for each occurrence,

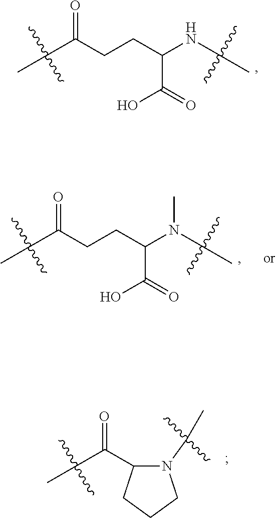 [SMILES: *NC(CCC(*)=O)C(=O)O ; *C(=O)CCC(C(=O)O)N(*)C ; *C(=O)C1CCCN1*]

p, independently for each occurrence, is 1, 2, 3, 4, 5, or 6;
q is 1, 2, 3, 4, 5, or 6;
r is an integer from 6 to 24;
v is 0 or 1; and
w, independently for each occurrence, is 0 or 1.

In some embodiments, the peptide comprises a lipophilic substituent having the following structure:

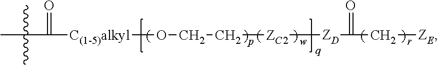

wherein:
Z_{C2} is

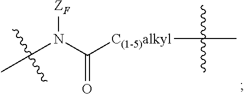

Z_{D} is

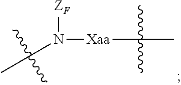

Z_{E} is —H, —COOH, or tetrazolyl;
Z_{F} is —H or —CH₃;
Xaa is

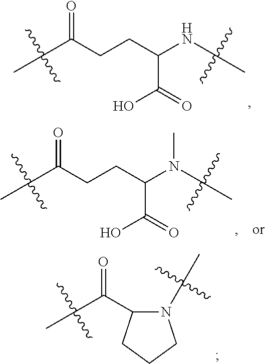

p, independently for each occurrence, is 1, 2, 3, 4, 5, or 6;
q is 1, 2, 3, 4, 5, or 6;
r is an integer from 6 to 24; and
w, independently for each occurrence, is 0 or 1.

In some embodiments, the peptide comprises a lipophilic substituent having the following structure:

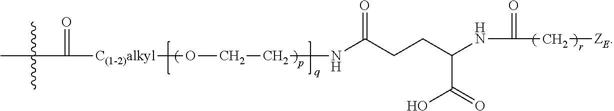

In some embodiments, the peptide comprises a lipophilic substituent having the following structure:

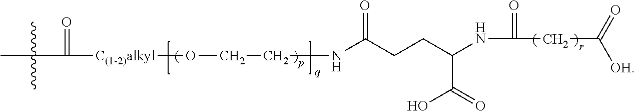

In some embodiments, the peptide comprises a lipophilic substituent having the following structure:

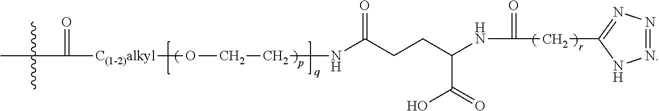

In some embodiments, the peptide comprises a lipophilic substituent having the following structure:

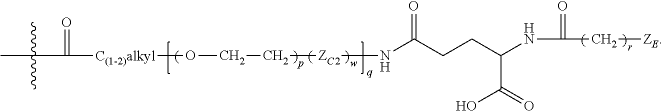

In some embodiments, the peptide comprises a lipophilic substituent having the following structure:

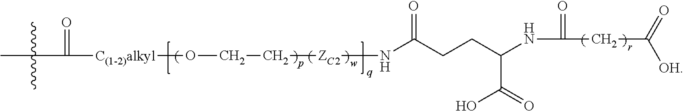

In some embodiments, the peptide comprises a lipophilic substituent having the following structure:

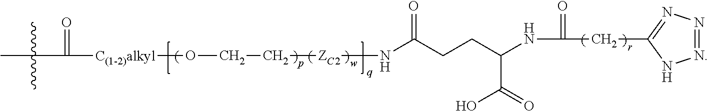

In some embodiments, Z_{c} is

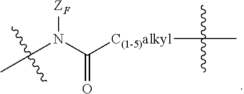

In some embodiments, Z_{D} is

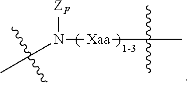

In some embodiments, Z_{D} is

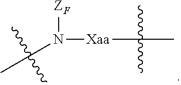

In some embodiments, Z_{E} is —H. In some embodiments, Z_{E} is —COOH. In some embodiments, Z_{E} is tetrazolyl.

In some embodiments, Z_{F} is —H. In some embodiments, Z_{F} is —CH₃.

In some embodiments, Xaa is, independently for each occurrence,

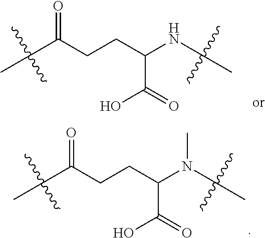

In some embodiments, Xaa is

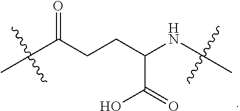 [SMILES: *NC(CCC(*)=O)C(=O)O]

In some embodiments, p is 1, 2, 3, or 4. In some embodiments, p is 1. In some embodiments, p is 2. In some embodiments, p is 3. In some embodiments, p is 4. In some embodiments, p is 5. In some embodiments, p is 6.

In some embodiments, q is 1, 2, 3, or 4. In some embodiments, q is 1. In some embodiments, q is 2. In some embodiments, q is 3. In some embodiments, q is 4. In some embodiments, q is 5. In some embodiments, q is 6.

In some embodiments, r is an integer between 10 and 20. In some embodiments, r is 10. In some embodiments, r is 11. In some embodiments, r is 12. In some embodiments, r is 13. In some embodiments, r is 14. In some embodiments, r is 15. In some embodiments, r is 16. In some embodiments, r is 17. In some embodiments, r is 18. In some embodiments, r is 19. In some embodiments, r is 20.

In some embodiments, w is 0. In some embodiments, w is 1.

In some embodiments, R₁, R₂, or any amino acid of the peptide is conjugated to a lipophilic substituent.

Non-limiting examples of lipophilic substituents are provided in Table 3A.

TABLE 3A
Exemplary lipophilic substituents

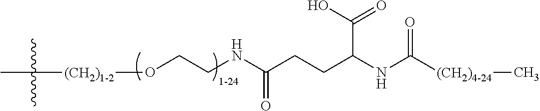

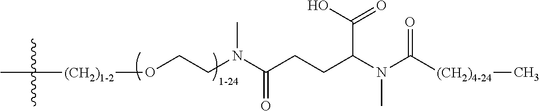

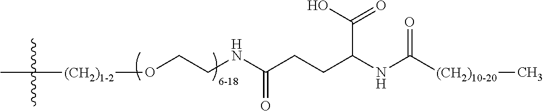

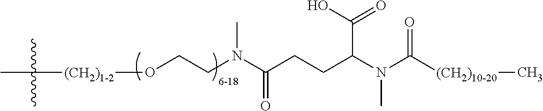

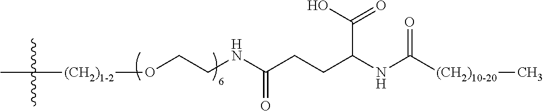

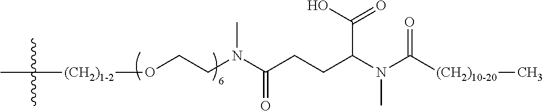

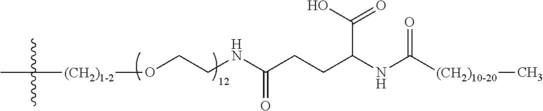

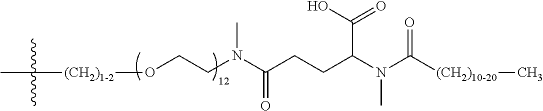

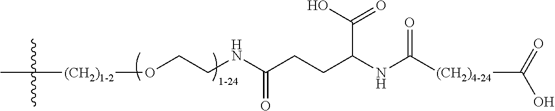

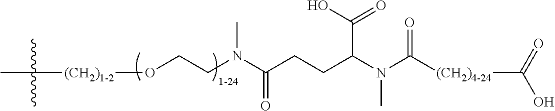

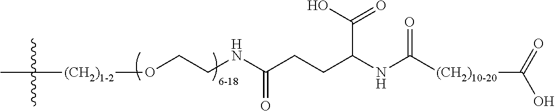

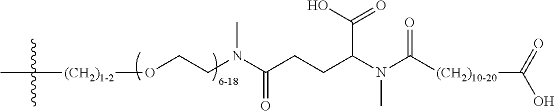

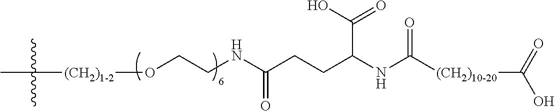

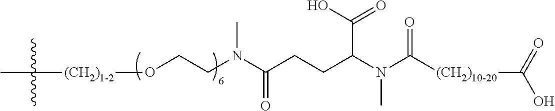

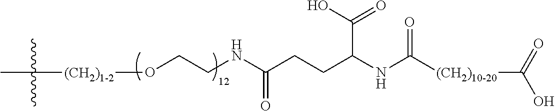

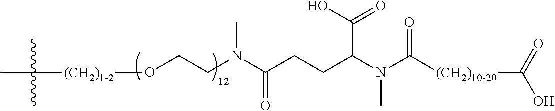

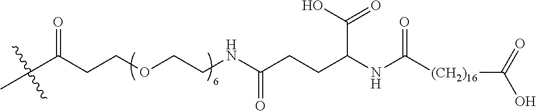

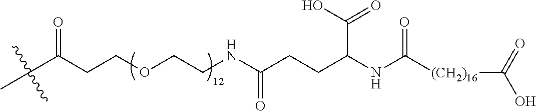

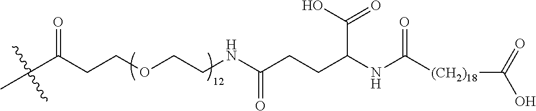

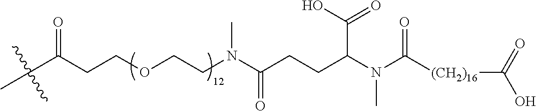

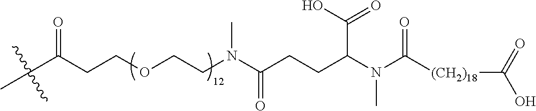

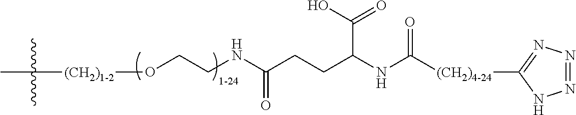

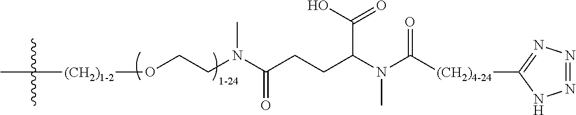

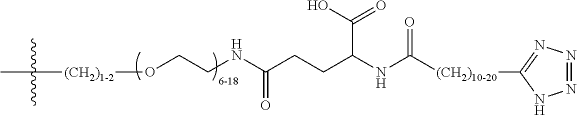

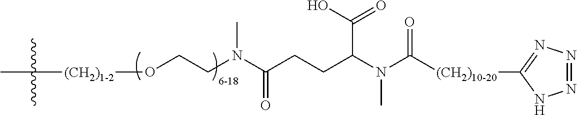

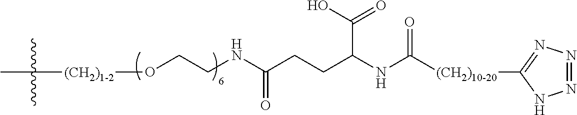

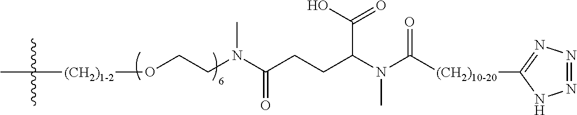

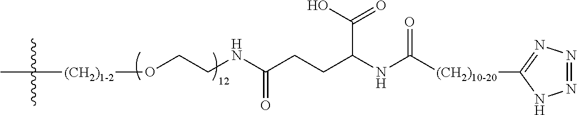

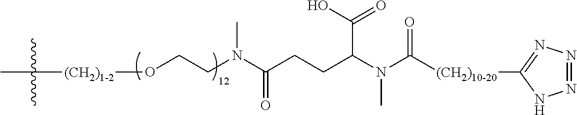

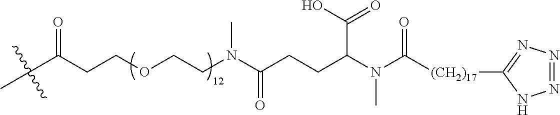

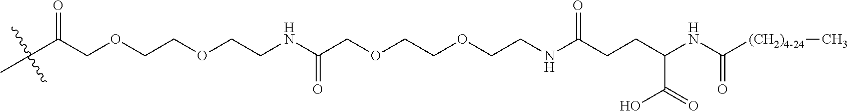 [SMILES: *C(=O)COCCOCCNC(=O)COCCOCCNC(=O)CCC(NC(=O)CC)C(=O)O]

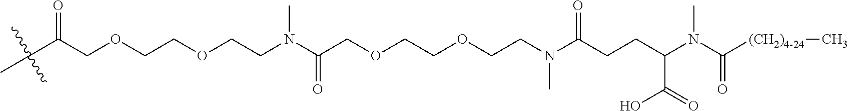 [SMILES: *C(=O)COCCOCCN(C)C(=O)COCCOCCN(C)C(=O)CCC(C(=O)O)N(C)C(=O)CC]

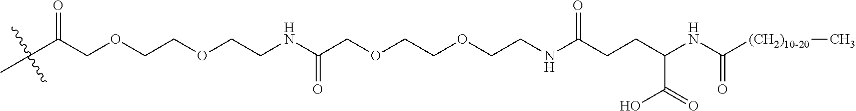 [SMILES: *C(=O)COCCOCCNC(=O)COCCOCCNC(=O)CCC(NC(=O)CC)C(=O)O]

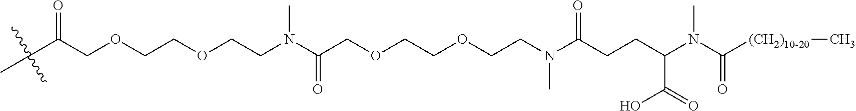 [SMILES: *C(=O)COCCOCCN(C)C(=O)COCCOCCN(C)C(=O)CCC(C(=O)O)N(C)C(=O)CC]

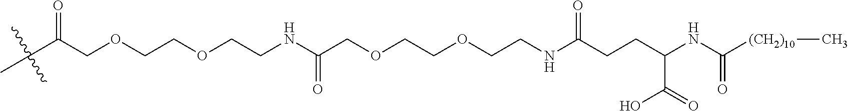

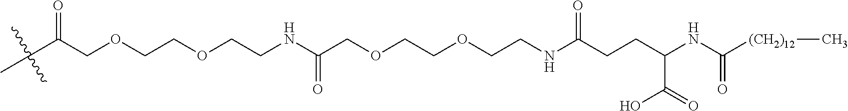

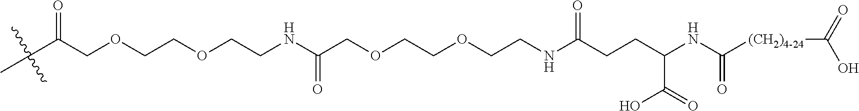 [SMILES: *C(=O)COCCOCCNC(=O)COCCOCCNC(=O)CCC(NC(=O)CC(=O)O)C(=O)O]

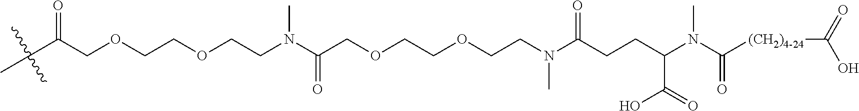 [SMILES: *C(=O)COCCOCCN(C)C(=O)COCCOCCN(C)C(=O)CCC(C(=O)O)N(C)C(=O)CC(=O)O]

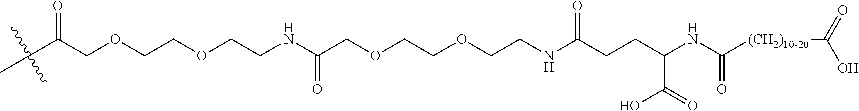 [SMILES: *C(=O)COCCOCCNC(=O)COCCOCCNC(=O)CCC(NC(=O)CC(=O)O)C(=O)O]

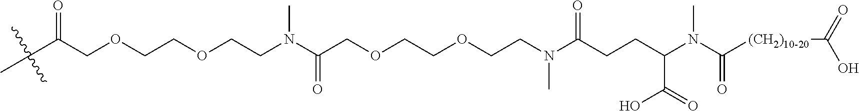 [SMILES: *C(=O)COCCOCCN(C)C(=O)COCCOCCN(C)C(=O)CCC(C(=O)O)N(C)C(=O)CC(=O)O]

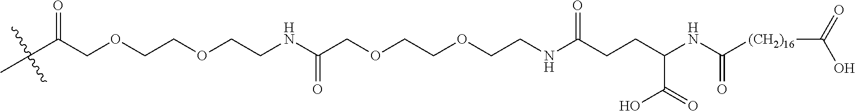

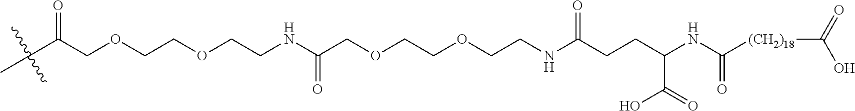

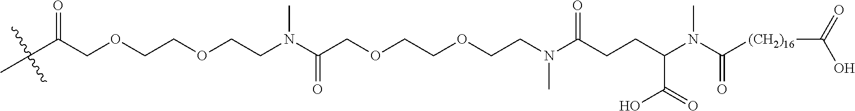

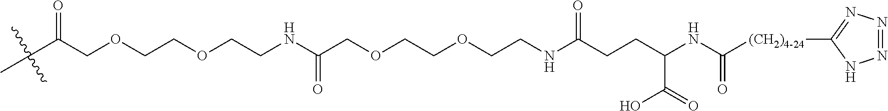 [SMILES: *C(=O)COCCOCCNC(=O)COCCOCCNC(=O)CCC(NC(=O)Cc1nnn[nH]1)C(=O)O]

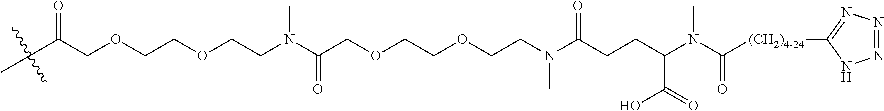 [SMILES: *C(=O)COCCOCCN(C)C(=O)COCCOCCN(C)C(=O)CCC(C(=O)O)N(C)C(=O)Cc1nnn[nH]1]

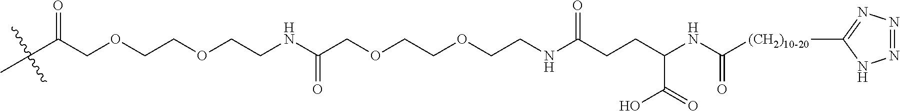 [SMILES: *C(=O)COCCOCCNC(=O)COCCOCCNC(=O)CCC(NC(=O)Cc1nnn[nH]1)C(=O)O]

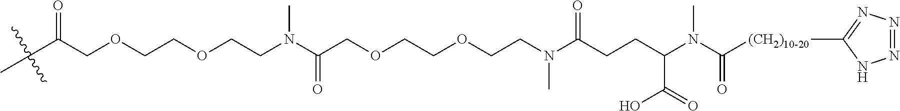 [SMILES: *C(=O)COCCOCCN(C)C(=O)COCCOCCN(C)C(=O)CCC(C(=O)O)N(C)C(=O)Cc1nnn[nH]1]

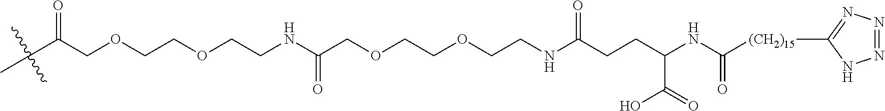

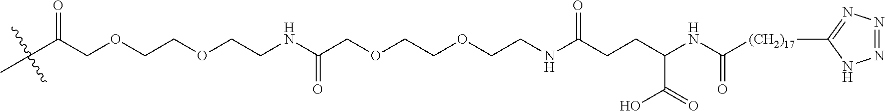

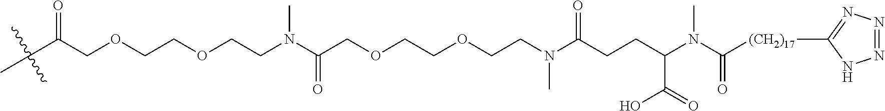

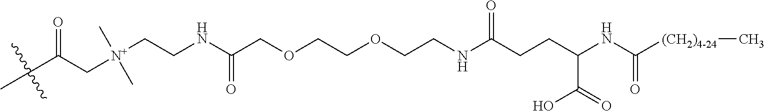 [SMILES: *C(=O)C[N+](C)(C)CCNC(=O)COCCOCCNC(=O)CCC(NC(=O)CC)C(=O)O]

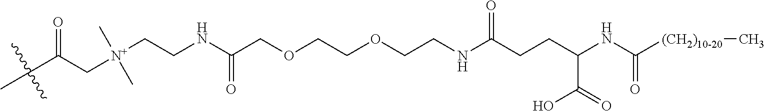 [SMILES: *C(=O)C[N+](C)(C)CCNC(=O)COCCOCCNC(=O)CCC(NC(=O)CC)C(=O)O]

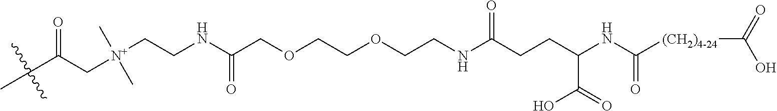 [SMILES: *C(=O)C[N+](C)(C)CCNC(=O)COCCOCCNC(=O)CCC(NC(=O)CC(=O)O)C(=O)O]

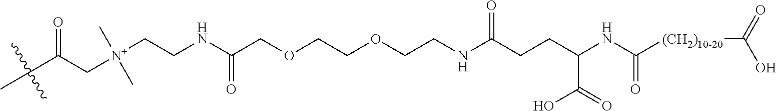 [SMILES: *C(=O)C[N+](C)(C)CCNC(=O)COCCOCCNC(=O)CCC(NC(=O)CC(=O)O)C(=O)O]

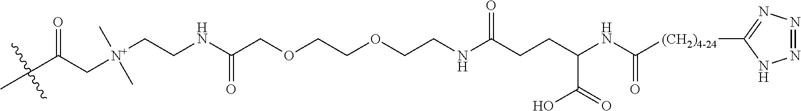 [SMILES: *C(=O)C[N+](C)(C)CCNC(=O)COCCOCCNC(=O)CCC(NC(=O)Cc1nnn[nH]1)C(=O)O]

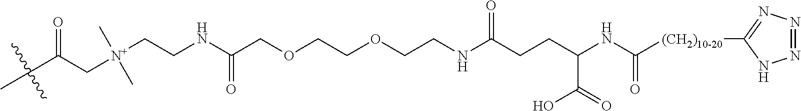 [SMILES: *C(=O)C[N+](C)(C)CCNC(=O)COCCOCCNC(=O)CCC(NC(=O)Cc1nnn[nH]1)C(=O)O]

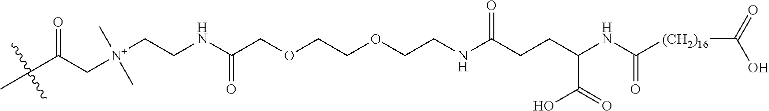

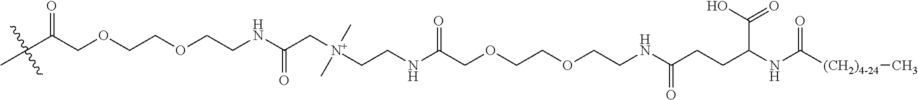 [SMILES: *C(=O)COCCOCCNC(=O)C[N+](C)(C)CCNC(=O)COCCOCCNC(=O)CCC(NC(=O)CC)C(=O)O]

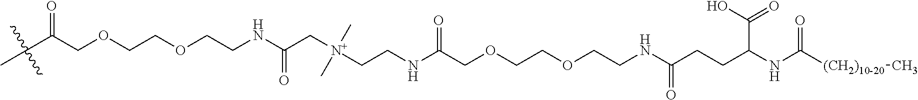 [SMILES: *C(=O)COCCOCCNC(=O)C[N+](C)(C)CCNC(=O)COCCOCCNC(=O)CCC(NC(=O)CC)C(=O)O]

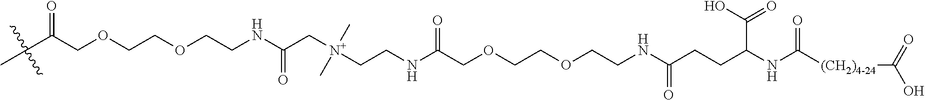 [SMILES: *C(=O)COCCOCCNC(=O)C[N+](C)(C)CCNC(=O)COCCOCCNC(=O)CCC(NC(=O)CC(=O)O)C(=O)O]

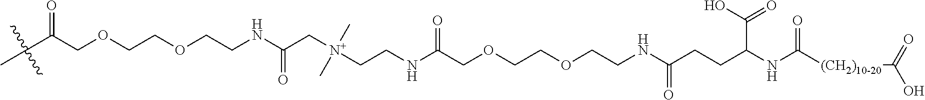 [SMILES: *C(=O)COCCOCCNC(=O)C[N+](C)(C)CCNC(=O)COCCOCCNC(=O)CCC(NC(=O)CC(=O)O)C(=O)O]

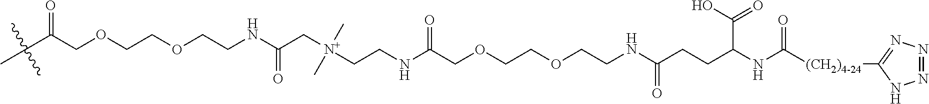 [SMILES: *C(=O)COCCOCCNC(=O)C[N+](C)(C)CCNC(=O)COCCOCCNC(=O)CCC(NC(=O)Cc1nnn[nH]1)C(=O)O]

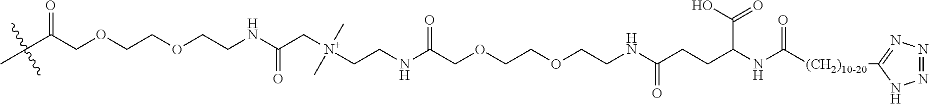 [SMILES: *C(=O)COCCOCCNC(=O)C[N+](C)(C)CCNC(=O)COCCOCCNC(=O)CCC(NC(=O)Cc1nnn[nH]1)C(=O)O]

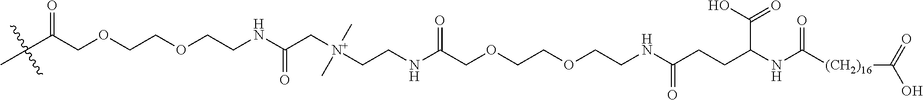

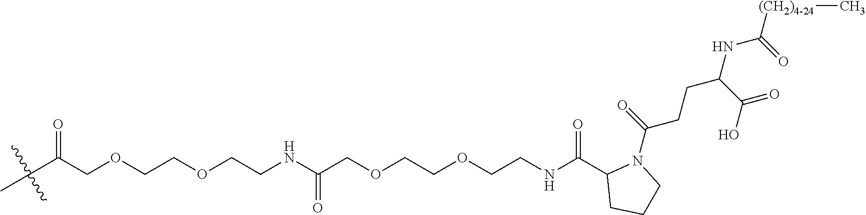 [SMILES: *C(=O)COCCOCCNC(=O)COCCOCCNC(=O)C1CCCN1C(=O)CCC(NC(=O)CC)C(=O)O]

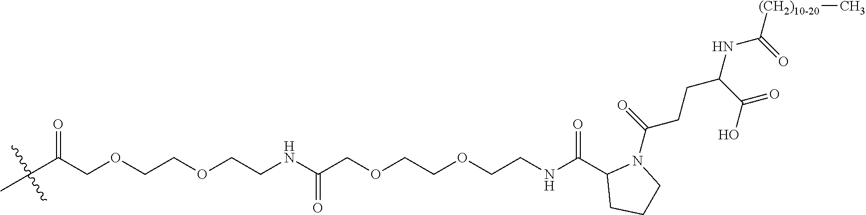 [SMILES: *C(=O)COCCOCCNC(=O)COCCOCCNC(=O)C1CCCN1C(=O)CCC(NC(=O)CC)C(=O)O]

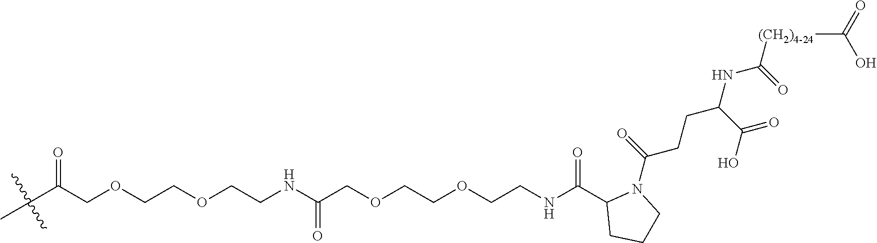 [SMILES: *C(=O)COCCOCCNC(=O)COCCOCCNC(=O)C1CCCN1C(=O)CCC(NC(=O)CC(=O)O)C(=O)O]

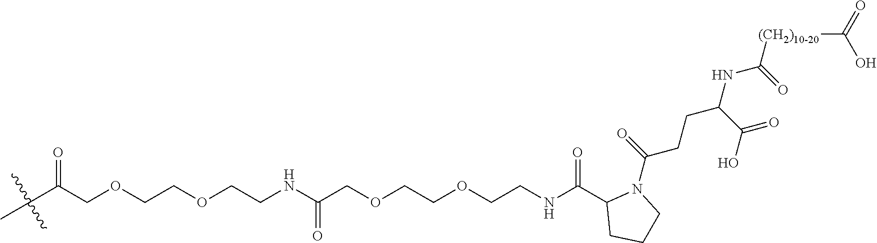 [SMILES: *C(=O)COCCOCCNC(=O)COCCOCCNC(=O)C1CCCN1C(=O)CCC(NC(=O)CC(=O)O)C(=O)O]

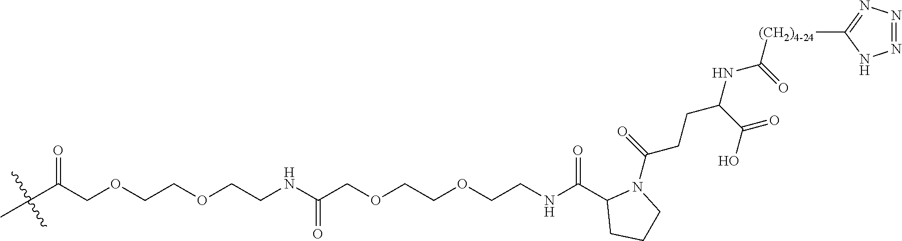 [SMILES: *C(=O)COCCOCCNC(=O)COCCOCCNC(=O)C1CCCN1C(=O)CCC(NC(=O)Cc1nnn[nH]1)C(=O)O]

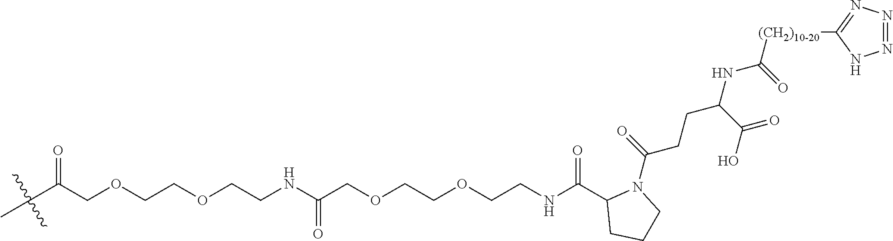 [SMILES: *C(=O)COCCOCCNC(=O)COCCOCCNC(=O)C1CCCN1C(=O)CCC(NC(=O)Cc1nnn[nH]1)C(=O)O]

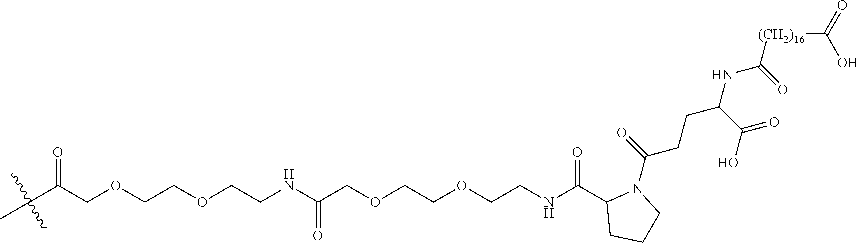

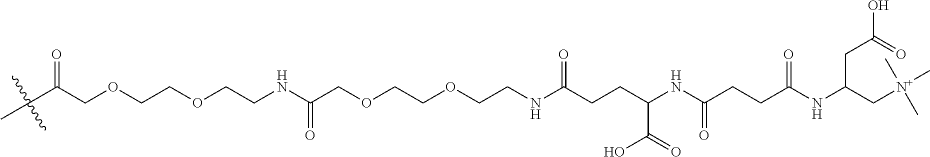 [SMILES: *C(=O)COCCOCCNC(=O)COCCOCCNC(=O)CCC(NC(=O)CCC(=O)NC(CC(=O)O)C[N+](C)(C)C)C(=O)O]

Further non-limiting examples of lipophilic substituents are provided in Table 3B.

TABLE 3B
Exemplary lipophilic substituents

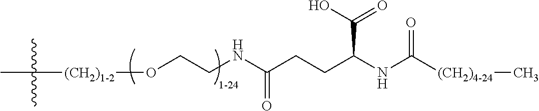

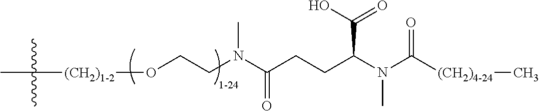

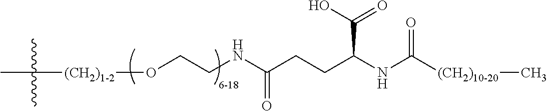

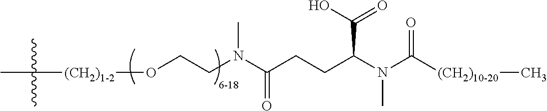

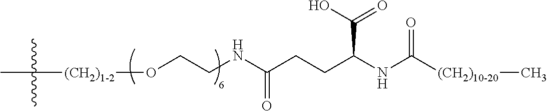

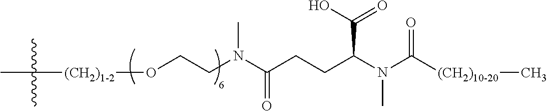

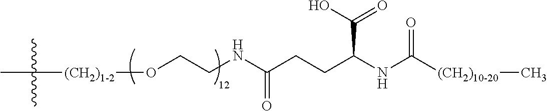

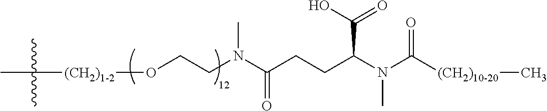

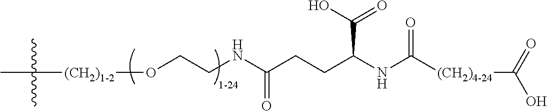

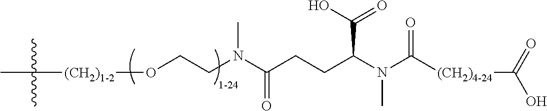

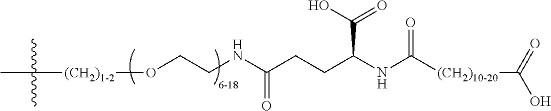

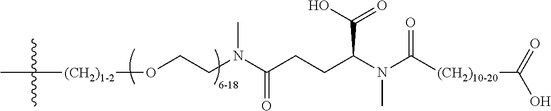

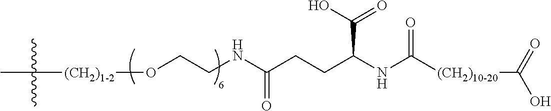

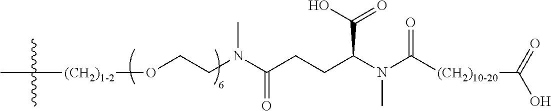

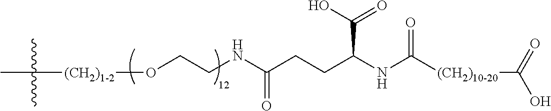

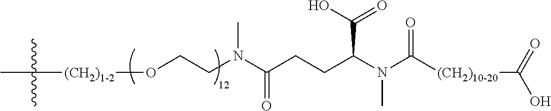

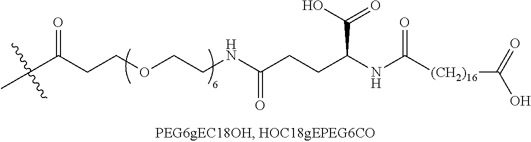

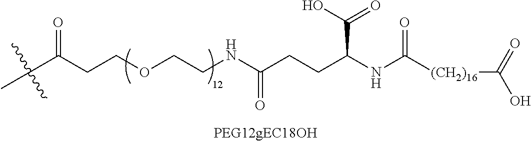

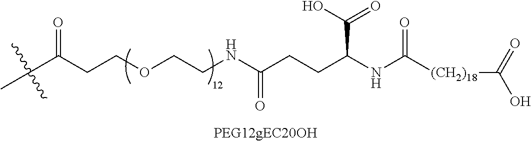

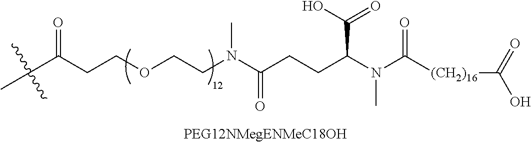

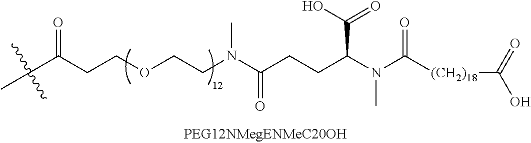

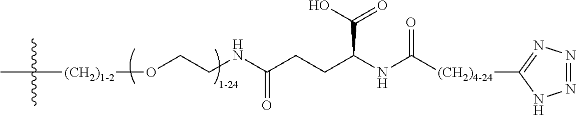

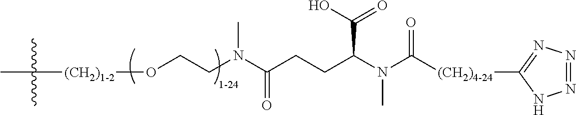

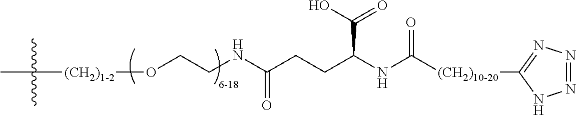

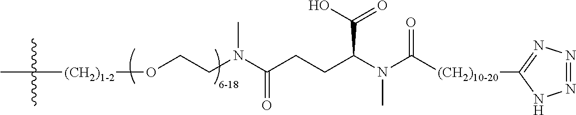

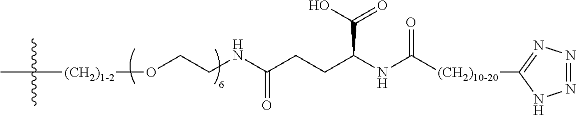

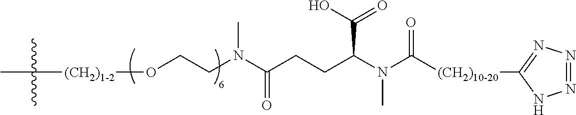

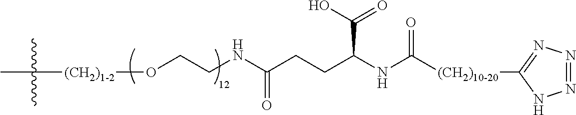

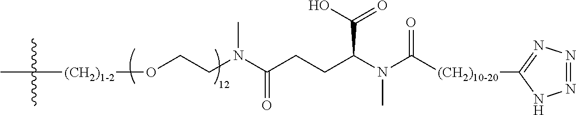

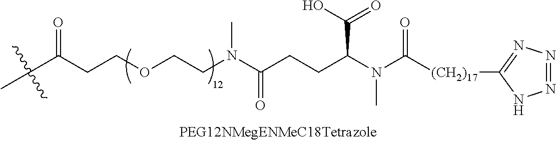

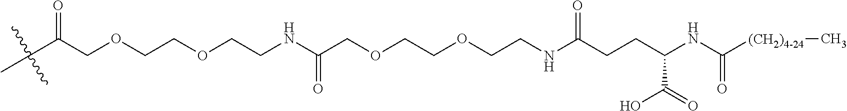 [SMILES: *C(=O)COCCOCCNC(=O)COCCOCCNC(=O)CC[C@H](NC(=O)CC)C(=O)O]

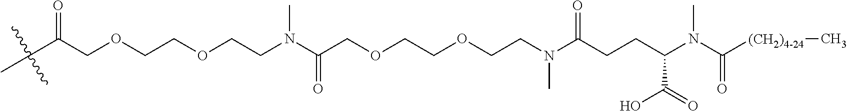 [SMILES: *C(=O)COCCOCCN(C)C(=O)COCCOCCN(C)C(=O)CC[C@@H](C(=O)O)N(C)C(=O)CC]

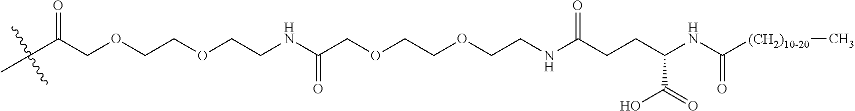 [SMILES: *C(=O)COCCOCCNC(=O)COCCOCCNC(=O)CC[C@H](NC(=O)CC)C(=O)O]

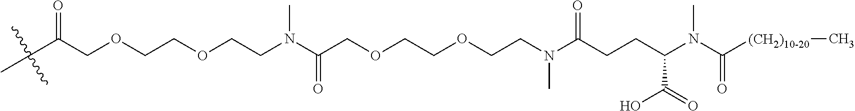 [SMILES: *C(=O)COCCOCCN(C)C(=O)COCCOCCN(C)C(=O)CC[C@@H](C(=O)O)N(C)C(=O)CC]

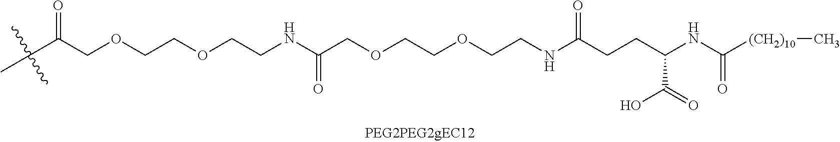

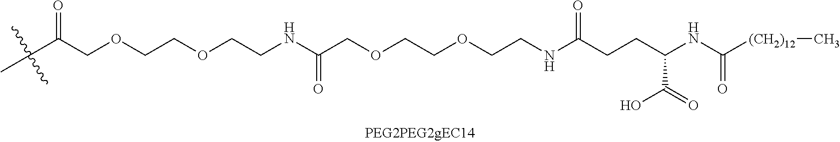

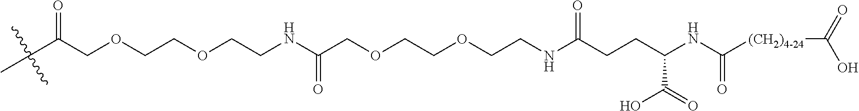 [SMILES: *C(=O)COCCOCCNC(=O)COCCOCCNC(=O)CC[C@H](NC(=O)CC(=O)O)C(=O)O]

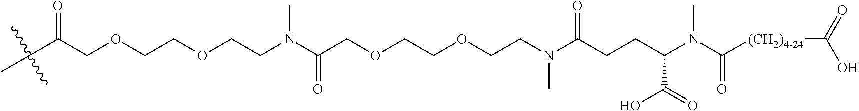 [SMILES: *C(=O)COCCOCCN(C)C(=O)COCCOCCN(C)C(=O)CC[C@@H](C(=O)O)N(C)C(=O)CC(=O)O]

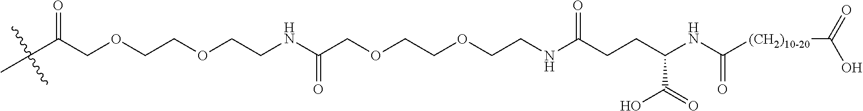 [SMILES: *C(=O)COCCOCCNC(=O)COCCOCCNC(=O)CC[C@H](NC(=O)CC(=O)O)C(=O)O]

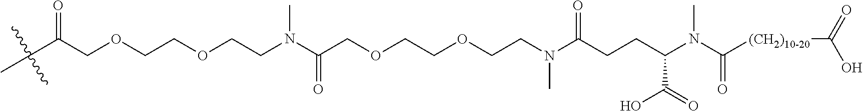 [SMILES: *C(=O)COCCOCCN(C)C(=O)COCCOCCN(C)C(=O)CC[C@@H](C(=O)O)N(C)C(=O)CC(=O)O]

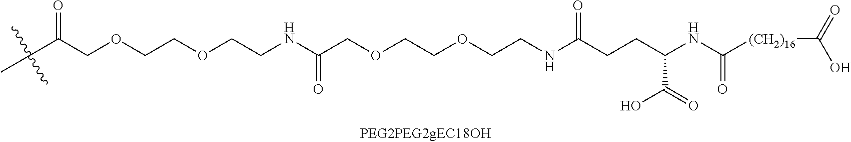

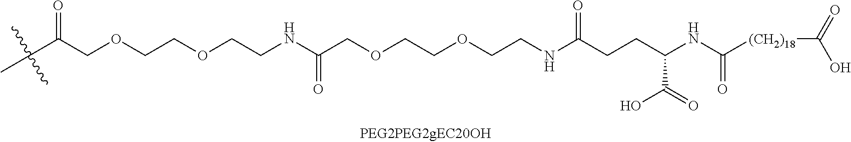

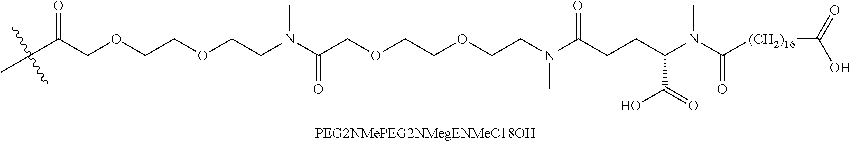

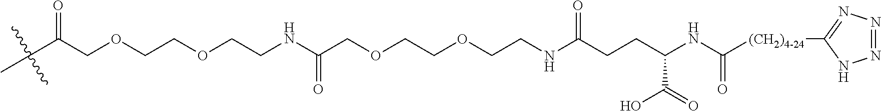 [SMILES: *C(=O)COCCOCCNC(=O)COCCOCCNC(=O)CC[C@H](NC(=O)Cc1nnn[nH]1)C(=O)O]

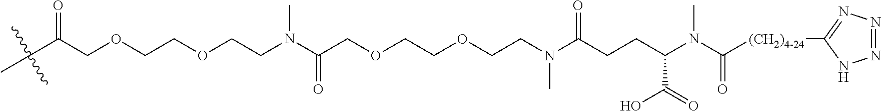 [SMILES: *C(=O)COCCOCCN(C)C(=O)COCCOCCN(C)C(=O)CC[C@@H](C(=O)O)N(C)C(=O)Cc1nnn[nH]1]

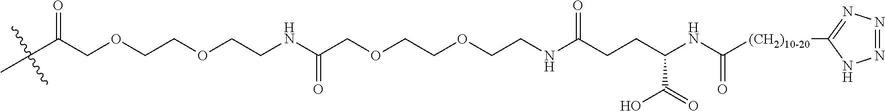 [SMILES: *C(=O)COCCOCCNC(=O)COCCOCCNC(=O)CC[C@H](NC(=O)Cc1nnn[nH]1)C(=O)O]

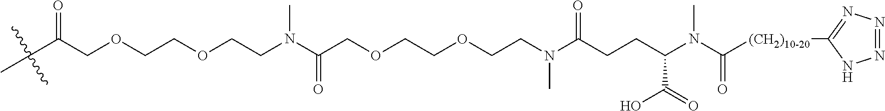 [SMILES: *C(=O)COCCOCCN(C)C(=O)COCCOCCN(C)C(=O)CC[C@@H](C(=O)O)N(C)C(=O)Cc1nnn[nH]1]

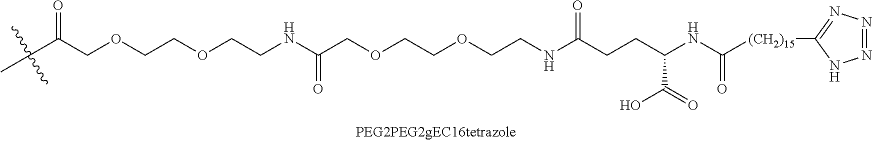

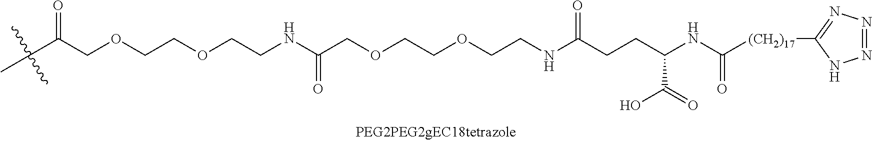

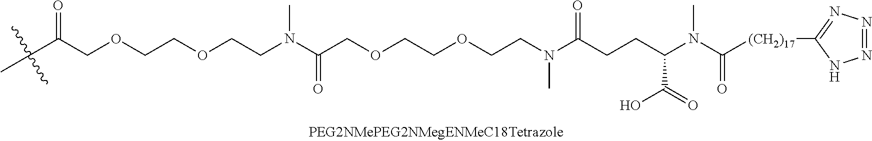

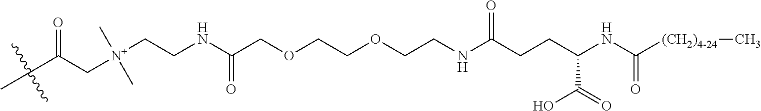 [SMILES: *C(=O)C[N+](C)(C)CCNC(=O)COCCOCCNC(=O)CC[C@H](NC(=O)CC)C(=O)O]

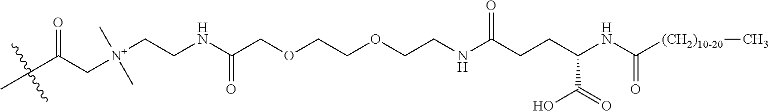 [SMILES: *C(=O)C[N+](C)(C)CCNC(=O)COCCOCCNC(=O)CC[C@H](NC(=O)CC)C(=O)O]

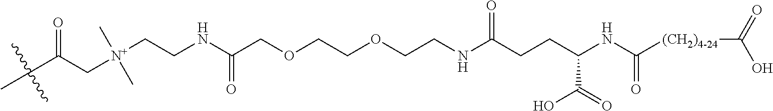 [SMILES: *C(=O)C[N+](C)(C)CCNC(=O)COCCOCCNC(=O)CC[C@H](NC(=O)CC(=O)O)C(=O)O]

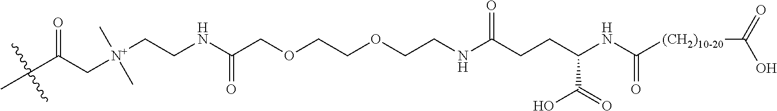 [SMILES: *C(=O)C[N+](C)(C)CCNC(=O)COCCOCCNC(=O)CC[C@H](NC(=O)CC(=O)O)C(=O)O]

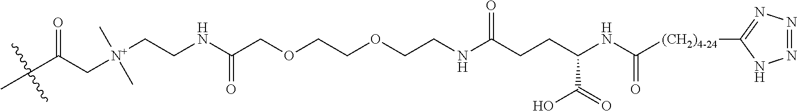 [SMILES: *C(=O)C[N+](C)(C)CCNC(=O)COCCOCCNC(=O)CC[C@H](NC(=O)Cc1nnn[nH]1)C(=O)O]

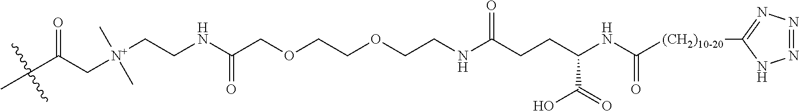 [SMILES: *C(=O)C[N+](C)(C)CCNC(=O)COCCOCCNC(=O)CC[C@H](NC(=O)Cc1nnn[nH]1)C(=O)O]

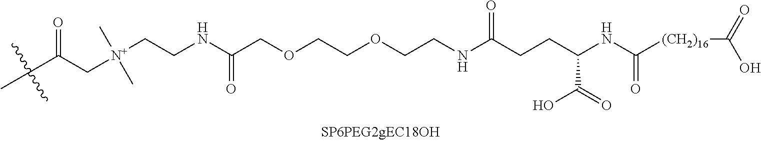

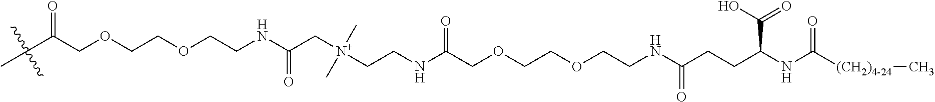 [SMILES: *C(=O)COCCOCCNC(=O)C[N+](C)(C)CCNC(=O)COCCOCCNC(=O)CC[C@H](NC(=O)CC)C(=O)O]

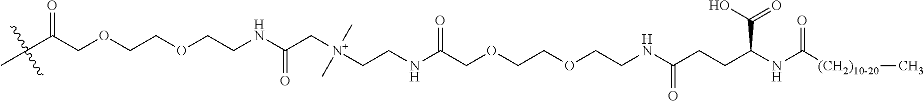 [SMILES: *C(=O)COCCOCCNC(=O)C[N+](C)(C)CCNC(=O)COCCOCCNC(=O)CC[C@H](NC(=O)CC)C(=O)O]

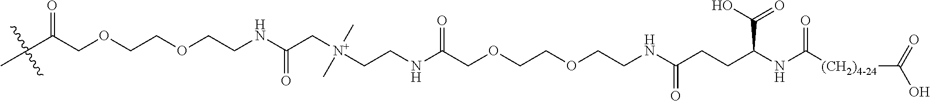 [SMILES: *C(=O)COCCOCCNC(=O)C[N+](C)(C)CCNC(=O)COCCOCCNC(=O)CC[C@H](NC(=O)CC(=O)O)C(=O)O]

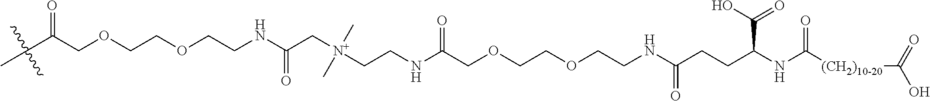 [SMILES: *C(=O)COCCOCCNC(=O)C[N+](C)(C)CCNC(=O)COCCOCCNC(=O)CC[C@H](NC(=O)CC(=O)O)C(=O)O]

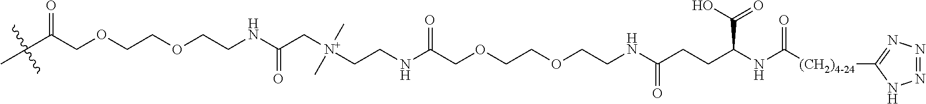 [SMILES: *C(=O)COCCOCCNC(=O)C[N+](C)(C)CCNC(=O)COCCOCCNC(=O)CC[C@H](NC(=O)Cc1nnn[nH]1)C(=O)O]

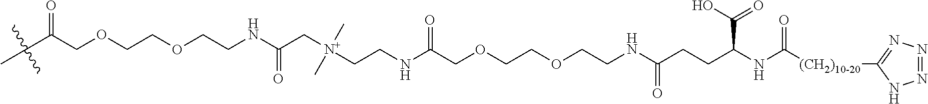 [SMILES: *C(=O)COCCOCCNC(=O)C[N+](C)(C)CCNC(=O)COCCOCCNC(=O)CC[C@H](NC(=O)Cc1nnn[nH]1)C(=O)O]

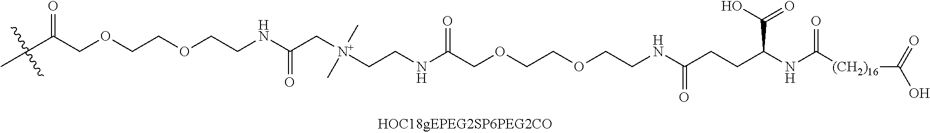

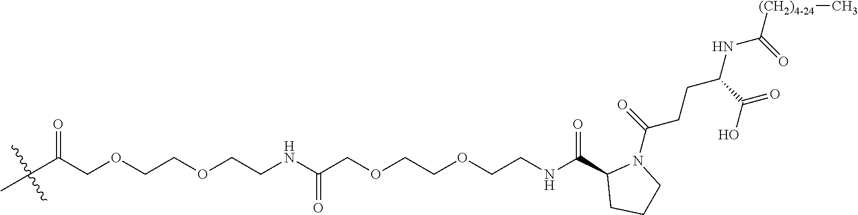 [SMILES: *C(=O)COCCOCCNC(=O)COCCOCCNC(=O)[C@@H]1CCCN1C(=O)CC[C@H](NC(=O)CC)C(=O)O]

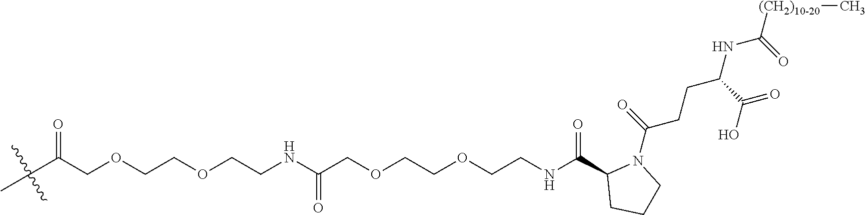 [SMILES: *C(=O)COCCOCCNC(=O)COCCOCCNC(=O)[C@@H]1CCCN1C(=O)CC[C@H](NC(=O)CC)C(=O)O]

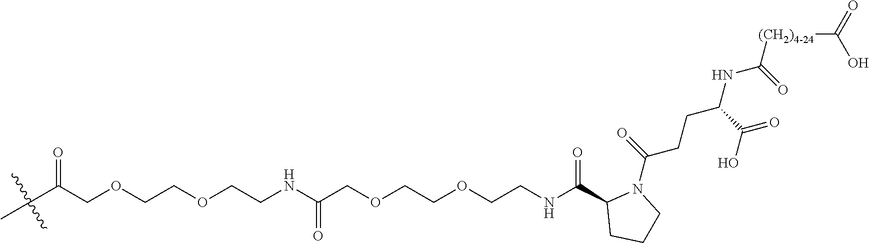 [SMILES: *C(=O)COCCOCCNC(=O)COCCOCCNC(=O)[C@@H]1CCCN1C(=O)CC[C@H](NC(=O)CC(=O)O)C(=O)O]

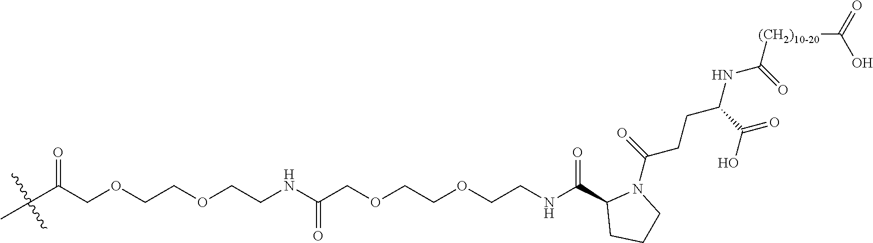 [SMILES: *C(=O)COCCOCCNC(=O)COCCOCCNC(=O)[C@@H]1CCCN1C(=O)CC[C@H](NC(=O)CC(=O)O)C(=O)O]

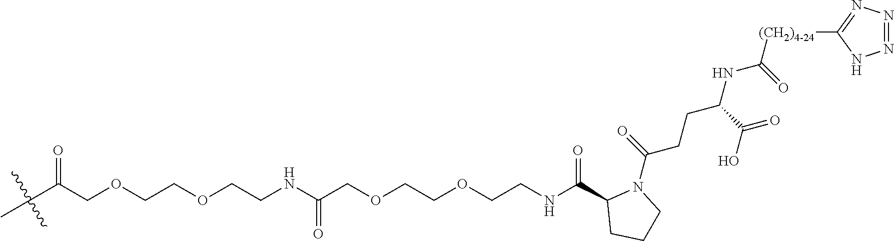 [SMILES: *C(=O)COCCOCCNC(=O)COCCOCCNC(=O)[C@@H]1CCCN1C(=O)CC[C@H](NC(=O)Cc1nnn[nH]1)C(=O)O]

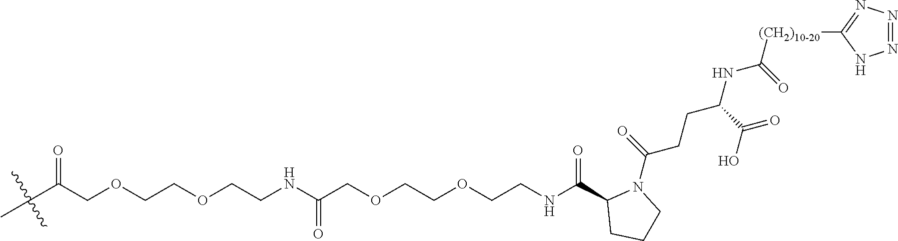 [SMILES: *C(=O)COCCOCCNC(=O)COCCOCCNC(=O)[C@@H]1CCCN1C(=O)CC[C@H](NC(=O)Cc1nnn[nH]1)C(=O)O]

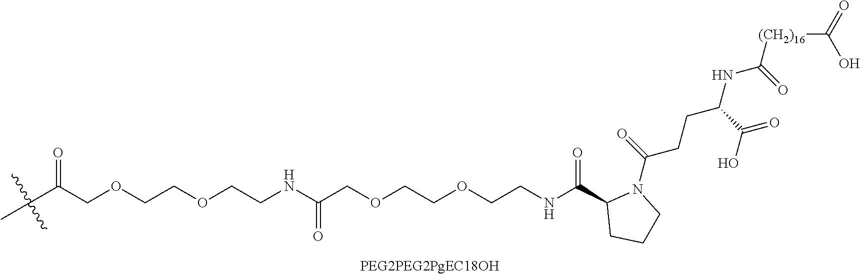

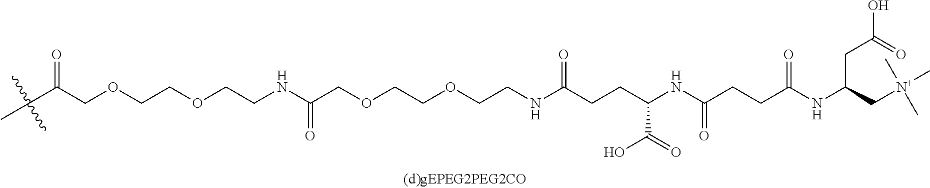 [SMILES: *C(=O)COCCOCCNC(=O)COCCOCCNC(=O)CC[C@H](NC(=O)CCC(=O)N[C@@H](CC(=O)O)C[N+](C)(C)C)C(=O)O]

In some embodiments, the peptide comprises a sequence according to any one of the following Formulas:

X₃-X₄-X₅-X₆-7MeW-X₈-X₉-X₁₀-X₁₁-X₁₂-
X₁₃-X₁₄-X₁₅-X₁₆-X₁₇ (I-Gla),
X₃-X₄-X₅-X₆-7(3NAcPh)W-X₈-X₉-X₁₀-X₁₁-
X₁₂-X₁₃-X₁₄-X₁₅-X₁₆-X₁₇ (I-Glb),
X₃-X₄-X₅-X₆-X₇-X₈-X₉-AEF-X₁₁-X₁₂-X₁₃-
X₁₄-X₁₅-X₁₆-X₁₇ (I-H1),
X₃-X₄-X₅-X₆-X₇-X₈-X₉-X₁₀-6OH2Nal-X₁₂-
X₁₃-X₁₄-X₁₅-X₁₆-X₁₇ (I-I1),
X₃-X₄-X₅-X₆-X₇-X₈-X₉-X₁₀-X₁₁-X₁₂-X₁₃-
X₁₄-3Pya-X₁₆-X₁₇ (I-J1),
X₃-X₄-X₅-X₆-7MeW-X₈-X₉-AEF-6OH2Nal-
X₁₂-X₁₃-X₁₄-3Pya-X₁₆-X₁₇ (I-Kla),
X₃-X₄-X₅-X₆-7(3NAcPh)W-X₈-X₉-AEF-6OH2Nal-
X₁₂-X₁₃-X₁₄-3Pya-X₁₆-X₁₇ (I-K1b),
r-X₄-X₅-X₆-7MeW-X₈-X₉-AEF-6OH2Nal-X₁₂-
X₁₃-X₁₄-3Pya-X₁₆-X₁₇ (I-Lla),
r-X₄-X₅-X₆-7(3NAcPh)W-X₈-X₉-AEF-6OH2Nal-
X₁₂-X₁₃-X₁₄-3Pya-X₁₆-X₁₇ (I-L1b),
X₃-Pen-X₅-X₆-7MeW-X₈-Pen-AEF-6OH2Nal-
X₁₂-X₁₃-X₁₄-3Pya-X₁₆-X₁₇ (I-Mla),
X₃-Pen-X₅-X₆-7(3NAcPh)W-X₈-Pen-AEF-6OH2Nal-
X₁₂-X₁₃-X₁₄-3Pya-X₁₆-X₁₇ (I-M1b),
X₃-X₄-X₅-X₆-7MeW-X₈-X₉-AEF-6OH2Nal-X₁₂-
X₁₃-X₁₄-3Pya-Sar-X₁₇ (I-Nla),
X₃-X₄-X₅-X₆-7(3NAcPh)W-X₈-X₉-AEF-6OH2Nal-
X₁₂-X₁₃-X₁₄-3Pya-Sar-X₁₇ (I-N1b),
X₃-X₄-X₅-X₆-7MeW-X₈-X₉-AEF-6OH2Nal-X₁₂-
X₁₃-X₁₄-3Pya-X₁₆ (I-Ola),
or
X₃-X₄-X₅-X₆-7(3NAcPh)W-X₈-X₉-AEF-60H2Nal-
X₁₂-X₁₃-X₁₄-3Pya-X₁₆ (I-O1b).

In some embodiments, the peptide comprises a sequence according to any one of the following Formulas:

R₁-X₃-X₄-X₅-T-7MeW-X₈-X₉-X₁₀-X₁₁-X₁₂-X₁₃-
N-X₁₅-X₁₆-X₁₇-R₂ (I-G2a),
R₁-X₃-X₄-X₅-T-7(3NAcPh)W-X₈-X₉-X₁₀-X₁₁-
X₁₂-X₁₃-N-X₁₅-X₁₆-X₁₇-R₂ (I-G2b),
R₁-X₃-X₄-X₅-T-X₇-X₈-X₉-AEF-X₁₁-X₁₂-X₁₃-
N-X₁₅-X₁₆-X₁₇-R₂ (I-H2),
R₁-X₃-X₄-X₅-T-X₇-X₈-X₉-X₁₀-6OH2Nal-X₁₂-
X₁₃-N-X₁₅-X₁₆-X₁₇-R₂ (1-12),
R₁-X₃-X₄-X₅-T-X₇-X₈-X₉-X₁₀-X₁₁-X₁₂-X₁₃-
N-3Pya-X₁₆-X₁₇-R₂ (I-J2),
R₁-X₃-X₄-X₅-T-7MeW-X₈-X₉-AEF-6OH2Nal-X₁₂-
X₁₃-N-3Pya-X₁₆-X₁₇-R₂ (I-K2a),
R₁-X₃-X₄-X₅-T-7(3NAcPh)W-X₈-X₉-AEF-6OH2Nal-
X₁₂-X₁₃-N-3Pya-X₁₆-X₁₇-R₂ (I-K2b),
R₁-r-X₄-X₅-T-7MeW-X₈-X₉-AEF-60H2Nal-X₁₂-
X₁₃-N-3Pya-X₁₆-X₁₇-R₂ (I-L2a),
R₁-r-X₄-X₅-T-7(3NAcPh)W-X₈-X₉-AEF-6OH2Nal-
X₁₂-X₁₃-N-3Pya-X₁₆-X₁₇-R₂ (I-L2b),
R₁-X₃-Pen-X₅-T-7MeW-X₈-Pen-AEF-6OH2Nal-X₁₂-
X₁₃-N-3Pya-X₁₆-X₁₇-R₂ (I-M2a),
R₁-X₃-Pen-X₅-T-7(3NAcPh)W-X₈-Pen-AEF-6OH2Nal-
X₁₂-X₁₃-N-3Pya-X₁₆-X₁₇-R₂ (I-M2b),
R₁-X₃-X₄-X₅-T-7MeW-X₈-X₉-AEF-6OH2Nal-X₁₂-X₁₃-
N-3Pya-Sar-X₁₇-R₂ (I-N2a),
R₁-X₃-X₄-X₅-T-7(3NAcPh)W-X₈-X₉-AEF-6OH2Nal-
X₁₂-X₁₃-N-3Pya-Sar-X₁₇-R₂ (I-N2b),
R₁-X₃-X₄-X₅-T-7MeW-X₈-X₉-AEF-6OH2Nal-X₁₂-X₁₃-
N-3Pya-X₁₆-R₂ (I-O2a),
or
R₁-X₃-X₄-X₅-T-7(3NAcPh)W-X₈-X₉-AEF-6OH2Nal-
X₁₂-X₁₃-N-3Pya-X₁₆-R₂ (I-O2b).

In some embodiments, the amino acid at X₃ is an L-amino acid. In some embodiments, the amino acid at X₃ is a D-amino acid.

In some embodiments, the amino acid at X₄ is an L-amino acid. In some embodiments, the amino acid at X₄ is a D-amino acid.

In some embodiments, the amino acid at X₅ is an L-amino acid. In some embodiments, the amino acid at X₅ is a D-amino acid.

In some embodiments, the amino acid at X₆ is an L-amino acid. In some embodiments, the amino acid at X₆ is a D-amino acid.

In some embodiments, the amino acid at X₇ is an L-amino acid. In some embodiments, the amino acid at X₇ is a D-amino acid.

In some embodiments, the amino acid at X₈ is an L-amino acid. In some embodiments, the amino acid at X₈ is a D-amino acid.

In some embodiments, the amino acid at X₉ is an L-amino acid. In some embodiments, the amino acid at X₉ is a D-amino acid.

In some embodiments, the amino acid at X₁₀ is an L-amino acid. In some embodiments, the amino acid at X₁₀ is a D-amino acid.

In some embodiments, the amino acid at X₁₁ is an L-amino acid. In some embodiments, the amino acid at X₁₁ is a D-amino acid.

In some embodiments, the amino acid at X₁₂ is an L-amino acid. In some embodiments, the amino acid at X₁₂ is a D-amino acid.

In some embodiments, the amino acid at X₁₃ is an L-amino acid. In some embodiments, the amino acid at X₁₃ is a D-amino acid.

In some embodiments, the amino acid at X₁₄ is an L-amino acid. In some embodiments, the amino acid at X₁₄ is a D-amino acid.

In some embodiments, the amino acid at X₁₅ is an L-amino acid. In some embodiments, the amino acid at X₁₅ is a D-amino acid.

In some embodiments, the amino acid at X₁₆ is an L-amino acid. In some embodiments, the amino acid at X₁₆ is a D-amino acid.

In some embodiments, the amino acid at X₁₇ is an L-amino acid. In some embodiments, the amino acid at X₁₇ is a D-amino acid.

In some embodiments, the present disclosure provides a peptide described herein provided the peptide retains activity as an inhibitor of interleukin-23 receptor.

The present disclosure further provides a peptide of any one of SEQ ID NOS: 1-447, as shown in Table 4, or a pharmaceutically acceptable salt thereof.

In some embodiments, when the peptide comprises a cationic group (such as a quaternary ammonium moiety), the peptide further comprises a counter-anion, such as, without limitation, acetate, adipate, benzoate, benzenesulfonate, citrate, decanoate, chloride, lactate, maleate, methanesulfonate, oxalate, pivalate, propionate, succinate, sulfate, tartrate, or trifluoroacetate.

Lengthy table referenced here
US20240279281A1-20240822-T00001
Please refer to the end of the specification for access instructions.

In the peptide sequences shown above, where a number in parenthesis follows a particular residue, that residue is linked to another residue in the sequence that is denoted with the same number. For example, in the sequence MeCO-k(d)-Pen(3)-E(2)-T-7MeW-K(Ac)-Pen(3)-AEF(2)-6OH2Nal-THP-K(Ac)-N-3Pya-Sar-CONH2 (SEQ ID NO: 133), the two Pen(3) residues are linked to one another, and the E(2) residue is linked to the AEF(2) residue.

In some embodiments, the present disclosure provides a peptide selected from the group consisting of:

(SEQ ID NO: 11)
MeCO-k(Me)3-Pen(3)-N-T-7(3NAcPh)W-K(Ac)-Pen(3)-
AEF(G)-6OH2Nal-THP-E-N-5MePyridinAla-Sar-CONH2,
(SEQ ID NO: 15)
MeCO-r-Pen(3)-N-T-7(3NAcPh)W-K(Ac)-Pen(3)-AEF(G)-
6OH2Nal-THP-E-N-5MePyridinAla-Sar-CONH2,
(SEQ ID NO: 34)
cPEG3aCO-Pen(3)-N-T-7(3NAcPh)W-K(Ac)-Pen(3)-AEF(G)-
6OH2Nal-THP-K(NMeAc)-N-3Pya-Sar-CONH2,
(SEQ ID NO: 59)
MeCO-r-Pen(3)-N-T-7MeW-K(Ac)-Pen(3)-TMAPF-
6OH2Nal-THP-E-N-3Pya-Sar-CONH2,
(SEQ ID NO: 68)
MeCO-k(Me)3-Pen(3)-N-T-7(3NAcPh)W-K(Ac)-Pen(3)-
AEF-6OH2Nal-THP-E-N-3Pya-Sar-CONH2,
(SEQ ID NO: 112)
5cpaCO-Pen(3)-K(5)-T-7MeW-K(Ac)-Pen(3)-AEF(5)-
6OH2Nal-THP-K(Ac)-N-3Pya-Sar-CONH2,
(SEQ ID NO: 287)
MeCO-r-Pen(3)-N-T-7(3NAcPh)W-K(Ac)-Pen(3)-AEF-
6OH2Nal-THP-E-N-3Pya-Sar-CONH2,
(SEQ ID NO: 315)
MeCO-Pen(3)-N(NMe2)-T-7MeW-K(NMeAc)-Pen(3)-
AEF(NMePEG3a)-6OH2Nal-THP-K(NMeAc)-N-3Pya-
Sar-CONH(PEG3a)
(SEQ ID NO: 319)
cPEG3aCO-Pen(3)-N-T-7MeW-K(NMecPEG3a)-Pen(3)-
AEF-6OH2Nal-THP-E-N-3Pya-Sar-CONH2,
(SEQ ID NO: 358)
PEG2NMe(2)-Pen(3)-N-T-7MeW-K(NMeAc)-Pen(3)-AEF-
6OH2Nal-THP-hE(2)-N-3Pya-Sar-CONH2,
(SEQ ID NO: 364)
cPEG3aCO-Pen(3)-E(2)-T-7MeW-K(Ac)-Pen(3)-AEF(2)-
6OH2Nal-THP-K(Ac)-N-3Pya-Sar-CONH2,
(SEQ ID NO: 371)
cPEG3aCO-Pen(3)-N(N(Me)2)-T-7MeW-K(NMeAc)-Pen(3)-
AEF-6OH2Nal-THP-Dab(NMeAc)-N-3Pya-Sar-CON(Me)2,
(SEQ ID NO: 373)
cPEG3aCO-k(2)-Pen(3)-N(N(Me)2)-T-7MeW-K(NMeAc)-
Pen(3)-AEF-6OH2Nal-THP-hE(2)-N-3Pya-Sar-CON(Me)2,
and
(SEQ ID NO: 392)
cPEG3aCO-Pen(3)-N-T-7MeW-K(NMeAc)-Pen(3)-AEF-
6OH2Nal-THP-Q(N(Me)2)-N-3Pya-Sar-CONH2,

or a pharmaceutically acceptable salt thereof.

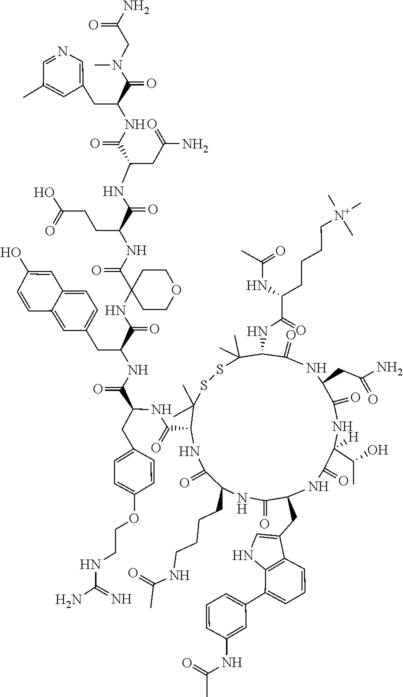 [SMILES: CC(=O)NCCCC[C@@H]1NC(=O)[C@H](Cc2c[nH]c3c(-c4cccc(NC(C)=O)c4)cccc23)NC(=O)[C@H]([C@@H](C)O)NC(=O)[C@H](CC(N)=O)NC(=O)[C@@H](NC(=O)[C@@H](CCCC[N+](C)(C)C)NC(C)=O)C(C)(C)SSC(C)(C)[C@@H](C(=O)N[C@@H](Cc2ccc(OCCNC(=N)N)cc2)C(=O)N[C@@H](Cc2ccc3cc(O)ccc3c2)C(=O)NC2(C(=O)N[C@@H](CCC(=O)O)C(=O)N[C@@H](CC(N)=O)C(=O)N[C@@H](Cc3cncc(C)c3)C(=O)N(C)CC(N)=O)CCOCC2)NC1=O]

or a pharmaceutically acceptable salt thereof.

In some embodiments, the present disclosure provides a peptide having the following structure:

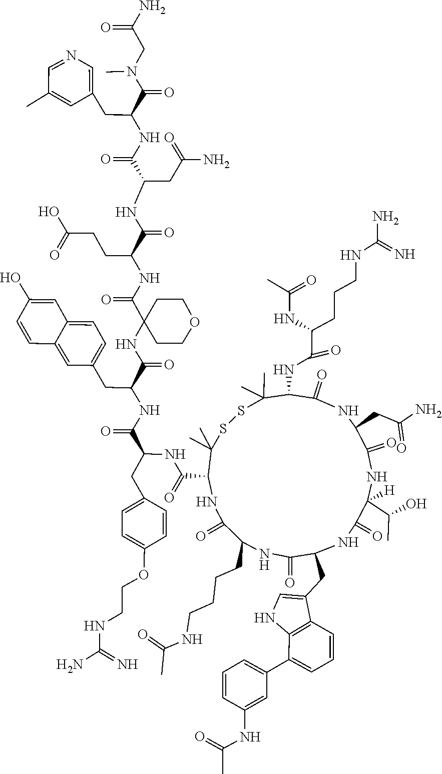 [SMILES: CC(=O)NCCCC[C@@H]1NC(=O)[C@H](Cc2c[nH]c3c(-c4cccc(NC(C)=O)c4)cccc23)NC(=O)[C@H]([C@@H](C)O)NC(=O)[C@H](CC(N)=O)NC(=O)[C@@H](NC(=O)[C@@H](CCCNC(=N)N)NC(C)=O)C(C)(C)SSC(C)(C)[C@@H](C(=O)N[C@@H](Cc2ccc(OCCNC(=N)N)cc2)C(=O)N[C@@H](Cc2ccc3cc(O)ccc3c2)C(=O)NC2(C(=O)N[C@@H](CCC(=O)O)C(=O)N[C@@H](CC(N)=O)C(=O)N[C@@H](Cc3cncc(C)c3)C(=O)N(C)CC(N)=O)CCOCC2)NC1=O]

or a pharmaceutically acceptable salt thereof.

In some embodiments, the present disclosure provides a peptide having the following structure:

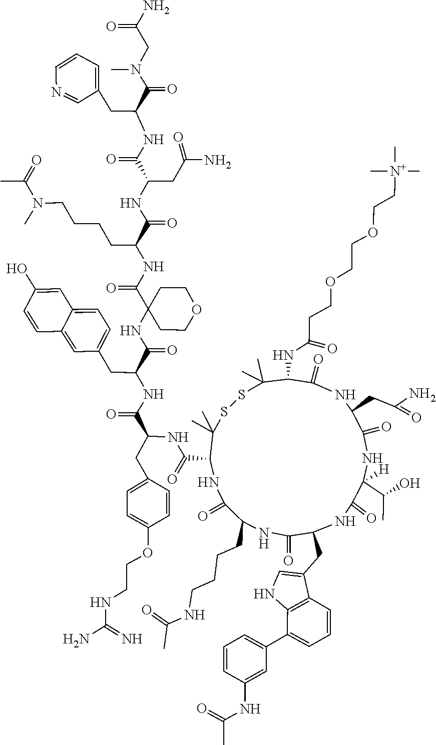 [SMILES: CC(=O)NCCCC[C@@H]1NC(=O)[C@H](Cc2c[nH]c3c(-c4cccc(NC(C)=O)c4)cccc23)NC(=O)[C@H]([C@@H](C)O)NC(=O)[C@H](CC(N)=O)NC(=O)[C@@H](NC(=O)CCOCCOCC[N+](C)(C)C)C(C)(C)SSC(C)(C)[C@@H](C(=O)N[C@@H](Cc2ccc(OCCNC(=N)N)cc2)C(=O)N[C@@H](Cc2ccc3cc(O)ccc3c2)C(=O)NC2(C(=O)N[C@@H](CCCCN(C)C(C)=O)C(=O)N[C@@H](CC(N)=O)C(=O)N[C@@H](Cc3cccnc3)C(=O)N(C)CC(N)=O)CCOCC2)NC1=O]

or a pharmaceutically acceptable salt thereof.

In some embodiments, the present disclosure provides a peptide having the following structure:

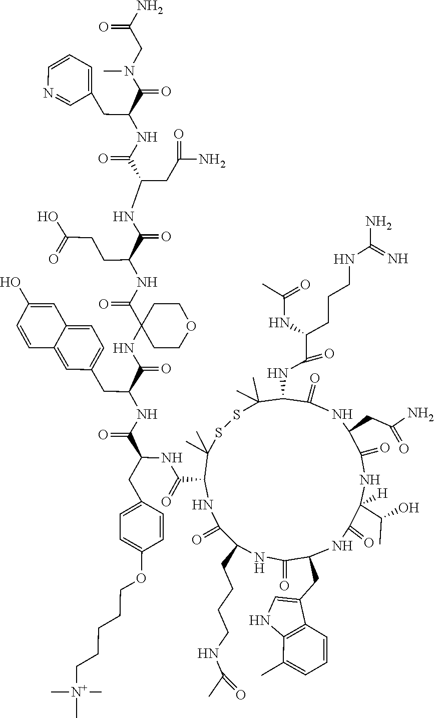 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)[C@@H](CCCNC(=N)N)NC(C)=O)C(C)(C)SSC(C)(C)[C@@H](C(=O)N[C@@H](Cc2ccc(OCCCCC[N+](C)(C)C)cc2)C(=O)N[C@@H](Cc2ccc3cc(O)ccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O]

or a pharmaceutically acceptable salt thereof.

In some embodiments, the present disclosure provides a peptide having the following structure:

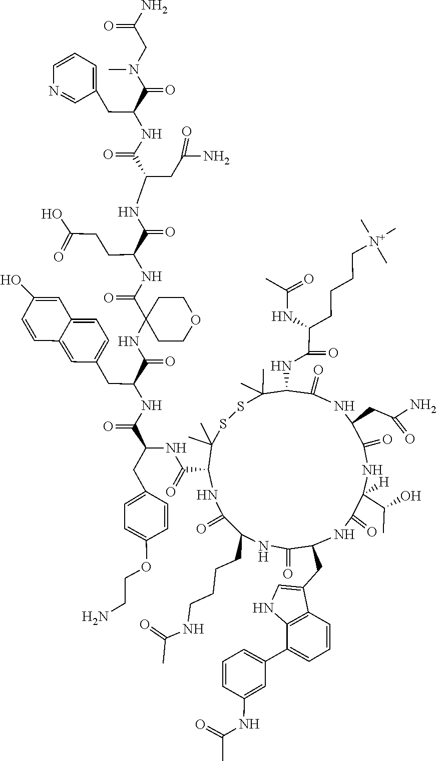 [SMILES: CC(=O)NCCCC[C@@H]1NC(=O)[C@H](Cc2c[nH]c3c(-c4cccc(NC(C)=O)c4)cccc23)NC(=O)[C@H]([C@@H](C)O)NC(=O)[C@H](CC(N)=O)NC(=O)[C@@H](NC(=O)[C@@H](CCCC[N+](C)(C)C)NC(C)=O)C(C)(C)SSC(C)(C)[C@@H](C(=O)N[C@@H](Cc2ccc(OCCN)cc2)C(=O)N[C@@H](Cc2ccc3cc(O)ccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O]

or a pharmaceutically acceptable salt thereof.

In some embodiments, the present disclosure provides a peptide having the following structure:

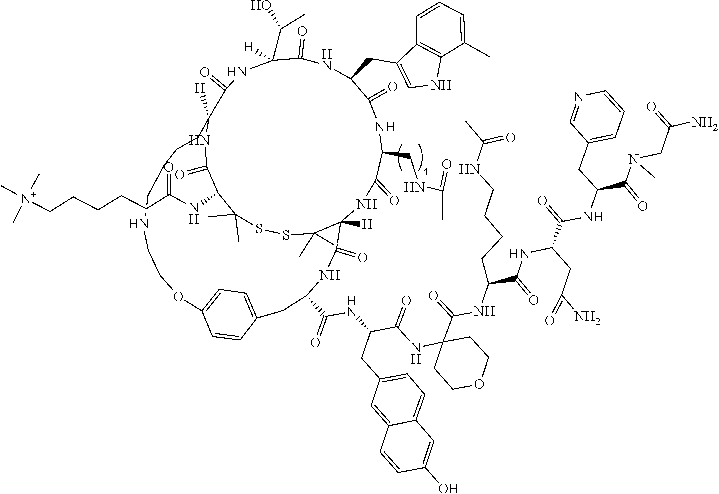

or a pharmaceutically acceptable salt thereof.

In some embodiments, the present disclosure provides a peptide having the following structure:

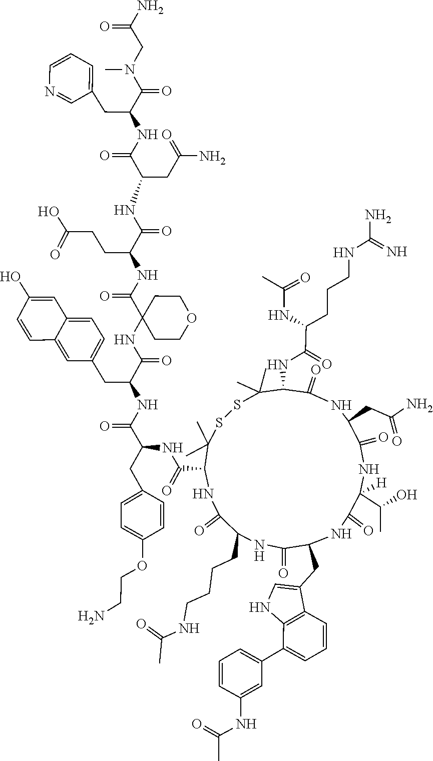 [SMILES: CC(=O)NCCCC[C@@H]1NC(=O)[C@H](Cc2c[nH]c3c(-c4cccc(NC(C)=O)c4)cccc23)NC(=O)[C@H]([C@@H](C)O)NC(=O)[C@H](CC(N)=O)NC(=O)[C@@H](NC(=O)[C@@H](CCCNC(=N)N)NC(C)=O)C(C)(C)SSC(C)(C)[C@@H](C(=O)N[C@@H](Cc2ccc(OCCN)cc2)C(=O)N[C@@H](Cc2ccc3cc(O)ccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O]

or a pharmaceutically acceptable salt thereof.

In some embodiments, the present disclosure provides a peptide having the following structure:

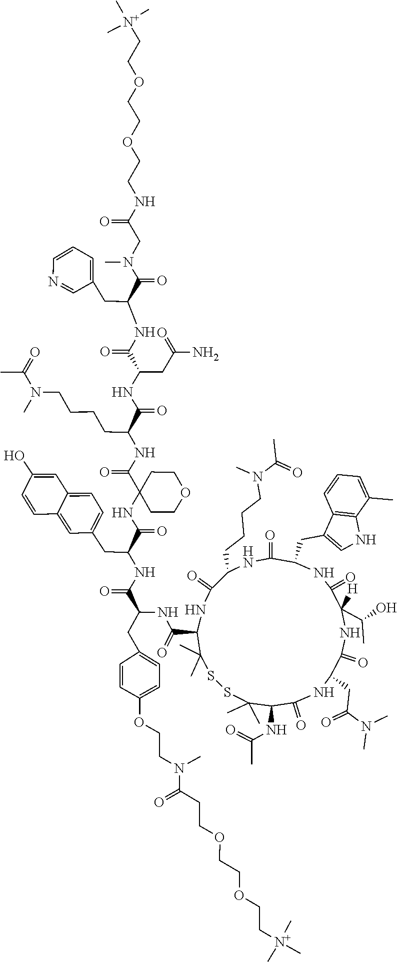 [SMILES: CC(=O)N[C@@H]1C(=O)N[C@@H](CC(=O)N(C)C)C(=O)N[C@@H]([C@@H](C)O)C(=O)N[C@@H](Cc2c[nH]c3c(C)cccc23)C(=O)N[C@@H](CCCCN(C)C(C)=O)C(=O)N[C@H](C(=O)N[C@@H](Cc2ccc(OCCN(C)C(=O)CCOCCOCC[N+](C)(C)C)cc2)C(=O)N[C@@H](Cc2ccc3cc(O)ccc3c2)C(=O)NC2(C(=O)N[C@@H](CCCCN(C)C(C)=O)C(=O)N[C@@H](CC(N)=O)C(=O)N[C@@H](Cc3cccnc3)C(=O)N(C)CC(=O)NCCOCCOCC[N+](C)(C)C)CCOCC2)C(C)(C)SSC1(C)C]

or a pharmaceutically acceptable salt thereof.

In some embodiments, the present disclosure provides a peptide having the following structure:

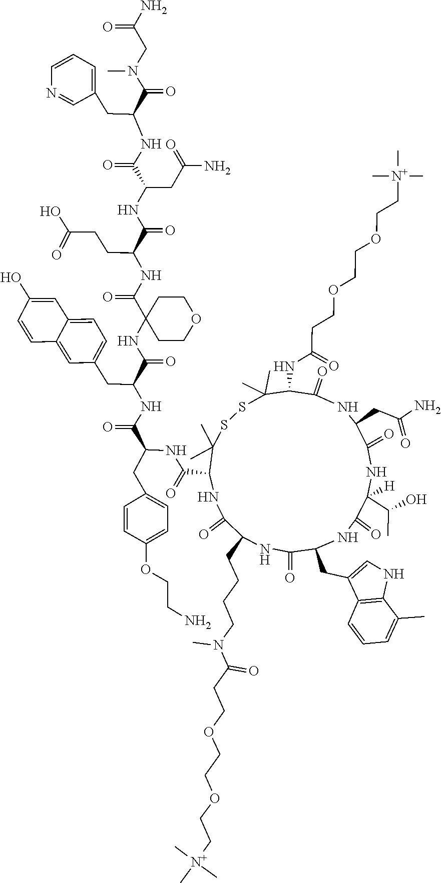 [SMILES: Cc1cccc2c(C[C@@H]3NC(=O)[C@H]([C@@H](C)O)NC(=O)[C@H](CC(N)=O)NC(=O)[C@@H](NC(=O)CCOCCOCC[N+](C)(C)C)C(C)(C)SSC(C)(C)[C@@H](C(=O)N[C@@H](Cc4ccc(OCCN)cc4)C(=O)N[C@@H](Cc4ccc5cc(O)ccc5c4)C(=O)NC4(C(=O)N[C@@H](CCC(=O)O)C(=O)N[C@@H](CC(N)=O)C(=O)N[C@@H](Cc5cccnc5)C(=O)N(C)CC(N)=O)CCOCC4)NC(=O)[C@H](CCCCN(C)C(=O)CCOCCOCC[N+](C)(C)C)NC3=O)c[nH]c12]

or a pharmaceutically acceptable salt thereof.

In some embodiments, the present disclosure provides a peptide having the following structure:

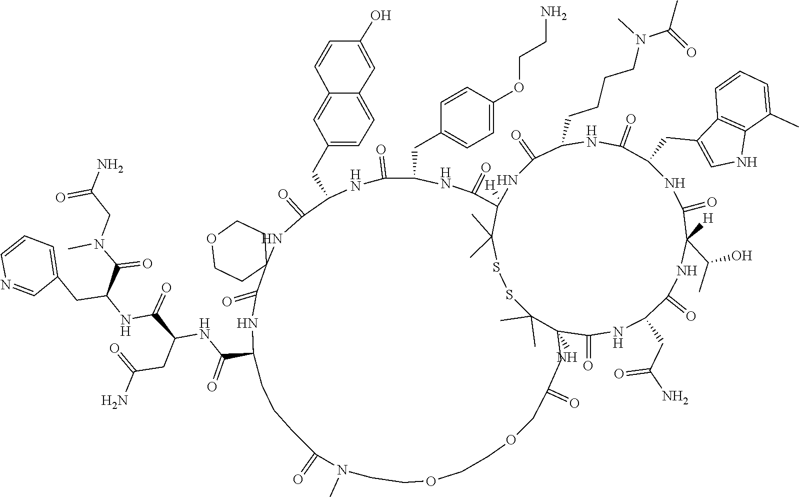

or a pharmaceutically acceptable salt thereof.

In some embodiments, the present disclosure provides a peptide having the following structure:

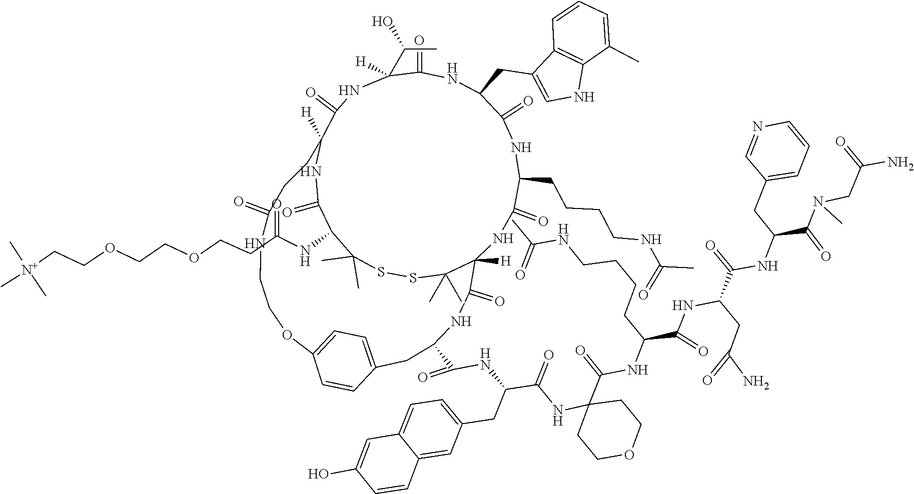 [SMILES: CC(=O)NCCCC[C@@H]1NC(=O)[C@H](Cc2c[nH]c3c(C)cccc23)NC(=O)[C@H]([C@@H](C)O)NC(=O)[C@@H]2CCC(=O)NCCOc3ccc(cc3)C[C@@H](C(=O)N[C@@H](Cc3ccc4cc(O)ccc4c3)C(=O)NC3(C(=O)N[C@@H](CCCCNC(C)=O)C(=O)N[C@@H](CC(N)=O)C(=O)N[C@@H](Cc4cccnc4)C(=O)N(C)CC(N)=O)CCOCC3)NC(=O)[C@@H](NC1=O)C(C)(C)SSC(C)(C)[C@H](NC(=O)CCCOCCOCC[N+](C)(C)C)C(=O)N2]

or a pharmaceutically acceptable salt thereof.

In some embodiments, the present disclosure provides a peptide having the following structure:

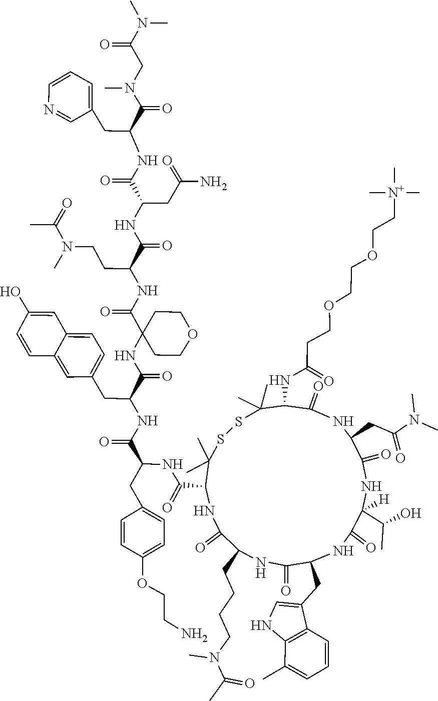 [SMILES: CC(=O)N(C)CCCC[C@@H]1NC(=O)[C@H](Cc2c[nH]c3c(C)cccc23)NC(=O)[C@H]([C@@H](C)O)NC(=O)[C@H](CC(=O)N(C)C)NC(=O)[C@@H](NC(=O)CCOCCOCC[N+](C)(C)C)C(C)(C)SSC(C)(C)[C@@H](C(=O)N[C@@H](Cc2ccc(OCCN)cc2)C(=O)N[C@@H](Cc2ccc3cc(O)ccc3c2)C(=O)NC2(C(=O)N[C@@H](CCN(C)C(C)=O)C(=O)N[C@@H](CC(N)=O)C(=O)N[C@@H](Cc3cccnc3)C(=O)N(C)CC(=O)N(C)C)CCOCC2)NC1=O]

or a pharmaceutically acceptable salt thereof.

In some embodiments, the present disclosure provides a peptide having the following structure:

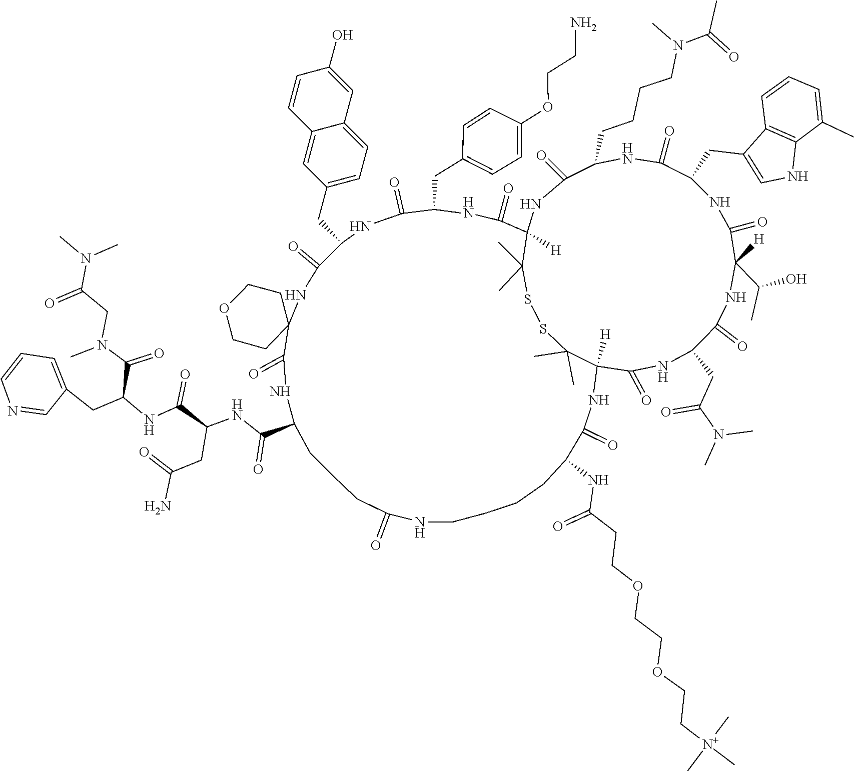 [SMILES: CC(=O)N(C)CCCC[C@@H]1NC(=O)[C@H](Cc2c[nH]c3c(C)cccc23)NC(=O)[C@H]([C@@H](C)O)NC(=O)[C@H](CC(=O)N(C)C)NC(=O)[C@H]2NC(=O)[C@H](NC(=O)CCOCCOCC[N+](C)(C)C)CCCCNC(=O)CCC[C@@H](C(=O)N[C@@H](CC(N)=O)C(=O)N[C@@H](Cc3cccnc3)C(=O)N(C)CC(=O)N(C)C)NC(=O)C3(CCOCC3)NC(=O)[C@H](Cc3ccc4cc(O)ccc4c3)NC(=O)[C@H](Cc3ccc(OCCN)cc3)NC(=O)[C@@H](NC1=O)C(C)(C)SSC2(C)C]

or a pharmaceutically acceptable salt thereof.

In some embodiments, the present disclosure provides a peptide having the following structure:

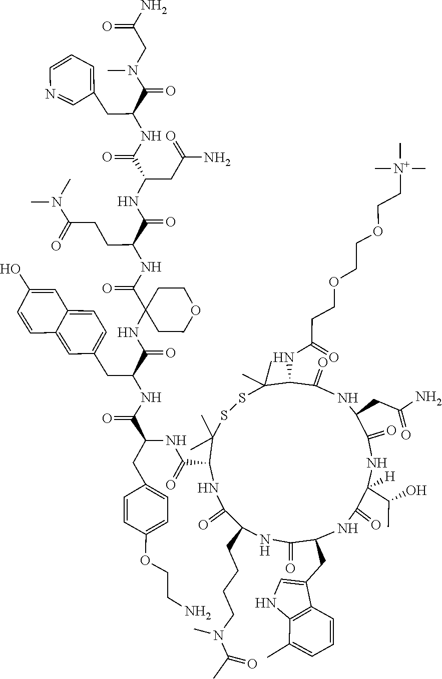 [SMILES: CC(=O)N(C)CCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)CCOCCOCC[N+](C)(C)C)C(C)(C)SSC(C)(C)[C@@H](C(=O)N[C@@H](Cc2ccc(OCCN)cc2)C(=O)N[C@@H](Cc2ccc3cc(O)ccc3c2)C(=O)NC2(C(=O)N[C@@H](CCC(=O)N(C)C)C(=O)N[C@@H](CC(N)=O)C(=O)N[C@@H](Cc3cccnc3)C(=O)N(C)CC(N)=O)CCOCC2)NC1=O]

or a pharmaceutically acceptable salt thereof.

### Methods of Synthesis

The compounds described herein may be synthesized by many techniques that are known to those skilled in the art. In some aspects, the present disclosure provides a method of chemically synthesizing a peptide of the present disclosure. In some embodiments, a portion of the peptide is recombinantly synthesized, instead of being chemically synthesized. In some aspects, methods of producing a peptide further include cyclizing the peptide precursor after the constituent subunits have been attached. In particular aspects, cyclization is accomplished via any of the various methods described herein.

The present disclosure further describes synthesis of compounds described herein. In some aspects, one or more of the amino acid residues or amino acid monomers are lipidated and then covalently attached to one another to form a peptide of the disclosure. In some aspects, one or more of the amino acid residues or amino acid monomers are covalently attached to one another and lipidated at an intermediate oligomer stage before attaching additional amino acids and cyclization to form a peptide of the disclosure. In some aspects, a cyclic peptide is synthesized and then lipidated to form a compound of the disclosure. Illustrative synthetic methods are described in the Examples.

### Pharmaceutical Compositions

The present disclosure further relates to a pharmaceutical composition comprising an IL-23R inhibitor described herein. In particular, the present disclosure includes pharmaceutical compositions comprising one or more peptides of the present disclosure and a pharmaceutically acceptable carrier, diluent or excipient. The pharmaceutically acceptable carrier, diluent or excipient may be a solid, semi-solid or liquid filler, diluent, encapsulating material or formulation auxiliary of any type. Prevention of the action of microorganisms may be ensured by the inclusion of various antibacterial and antifungal agents, for example, paraben, chlorobutanol, phenol sorbic acid, and the like. It may also be desirable to include isotonic agents such as sugars, sodium chloride, and the like.

The pharmaceutical compositions may be administered orally, parenterally, intracisternally, intravaginally, intraperitoneally, intrarectally, topically (as by powders, ointments, drops, suppository, or transdermal patch), by inhalation (such as intranasal spray), ocularly (such as intraocularly) or buccally. The term “parenteral” as used herein refers to modes of administration which include intravenous, intramuscular, intraperitoneal, intrasternal, subcutaneous, intradermal and intraarticular injection and infusion. Accordingly, in certain embodiments, the compositions are formulated for delivery by any of these routes of administration. A pharmaceutical composition may be formulated for and administered orally. A pharmaceutical composition may be formulated for and administered parenterally. In some embodiments, the pharmaceutical composition is administered orally.

The IL-23R inhibitors of the present disclosure may be prepared and/or formulated as pharmaceutically acceptable salts and/or other forms thereof or when appropriate in neutral form. Pharmaceutically acceptable salts are non-toxic salts of a neutral form of a compound that possess the desired pharmacological activity of the neutral form. These salts may be derived from inorganic or organic acids or bases. For example, a compound that contains a basic nitrogen may be prepared as a pharmaceutically acceptable salt by contacting the compound with an inorganic or organic acid. Non-limiting examples of pharmaceutically acceptable salts can be found in Remington: The Science and Practice of Pharmacy, 21^{st} Edition, Lippincott Williams and Wilkins, Philadelphia, Pa., 2006.

The present disclosure relates to pharmaceutical compositions comprising an IL-23R inhibitor described herein or pharmaceutically acceptable salts, isomers, or a mixture thereof, in which one or more hydrogen atoms attached to a carbon atom may be replaced by a deuterium atom or D. As known in the art, the deuterium atom is a non-radioactive isotope of the hydrogen atom. Such compounds may increase resistance to metabolism, and thus may be useful for increasing the half-life of the compounds described herein or pharmaceutically acceptable salts, isomer, or a mixture thereof when administered to a mammal. See, e.g., Foster, “Deuterium Isotope Effects in Studies of Drug Metabolism,” Trends Pharmacol. Sci., 5(12):524-527 (1984). Such compounds are synthesized by means well known in the art, for example by employing starting materials in which one or more hydrogen atoms have been replaced by deuterium.

Examples of isotopes that can be incorporated into the disclosed compounds also include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Substitution with positron emitting isotopes, such as ¹¹C, ¹⁸F, ¹⁵O and ¹³N, can be useful in Positron Emission Topography (PET) studies for examining substrate receptor occupancy. Isotopically-labeled peptides of the present disclosure can generally be prepared by conventional techniques known to those skilled in the art or by processes analogous to those described in the Examples as set out below using an appropriate isotopically-labeled reagent in place of the non-labeled reagent previously employed.

When used in at least one of the treatments or delivery systems described herein, a peptide inhibitor of the present disclosure may be employed in pure form or, where such forms exist, in pharmaceutically acceptable salt form.

The total daily usage of the IL-23R inhibitor and compositions of the present disclosure can be decided by the attending physician within the scope of sound medical judgment. The specific therapeutically effective dose level for any particular subject will depend upon a variety of factors including: a) the disorder being treated and the severity of the disorder; b) activity of the specific compound employed; c) the specific composition employed, the age, body weight, general health, sex and diet of the patient; d) the time of administration, route of administration, and rate of excretion of the specific peptide inhibitor employed; e) the duration of the treatment; f) drugs used in combination or coincidental with the specific peptide inhibitor employed, and like factors well known in the medical arts.

The compositions may conveniently be presented in unit dosage form and can be prepared by any of the methods well known in the art of pharmacy. Techniques and compositions generally are found in Remington's Pharmaceutical Sciences (Mack Publishing Co., Easton, PA). Such methods include the step of bringing into association the active ingredient with the carrier which constitutes one or more accessory ingredients. In general the compositions are prepared by uniformly and intimately bringing into association the active ingredient with liquid carriers or finely divided solid carriers or both, and then, if necessary, shaping the product.

### Non-Invasive Detection of Intestinal Inflammation

The IL-23R inhibitors of the present disclosure may be used for detection, assessment and diagnosis of intestinal inflammation by microPET imaging, wherein the peptide inhibitor is labeled with a chelating group or a detectable label, as part of a non-invasive diagnostic procedure. In certain embodiments, an IL-23R inhibitor of the present disclosure is conjugated with a bifunctional chelator. In certain embodiments, an IL-23R inhibitor of the present disclosure is radiolabeled. The labeled IL-23R inhibitor is then administered to a subject orally or rectally. In certain embodiments, the IL-23R inhibitor is included in drinking water. Following uptake of the IL-23R inhibitor, microPET imaging may be used to visualize inflammation throughout the subject's bowels and digestive track.

### Methods of Treatment and Uses

The present disclosure relates to methods for treating a subject afflicted with a condition or indication associated with IL-23 or IL-23R activity (e.g., activation of the IL-23/IL-23R signaling pathway), wherein the method comprises administering to the subject an IL-23R inhibitor disclosed herein. In one aspect, the present disclosure provides a method for treating a subject afflicted with a condition or indication characterized by aberrant or dysregulated IL-23 or IL-23R activity or signaling, comprising administering to the subject a peptide inhibitor of the present disclosure in an amount sufficient to inhibit (partially or fully) binding of IL-23 to an IL-23R in the subject. The inhibition of IL-23 binding to IL-23R may occur in particular organs or tissues of the subject, e.g., the stomach, small intestine, large intestine/colon, intestinal mucosa, lamina propria, Peyer's Patches, mesenteric lymph nodes, or lymphatic ducts.

The present disclosure relates to methods comprising providing a peptide inhibitor described herein to a subject in need thereof. The subject in need thereof may be a subject that has been diagnosed with or has been determined to be at risk of developing a disease or disorder associated with IL-23/IL-23R. The subject may be a mammal. The subject may be, in particular, a human.

The disease or disorder to be treated by treatment with an IL-23R inhibitor of the present disclosure may be an inflammatory disease or disorder, an autoimmune inflammation diseases or disorder, and/or related disorders, including multiple sclerosis, asthma, rheumatoid arthritis, inflammation of the gut, inflammatory bowel diseases (IBDs), juvenile IBD, adolescent IBD, Crohn's disease, ulcerative colitis, sarcoidosis, Systemic Lupus Erythematosus, ankylosing spondylitis (axial spondyloarthritis), psoriatic arthritis, or psoriasis. In particular, the disease or disorder may be psoriasis (e.g., plaque psoriasis, guttate psoriasis, inverse psoriasis, pustular psoriasis, Palmo-Plantar Pustulosis, psoriasis vulgaris, or erythrodermic psoriasis), atopic dermatitis, acne ectopica, ulcerative colitis, Crohn's disease, Celiac disease (nontropical Sprue), enteropathy associated with seronegative arthropathies, microscopic colitis, collagenous colitis, eosinophilic gastroenteritis/esophagitis, colitis associated with radio- or chemo-therapy, colitis associated with disorders of innate immunity as in leukocyte adhesion deficiency-1, chronic granulomatous disease, glycogen storage disease type 1b, Hermansky-Pudlak syndrome, Chediak-Higashi syndrome, Wiskott-Aldrich Syndrome, pouchitis, pouchitis resulting after proctocolectomy and ileoanal anastomosis, gastrointestinal cancer, pancreatitis, insulin-dependent diabetes mellitus, mastitis, cholecystitis, cholangitis, primary biliary cirrhosis, viral-associated enteropathy, pericholangitis, chronic bronchitis, chronic sinusitis, asthma, uveitis, or graft versus host disease.

The present disclosure provides a method or use of an IL-23R inhibitor for treating an inflammatory disease or disorder in a subject in need thereof that includes administering to the subject a therapeutically effective amount of an IL-23R inhibitor of the present disclosure, a pharmaceutically acceptable salt thereof, or a composition disclosed herein comprising an IL-23 inhibitor of the present disclosure.

The present disclosure provides a method or use of an IL-23R inhibitor for treating an autoimmune disease or disorder in a subject in need thereof that includes administering to the subject a therapeutically effective amount of an IL-23R inhibitor of the present disclosure, a pharmaceutically acceptable salt thereof, or a composition disclosed herein comprising an IL-23 inhibitor of the present disclosure.

The present disclosure provides a method or use of an IL-23R inhibitor for treating an autoimmune inflammation disease or disorder in a subject in need thereof that includes administering to the subject a therapeutically effective amount of an IL-23R inhibitor of the present disclosure, a pharmaceutically acceptable salt thereof, or a composition disclosed herein comprising an IL-23 inhibitor of the present disclosure.

Suitable inflammatory diseases, autoimmune inflammation diseases, and/or related disorders for treatment with a compound or pharmaceutically acceptable salt thereof, or a composition of the present disclosure, may include, but are not limited to inflammatory bowel disease (IBD), Crohn's disease (CD), ulcerative colitis (UC), psoriasis (PsO), or psoriatic arthritis (PsA) and the like. The inflammatory disease to be treated may be inflammatory bowel disease (IBD), Crohn's disease, or ulcerative colitis. The inflammatory disease to be treated may be selected from psoriasis or psoriatic arthritis. The inflammatory disease to be treated may be psoriasis The inflammatory disease to be treated may be psoriatic arthritis. The inflammatory disease to be treated may be IBD. The inflammatory disease to be treated may be Crohn's disease. The inflammatory disease to be treated may be ulcerative colitis.

Production of IL-23 is enriched in the intestine, where it is believed to play a key role in regulating the balance between tolerance and immunity through T-cell-dependent and T-cell-independent pathways of intestinal inflammation through effects on T-helper 1 (Th1) and Th17-associated cytokines, as well as restraining regulatory T-cell responses in the gut, favoring inflammation. In addition, polymorphisms in the IL-23 receptor (IL-23R) have been associated with susceptibility to inflammatory bowel diseases (IBDs), further establishing the critical role of the IL-23 pathway in intestinal homeostasis. Peptides and methods for specific targeting of the IL-23R from the luminal side of the gut may provide therapeutic benefit to IBD patients suffering from local inflammation of the intestinal tissue.

Accordingly, the present disclosure also provides a method of treating or preventing inflammatory bowel disease (IBD), Crohn's disease (CD), or ulcerative colitis (UC), in a subject in need thereof, said method comprising administering to the subject a therapeutically effective amount of a peptide of the present disclosure, a pharmaceutically acceptable salt thereof, or a pharmaceutical composition described herein comprising an IL-23 inhibitor. In some embodiments, the method is for treating or preventing inflammatory bowel disease (IBD). In some embodiments, the method is for treating or preventing Crohn's disease (CD). In some embodiments, the method is for treating or preventing ulcerative colitis (UC).

Psoriasis, a chronic skin disease affecting about 2%-3% of the general population has been shown to be mediated by the body's T cell inflammatory response mechanisms. IL-23 is one of several interleukins implicated as a key player in the pathogenesis of psoriasis, purportedly by maintaining chronic autoimmune inflammation via the induction of interleukin-17, regulation of T memory cells, and activation of macrophages. Expression of IL-23 and IL-23R has been shown to be increased in tissues of patients with psoriasis, and antibodies that neutralize IL-23 showed IL-23-dependent inhibition of psoriasis development in animal models of psoriasis. Orally bioavailable peptide inhibitors of IL-23 may provide both a non-steroidal treatment option for patients with mild to moderate psoriasis and treatment for moderate to severe psoriasis that does not require delivery by infusion.

Accordingly, the present disclosure also provides a method of treating or preventing psoriasis (PsO) or psoriatic arthritis (PsA) in a subject in need thereof, said method comprising administering to the subject a therapeutically effective amount of a peptide of the present disclosure, a pharmaceutically acceptable salt thereof, or a pharmaceutical composition described herein comprising an IL-23 inhibitor. In some embodiments, the method is for treating or preventing psoriasis (PsO). In some embodiments, the method is for treating or preventing psoriatic arthritis (PsA).

The present disclosure further relates to a method of selectively inhibiting IL-23 or IL-23R signaling (or the binding of IL-23 to IL-23R) in a subject (e.g., in a subject in need thereof), comprising administering to the subject a peptide inhibitor of the IL-23R described herein. In some embodiments, the present disclosure includes and provides a method of selectively inhibiting IL-23 or IL-23R signaling (or the binding of IL-23 to IL-23R) in the GI tract of a subject (e.g., a subject in need thereof), comprising providing to the subject a peptide inhibitor of the IL-23R of the present disclosure by oral administration. The exposure of GI tissues (e.g., small intestine or colon) to the administered peptide inhibitor may be at least 10-fold, at least 20-fold, at least 50-fold, or at least 100-fold greater than the exposure (level) in the blood. In particular embodiments, the present disclosure includes a method of selectively inhibiting IL23 or IL23R signaling (or the binding of IL23 to IL23R) in the GI tract of a subject (e.g., a subject in need thereof), comprising providing to the subject a peptide inhibitor, wherein the peptide inhibitor does not block the interaction between IL-6 and IL-6R or antagonize the IL-12 signaling pathway. In a further related embodiment, the present disclosure provides a method of inhibiting GI inflammation and/or neutrophil infiltration to the GI, comprising providing to a subject in need thereof a peptide inhibitor of the present disclosure. In some embodiments, methods of the present disclosure comprise providing a peptide inhibitor of the present disclosure (i.e., a first therapeutic agent) to a subject (e.g., a subject in need thereof) in combination with a second therapeutic agent. In certain embodiments, the second therapeutic agent is provided to the subject before and/or simultaneously with and/or after the peptide inhibitor is administered to the subject. In particular embodiments, the second therapeutic agent is an anti-inflammatory agent. In certain embodiments, the second therapeutic agent is a non-steroidal anti-inflammatory drug, steroid, or immune modulating agent. In certain embodiments, the method comprises administering to the subject a third therapeutic agent. In certain embodiments, the second therapeutic agent is an antibody that binds IL-23 or IL-23R.

The present disclosure also relates to methods of inhibiting IL-23 binding to an IL-23R on a cell, comprising contacting the IL-23R with a peptide inhibitor of the receptor disclosed herein. The cell may be a mammalian cell. The method may be performed in vitro or in vivo. Inhibition of binding may be determined by a variety of routine experimental methods and assays known in the art.

The present disclosure relates to methods of inhibiting IL-23 signaling by a cell, comprising contacting the IL-23R with a peptide inhibitor described herein. In certain embodiments, the cell is a mammalian cell. In particular embodiments, the method is performed in vitro or in vivo. In particular embodiments, the inhibition of IL-23 signaling may be determined by measuring changes in phospho-STAT3 levels in the cell.

In any of the foregoing methods, IL-23R inhibitor administration to the subject may be conducted orally, but other routes of administration are not excluded. Other routes of administration include, but are not limited to, parenteral, subcutaneous, intravenous, intramuscular, intraperitoneal, transdermal, topical, buccal or ocular routes. Dosages of a peptide inhibitor of IL-23R described herein, or salt thereof to be administered to a subject may be determined by a person of skill in the art taking into account the disease or condition being treated, including its severity, and factors including age, weight, sex, and the like.

### EXAMPLES

The following examples are not intended to limit the scope of the present disclosure, but rather to provide guidance to the skilled artisan to prepare and use the peptides, compositions, and methods of the present disclosure. While particular aspects of the present disclosure are described, the skilled artisan will appreciate that various changes and modifications can be made without departing from the spirit and scope of the disclosure.

Some abbreviations useful in describing the disclosure are defined below in the following tables.

Herein and throughout the application, the following abbreviations may be used.

| Abbreviation | Definition |
|---|---|
| ACN | acetonitrile |
| Boc | tert-butoxy-carbonyl |
| carnitine- | (R)-4-(tert-butoxy)-2-(3-(hydroxy-12- |
| succinate, carn | methoxy)propanamido)-N,N,N-trimethyl-4- |
| | oxobutan-1-aminium |
| DCM | dichloromethane |
| Dde | N-(1-(4,4-dimethyl-2,6-dioxocyclohexylidene)ethyl |
| DIC | N,N′-diisopropylcarbodiimide |
| DIPEA | N,N-diisopropylethylamine |
| DMF | N,N-dimethylformamide |
| DMSO | dimethyl sulfoxide |
| DTT | dithiothreitol |
| ESI | electrospray ionization |
| Et₂O | diethyl ether |
| FMOC or Fmoc | ((9H-fluoren-9-yl)methoxy)carbonyl |
| h | hour |
| HATU | 1-[bis(dimethylamino)methylene]-1H-1,2,3- |
| | triazolo[4,5-b]pyridinium 3-oxide hexafluorophosphate |
| HOAT or HOAt | 1-hydroxy-7-azabenzotriazole |
| HPLC | high-performance liquid chromatography |
| MeOH | methanol |
| min | minute |
| MS | mass spectrometry |
| MTBE | methyl tert-butyl ether |
| MW | microwave |
| NDMBA | 1,3-dimethylbarbituric acid |
| Oxyma | ethyl cyanohydroxyiminoacetate |
| RT | room temperature |
| SPPS | solid-phase peptide synthesis |
| TBAF | tetrabutylammonium fluoride |
| TEA | triethylamine |
| TFA | trifluoroacetic acid |
| TFE | tetrafluoroethylene |
| THF | tetrahydrofuran |
| TIPS | triisopropyl silane |
| TIS | triisopropyl silane |
| UPLC | ultra performance liquid chromatography |

TABLE 5
N-Terminal Modification Abbreviations

| Abbreviation | Structure |
|---|---|
| 5Ava | 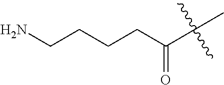 [SMILES: *C(=O)CCCCN] |
| | 5-aminovaleric acid |
| 5cpaCO | 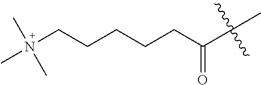 |
| 6Ahx | 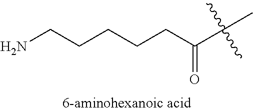 [SMILES: *C(=O)CCCCCN] |
| 7Ahp | 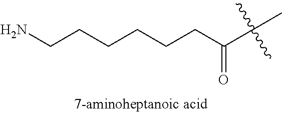 [SMILES: *C(=O)CCCCCCN] |
| CF3CO | 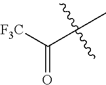 [SMILES: *C(=O)C(F)(F)F] |
| CF3Propylamide | 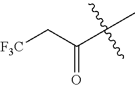 [SMILES: *C(=O)CC(F)(F)F] |
| EtCO | 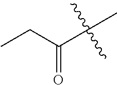 [SMILES: *C(=O)CC] |
| MeCO | 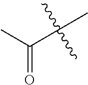 [SMILES: *C(C)=O] |
| PEG2 | 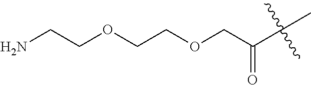 [SMILES: *C(=O)COCCOCCN] |
| PEG2NMe | 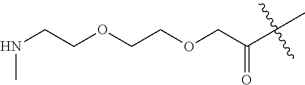 [SMILES: *C(=O)COCCOCCNC] |
| cPEG3aCO | 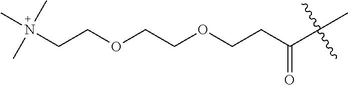 |
| cPEG5aCO | 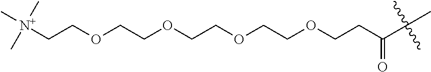 [SMILES: *C(=O)CCOCCOCCOCCOCC[N+](C)(C)C] |
| mPEG12CO | 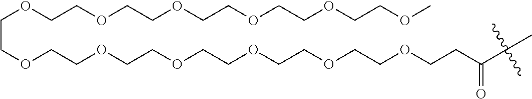 [SMILES: *C(=O)CCOCCOCCOCCOCCOCCOCCOCCOCCOCCOCCOCCOC] |
| C12gEPEG2PEG2CO | 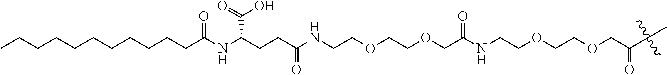 [SMILES: *C(=O)COCCOCCNC(=O)COCCOCCNC(=O)CC[C@H](NC(=O)CCCCCCCCCCC)C(=O)O] |
| C14gEPEG2PEG2CO | 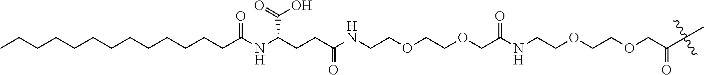 [SMILES: *C(=O)COCCOCCNC(=O)COCCOCCNC(=O)CC[C@H](NC(=O)CCCCCCCCCCCCC)C(=O)O] |
| HOC18gEPEG2PEG2CO | 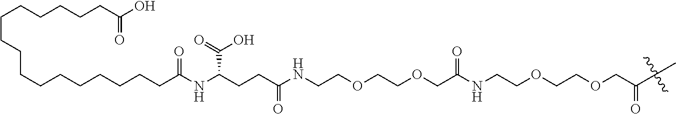 [SMILES: *C(=O)COCCOCCNC(=O)COCCOCCNC(=O)CC[C@H](NC(=O)CCCCCCCCCCCCCCCCC(=O)O)C(=O)O] |
| HOC20gEPEG2PEG2CO | 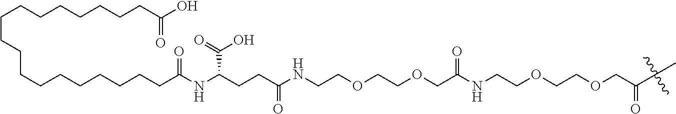 [SMILES: *C(=O)COCCOCCNC(=O)COCCOCCNC(=O)CC[C@H](NC(=O)CCCCCCCCCCCCCCCCCCC(=O)O)C(=O)O] |
| HOC18gEPEG6CO | 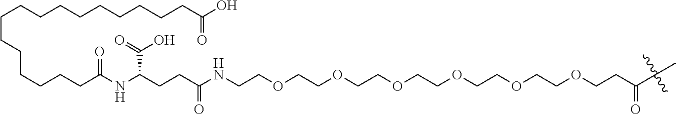 [SMILES: *C(=O)CCOCCOCCOCCOCCOCCOCCNC(=O)CC[C@H](NC(=O)CCCCCCCCCCCCCCCCC(=O)O)C(=O)O] |
| HOC18gEPEG12CO | 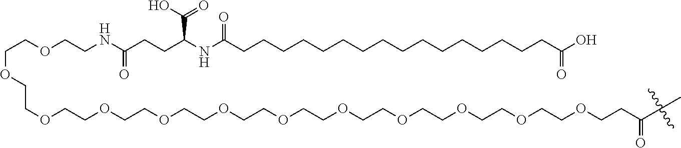 [SMILES: *C(=O)CCOCCOCCOCCOCCOCCOCCOCCOCCOCCOCCOCCOCCNC(=O)CC[C@H](NC(=O)CCCCCCCCCCCCCCCCC(=O)O)C(=O)O] |
| HOC18gEPEG2SP6PEG2CO | 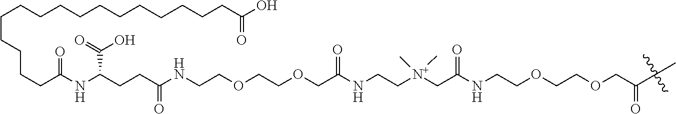 [SMILES: *C(=O)COCCOCCNC(=O)C[N+](C)(C)CCNC(=O)COCCOCCNC(=O)CC[C@H](NC(=O)CCCCCCCCCCCCCCCCC(=O)O)C(=O)O] |
| (d)gEPEG2PEG2CO | 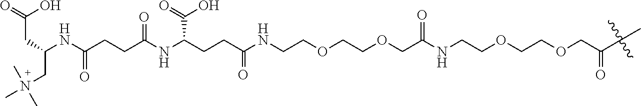 |

TABLE 6
C-Terminal Modification Abbreviations

| Abbre- | |
|---|---|
| viation | Structure |
| CONH2 | 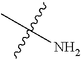 [SMILES: *N] |
| CO(DiFPip) | 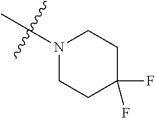 [SMILES: *N1CCC(F)(F)CC1] |
| CON(Me)2 | 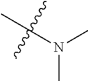 [SMILES: *N(C)C] |
| CONH (PEG3a) | 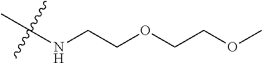 [SMILES: *NCCOCCOC] |
| CONH (PEG3a) | 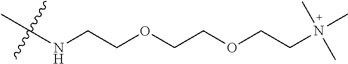 |

The amino acid structures provided in Table 7, below, are presented without stereochemical indicators at the alpha carbon; however, it is to be understood that these amino acids occur as either the L-amino acid or the D-amino acid. Unless an amino acid residue of Table 7 is represented with a lower-case abbreviation or with the letter D before it, all amino acids abbreviations described in the table refer to the L-amino acid configuration. For example, “Dab” refers to the L-stereoisomer:

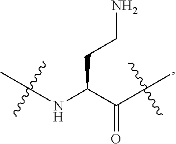

and the corresponding D-stereoisomer is represented as “dab,” “dDab,” or “D-Dab”:

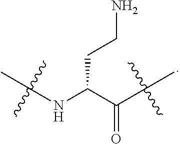 [SMILES: *N[C@H](CCN)C(*)=O]

TABLE 7
Monomer Abbreviations

| Abbreviation | Structure |
|---|---|
| 2Nal6((5CF3)3Pyrazole) | 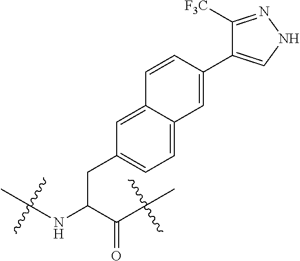 [SMILES: *NC(Cc1ccc2cc(-c3c[nH]nc3C(F)(F)F)ccc2c1)C(*)=O] |
| 2Nal6(3Pyrazole) | 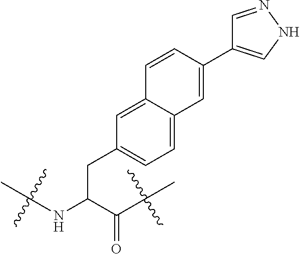 [SMILES: *NC(Cc1ccc2cc(-c3cn[nH]c3)ccc2c1)C(*)=O] |
| 2Nal6(4OMePh) | 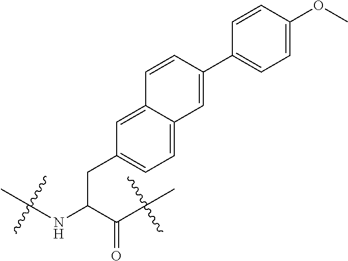 [SMILES: *NC(Cc1ccc2cc(-c3ccc(OC)cc3)ccc2c1)C(*)=O] |
| 2Nal6(Ph2OH) | 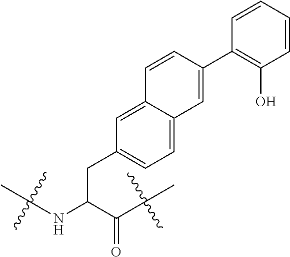 [SMILES: *NC(Cc1ccc2cc(-c3ccccc3O)ccc2c1)C(*)=O] |
| 2Nal6(Ph4(NMorph)) | 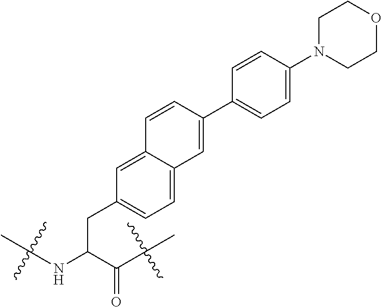 [SMILES: *NC(Cc1ccc2cc(-c3ccc(N4CCOCC4)cc3)ccc2c1)C(*)=O] |
| 3AmPyrazolAla | 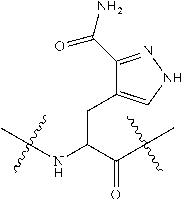 [SMILES: *NC(Cc1c[nH]nc1C(N)=O)C(*)=O] |
| 3FTyr | 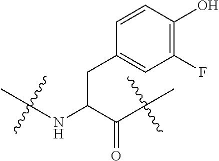 [SMILES: *NC(Cc1ccc(O)c(F)c1)C(*)=O] |
| 3Pya | 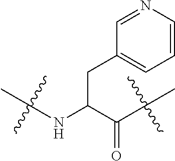 [SMILES: *NC(Cc1cccnc1)C(*)=O] |
| 4AmF | 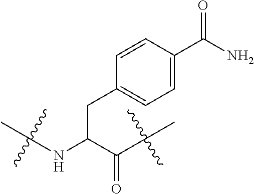 [SMILES: *NC(Cc1ccc(C(N)=O)cc1)C(*)=O] |
| 4AminoPro | 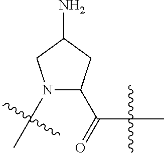 [SMILES: *C(=O)C1CC(N)CN1*] |
| 4CNF | 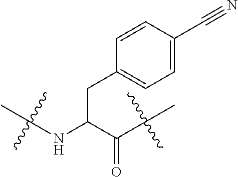 [SMILES: *NC(Cc1ccc(C#N)cc1)C(*)=O] |
| 4DMPzEF | 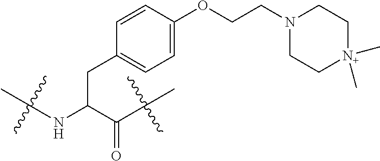 [SMILES: *NC(Cc1ccc(OCCN2CC[N+](C)(C)CC2)cc1)C(*)=O] |
| 4MeF | 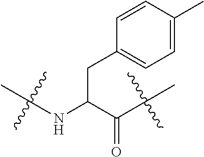 [SMILES: *NC(Cc1ccc(C)cc1)C(*)=O] |
| 4OMeF | 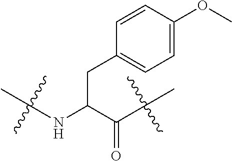 [SMILES: *NC(Cc1ccc(OC)cc1)C(*)=O] |
| 4PipPhe | 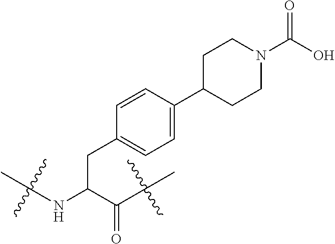 [SMILES: *NC(Cc1ccc(C2CCN(C(=O)O)CC2)cc1)C(*)=O] |
| 5amido2Nal | 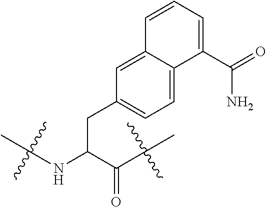 [SMILES: *NC(Cc1ccc2c(C(N)=O)cccc2c1)C(*)=O] |
| 5AmPyridinAla | 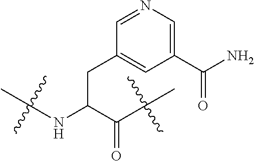 [SMILES: *NC(Cc1cncc(C(N)=O)c1)C(*)=O] |
| 5Br2Nal | 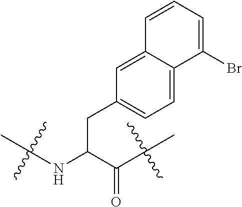 [SMILES: *NC(Cc1ccc2c(Br)cccc2c1)C(*)=O] |
| 5Me2Nal | 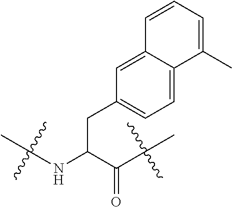 [SMILES: *NC(Cc1ccc2c(C)cccc2c1)C(*)=O] |
| 5MePyridinAla | 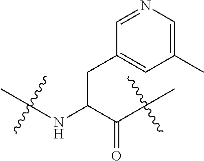 [SMILES: *NC(Cc1cncc(C)c1)C(*)=O] |
| 5OMe2Nal | 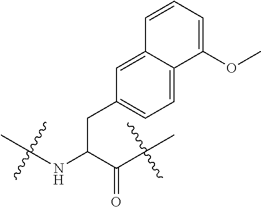 [SMILES: *NC(Cc1ccc2c(OC)cccc2c1)C(*)=O] |
| 6Br2Nal | 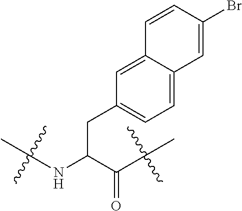 [SMILES: *NC(Cc1ccc2cc(Br)ccc2c1)C(*)=O] |
| 6F2Nal | 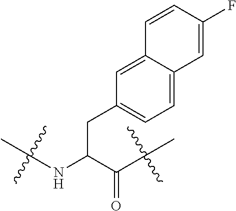 [SMILES: *NC(Cc1ccc2cc(F)ccc2c1)C(*)=O] |
| 6MeQui | 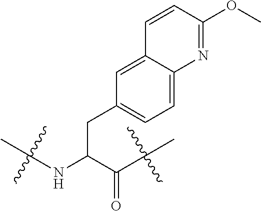 [SMILES: *NC(Cc1ccc2nc(OC)ccc2c1)C(*)=O] |
| 6O(COCF3)2Nal | 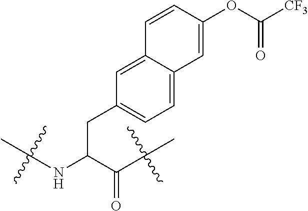 [SMILES: *NC(Cc1ccc2cc(OC(=O)C(F)(F)F)ccc2c1)C(*)=O] |
| 6OH2Nal | 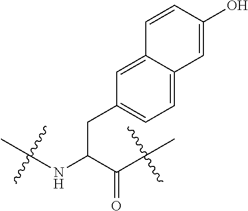 [SMILES: *NC(Cc1ccc2cc(O)ccc2c1)C(*)=O] |
| 6OHQui | 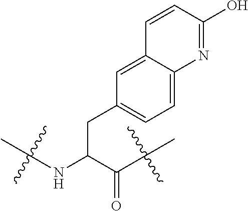 [SMILES: *NC(Cc1ccc2nc(O)ccc2c1)C(*)=O] |
| 7(3NacPh)W | 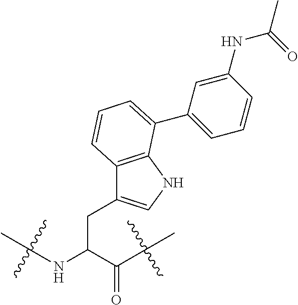 [SMILES: *NC(Cc1c[nH]c2c(-c3cccc(NC(C)=O)c3)cccc12)C(*)=O] |
| 7BrW | 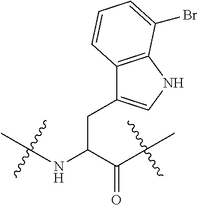 [SMILES: *NC(Cc1c[nH]c2c(Br)cccc12)C(*)=O] |
| 7MeW | 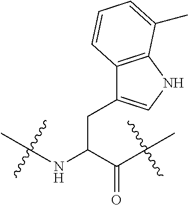 [SMILES: *NC(Cc1c[nH]c2c(C)cccc12)C(*)=O] |
| 7OH2Nal | 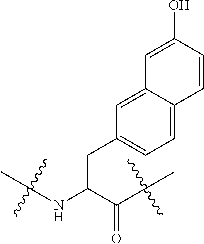 [SMILES: *NC(Cc1ccc2ccc(O)cc2c1)C(*)=O] |
| Abu | 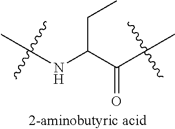 [SMILES: *NC(CC)C(*)=O] |
| ACIPA | 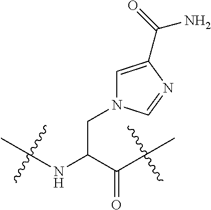 [SMILES: *NC(Cn1cnc(C(N)=O)c1)C(*)=O] |
| AEF | 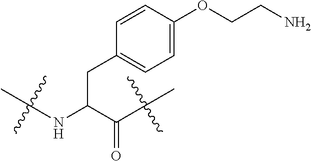 [SMILES: *NC(Cc1ccc(OCCN)cc1)C(*)=O] |
| AEF(Ac) | 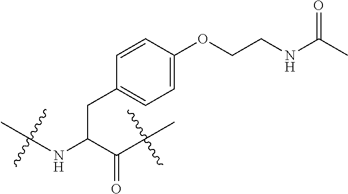 [SMILES: *NC(Cc1ccc(OCCNC(C)=O)cc1)C(*)=O] |
| AEF(BH) | 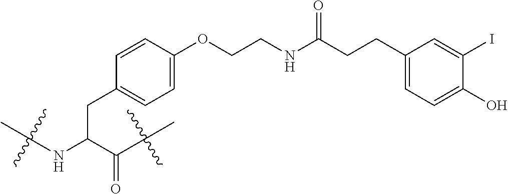 [SMILES: *NC(Cc1ccc(OCCNC(=O)CCc2ccc(O)c(I)c2)cc1)C(*)=O] |
| AEF(Boc) | 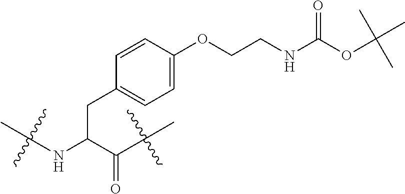 [SMILES: *NC(Cc1ccc(OCCNC(=O)OC(C)(C)C)cc1)C(*)=O] |
| AEF(EtCO) | 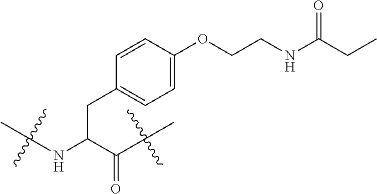 [SMILES: *NC(Cc1ccc(OCCNC(=O)CC)cc1)C(*)=O] |
| AEF(G) | 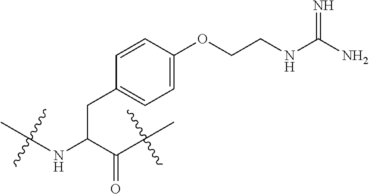 [SMILES: *NC(Cc1ccc(OCCNC(=N)N)cc1)C(*)=O] |
| AEF(NMe) | 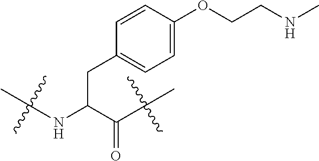 [SMILES: *NC(Cc1ccc(OCCNC)cc1)C(*)=O] |
| AEF(NMe)—Z_{peg} | 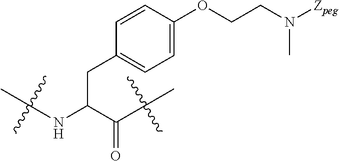 |
| AEF(cPEG3a) | 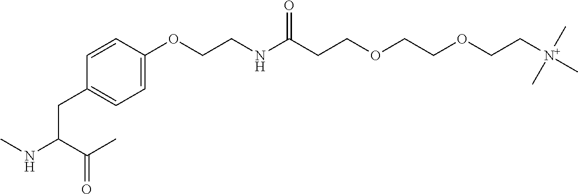 |
| AEF(NMe2) | 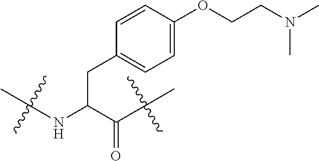 [SMILES: *NC(Cc1ccc(OCCN(C)C)cc1)C(*)=O] |
| AEF(NMe3) | 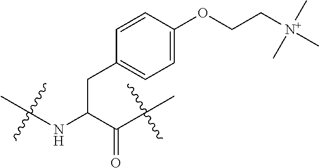 [SMILES: *NC(Cc1ccc(OCC[N+](C)(C)C)cc1)C(*)=O] |
| AEF(SMSB) | 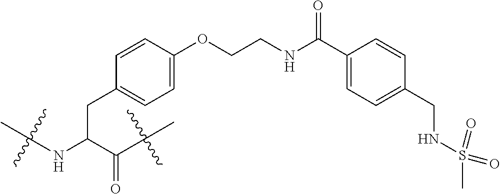 [SMILES: *NC(Cc1ccc(OCCNC(=O)c2ccc(CNS(C)(=O)=O)cc2)cc1)C(*)=O] |
| AEF—Z_{peg} | 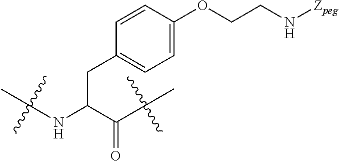 |
| AEF(NHPEG3a) | 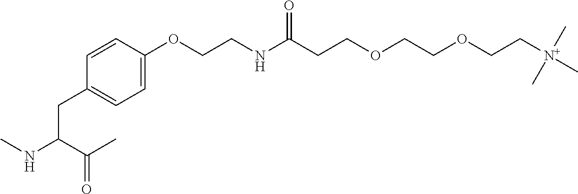 |
| aG | 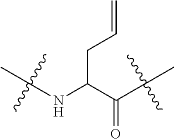 [SMILES: *NC(CC=C)C(*)=O] |
| aMeC | 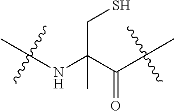 [SMILES: *NC(C)(CS)C(*)=O] |
| aMeL | 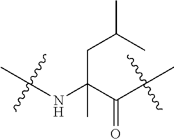 [SMILES: *NC(C)(CC(C)C)C(*)=O] |
| aMePhe | 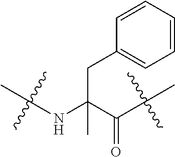 [SMILES: *NC(C)(Cc1ccccc1)C(*)=O] |
| APEG3F | 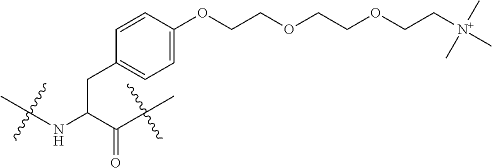 [SMILES: *NC(Cc1ccc(OCCOCCOCC[N+](C)(C)C)cc1)C(*)=O] |
| bMeAEF | 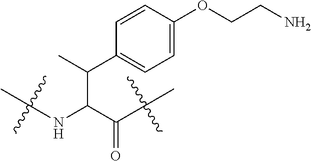 [SMILES: *NC(C(*)=O)C(C)c1ccc(OCCN)cc1] |
| BT | 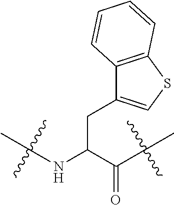 [SMILES: *NC(Cc1csc2ccccc12)C(*)=O] |
| Dab | 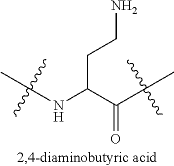 [SMILES: *NC(CCN)C(*)=O] |
| Dab(COCH2) | 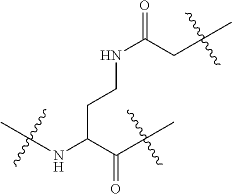 [SMILES: *CC(=O)NCCC(N*)C(*)=O] |
| Dab(NMeAc) | 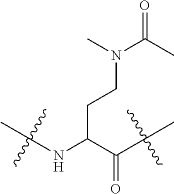 [SMILES: *NC(CCN(C)C(C)=O)C(*)=O] |
| Dab(NMe)—Z_{lipid} | 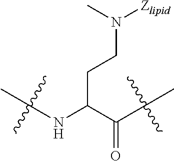 |
| Dab(NMe)—Z_{peg} | 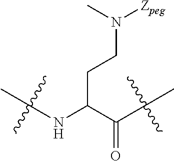 |
| Dab(NMecPEG3a) | 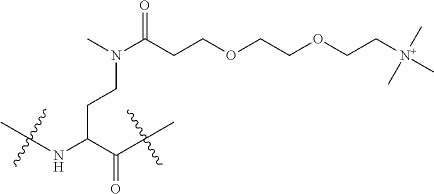 [SMILES: *NC(CCN(C)C(=O)CCOCCOCC[N+](C)(C)C)C(*)=O] |
| Dab(NMecPEG5a) | 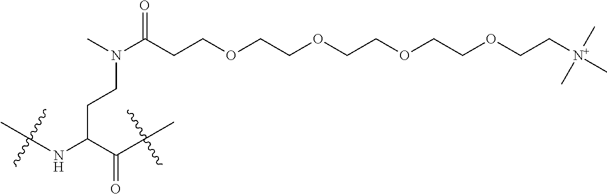 [SMILES: *NC(CCN(C)C(=O)CCOCCOCCOCCOCC[N+](C)(C)C)C(*)=O] |
| Dab(NMecarn) | 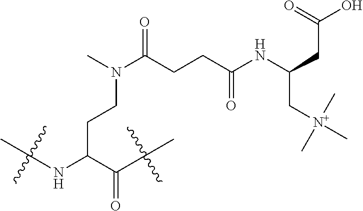 [SMILES: *NC(CCN(C)C(=O)CCC(=O)N[C@@H](CC(=O)O)C[N+](C)(C)C)C(*)=O] |
| Dab-Z_{lipid} | 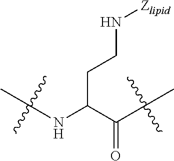 |
| Dab-Z_{peg} | 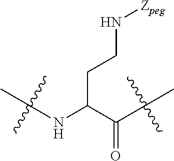 |
| Dab(cPEG3a) | 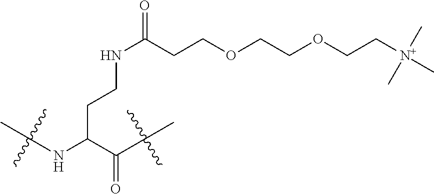 [SMILES: *NC(CCNC(=O)CCOCCOCC[N+](C)(C)C)C(*)=O] |
| Dab(cPEG5a) | 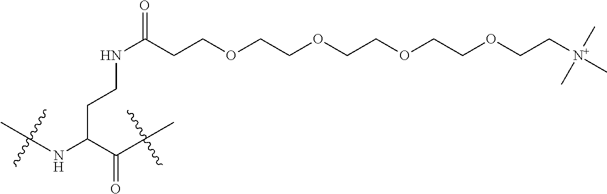 [SMILES: *NC(CCNC(=O)CCOCCOCCOCCOCC[N+](C)(C)C)C(*)=O] |
| Dap | 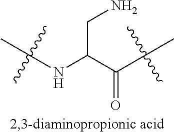 [SMILES: *NC(CN)C(*)=O] |
| Dap(N3) | 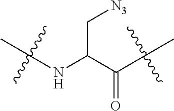 [SMILES: *NC(CN=[N+]=[N-])C(*)=O] |
| diFCpx | 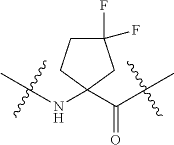 [SMILES: *NC1(C(*)=O)CCC(F)(F)C1] |
| E(COcPEG3a) | 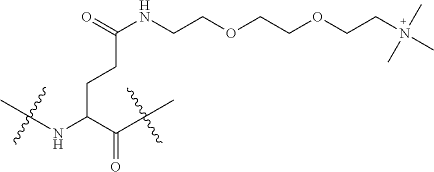 |
| hE | 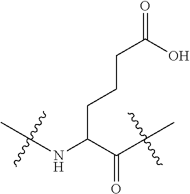 [SMILES: *NC(CCCC(=O)O)C(*)=O] |
| hK(Me)3) or hK(Me)3 | 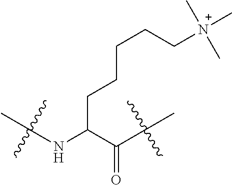 [SMILES: *NC(CCCCC[N+](C)(C)C)C(*)=O] |
| K(5cpa) | 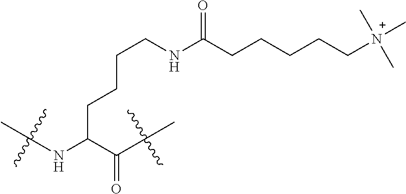 [SMILES: *NC(CCCCNC(=O)CCCCC[N+](C)(C)C)C(*)=O] |
| K(a) | 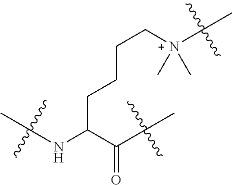 [SMILES: *NC(CCCC[N+](*)(C)C)C(*)=O] |
| K(Ac) | 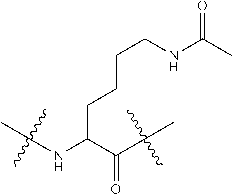 [SMILES: *NC(CCCCNC(C)=O)C(*)=O] |
| K(COCH2CH2) | 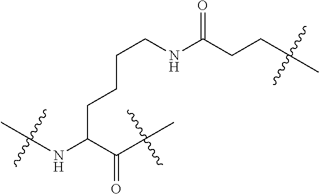 [SMILES: *CCC(=O)NCCCCC(N*)C(*)=O] |
| K(d) | 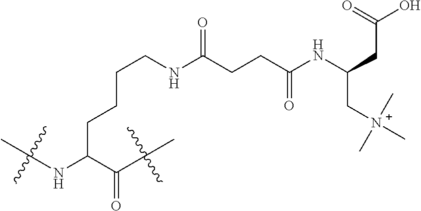 [SMILES: *NC(CCCCNC(=O)CCC(=O)N[C@@H](CC(=O)O)C[N+](C)(C)C)C(*)=O] |
| K(G) | 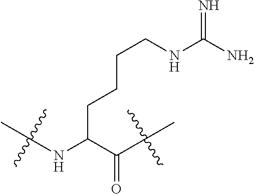 [SMILES: *NC(CCCCNC(=N)N)C(*)=O] |
| K(Me)3 | 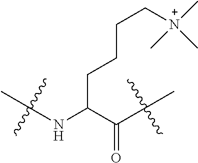 [SMILES: *NC(CCCC[N+](C)(C)C)C(*)=O] |
| K(NMe) | 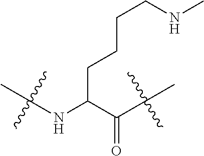 [SMILES: *NC(CCCCNC)C(*)=O] |
| K(NMeAc) | 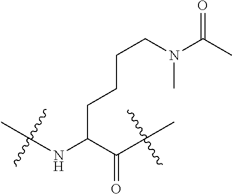 [SMILES: *NC(CCCCN(C)C(C)=O)C(*)=O] |
| K(NNs) | 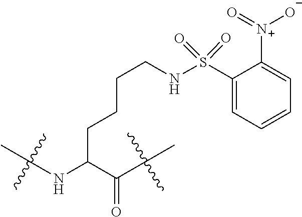 [SMILES: *NC(CCCCNS(=O)(=O)c1ccccc1[N+](=O)[O-])C(*)=O] |
| K—Z_{lipid} | 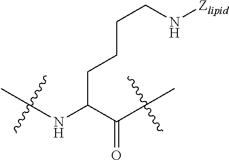 |
| K(PEG2PEG2gEC12) | 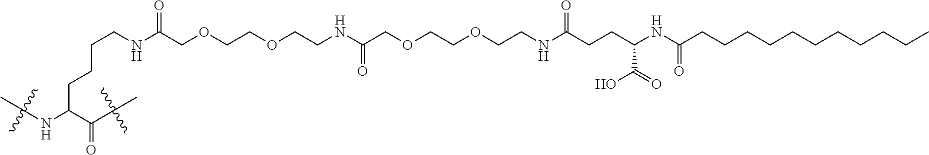 [SMILES: *NC(CCCCNC(=O)COCCOCCNC(=O)COCCOCCNC(=O)CC[C@H](NC(=O)CCCCCCCCCCC)C(=O)O)C(*)=O] |
| K(PEG2PEG2gEC14) | 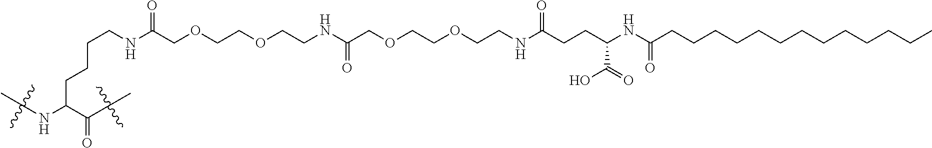 [SMILES: *NC(CCCCNC(=O)COCCOCCNC(=O)COCCOCCNC(=O)CC[C@H](NC(=O)CCCCCCCCCCCCC)C(=O)O)C(*)=O] |
| K(PEG2PEG2gEC16tetrazole) | 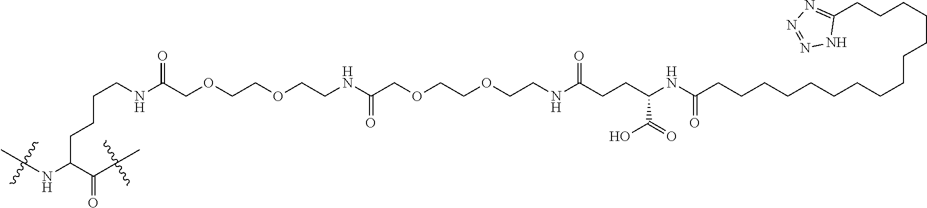 [SMILES: *NC(CCCCNC(=O)COCCOCCNC(=O)COCCOCCNC(=O)CC[C@H](NC(=O)CCCCCCCCCCCCCCCc1nnn[nH]1)C(=O)O)C(*)=O] |
| K(PEG2PEG2gEC18OH) | 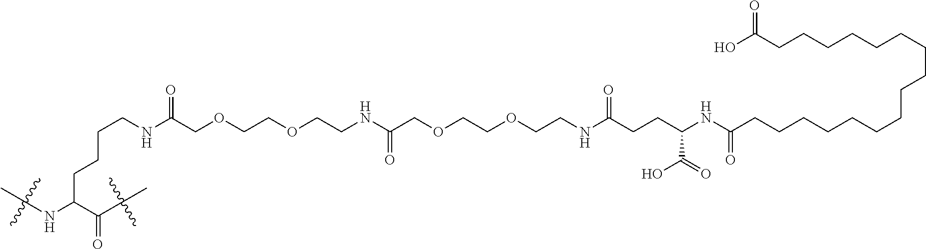 [SMILES: *NC(CCCCNC(=O)COCCOCCNC(=O)COCCOCCNC(=O)CC[C@H](NC(=O)CCCCCCCCCCCCCCCCC(=O)O)C(=O)O)C(*)=O] |
| K(PEG2NMePEG2NMegENMeC18OH) | 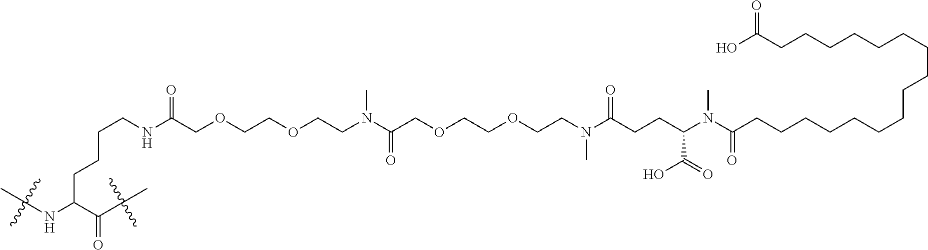 [SMILES: *NC(CCCCNC(=O)COCCOCCN(C)C(=O)COCCOCCN(C)C(=O)CC[C@@H](C(=O)O)N(C)C(=O)CCCCCCCCCCCCCCCCC(=O)O)C(*)=O] |
| K(PEG2PEG2PgEC18OH) | 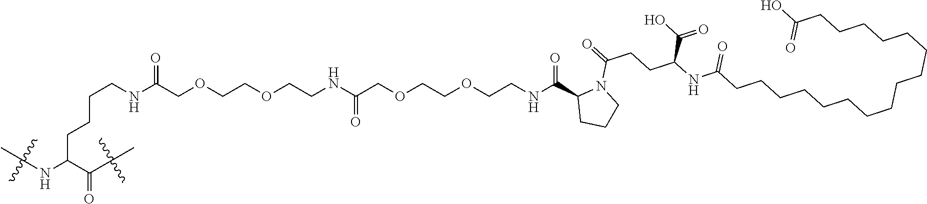 [SMILES: *NC(CCCCNC(=O)COCCOCCNC(=O)COCCOCCNC(=O)[C@@H]1CCCN1C(=O)CC[C@H](NC(=O)CCCCCCCCCCCCCCCCC(=O)O)C(=O)O)C(*)=O] |
| K(PEG2PEG2gEC18tetrazole) | 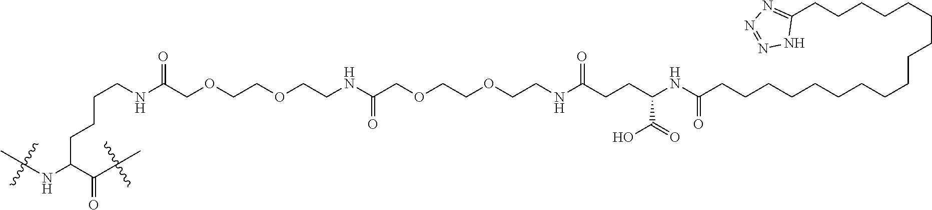 [SMILES: *NC(CCCCNC(=O)COCCOCCNC(=O)COCCOCCNC(=O)CC[C@H](NC(=O)CCCCCCCCCCCCCCCCCc1nnn[nH]1)C(=O)O)C(*)=O] |
| K(PEG2NMePEG2NMegENMeC18Tetrazole) | 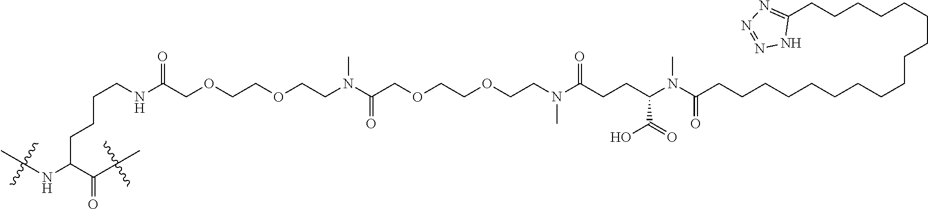 [SMILES: *NC(CCCCNC(=O)COCCOCCN(C)C(=O)COCCOCCN(C)C(=O)CC[C@@H](C(=O)O)N(C)C(=O)CCCCCCCCCCCCCCCCCc1nnn[nH]1)C(*)=O] |
| K(PEG2PEG2gEC20OH) | 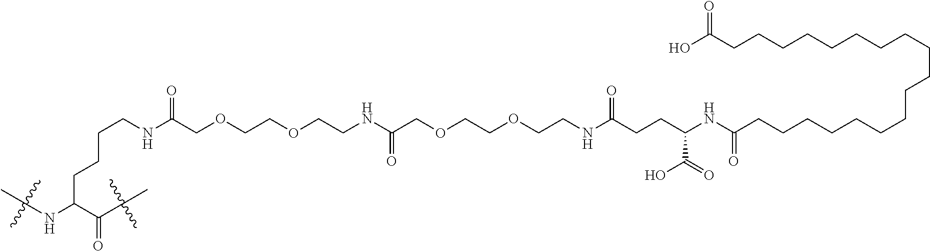 [SMILES: *NC(CCCCNC(=O)COCCOCCNC(=O)COCCOCCNC(=O)CC[C@H](NC(=O)CCCCCCCCCCCCCCCCCCC(=O)O)C(=O)O)C(*)=O] |
| K(SP6PEG2gEC18OH) | 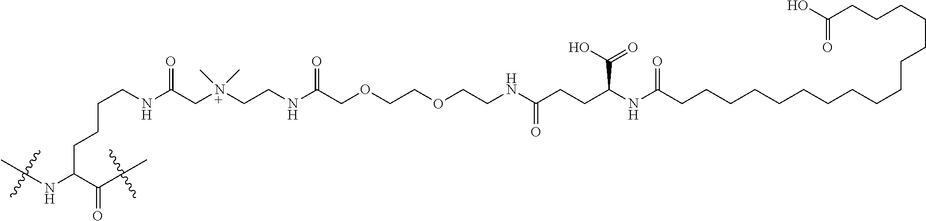 [SMILES: *NC(CCCCNC(=O)C[N+](C)(C)CCNC(=O)COCCOCCNC(=O)CC[C@H](NC(=O)CCCCCCCCCCCCCCCCC(=O)O)C(=O)O)C(*)=O] |
| K(PEG2gEgEPEG24SBC16Tetrazole) | 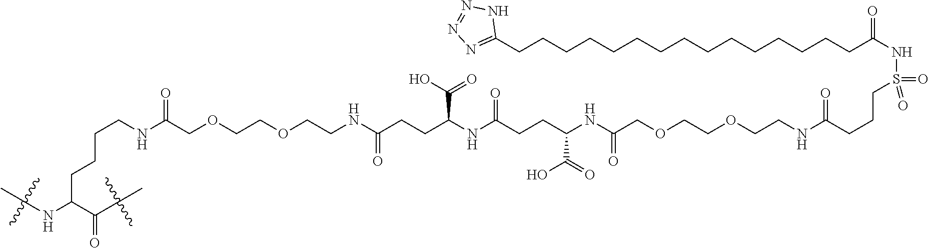 [SMILES: *NC(CCCCNC(=O)COCCOCCNC(=O)CC[C@H](NC(=O)CC[C@H](NC(=O)COCCOCCNC(=O)CCCS(=O)(=O)NC(=O)CCCCCCCCCCCCCCCc1nnn[nH]1)C(=O)O)C(=O)O)C(*)=O] |
| K(PEG6gEC18OH) | 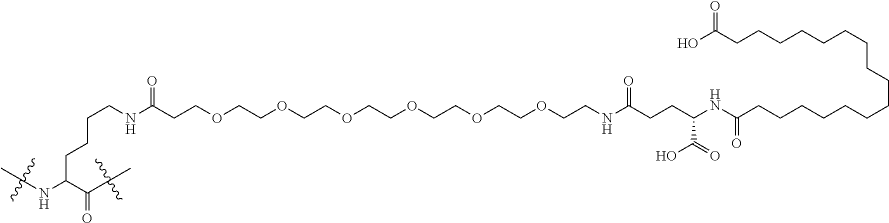 [SMILES: *NC(CCCCNC(=O)CCOCCOCCOCCOCCOCCOCCNC(=O)CC[C@H](NC(=O)CCCCCCCCCCCCCCCCC(=O)O)C(=O)O)C(*)=O] |
| K(PEG12gEC18OH) | 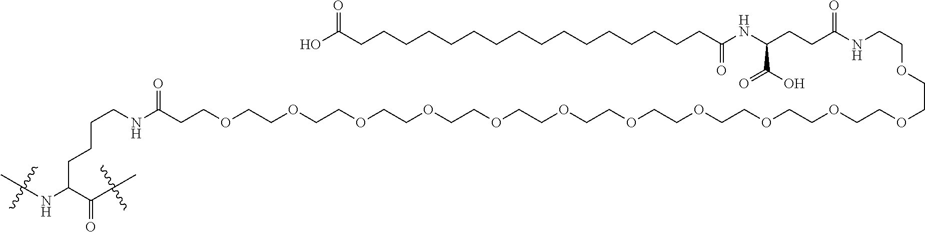 [SMILES: *NC(CCCCNC(=O)CCOCCOCCOCCOCCOCCOCCOCCOCCOCCOCCOCCOCCNC(=O)CC[C@H](NC(=O)CCCCCCCCCCCCCCCCC(=O)O)C(=O)O)C(*)=O] |
| K(PEG12NMegENMeC18OH) | 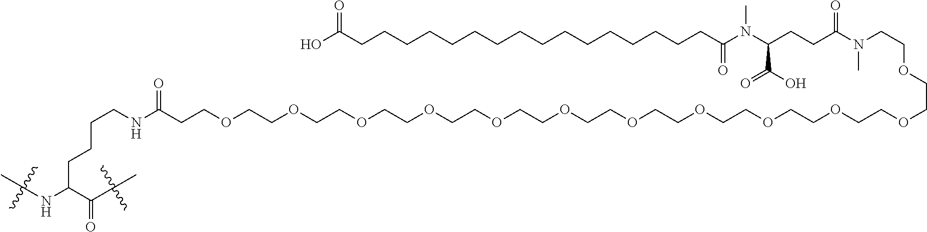 [SMILES: *NC(CCCCNC(=O)CCOCCOCCOCCOCCOCCOCCOCCOCCOCCOCCOCCOCCN(C)C(=O)CC[C@@H](C(=O)O)N(C)C(=O)CCCCCCCCCCCCCCCCC(=O)O)C(*)=O] |
| K(PEG12NMegENMeC18Tetrazole) | 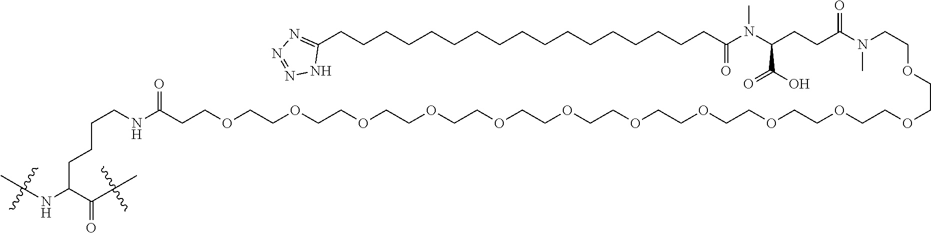 [SMILES: *NC(CCCCNC(=O)CCOCCOCCOCCOCCOCCOCCOCCOCCOCCOCCOCCOCCN(C)C(=O)CC[C@@H](C(=O)O)N(C)C(=O)CCCCCCCCCCCCCCCCCc1nnn[nH]1)C(*)=O] |
| K(PEG12gEC20OH) | 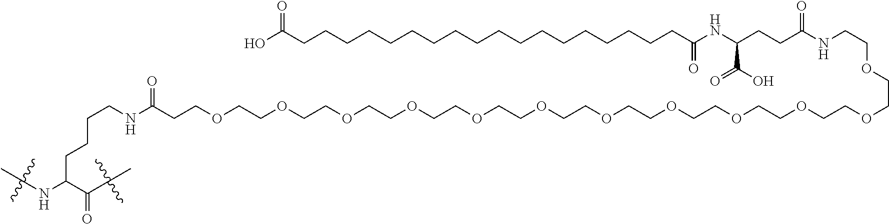 [SMILES: *NC(CCCCNC(=O)CCOCCOCCOCCOCCOCCOCCOCCOCCOCCOCCOCCOCCNC(=O)CC[C@H](NC(=O)CCCCCCCCCCCCCCCCCCC(=O)O)C(=O)O)C(*)=O] |
| K—Z_{peg} | 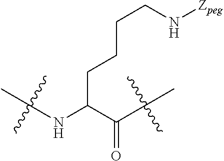 |
| K(cPEG3a) | 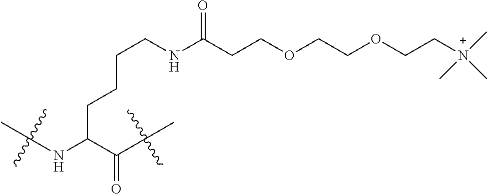 [SMILES: *NC(CCCCNC(=O)CCOCCOCC[N+](C)(C)C)C(*)=O] |
| K(mPEG12) | 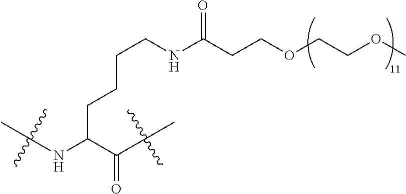 |
| K(NMe)—Z_{lipid} | 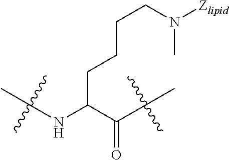 |
| K(NMe)—Z_{peg} | 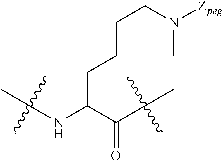 |
| K(NMecPEG3a) | 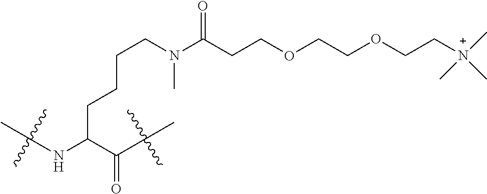 [SMILES: *NC(CCCCN(C)C(=O)CCOCCOCC[N+](C)(C)C)C(*)=O] |
| K(NMePEG12) | 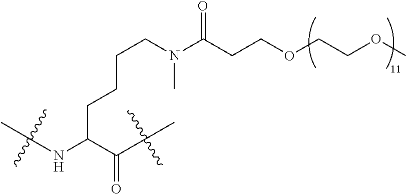 |
| Lys(N + Me2)—Z_{lipid} | 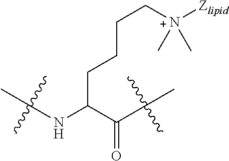 |
| Lys(N + Me2)—Z_{peg} | 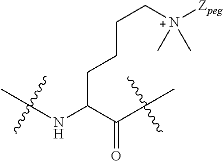 |
| Lys(N + Me2mPEG3) | 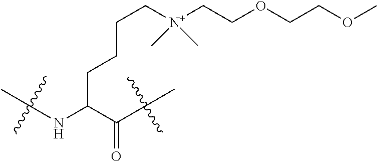 [SMILES: *NC(CCCC[N+](C)(C)CCOCCOC)C(*)=O] |
| NMeK—Z_{lipid} | 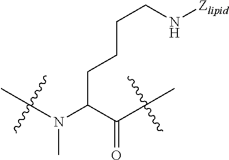 |
| NMeK(PEG2PEG2gEC12) | 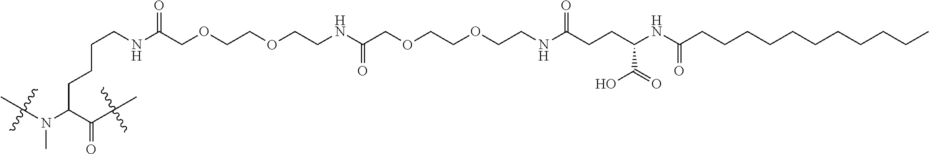 [SMILES: *C(=O)C(CCCCNC(=O)COCCOCCNC(=O)COCCOCCNC(=O)CC[C@H](NC(=O)CCCCCCCCCCC)C(=O)O)N(*)C] |
| NMeK(PEG2PEG2gEC14) | 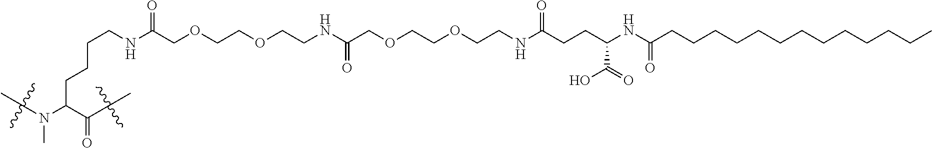 [SMILES: *C(=O)C(CCCCNC(=O)COCCOCCNC(=O)COCCOCCNC(=O)CC[C@H](NC(=O)CCCCCCCCCCCCC)C(=O)O)N(*)C] |
| NMeK(PEG2PEG2gEC16tetrazole) | 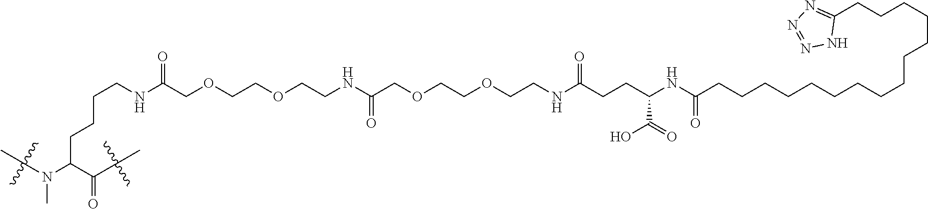 [SMILES: *C(=O)C(CCCCNC(=O)COCCOCCNC(=O)COCCOCCNC(=O)CC[C@H](NC(=O)CCCCCCCCCCCCCCCc1nnn[nH]1)C(=O)O)N(*)C] |
| NMeK(PEG2PEG2gEC18OH) | 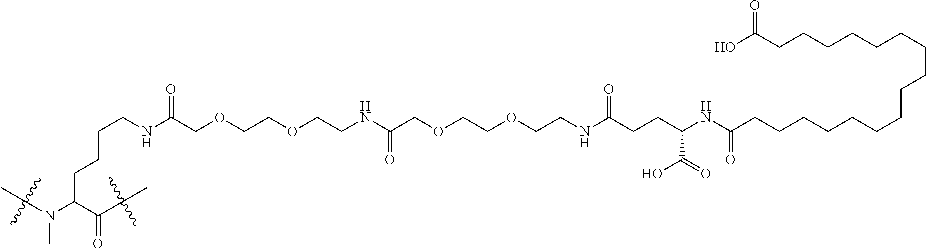 [SMILES: *C(=O)C(CCCCNC(=O)COCCOCCNC(=O)COCCOCCNC(=O)CC[C@H](NC(=O)CCCCCCCCCCCCCCCCC(=O)O)C(=O)O)N(*)C] |
| NMeK(PEG2NMePEG2NMegENMeC18OH) | 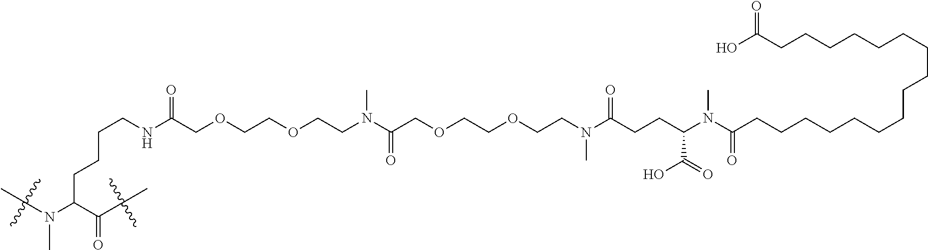 [SMILES: *C(=O)C(CCCCNC(=O)COCCOCCN(C)C(=O)COCCOCCN(C)C(=O)CC[C@@H](C(=O)O)N(C)C(=O)CCCCCCCCCCCCCCCCC(=O)O)N(*)C] |
| NMeK(PEG2PEG2PgEC18OH) | 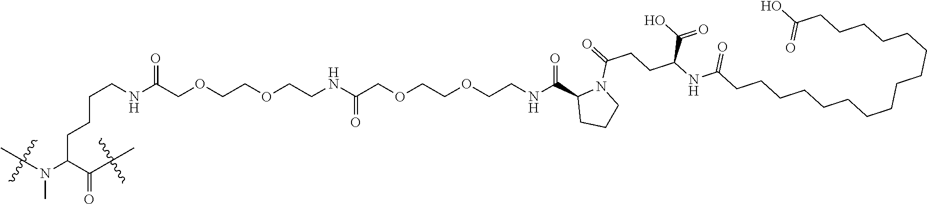 [SMILES: *C(=O)C(CCCCNC(=O)COCCOCCNC(=O)COCCOCCNC(=O)[C@@H]1CCCN1C(=O)CC[C@H](NC(=O)CCCCCCCCCCCCCCCCC(=O)O)C(=O)O)N(*)C] |
| NMeK(PEG2PEG2gEC18tetrazole) | 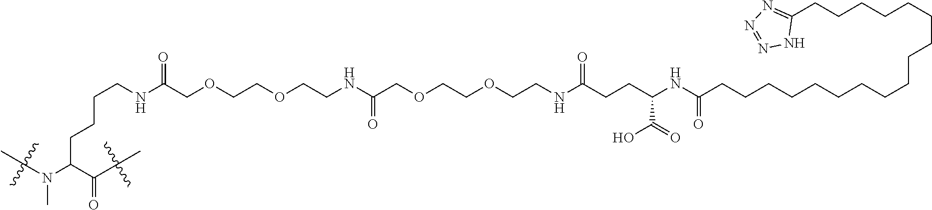 [SMILES: *C(=O)C(CCCCNC(=O)COCCOCCNC(=O)COCCOCCNC(=O)CC[C@H](NC(=O)CCCCCCCCCCCCCCCCCc1nnn[nH]1)C(=O)O)N(*)C] |
| NMeK (PEG2NMePEG2NMegENMeC18Tetrazole) | 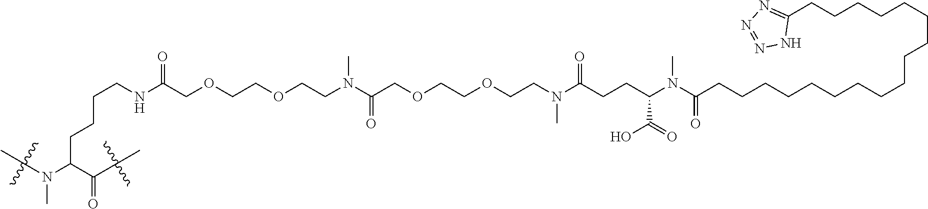 [SMILES: *C(=O)C(CCCCNC(=O)COCCOCCN(C)C(=O)COCCOCCN(C)C(=O)CC[C@@H](C(=O)O)N(C)C(=O)CCCCCCCCCCCCCCCCCc1nnn[nH]1)N(*)C] |
| NMeK(PEG2PEG2gEC20OH) | 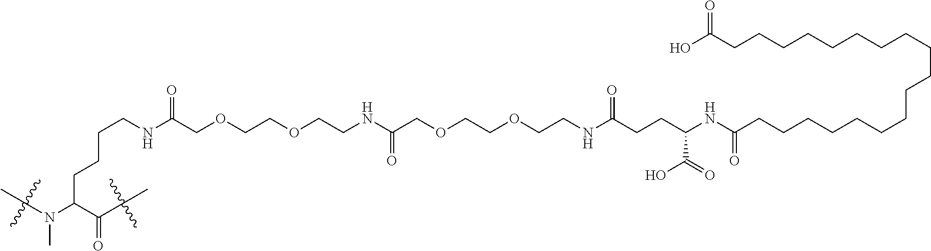 [SMILES: *C(=O)C(CCCCNC(=O)COCCOCCNC(=O)COCCOCCNC(=O)CC[C@H](NC(=O)CCCCCCCCCCCCCCCCCCC(=O)O)C(=O)O)N(*)C] |
| NMeK(SP6PEG2gEC18OH) | 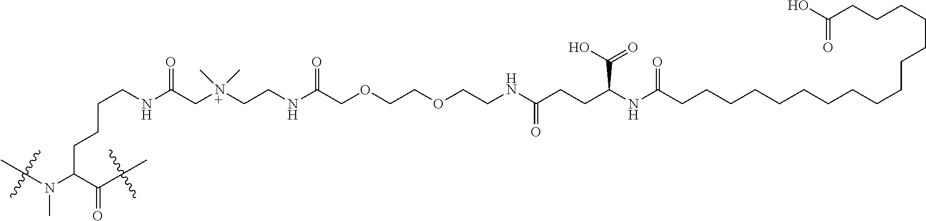 [SMILES: *C(=O)C(CCCCNC(=O)C[N+](C)(C)CCNC(=O)COCCOCCNC(=O)CC[C@H](NC(=O)CCCCCCCCCCCCCCCCC(=O)O)C(=O)O)N(*)C] |
| NMeK(PEG2gEgEPEG24SBC16Tetrazole) | 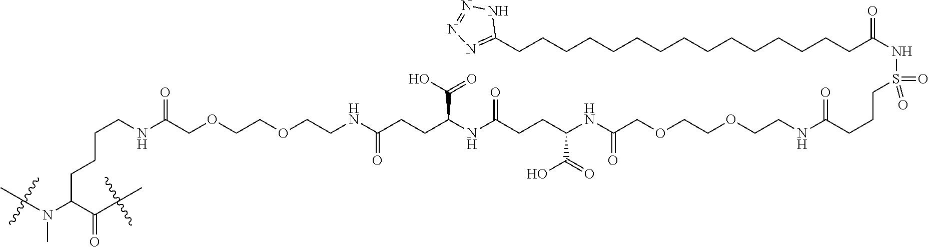 [SMILES: *C(=O)C(CCCCNC(=O)COCCOCCNC(=O)CC[C@H](NC(=O)CC[C@H](NC(=O)COCCOCCNC(=O)CCCS(=O)(=O)NC(=O)CCCCCCCCCCCCCCCc1nnn[nH]1)C(=O)O)C(=O)O)N(*)C] |
| NMeK(PEG6gEC18OH) | 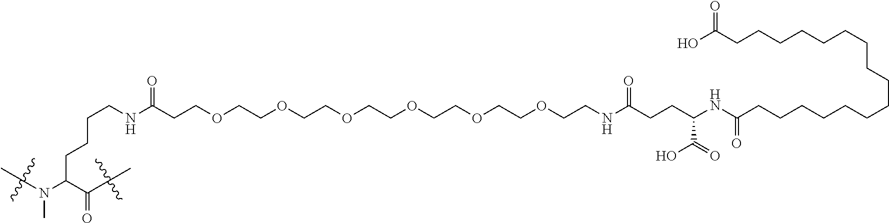 [SMILES: *C(=O)C(CCCCNC(=O)CCOCCOCCOCCOCCOCCOCCNC(=O)CC[C@H](NC(=O)CCCCCCCCCCCCCCCCC(=O)O)C(=O)O)N(*)C] |
| NMeK(PEG12gEC18OH) | 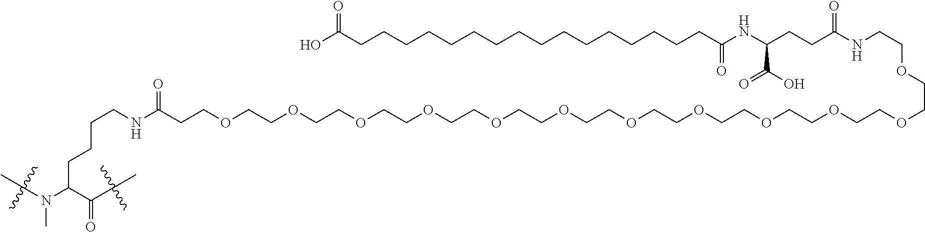 [SMILES: *C(=O)C(CCCCNC(=O)CCOCCOCCOCCOCCOCCOCCOCCOCCOCCOCCOCCOCCNC(=O)CC[C@H](NC(=O)CCCCCCCCCCCCCCCCC(=O)O)C(=O)O)N(*)C] |
| NMeK(PEG12NMegENMeC18OH) | 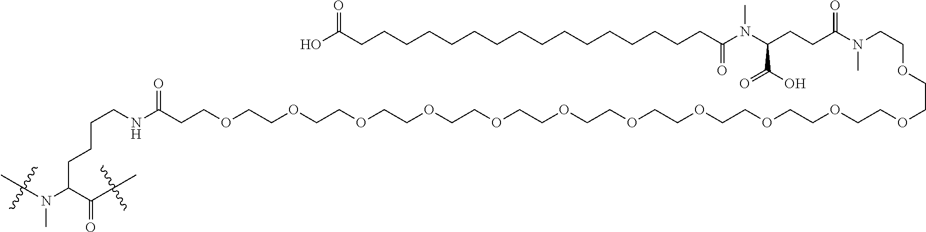 [SMILES: *C(=O)C(CCCCNC(=O)CCOCCOCCOCCOCCOCCOCCOCCOCCOCCOCCOCCOCCN(C)C(=O)CC[C@@H](C(=O)O)N(C)C(=O)CCCCCCCCCCCCCCCCC(=O)O)N(*)C] |
| NMeK(PEG12NMegENMeC18Tetrazole) | 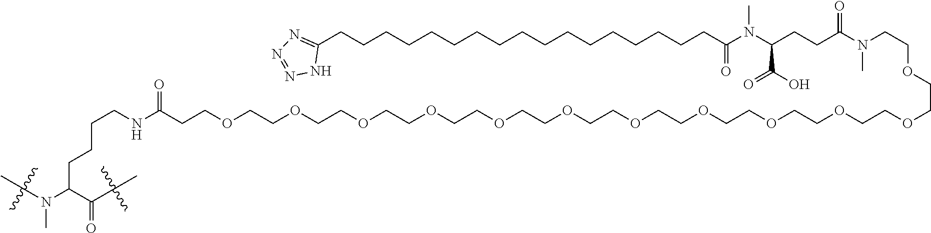 [SMILES: *C(=O)C(CCCCNC(=O)CCOCCOCCOCCOCCOCCOCCOCCOCCOCCOCCOCCOCCN(C)C(=O)CC[C@@H](C(=O)O)N(C)C(=O)CCCCCCCCCCCCCCCCCc1nnn[nH]1)N(*)C] |
| NMeK(PEG12gEC20OH) | 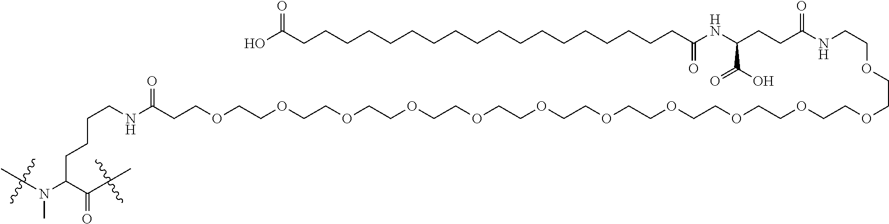 [SMILES: *C(=O)C(CCCCNC(=O)CCOCCOCCOCCOCCOCCOCCOCCOCCOCCOCCOCCOCCNC(=O)CC[C@H](NC(=O)CCCCCCCCCCCCCCCCCCC(=O)O)C(=O)O)N(*)C] |
| NMeK—Z_{peg} | 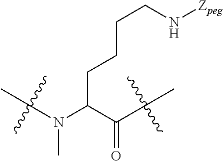 |
| MMoEF | 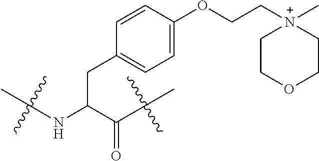 [SMILES: *NC(Cc1ccc(OCC[N+]2(C)CCOCC2)cc1)C(*)=O] |
| N(NMe) | 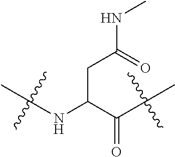 [SMILES: *NC(CC(=O)NC)C(*)=O] |
| N(NMe2) | 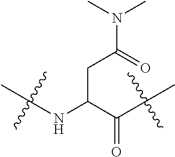 [SMILES: *NC(CC(=O)N(C)C)C(*)=O] |
| Pen | 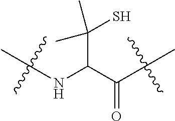 [SMILES: *NC(C(*)=O)C(C)(C)S] |
| Pen(mXyl) | 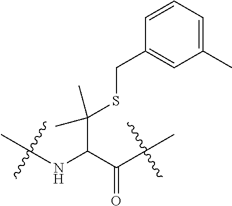 [SMILES: *NC(C(*)=O)C(C)(C)SCc1cccc(C)c1] |
| Pen(oXyl) | 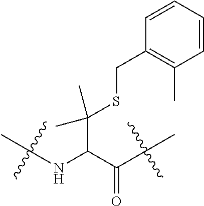 [SMILES: *NC(C(*)=O)C(C)(C)SCc1ccccc1C] |
| Pen(pXyl) | 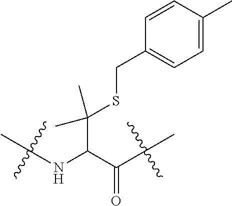 [SMILES: *NC(C(*)=O)C(C)(C)SCc1ccc(C)cc1] |
| Pip(NMe2) | 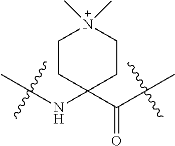 [SMILES: *NC1(C(*)=O)CC[N+](C)(C)CC1] |
| Pra | 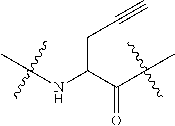 [SMILES: *NC(CC#C)C(*)=O] |
| Q(NMe) | 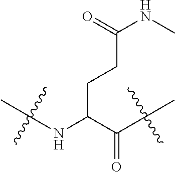 [SMILES: *NC(CCC(=O)NC)C(*)=O] |
| Q(NMe2) | 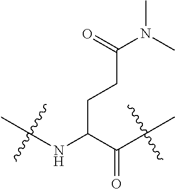 [SMILES: *NC(CCC(=O)N(C)C)C(*)=O] |
| Sar | 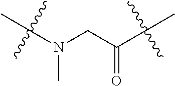 [SMILES: *C(=O)CN(*)C] |
| Ser(MePEG2) | 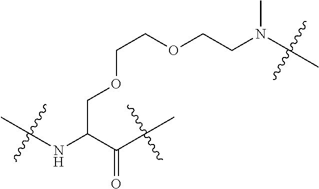 [SMILES: *NC(COCCOCCN(*)C)C(*)=O] |
| SP6 | 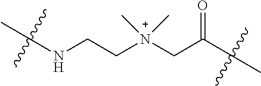 [SMILES: *NCC[N+](C)(C)CC(*)=O] |
| THP | 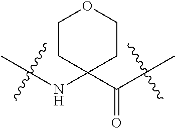 [SMILES: *NC1(C(*)=O)CCOCC1] |
| TMAPF | 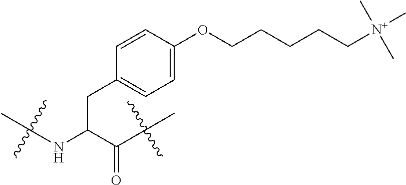 [SMILES: *NC(Cc1ccc(OCCCCC[N+](C)(C)C)cc1)C(*)=O] |
| Y(OTzl(mPEG3)) | 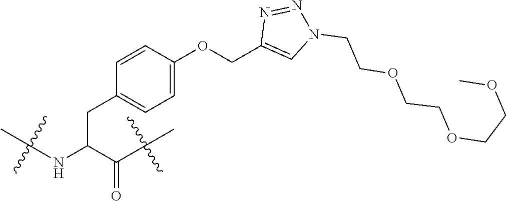 [SMILES: *NC(Cc1ccc(OCc2cn(CCOCCOCCOC)nn2)cc1)C(*)=O] |
| Y(OTzl) | 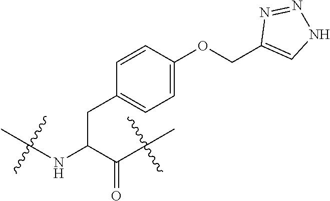 [SMILES: *NC(Cc1ccc(OCc2c[nH]nn2)cc1)C(*)=O] |

### Example 1: General Procedure for Solid-Phase Synthesis of Peptides

Peptides were chemically synthesized using optimized 9-fluorenylmethoxy carbonyl (Fmoc) solid phase peptide synthesis protocols. For C-terminal amides, Rink-amide MBHA resin or CTC resin which was then coupled to an amide after cleavage was used. The side chain protecting groups were as follows: D-Arg: Pbf; Thr, 6OH2Nal: O-tButyl; E: O-tButyl or OAll; Asn, Pen: Trityl; AEF: Boc; D-Lys: Fmoc; Lys(NMe): Alloc; Lys(Dde): Dde. For coupling, a two to five-fold excess of a solution containing Fmoc amino acid, HATU and DIEA (1:0.95:2) in DMF was added to swelled resin for 1 to 16 hours. Double coupling was employed when coupling 6OH2Nal, 6F2Nal, or other sterically hindered amino acids (i.e., the resin was treated with the amino acid and coupling reagents a second time to promote reaction completion). Fmoc protecting group removal was achieved by treatment with a DMF:piperidine (4:1) solution for 30 mi. The cycles were repeated until the full-length peptide is obtained.

Dde protecting group removal was achieved by treatment with a 300 hydrazine hydrate in DMF for 20 mi (this process was repeated 3 times). Alloc and OAll protecting group removal was achieved by treatment with a mixture of PdTetrakis/NDMBA (1,3-Dimethylbarbituric acid)/phenylsilane (0.3:10:50 eq) in dry DCM under N2 atmosphere for 30 min or in a mixture of PdTetrakis/phenylsilane (0.1:10) in dry DCM under N2 atmosphere for 15 min. The resin was then washed with DCM and cycle repeated 2 times.

Certain materials and reagents are listed below.

| | | Coupling | Reaction | | |
|---|---|---|---|---|---|
| # | Materials | reagents | time | | Note |
| 1 | Fmoc-Asp-OAll | HATU (1.90 eq) | 1 | h | |
| | (2.0 eq.) | and DIEA (4.0 | | | |
| | | eq) | | | |
| 2 | NH2-3Pya-Sar- | HATU (1.90 eq) | 2 | h | |
| | CON(Me)2 (2 eq) | and DIEA (4.0 | | | |
| | | eq) | | | |
| 3 | Fmoc-Lys(Dde)- | HATU (1.90 eq) | 2 | h | |
| | OH (2.0 eq) | and DIEA (4.0 | | | |
| | | eq) | | | |
| 4 | Fmoc-Sar-OH (3.0 | HATU (2.85 eq) | 1-2 | h | |
| | eq) | and DIEA (6.0 | | | |
| | | eq) | | | |
| 5 | Fmoc-3Pya-OH | HATU (1.90- | 1-3 | h | |
| | (2.0-3.0 eq) | 2.85 eq) and | | | |
| | | DIEA (4.0-6.0 | | | |
| | | eq) | | | |
| 6 | Fmoc-Asn(Trt)-OH | HATU (2.85- | 1-2 | h | |
| | (3.0-5.0 eq) | 4.75 eq) and | | | |
| | | DIEA (6.0-10 | | | |
| | | eq) | | | |
| 7 | Fmoc-Glu(OtBu)- | HATU (2.85- | 1-2 | h | |
| | OH (3.0-5.0 eq) | 4.75 eq) and | | | |
| | | DIEA (6.0-10.0 | | | |
| | | eq) | | | |
| 8 | Fmoc-Q(N(Me)2)- | HATU (1.90 eq) | 1-2 | h | |
| | OH (2.0 eq) | and DIEA (4.0 | | | |
| | | eq) | | | |
| 9 | Fmoc- | HATU (1.90 eq) | 1-3 | h | |
| | K(NMeAlloc)-OH | and DIEA (4.0 | | | |
| | (2.0 eq) | eq) | | | |
| 10 | Fmoc-K(NMeAc)- | HATU (1.90- | 1-2 | h | |
| | OH (2.0-3.0 eq) | 2.85 eq) and | | | |
| | | DIEA (4.0-6.0 | | | |
| | | eq) | | | |
| 11 | Fmoc-THP-OH | HATU (1.90- | 2 | h | |
| | (2.0-3.0 eq) | 2.85 eq) and | | | |
| | | DIEA (4.0-6.0 | | | |
| | | eq) | | | |
| 12 | Fmoc-6OH2Nal- | HATU (1.90- | 2 h*2 | | Double |
| | OH (2.0-3.0 eq) | 2.85 eq) and | | | cou- |
| | | DIEA (4.0-6 eq) | | | pling |
| 13 | Fmoc-6F2Nal-OH | HATU (1.9 eq) | 2 h*2 | | Double |
| | (2.0 eq) | and DIEA (4.0 | | | cou- |
| | | eq) | | | pling |
| 13 | Fmoc-6Br2Nal-OH | HATU (1.9 eq) | 2 h*2 | | Double |
| | (2.0 eq) | and DIEA (4.0 | | | cou- |
| | | eq) | | | pling |
| 14 | Fmoc-AEF(Boc)- | HATU (1.90- | 1-2 | h | |
| | OH (2.0-3.0 eq) | 2.85 eq) and | | | |
| | | DIEA (4.0-6.0 | | | |
| | | eq) | | | |
| 15 | Fmoc- | HATU (1.90 eq) | 1-2 | h | |
| | AEF(N(Me)2)-OH | and DIEA (4.0 | | | |
| | (2.0 eq) | eq) | | | |
| 16 | Fmoc-AEF(Alloc)- | HATU (2.85 eq) | 1 | h | |
| | OH (3.0 eq) | and DIEA (6.0 | | | |
| | | eq) | | | |
| 17 | Fmoc-Pen(Trt)-OH | HATU (1.90- | 2 | h | |
| | (2.0-3.0 eq) | 2.85 eq) and | | | |
| | | DIEA (4.0-6.0 | | | |
| | | eq) | | | |
| 18 | Fmoc-K(Ac)-OH | HATU (1.90- | 1-2 | h | |
| | (2.0-3.0 eq) | 2.85 eq) and | | | |
| | | DIEA (4.0-6 eq) | | | |
| 19 | Fmoc-7MeTrp-OH | HATU (1.90- | 1-2 | h | |
| | (2.0-3.0 eq) | 2.85 eq) and | | | |
| | | DIEA (4.0-6.0 | | | |
| | | eq) | | | |
| 20 | Fmoc- | HATU (1.90- | 2 | h | |
| | 7(3NAcPh)Trp-OH | 2.85 eq) and | | | |
| | (2.0 eq) | DIEA (4.0-6 eq) | | | |
| 21 | Fmoc-Thr(tBu)-OH | HATU (2.85- | 1-2 | h | |
| | (3.0-5.0 eq) | 4.75 eq) and | | | |
| | | DIEA (6.0-10 | | | |
| | | eq) | | | |
| 22 | Fmoc-Asn(Trt)-OH | HATU (2.85 eq) | 2 | h | |
| | (3.0 eq) | and DIEA (6.0 | | | |
| | | eq) | | | |
| 23 | Fmoc-N(N(Me)2)- | HATU (1.90 eq) | 1-2 | h | |
| | OH (2.0 eq) | and DIEA (4.0 | | | |
| | | eq) | | | |
| 24 | Fmoc-Glu(OAll)- | HATU (1.9 eq) | 2 | h | |
| | OH (2.0 eq) | and DIEA (4.0 | | | |
| | | eq) | | | |
| 25 | Pd(PPh3)4 (0.1 eq), | DCM | 15 min*3 | | De- |
| | PhSiH3 (10 eq) | | times | | OAll |
| | | | | | and De- |
| | | | | | Alloc |
| 26 | HATU (2.0 eq) and | DMF | 1 | h | Cycliza- |
| | DIEA (4.0 eq) | | | | tion |
| | | | | | resin |
| 27 | Fmoc-Pen(Trt)-OH | HATU (2.85 eq) | 2 | h | |
| | (3.0 eq) | and DIEA (6.0 | | | |
| | | eq) | | | |
| 28 | Fmoc-d-Arg(Pbf)- | HATU (2.85 eq) | 2 | h | |
| | OH (3.0 eq) | and DIEA (6.0 | | | |
| | | eq) | | | |
| 29 | Dde-d-Lys(Fmoc)- | HATU (1.90 eq) | 1 | h | |
| | OH (2.0 eq) | and DIEA (4.0 | | | |
| | | eq) | | | |
| 30 | 5cpa (2.0 eq) | HATU (1.90 eq) | 2 | h | |
| | | and DIEA (4.0 | | | |
| | | eq) | | | |
| 31 | cPEG3a (2.0 eq) | HATU (1.90 eq) | 2-3 | h | |
| | | and DIEA (4.0 | | | |
| | | eq) | | | |
| 32 | Fmoc-hk(Me)3-OH | HATU (1.90 eq) | 2 | h | |
| | (2.0 eq) | and DIEA (4.0 | | | |
| | | eq) | | | |
| 33 | PEG2NMePEG2N | HATU (1.9 eq) | 16 | h | |
| | MegENMeC18Tetra- | and DIEA (4.0 | | | |
| | zole(2.0 eq) | eq) | | | |
| 34 | PEG2PEG2gEC18 | DIC (2.0 eq) and | 16 | h | |
| | OH(2.0 eq) | HOAT (2.0 eq) | | | |
| 35 | 10% Ac₂O/5% | N/A | 1 | h | |
| | NMM/85% DMF | | | | |
| 36 | ETFA(10 eq + 10 | DBU (15 eq) | 1 h*4 times | | |
| | eq) | DMF | and overnight | | |
| 37 | 3% hydrazine | N/A | 20 | min | 3 times |
| | hydrate in DMF | | | | |

### General Procedure for Cleavage of Peptides Off Resin

Side chain deprotection and cleavage of the peptides was achieved by stirring the dry resin in a solution of trifluoroacetic acid, water, DTT and tri-isopropylsilane (90:2.5:5:2.5) for 3 hours. The mixture was then filtered and cold methyl tert-butyl ether (MTBE) was added to the combined filtrate to precipitate the peptide. The resulting mixture was centrifuged (3000 rpm, 3 min) and decanted. The pellet was washed with MTBE and centrifuged. The pellet was lyophilized to provide the linear peptide.

### General Procedure for Cyclization

To effect cyclization of thiol-containing residues, iodine solution in MeOH (0.1M) was added to a solution of the linear peptide (20% MeCN/H2O (1 mmol/L)) drop-wise until a yellow color persisted. After about 2 h, analysis by LCMS showed that the linear peptide was no longer present. The excess iodine was quenched by the addition of 1M Na₂S₂O₃ in water (turned colorless instantly).

### General Procedure for Purification of Peptides

Purification of the peptides was achieved using reverse-phase high performance liquid chromatography (RP-HPLC). Purification of the cyclized peptides was achieved using preparative RP-HPLC with a C18 column with a flow rate of 20-250 mL/min. Separation was achieved using gradients of buffer B in A (Buffer A: 0.075% TFA in water; Buffer B: ACN). (Note 1). Analysis was performed using a C18 column with a flow rate of 1 mL/min (Note 2).

### Note 1: Preparative HPLC Methods

Prep. HPLC Method A: Description: Mobile Phase: 0.075% TFA in water (solvent A) and acetonitrile (solvent B) Column: Phenomenex Luna® C18, 250*100 mm, 10 um, 120 Å column; Flow Rate: 250 mL/min; Wavelength: UV 220 nm&254 nm; Oven Temp. Room temperature

Prep. HPLC Method B: Description: Mobile Phase: 0.5% AcOH in water (solvent A) and acetonitrile (solvent B) Column: Phenomenex Luna® C18, 250*100 mm, 10 um, 120 Å column; Flow Rate: 250 mL/min; Wavelength: UV 220 nm&254 nm; Oven Temp. Room temperature

Prep. HPLC Method C: Description: Mobile Phase: 0.075% TFA in water (solvent A) and acetonitrile (solvent B) Column: Luna 100*25 mm, C18, 10 um, 100 Å+Gemini® 150*30 mm, C18, 5 um, 110 Å column; Flow Rate: 20 mL/min; Wavelength: UV 220 nm&254 nm; Oven Temp. Room temperature

Prep. HPLC Method D: Description: Mobile Phase: 0.5% AcOH in water (solvent A) and acetonitrile (solvent B) Column: Luna 100*25 mm, C18, 10 um, 100 Å+Gemini® 150*30 mm, C18, 5 um, 110 Å column; Flow Rate: 20 mL/min; Wavelength: UV 220 nm&254 nm; Oven Temp. Room temperature

Prep. HPLC Method E: Description: Mobile Phase: 0.075% TFA in water (solvent A) and acetonitrile (solvent B) Column: Welch Ultimate XB-C18, 250*50 mm, 7 um, 120 Å+Welch Xtimate C18, 250*50 mm, 10 um, 120 Å column; Flow Rate: 80 mL/min; Wavelength: UV 220 nm&254 nm; Oven Temp. Room temperature

Prep. HPLC Method F: Description: Mobile Phase: 0.5% AcOH in water (solvent A) and acetonitrile (solvent B) Column: Welch Ultimate® XB-C18, 250*50 mm, 10 um, 120 Å+Welch Xtimate®C18, 250*50 mm, 10 um, 120 Å column; Flow Rate: 80 mL/min; Wavelength: UV 220 nm&254 nm; Oven Temp. Room temperature

Prep. HPLC Method G: Description: Mobile Phase: 0.075% TFA in water (solvent A) and acetonitrile (solvent B) Column: Luna C18, 200*25 mm, 10 um, 100 Å+Gemin C18, 150*30 mm, 5 um, 110 Å column; Flow Rate: 20 mL/min; Wavelength: UV 220 nm&254 nm; Oven Temp. Room temperature

Prep. HPLC Method H: Description: Mobile Phase: 0.075% TFA in water (solvent A) and acetonitrile (solvent B) Column: YMC-Actus Triart C18, 250*30 mm, 5 um, 120 Å column; Flow Rate: 20 mL/min; Wavelength: UV 220 nm&254 nm; Oven Temp. Room temperature

Prep. HPLC Method I: Description: Mobile Phase: 0.075% TFA in water (solvent A) and acetonitrile (solvent B) Column: Luna 8 cm*250 mm, C18, 5 um, 100 Å column; Flow Rate: 150 mL/min; Wavelength: UV 220 nm&254 nm; Oven Temp. Room temperature

### Note 2: Analytical HPLC Method:

Mobile Phase: 0.1% TFA in water (solvent A) and 0.1% TFA in acetonitrile (solvent B), using the elution gradient 10%-80% (solvent B) over 0.9 minutes and using the elution gradient 80%-90% for 0.6 minutes at a flow rate of 1.0 ml/min; Column: Xbridge C18, 3.5 um, 2.1*30 mm; Wavelength: UV 220 nm&254 nm; Column temperature: 30° C.; MS ionization: ESI

### Example 2: Synthesis of SEQ ID NO: 349—MeCO-k(d)-Pen(3)-N-T-7MeW-K(Ac)-Pen(3)-AEF-6OH2Nal-THP-E-N-3Pya-Sar-CONH2

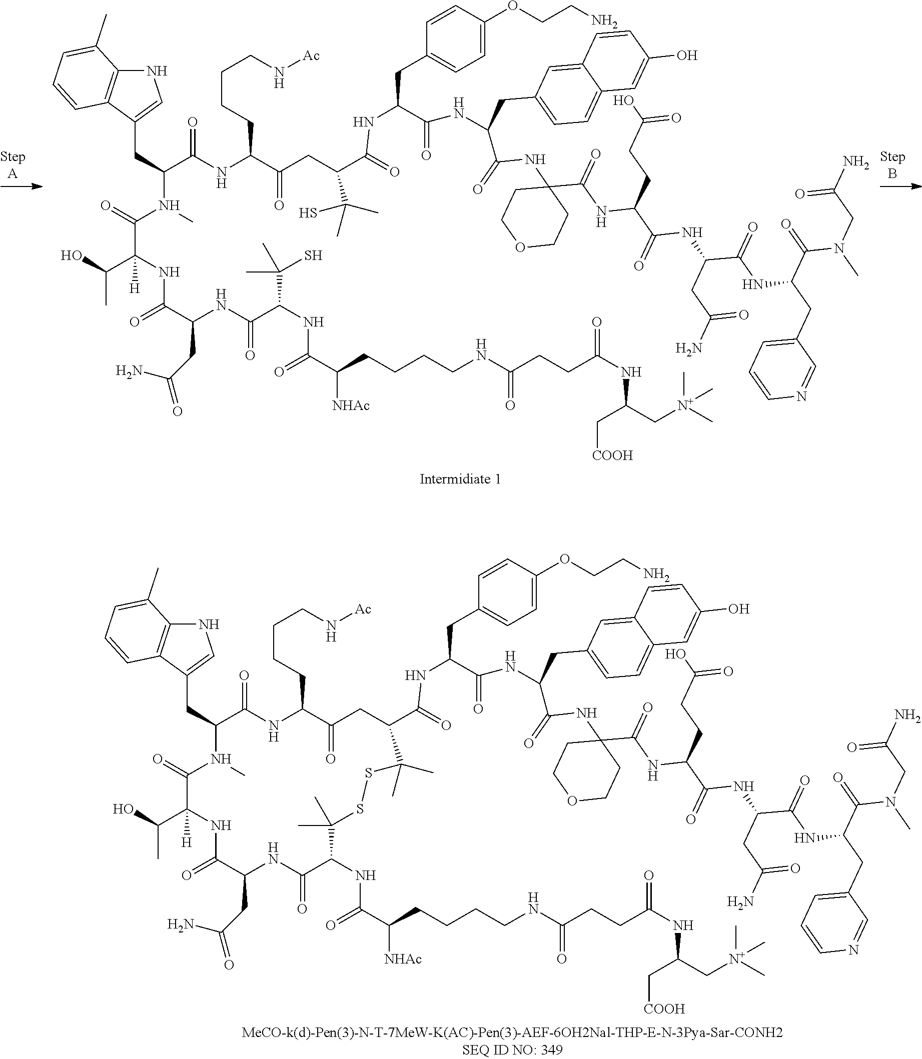

Step A—Synthesis of Intermediate 1: The peptide was synthesized by Solid-phase Peptide Synthesis (SPPS) using Fmoc/t-Bu chemistry. The assembly was performed on a Rink-amide AM resin (220 μmol, 100-200 Mesh; loading 0.35 mmol/g) on the CEM Liberty Blue microwave peptide synthesizer (CEM Inc.). During peptide assembly on solid phase, the side chain protecting groups were: tert-butyl for Thr and Glu; trityl for Pen and Asn; tert-butoxy-carbonyl for AEF. The D-Lys at position X₃ was protected by the orthogonal Dde protecting group.

All the amino acids were dissolved at a 0.4 M concentration in DMF. The acylation reactions were performed for 3 min at 90° C. under MW irradiation with 5 fold excess of activated amino acids over the resin free amino groups. The amino acids were activated with equimolar amounts of 0.5 M solution of DIC in DMF and Oxyma solution 1 M in DMF. Double acylation reactions were performed for 3Pya at X₁₅. Manual coupling was performed for 6OH2Nal at X₁₁ using DIC-HOAT (3 eq, 1:1:1) at room temperature. Fmoc deprotections were performed using 20% (V/V) piperidine in DMF. Capping of the free amino group was performed manually using 10 eq of acetic anhydride in DMF.

At the end of the peptide assembly on solid phase, the resin was treated with 100 ml of 3% hydrazine solution in DMF. The solution was drained, and the resin washed with DCM (3×5 mL) and DMF (5×5 mL). Carnitine-succinate ((3 eq, d) was coupled to the lysine residue at the X₃ position using HATU/DIPEA (1:1:2) at room temperature.

At the end of the assembly the resin was washed with DMF, MeOH, DCM, Et₂O. The peptide was cleaved from solid support using 30 ml of TFA solution (v/v) (87.5% TFA, 5% H₂O, 2.5% TIPS, 5% Phenol) for approximately 1.5 hours, at room temperature. The resin was then filtered and the filtrate was triturated in cold MTBE (135 mL). After centrifugation, the peptide pellets were washed with fresh cold diethyl ether The process was repeated twice. Final pellets were dried, resuspended in H₂O and acetonitrile 1:1+0.10% TFA and stirred overnight. The suspension was then lyophilized to afford intermediate 1 (Yield=88%). LCMS anal. calc. For C₁₀₇H₁₅₃N₂₄O₂₈S²⁺: 2287.66 Da. found; 1144.6 (M+2)²⁺

Step B—Synthesis of SEQ ID NO: 349: MeCO-k(d)-Pen(3)-N-T-7MeW-K(Ac)-Pen(3)-AEF-6OH2Nal-THP-E-N-3Pya-Sar-CONH2:

Intermediate 1 was dissolved in ACN\H₂O (5 mg\ml). Saturated iodine in acetic acid was then added dropwise to the solution while stirring until yellow color persisted. The reaction was completed in 20 min (as monitored by UPLC-MS). Solid ascorbic acid was added until the solution became clear. After lyophilization the cyclized peptide was purified by reverse-phase HPLC using preparative Waters DeltaPak C4 (200×40 mm, 300 Å, 15 μm). Mobile phase A: +0.1% TFA, mobile phase B: Acetonitrile (ACN)+0.1% TFA. The following gradient of eluent B was used: 10% B to 10% B over 5 min, to 25% B over 25 min, flow rate 80 mL/min, wavelength 214 nm. The collected fractions were lyophilized to afford SEQ ID NO: 349: MeCO-k(d)-Pen(3)-N-T-7MeW-K(Ac)-Pen(3)-AEF-6OH2Nal-THP-E-N-3Pya-Sar-CONH2(Yield=31%) LCMS anal. calc. For C₁₀₇H₁₅₁N₂₄O₂₈S₂⁺: 2285.64 Da. found; 1143.3 (M+2)²⁺

### Example 3: Synthesis of SEQ ID NO: 339—MeCO-Pen(3)-N(N(Me)₂)-T-7MeW-Dab(NMecarn)-Pen(3)-AEF-6OH2Nal-THP-Dab(NMecarn)-N-3Pya-Sar-CON(Me)2

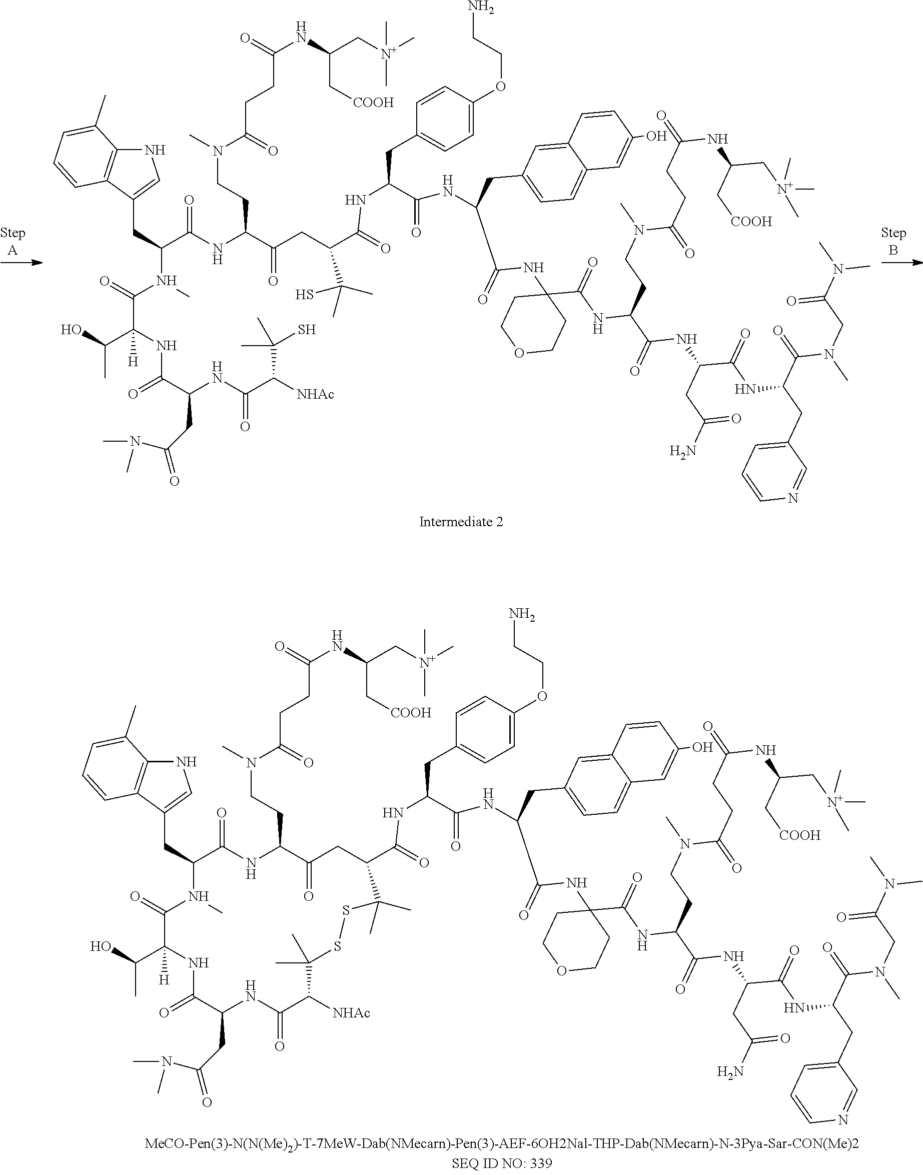

Step A—Synthesis of Intermediate 2: The peptide was synthesized by Solid-phase Peptide Synthesis (SPPS) using Fmoc/t-Bu chemistry. The assembly was performed on a Rink-amide AM resin (220 μmol, 100-200 Mesh; loading 0.35 mmol/g). The first amino acid was loaded manually using an equimolar solution of Fmoc-Asp-OAll, HOAt and DIC in DMF, at room temperature. Complete acylation was monitored by ninhydrin test. The resin was then treated with 0.25 eq of Pd Tetrakis, 24 eq of phenylsilane in 5 ml of dry DCM under N2 atmosphere for 30 min (process repeated 2 times); washed with DCM, DMF, and a solution of 0.5% sodium dimethyldithiocarbamate (0.5%) and DIPEA (0.5%) in DMF. Fmoc-OSu and DIPEA (1:1, 2 eq) in DCM were added and stirred at room temperature for 30 minutes. Further peptide elongation was performed by manually pre-activating the resin with HATU (1.2 eq), DIPEA (2.2 eq) and then adding a solution of DIPEA (2.2 eq) and 3Pya-Sar-CON(Me)2 dimer (2.2 eq in DMF. The reaction mixture was stirred for 1 h at room temperature. Complete acylation was monitored by test cleavage. The resin was then placed in a microwave reaction vessel and the assembly was continued on the CEM Liberty Blue microwave peptide synthesizer (CEM Inc.). During peptide assembly on solid phase, the side chain protecting groups were: tert-butyl for Thr and Glu; trityl for Pen and Asn. All the amino acids were dissolved at a 0.4 M concentration in DMF. The acylation reactions were performed for 3 min at 90° C. under MW irradiation with 5 folds excess of activated amino acids over the resin free amino groups. The amino acids were activated with equimolar amounts of 0.5 M solution of DIC in DMF and Oxyma solution 1M in DMF. Double acylation reactions were performed for 3Pya at X₁₅. 60H2Nal at X₁₁, DabNMeAlloc at X₈ and DabNMeAlloc at X₁₃ were coupled manually using DIC-HOAT (3 eq, 1:1:1) at room temperature and complete acylation was monitored by ninhydrin test. Fmoc deprotections were performed using 20% (V/V) piperidine in DMF. Capping of the free amino group was performed manually using 10 eq of acetic anhydride in DMF.

The resin was then treated with 0.3 eq of Pd Tetrakis, 50 eq of phenylsilane and 10 eq of barbituric acid in 5 ml of dry DCM under N2 atmosphere for 20 min (process repeated 2 times); washed with DCM, DMF, and a solution of sodium dimethyldithiocarbamate (0.5%) and DIPEA (0.5%) in DMF. Further side chain derivatization was performed manually using HATU (4 eq), DIPEA (8 eq) and then adding 4 eq of carnitine-succinate. The reaction was complete after 1 h.

At the end of the assembly, the resin was washed with DMF, MeOH, DCM, and Et₂O. The peptide was cleaved from solid support using 30 ml of TFA solution (v/v) (87.5% TFA, 5% H₂O, 2.5% TIPS, 5% phenol) for approximately 1.5 hours, at room temperature. The resin was then filtered and the filtrate was triturated in cold MTBE (135 mL). After centrifugation, the peptide pellets were washed with cold diethyl ether. The process was repeated twice. Final pellets were dried, resuspended in H₂O and acetonitrile 1:1+0.10% TFA and stirred overnight. The suspension was then lyophilized to afford the desired linear Intermediate 2 (Yield=78%). LCMS anal. calc. For C₁₁₃H₁₆₇N₂₅O₂₈S₂⁺: 2387.84 Da. found; 1193.7 (M+2)²⁺

Step B—Synthesis of MeCO-Pen(3)-N(N(Me)2)-T-7MeW-Dab(NMecarn)-Pen(3)-AEF-6OH2Nal-THP-Dab(NMecarn)-N-3Pya-Sar-CON(Me)2 SEQ ID NO: 339: The peptide (Intermediate 2) was dissolved in ACN\H₂O (5 mg\ml). Saturated iodine in acetic acid was then added dropwise to the solution while stirring until yellow color persisted. The reaction was complete in 30 min (monitored by UPLC-MS). Solid ascorbic acid was added until the solution became clear. After lyophilization, the cyclized peptide was purified by reverse-phase HPLC using preparative Waters DeltaPak C4 (200×40 mm, 300 Å, 15 μm). Mobile phase A: +0.1% TFA, mobile phase B: Acetonitrile (ACN)+0.1% TFA. The following gradient of eluent B was used: 10% B to 10% B over 5 min, to 25% B over 25 min, flow rate 80 mL/min, wavelength 214 nm. Collected fractions were lyophilized to afford SEQ ID NO: 339 (Yield=23%) LCMS anal. calc. For C₁₁₃H₁₆₅N₂₅O₂₈S₂⁺: 2384.17. found; 1192.5 (M+2)²⁺

### Example 4: Synthesis of SEQ ID NO: 112—5cpaCO-Pen(3)-K(5)-T-7MeW-K(Ac)-Pen(3)-AEF(5)-6OH2Nal-THP-K(Ac)-N-3Pya-Sar-CONH2

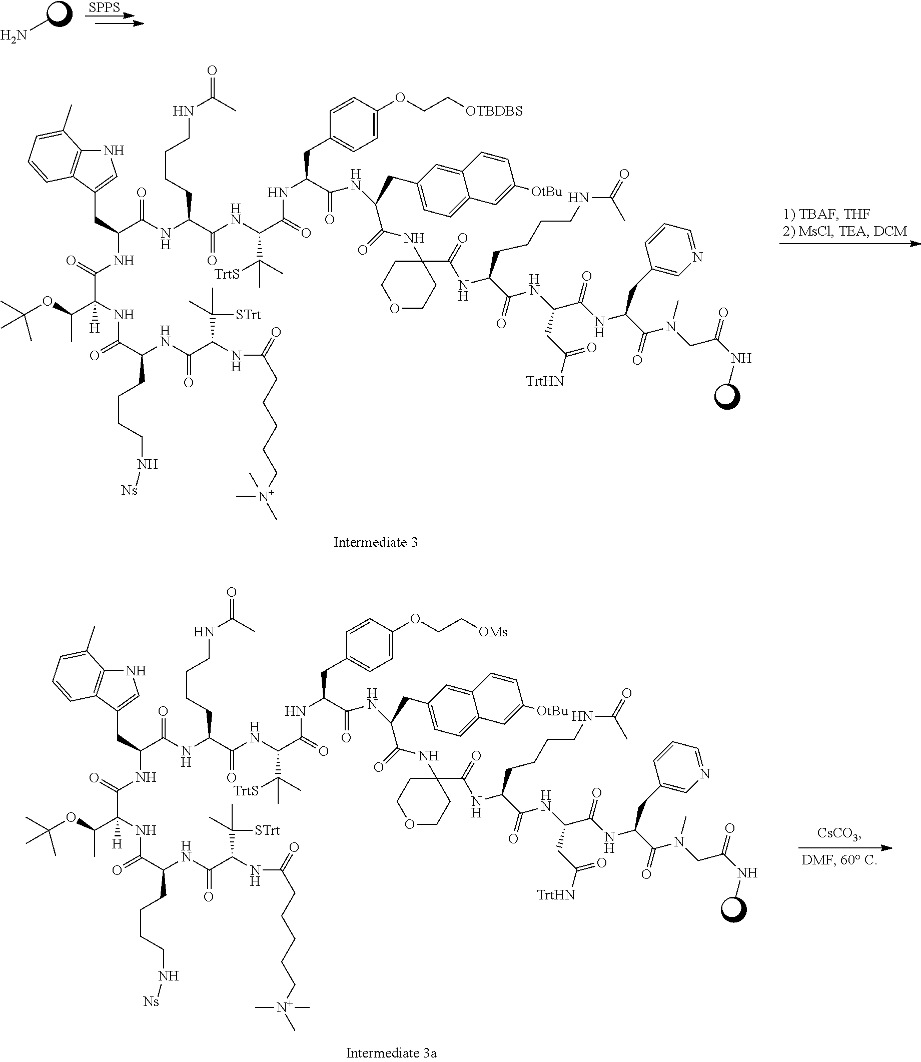

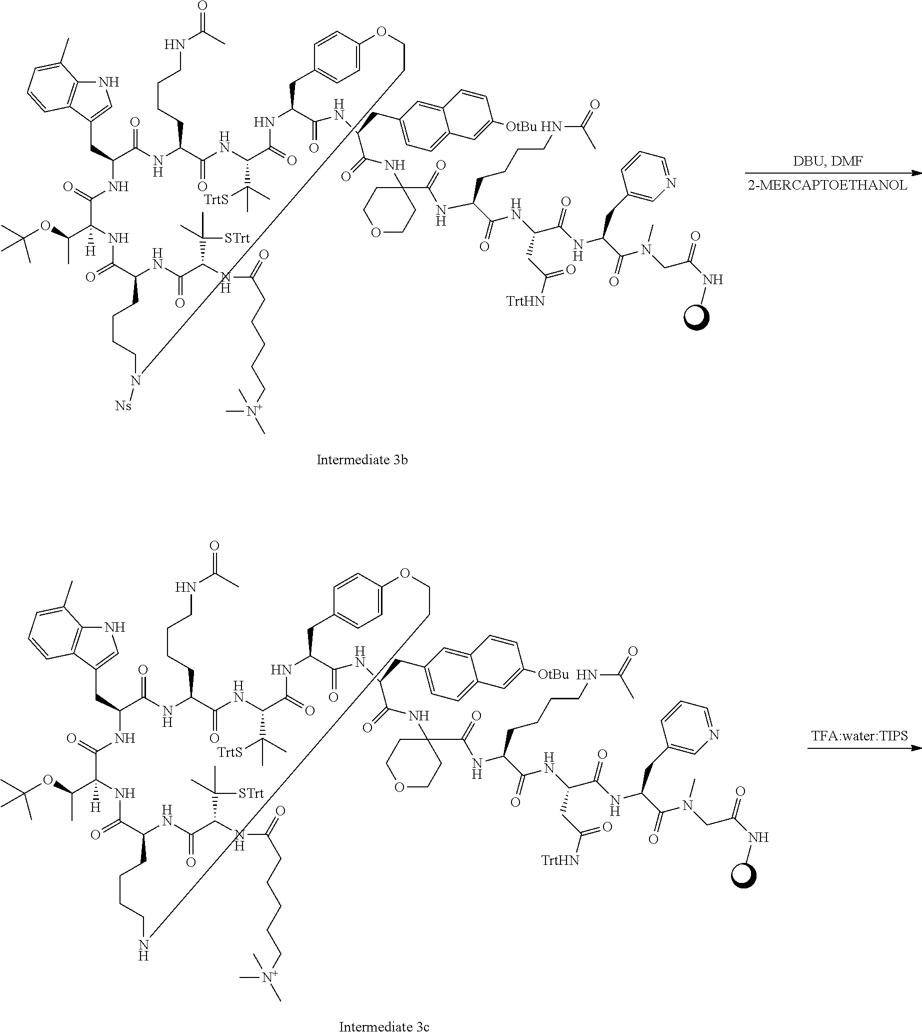

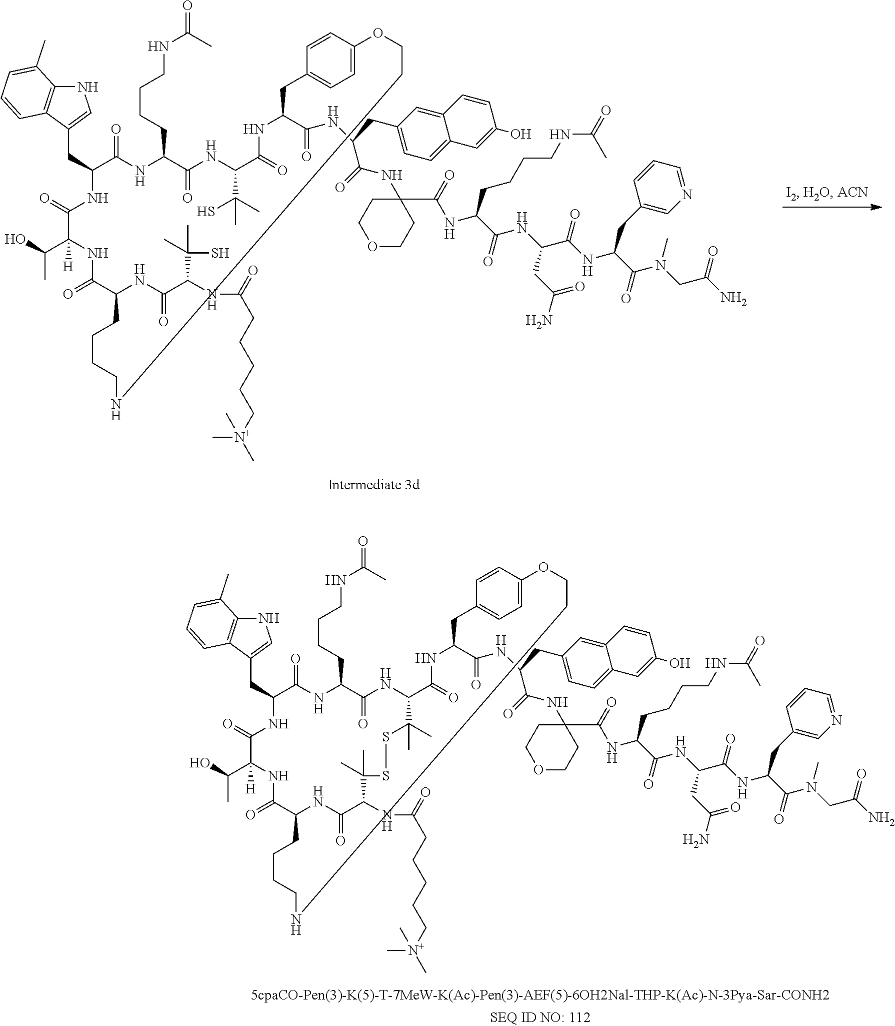

Step A—Synthesis of Intermediate 3: The synthesis was performed using Fmoc-protected amino acids on a solid-phase Rink amide MBHA resin (Novabiochem, 0.42 mmol/g, 100-200 mesh) with a CEM Liberty Blue automated microwave peptide synthesizer. The peptide was synthesized on a 0.25 mmol scale. Typical reaction conditions were as follows:

Deprotection Conditions: Fmoc deprotection was carried out using 20% piperidine in DMF (10 mL) under microwave conditions (90° C., 1 min).

Residue Coupling Conditions: Fmoc-protected amino acid (5 mL of a 0.2 M amino acid stock solution in DMF, 1 mmol) was delivered to the resin, followed by DIC (2.041 mL, 0.5 M, 1 mmol) and ethyl (hydroxyimino)cyanoacetate (1 mL, 1 M, 1 mmol) at 90° C. for 3.5 min. Double couplings were used for 3Pya, THP and Thr and for residues incorporated after THP and Thr (6OH2Nal and K(NNs)). Residue Y(OEtOTBDMS) and 5cpa were coupled using manual coupling conditions: the mixture of Fmoc-protected amino acid (0.75 mmol), HATU (0.75 mmol) and 4-methylmorpholine (1.5 mmol) in DMF (8 mL) was added to the resin (0.25 mmol) and then mixed for 2 hours at room temperature. At the end of the assembly, the peptide resin intermediate 3 was washed with DCM.

Step B—Synthesis of Intermediate 3a: Intermediate 3 (0.25 mmol) was swelled in THF (8 mL) for 15 min, then TBAF (2.5 mL, 1 M in THF, 2.5 mmol) was added. The reaction was mixed at room temperature for 1 hr. The resin was then drained, washed with DMF (8 mL, 3 times) and DCM (8 mL, 3 times). To the resulting resin in DCM (20 mL) was added TEA (0.523 mL, 0.728 g/mL, 3.75 mmol) in DCM (5 ml) and then followed by the slow addition of solution of methanesulfonyl chloride (0.195 mL, 1.48 g/mL, 2.5 mmol) in DCM (5 mL). The reaction was mixed at room temperature for 1 hr, then drained and washed with DCM (3 x). Microcleavage of the resin with TFA showed the desired product. LCMS anal. calc. for C₁₀₉H₁₅₅N₂₂O₂₈S₄ 2349.824. found: 784 (M+3)²⁺

Step C—Synthesis of Intermediate 3b: Intermediate 3a (0.25 mmol) was swelled in DMF (10 mL) for 15 min, then was added to a saturated solution of Cs₂CO₃ in DMF (200 mL). The reaction mixture was heated at 68° C. for 1 hr. The resin was then cooled to room temperature, drained, washed with water (3×), DMF (3×) and DCM (3×). Microcleavage of the resin with TFA showed the desired product. LCMS anal. calc. for C₁₀₈H₁₅₁N₂₂O₂₅S₃⁺ 2252.03. found: 752 (M+3)³⁺

Step D—Synthesis of Intermediate 3c: To intermediate 3b (0.5 mmol) in DMF (20 mL) was added a solution of 1,8-diazabicyclo[5.4.0]undec-7-ene (373.5 μL, 1.019 g/mL, 2.5 mmol) in DMF (3 mL), followed by 2-mercaptoethanol (350.7 μL, 1.114 g/mL, 5 mmol) in DMF (3 mL). The reaction mixture was mixed for 20 min. The resin was washed with DCM and DMF. The procedure was repeated once more. Microcleavage of the resin with TFA showed the desired product. The resin was washed with DMF, MeOH, and DCM. LCMS anal. calc. for C₁₀₂H₁₄₈N₂₁O₂₁S₂⁺ 2067.06. found: 689.9 (M+3)³⁺

Step E—Synthesis of Intermediate 3d: Intermediate 3c (0.15 mmol) was treated with a solution of TFA/H₂O/TIPS 92.5/5/2.5 for 30 mins at 42° C. on a CEM Razor cleavage station. The mixture was then concentrated and added to cold methyl-t-butyl ether in order to precipitate the peptide. After centrifugation, the peptide pellets were washed with fresh cold methyl-t-butyl ether. This process was repeated twice. Final pellets were dried, resuspended in H₂O and acetonitrile, then lyophilized to afford the desired protected intermediate 3d as a light yellow solid. LCMS anal. calc. for C₁₀₂H₁₄₈N₂₁O₂₁S₂⁺: 2067.06. found: 1034.3 (M+2)²⁺

Step E—Synthesis of SEQ ID NO: 112: Intermediate 3d was dissolved in 40% ACN/water (50 mL). Iodine in methanol (0.1 M) was then added drop wise with stirring until a yellow color persisted. The reaction was monitored with UPLC-MS. When the reaction was complete, solid ascorbic acid was added until the solution became clear. The solvent mixture was then lyophilized, and the resulting material was then dissolved in DMSO and was purified by C18 reverse-phase HPLC (Waters XBridge OBD C18, 50×150 mm, 5 μm, 130 Å), using as eluents (A) 0.1% TFA in water and (B) 0.1% TFA in acetonitrile, gradient began with 15% B, and changed to 30% B over 25 minutes at a flow rate of 80 ml/min). Fractions containing pure product were collected and then freeze-dried to afford the desired product as a white powder. LCMS anal. Calcd. C₁₀₂H₁₄₆N₂₁O₂₁S₂⁺: 2065.04 found: 1033.3 (M+2)²⁺

### Example 5: Synthesis of SEQ ID NO: 287—MeCO-r-Pen(3)-N-T-7(3NAcPh)W-K(Ac)-Pen(3)-AEF-6OH2Nal-THP-E-N-3Pya-Sar-CONH2

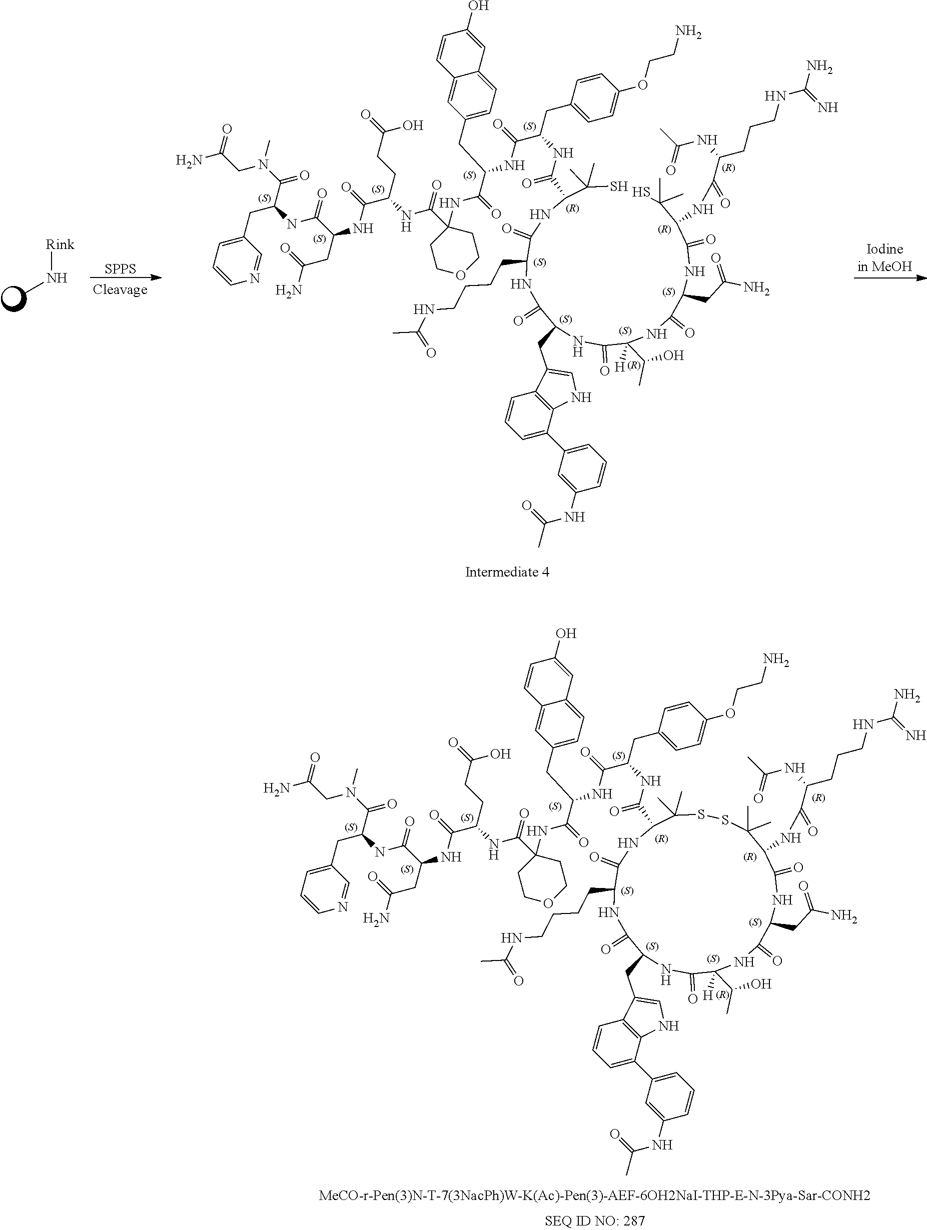

Step A—Synthesis of Intermediate 4: The peptide was synthesized by solid-phase peptide synthesis using Fmoc chemistry. DMF was added to a vessel containing Rink Amide MBHA Resin (2.0 mmol, 6.0 g, sub: 0.33 mmol/g), and the resin was allowed to swell for 2 hours. Then, 20% piperidine/DMF was added to the resin and mixed for 30 minutes. The mixture was drained and washed five times with DMF (for 30 seconds each). Fmoc-amino acid solution was then added to the resin and mixed for 30 seconds before the addition of activation buffer. The amino acid was allowed to react with the resin for 1-4 hours under N2. Then, 20% piperidine/DMF was added and mixed for 30 min. The procedure was repeated for subsequent amino acid coupling(s). Coupling reactions were monitored by ninhydrin (A: 5% ninhydrin/EtOH; B: 80% phenol/EtOH; C: pyridine) or tetrachlor (A: 2% tetrachlor/DMF; B: 2% aldehyde/DMF 110° C. for 3 min) color test. Subsequently, the resin was washed with DMF 5 times, and then with MeOH 3 times before drying under vacuum.

To cleave the peptide from the resin, 120 mL of cleavage buffer (5.0% DTT/2.5% H₂O/2.5% TIS/90% TFA) was added to the flask containing the side chain protected peptide on resin at room temperature, and the solution was stirred for 3 hrs. The mixture was then filtered and washed with 5 mL TFA. The combined filtrate was triturated with cold methyl tertbutyl ether (MTBE). The mixture was centrifuged (3000 rpm, 3 min) and decanted. The pellet was washed with MTBE and centrifuged. The residue was lyophilized to give Intermediate 4 (3.6 g).

Step B—Peptide cyclization and purification: Intermediate 4 (3.6 g, 1.6 mmol) was dissolved in 20% MeCN/H₂O (2000 mL). To a stirred solution of the peptide was added iodine in MeOH (0.1M, 12.0 mL) drop-wise until the color of the solution remained yellow. After ˜2 h, LCMS showed the reaction was complete. Excess iodine was quenched by the addition of 1M Na₂S₂O₃ in water (15 uL) (turned colorless instantly). MeCN (10-20 mL) was then added to reduce turbidity. The solution was purified by Prep-HPLC (A: 0.075% TFA in H₂O, B: ACN) (Note 1: Method A) and Prep-HPLC (A: 0.5% AcOH in H₂O, B: ACN) (Note 1: Method B) to give SEQ ID NO: 287 (1117.4 mg, 90.8% purity, 26.0% yield for this step; over all yield: 21.4%) obtained as white solid. Analysis was performed using a C18 column with a flow rate of 1 mL/min (Note 2).

LCMS Summary: calculated MW: 2189.47, observed MW: 1095.5 (M+2H)²⁺.

### Example 6: Synthesis of SEQ ID NO: 407—MeCO-r-Pen(3)-N-T-7MeW-K(Ac)-Pen(3)-AEF-6F2Nal-THP-E-N-3Pya-Sar-CONH2

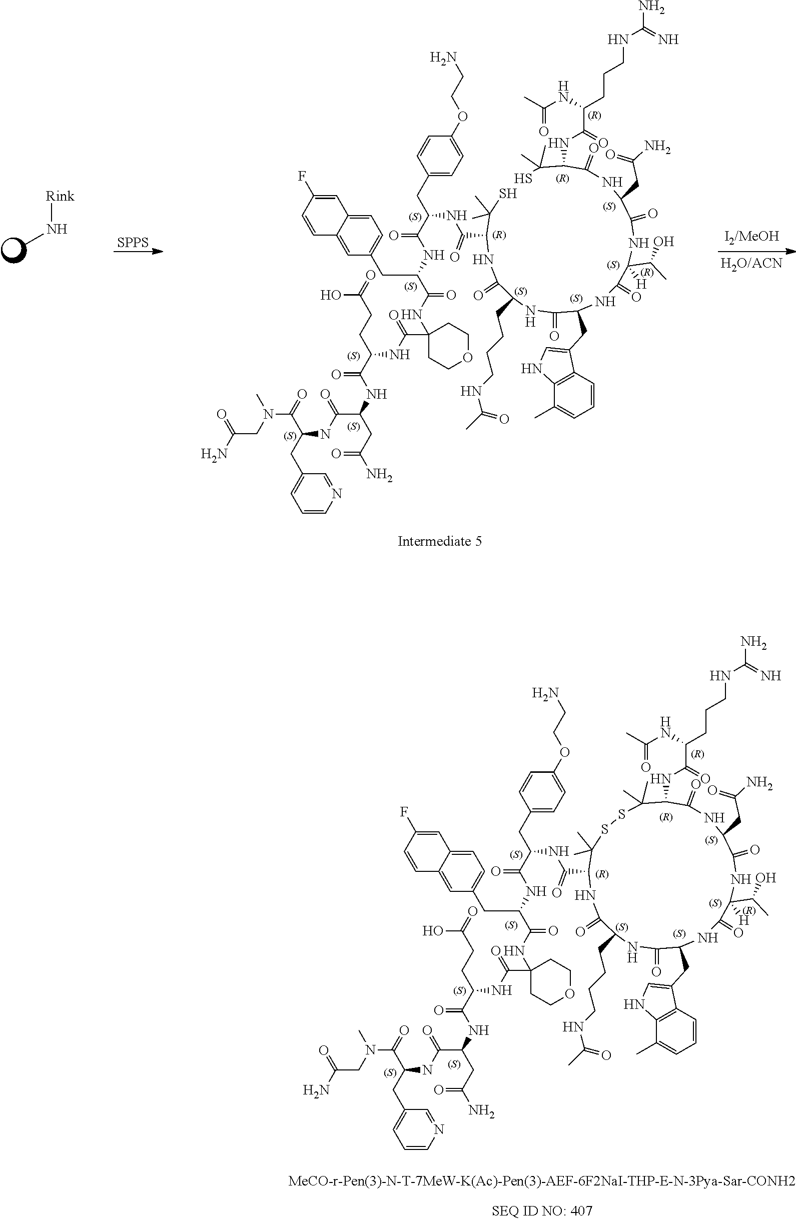

Step A—Synthesis of Intermediate 5: The peptide was synthesized by solid-phase peptide synthesis using Fmoc chemistry. DMF was added to a vessel containing MBHA Resin (0.20 mmol, 0.64 g, sub: 0.31 mmol/g), and the resin was allowed to swell for 2 hours. Then, 20% piperidine/DMF was added to the resin and mixed for 30 minutes. The mixture was drained and washed five times with DMF (for 30 seconds each). Fmoc-amino acid solution was then added to the resin and mixed for 30 seconds before the addition of activation buffer. The amino acid was allowed to react with the resin for 1-4 hours under N2. Then, 20% piperidine/DMF was added and mixed for 30 min. The procedure was repeated for subsequent amino acid coupling(s). Coupling reactions were monitored by ninhydrin (A: 5% ninhydrin/EtOH; B: 80% phenol/EtOH; C: pyridine) or tetrachlor (A: 2% tetrachlor/DMF; B: 2% aldehyde/DMF 110° C. for 3 min) color test. Subsequently, the resin was washed with DMF 5 times, and then with MeOH 3 times before drying under vacuum.

To cleave the peptide from the resin, 12 mL of cleavage buffer (5.0% DTT/2.5% H₂O/2.5% TIS/90% TFA) was added to the flask containing the side chain protected peptide on resin at room temperature, and the solution was stirred for 3 hrs. The mixture was then filtered and washed with 5 mL TFA. The combined filtrate was triturated with cold methyl tertbutyl ether (MTBE). The mixture was centrifuged (3000 rpm, 3 min) and decanted. The pellet was washed with MTBE and centrifuged. The residue was lyophilized to give Intermediate 5 (350 mg).

Step B—Peptide cyclization and purification: Intermediate 5 (350 mg, 0.17 mmol) was dissolved in 20% MeCN/H2O (200 mL). To a stirred solution of the peptide was added iodine in MeOH (0.1M, 0.8 mL) drop-wise until the color of the solution remained yellow. After ˜2 h LCMS showed the reaction was complete. Excess iodine was quenched by the addition of 1M Na₂S₂O₃ in water (15 uL) (turned colorless instantly). MeCN (10-20 mL) was then added to reduce turbidity. The solution was purified by Prep-HPLC (A: 0.075% TFA in H₂O, B: ACN) (Note 1: Method C) and repurified by Prep-HPLC (A: 0.5% AcOH in H₂O, B: ACN) (Note 1: Method D), then the product was relyophilized with 0.1% TFA mobile phase to give SEQ ID NO: 407 (92.8 mg, 97.0% purity, 22.1% yield for this step; over all yield: 18.6%) obtained as white solid.

Analysis was performed using a C18 column with a flow rate of 1 mL/min (Note 2). LCMS Summary: calculated MW: 2072.3, observed MW: 1036.9 (M+2H)²⁺, 691.8 (M+3H)³⁺

### Example 7: Synthesis of SEQ ID NO: 405—CF₃CO-k(5cpa)-Pen(3)-N(N(Me)2)-T-7MeW-Q(N(Me)2)-Pen(3)-AEF(N(Me)2)-6OH2Nal-THP-Q(N(Me)2)-N-3Pya-Sar-CON(Me)2

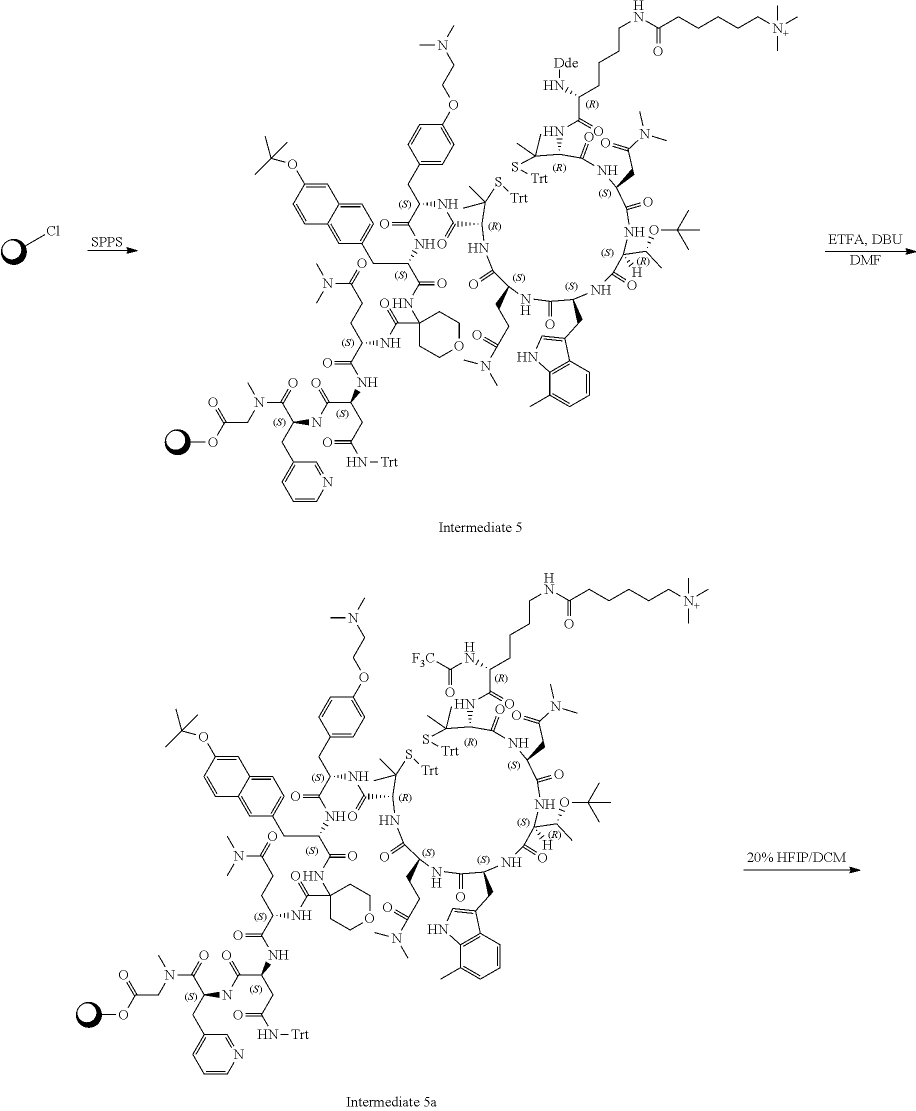

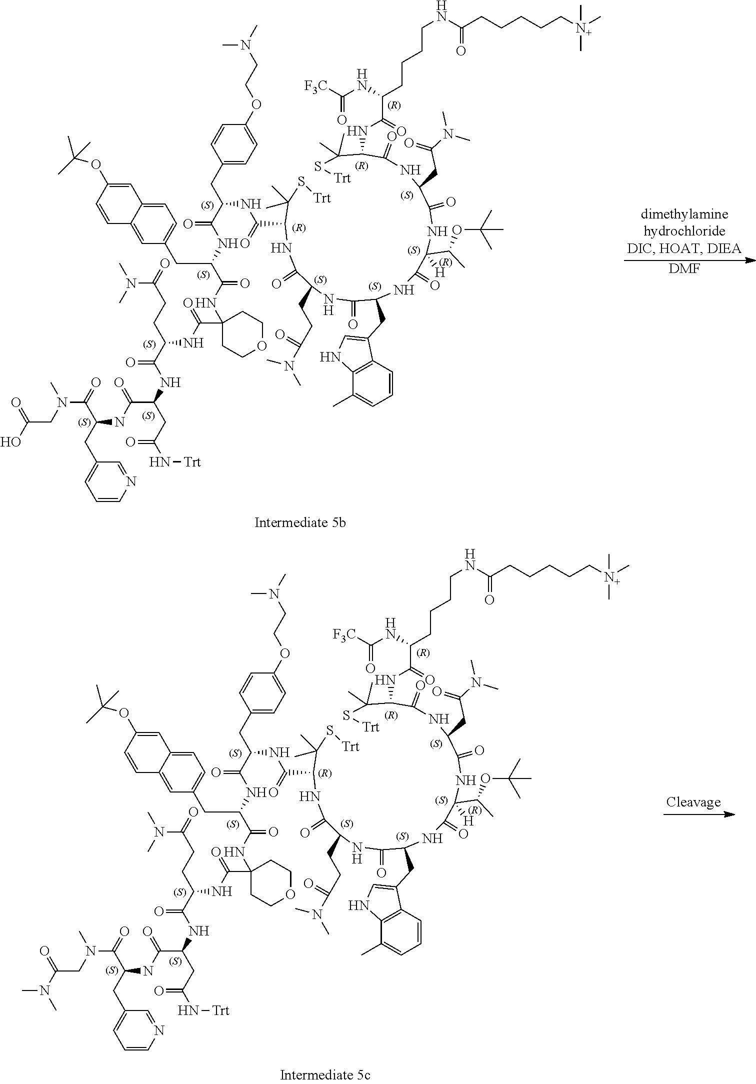

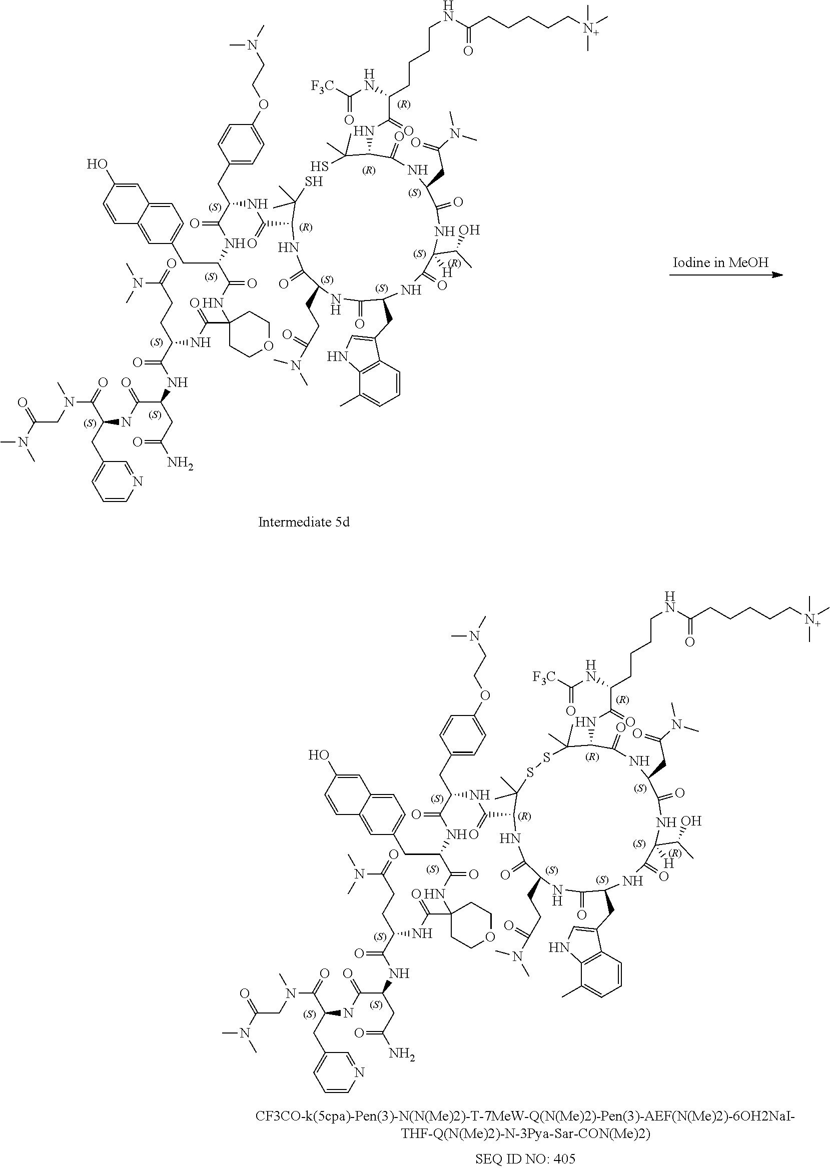

Step A—Synthesis of Intermediate 6: The peptide was synthesized by solid-phase peptide synthesis using Fmoc chemistry. DMF was added to a vessel containing Sar-CTC Resin (0.50 mmol, 1.6 g, sub: 0.31 mmol/g), and the resin was allowed to swell for 2 hours. Then, 20% piperidine/DMF was added to the resin and mixed for 30 minutes. The mixture was drained and washed five times with DMF (for 30 seconds each). Fmoc-amino acid solution was then added to the resin and mixed for 30 seconds before the addition of activation buffer. The amino acid was allowed to react with the resin for 1-4 hours under N₂. Then, 20% piperidine/DMF was added and mixed for 30 min. The procedure was repeated for subsequent amino acid coupling(s). Coupling reactions were monitored by ninhydrin (A: 5% ninhydrin/EtOH; B: 80% phenol/EtOH; C: pyridine) or tetrachlor (A: 2% tetrachlor/DMF; B: 2% aldehyde/DMF 110° C. for 3 min) color test. Subsequently, the resin was washed with DMF 5 times, and then with MeOH 3 times before drying under vacuum.

To cleave the peptide from the resin, 80 mL of cleavage buffer (20% HFIP/DCM) was added to the resin and stirred for 30 minutes. The mixture was then concentrated under reduced pressure. The peptide was dissolved in ACN/water and lyophilized overnight to give Intermediate 5b (1.2 g).

Step B—Preparation of Intermediate 6c: To a solution of Intermediate 6b (1.2 g, 0.40 mmol) in DMF (10.0 mL) was added dimethylamine hydrochloride (65.2 mg, 2.00 eq), DIC (123 uL, 2.00 eq), HOAT (108 mg, 2.00 eq) and DIEA (136 uL, 2.00 eq). The mixture was stirred at 25° C. for 1 h. The reaction was monitored by LCMS. Because LCMS showed the starting material didn't react completely, one additional equivalent of each of dimethylamine hydrochloride, DIC, HOAT and DIEA was added, and the reaction allowed to continue for 16 h. Afterward, LCMS showed the starting material was consumed. The filtrate was triturated with cold methyl tertbutyl ether (MTBE) (50 mL) and centrifuged (3000 rpm, 3 min) to give an oily liquid. The ether was dried with nitrogen to afford Intermediate 5c.

Step C—Preparation of Intermediate 6d: To a flask containing the side chain-protected peptide at room temperature was added 35 mL cleavage buffer (5.0% DTT/2.5% H₂O/2.5% TIS/90% TFA) at room temperature. The mixture was stirred for 3 hours. The mixture was then filtered and washed with 5 mL TFA. The combined filtrate was triturated with cold methyl tertbutyl ether (MTBE). The mixture was centrifuged (3000 rpm, 3 min) and decanted. The pellet was washed with MTBE and centrifuged. The residue was lyophilized to give Intermediate 5d (600 mg).

Step D—Synthesis of SEQ ID NO: 405: Intermediate 6d (600 mg, 0.255 mmol) was dissolved in 20% MeCN/H2O (500 mL). To a stirred solution of the peptide was added iodine in MeOH (0.1M, 3.5 mL) drop-wise until the color of the solution remained yellow. After ˜2 h LCMS showed the reaction was complete. Excess iodine was quenched by the addition of 1M Na₂S₂O₃ in water (15 uL) (turned colorless instantly). MeCN (10-20 mL) was added to reduce turbidity. The solution was purified by Prep-HPLC (A: 0.075% TFA in H2O, B: ACN) (Note 1: Method C) to give SEQ ID NO: 405 (99 mg, 93.43% purity, 13.4% yield for this step; over all yield: 6.87%) obtained as white solid.

Analysis was performed using a C18 column with a flow rate of 1 mL/min (Note 2). LCMS Summary: calculated MW: 2349.76, observed MW: 784.0 [(M+3H)/3].

### Example 8: Synthesis of SEQ ID NO: 389—MeCO-hk(Me)3-Pen(3)-N(N(Me)2)-T-7MeW-K(NMeAc)-Pen(3)-AEF-6OH2Nal-THP-E-N-3Pya-Sar-CON(Me)2

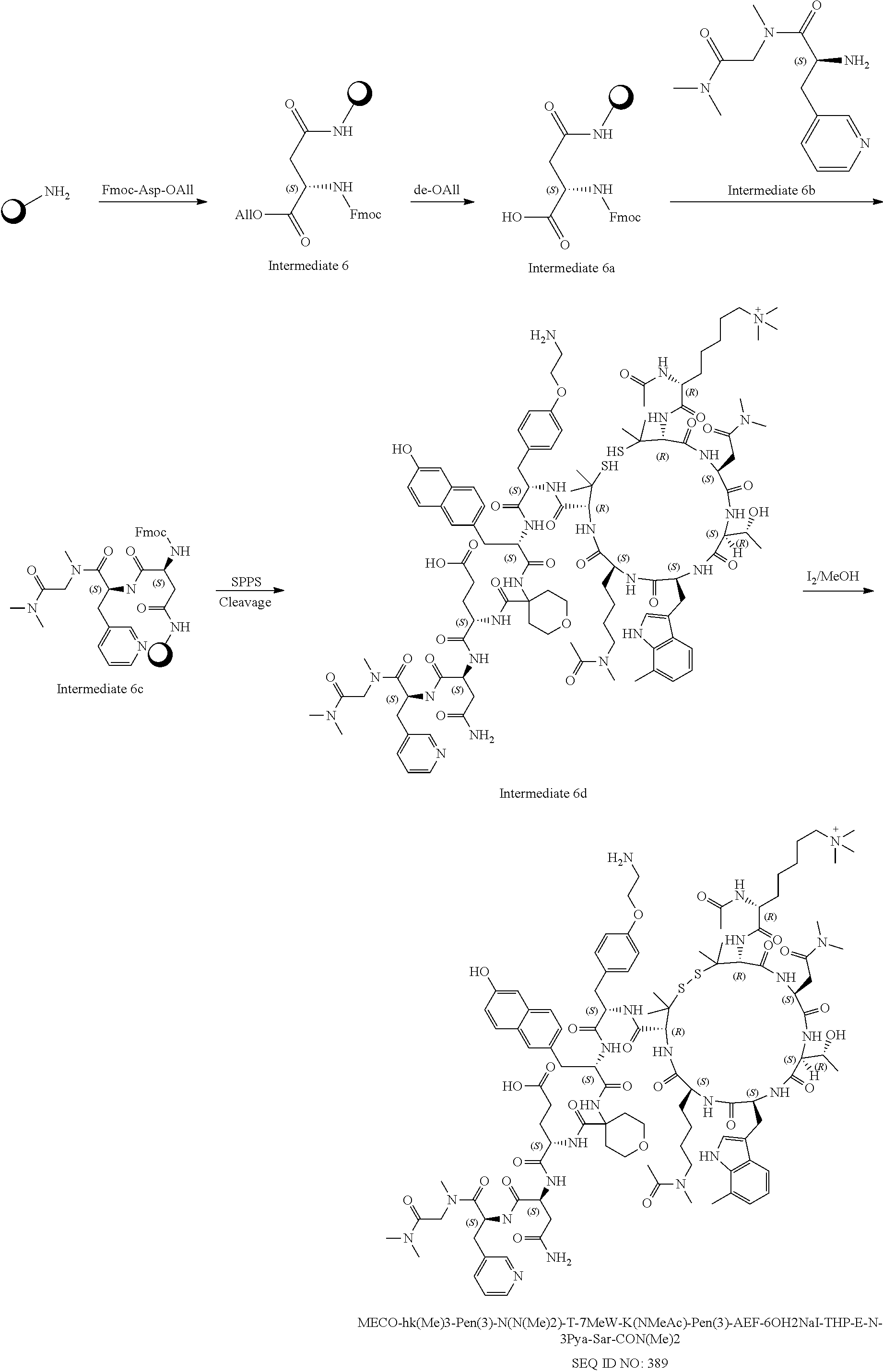

Step A—Synthesis of Intermediate 6d: The peptide was synthesized by solid-phase peptide synthesis using Fmoc chemistry. DMF was added to a vessel containing Rink Amide MBHA resin (1.0 mmol, 3.33 g, sub: 0.3 mmol/g), and the resin was allowed to swell for 2 hours. Then, 20% piperidine/DMF was added to the resin and mixed for 30 minutes. The mixture was drained and washed five times with DMF (for 30 seconds each). Fmoc-amino acid solution was then added to the resin and mixed for 30 seconds before the addition of activation buffer. The amino acid was allowed to react with the resin for 1-4 hours under N2. Then, 20% piperidine/DMF was added and mixed for 30 min. The procedure was repeated for subsequent amino acid coupling(s). Coupling reactions were monitored by ninhydrin (A: 5% ninhydrin/EtOH; B: 80% phenol/EtOH; C: pyridine) or tetrachlor (A: 2% tetrachlor/DMF; B: 2% aldehyde/DMF 110° C. for 3 min) color test. Subsequently, the resin was washed with DMF 5 times, and then with MeOH 3 times before drying under vacuum.

To cleave the peptide from the resin, 60 mL of cleavage buffer (5.0% DTT/2.5% H₂O/2.5% TIS/90% TFA) was added to the flask containing the side chain protected peptide on resin at room temperature, and the solution was stirred for 3 hrs. The mixture was then filtered and washed with 15 mL TFA. The combined filtrate was triturated with cold methyl tertbutyl ether (MTBE). The mixture was centrifuged (3000 rpm, 3 min) and decanted. The pellet was washed with MTBE and centrifuged. The residue was lyophilized to give Intermediate 6d (2.0 g).

Step B—Peptide Cyclization and Purification: Intermediate 6d (2.0 g, 0.92 mmol) was dissolved in 20% MeCN/H2O (1000 mL). To a stirred solution of the peptide was added iodine in MeOH (0.1M, 2.5 mL) drop-wise until the color of the solution remained yellow. After ˜2 h LCMS showed the reaction was complete. Excess iodine was quenched by the addition of 1M Na₂S₂O₃ in water (15 uL) (turned colorless instantly). MeCN (10-20 mL) was added to reduce turbidity. The solution was purified by Prep-HPLC (A: 0.075% TFA in H₂O, B: ACN) (Note 1: Method E) to give SEQ ID NO: 389 (270.4 mg, 94.6% purity, 11.0% yield for this step; over all yield: 10.1%) obtained as white solid. Analysis was performed using a C18 column with a flow rate of 1 m/min (Note 2).

LCMS Summary: calculated MW: 2169.5, observed MW: 1085.1 (M+2H)²+, 724.1 (M+3H)³⁺

### Example 9: Synthesis of SEQ ID NO: 328—cPEG3aCO-Pen(3)-N-T-7MeW-K(NMecPEG3a)-Pen(3)-AEF-6OH2Nal-THP-K(NMecPEG3a)-N-3Pya-Sar-CONH2

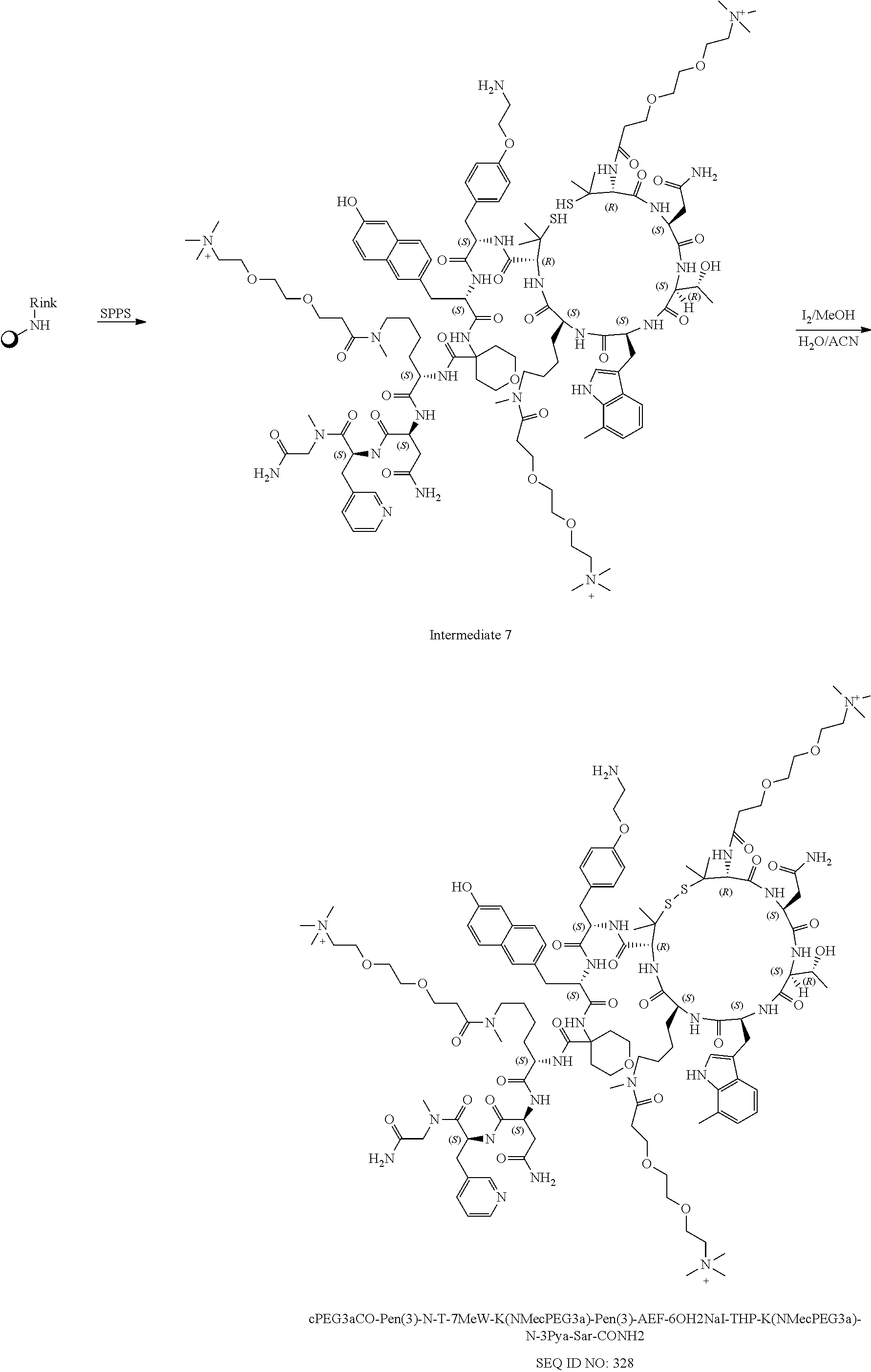

Step A—Synthesis of Intermediate 7: The peptide was synthesized by solid-phase peptide synthesis using Fmoc chemistry. DMF was added to a vessel containing MBHA Resin (0.30 mmol, 1.15 g, sub: 0.26 mmol/g), and the resin was allowed to swell for 2 hours. Then, 20% piperidine/DMF was added to the resin and mixed for 30 minutes. The mixture was drained and washed five times with DMF (for 30 seconds each). Fmoc-amino acid solution was then added to the resin and mixed for 30 seconds before the addition of activation buffer. The amino acid was allowed to react with the resin for 1-4 hours under N2. Then, 20% piperidine/DMF was added and mixed for 30 min. The procedure was repeated for subsequent amino acid coupling(s). Coupling reactions were monitored by ninhydrin (A: 5% ninhydrin/EtOH; B: 80% phenol/EtOH; C: pyridine) or tetrachlor (A: 2% tetrachlor/DMF; B: 2% aldehyde/DMF 110° C. for 3 min) color test. Subsequently, the resin was washed with DMF 5 times, and then with MeOH 3 times before drying under vacuum.

To cleave the peptide from the resin, 25 mL of cleavage buffer (5.0% DTT/2.5% H₂O/2.5% TIS/90% TFA) was added to the flask containing the side chain protected peptide on resin at room temperature, and the solution was stirred for 3 hrs. The mixture was then filtered and washed with 5 mL TFA. The combined filtrate was triturated with cold methyl tertbutyl ether (MTBE). The mixture was centrifuged (3000 rpm, 3 min) and decanted. The pellet was washed with MTBE and centrifuged. The residue was lyophilized to give Intermediate 7 (600 mg).

Step B—Peptide Cyclization and Purification: Intermediate 7 (600 mg, 0.24 mmol) was dissolved in 20% MeCN/H2O (400 mL). To a stirred solution of the peptide was added iodine in MeOH (0.1M, 3.0 mL) drop-wise until the color of the solution remained yellow. After ˜2 h LCMS showed the reaction was complete. Excess iodine was quenched by the addition of 1M Na₂S₂O₃ in water (turned colorless instantly). MeCN (10-20 mL) was added to reduce turbidity. The solution was purified by Prep-HPLC (A: 0.075% TFA in H₂O, B: ACN) (Note 1: Method C) to give SEQ ID NO: 328 (89.6 mg, 96.9% purity, 11.7% yield for this step, over all yield: 9.54% yield) obtained as white solid. Analysis was performed using a C18 column with a flow rate of 1 m/min (Note 2).

LCMS Summary: calculated MW: 2464.0, observed MW: 821.0 [(M+3H)/3].

### Example 10: Synthesis of SEQ ID NO: 414—MeCO-Pen(3)-E(2)-T-7MeW-K(Ac)-Pen(3)-AEF(2)-6OH2Nal-THP-K(Ac)-N-3Pya-Sar-CONH2

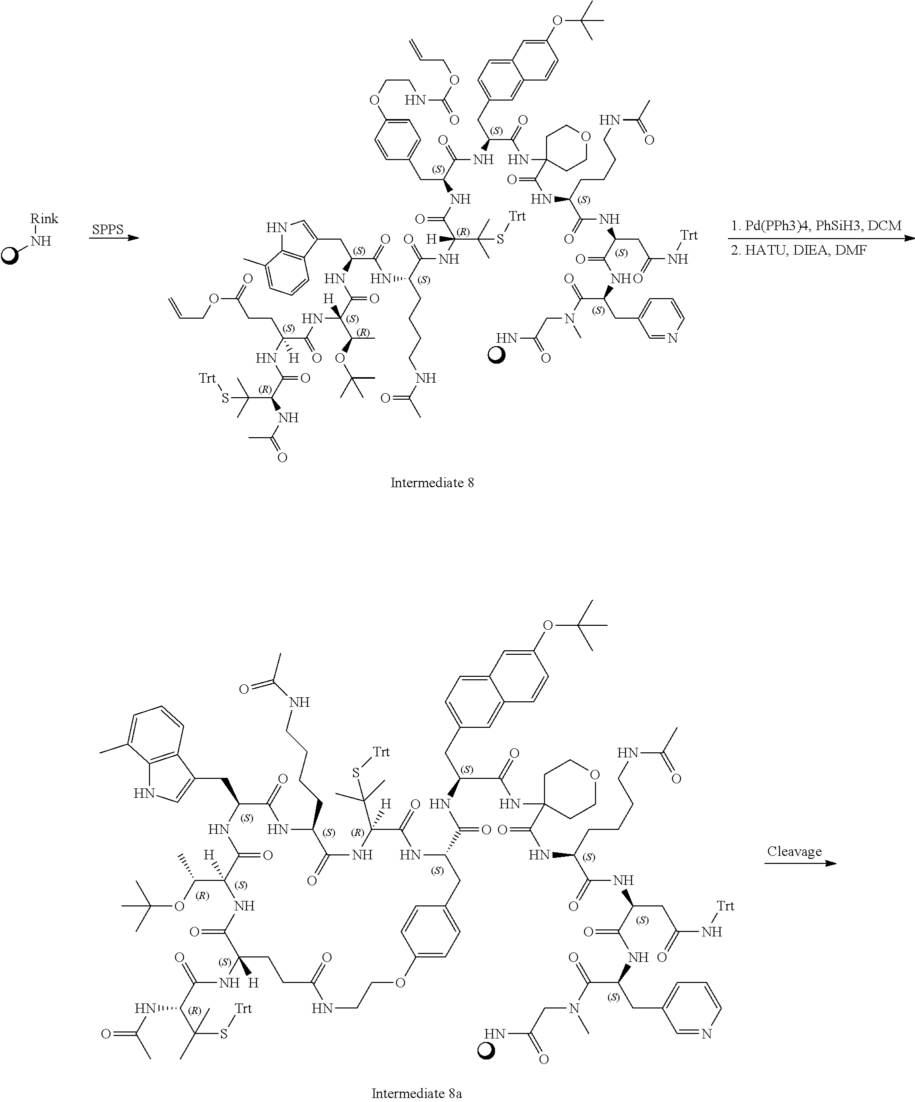

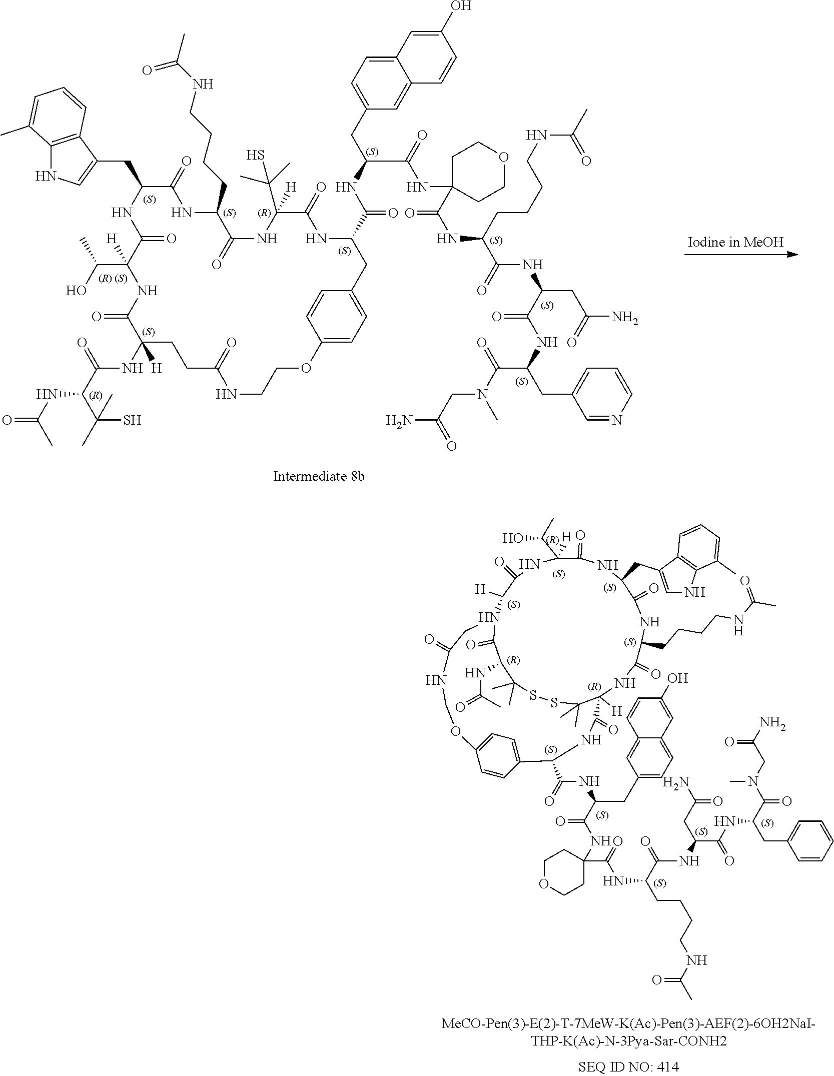

Step A—Synthesis of Intermediate 8b: The peptide was synthesized by solid-phase peptide synthesis using Fmoc chemistry. DMF was added to a vessel containing MBHA Resin (0.2 mmol, 0.65 g, sub: 0.31 mmol/g), and the resin was allowed to swell for 2 hours. Then, 20% piperidine/DMF was added to the resin and mixed for 30 minutes. The mixture was drained and washed five times with DMF (for 30 seconds each). Fmoc-amino acid solution was then added to the resin and mixed for 30 seconds before the addition of activation buffer. The amino acid was allowed to react with the resin for 1-4 hours under N2. Then, 20% piperidine/DMF was added and mixed for 30 min. The procedure was repeated for subsequent amino acid coupling(s). Coupling reactions were monitored by ninhydrin (A: 5% ninhydrin/EtOH; B: 80% phenol/EtOH; C: pyridine) or tetrachlor (A: 2% tetrachlor/DMF; B: 2% aldehyde/DMF 110° C. for 3 min) color test. Subsequently, the resin was washed with DMF 5 times, and then with MeOH 3 times before drying under vacuum.

To cleave the peptide from the resin, 12 mL of cleavage buffer (5.0% DTT/2.5% H₂O/2.5% TIS/90% TFA) was added to the flask containing the side chain protected peptide on resin at room temperature, and the solution was stirred for 3 hrs. The mixture was then filtered and washed with 5 mL TFA. The combined filtrate was triturated with cold methyl tertbutyl ether (MTBE). The mixture was centrifuged (3000 rpm, 3 min) and decanted. The pellet was washed with MTBE and centrifuged. The residue was lyophilized to give Intermediate 8b (350 mg).

Step B—Peptide Cyclization and Purification: Intermediate 8b (350 mg, 0.17 mmol) was dissolved in 20% MeCN/H2O (200 mL). To a stirred solution of the peptide was added iodine in MeOH (0.1M, 0.8 mL) drop-wise until the color of the solution remained yellow. After ˜2 h LCMS showed the reaction was complete. Excess iodine was quenched by the addition of 1M Na₂S₂O₃ in water (15 uL) (turned colorless instantly). MeCN (10-20 mL) was added to reduce turbidity. The solution was purified by Prep-HPLC (A: 0.075% TFA in H₂O, B: ACN) (Note 1: Method G) to give SEQ ID NO: 414(31.2 mg, 98.2% purity, 8.28% yield for this step; over all yield: 7.41%) obtained as white solid. Analysis was performed using a C18 column with a flow rate of 1 m/min (Note 2).

LCMS Summary: calculated MW: 1952.2, observed MW: 976.9 (M+2H)²⁺.

### Example 11: Synthesis of SEQ ID NO: 115—MeCO-r-Pen(3)-N-T-7MeW-K(Ac)-Pen(3)-AEF-6OH2Nal-THP-E-N-3Pya-Sar-K(PEG2NMePEG2NMegENMeCl8Tetrazole)-CONH2

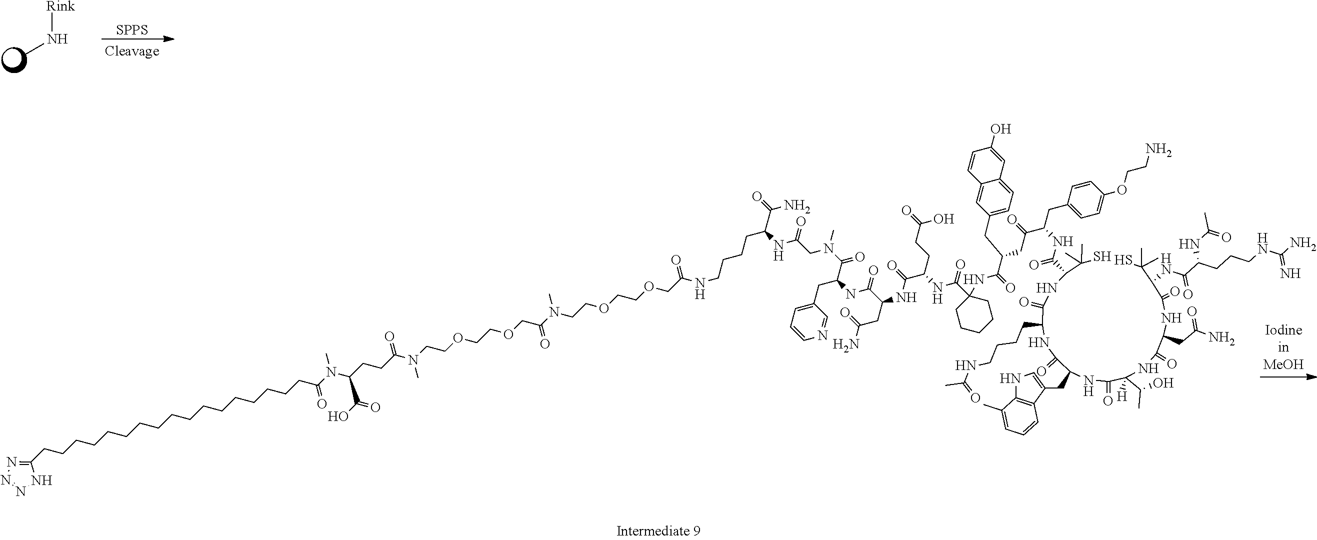

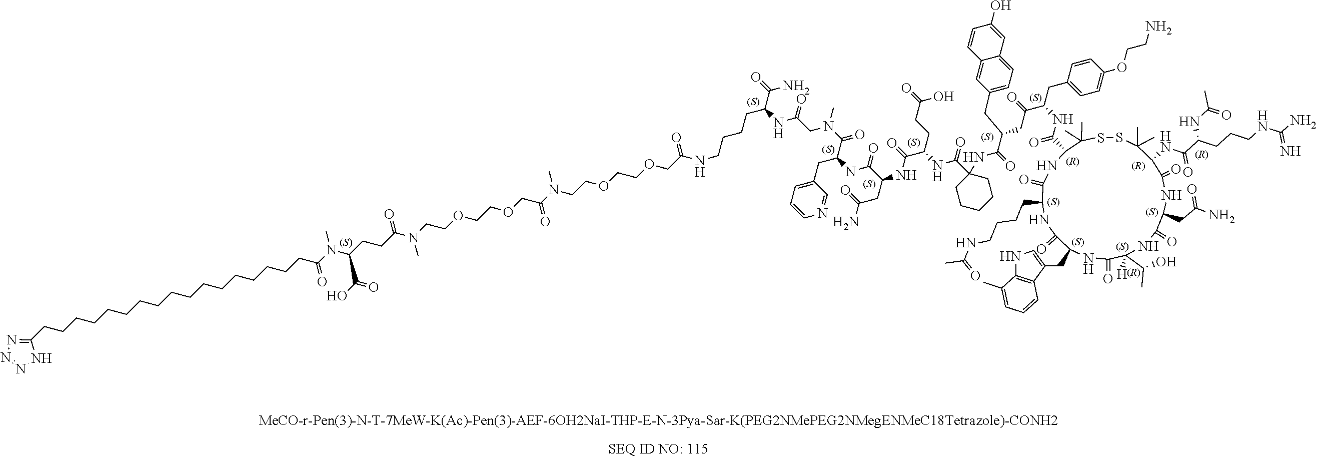

Step A—Synthesis of Intermediate 9: The peptide was synthesized by solid-phase peptide synthesis using Fmoc chemistry. DMF was added to a vessel containing MBHA Resin (0.2 mmol, 0.65 g, sub: 0.31 mmol/g), and the resin was allowed to swell for 2 hours. Then, 20% piperidine/DMF was added to the resin and mixed for 30 minutes. The mixture was drained and washed five times with DMF (for 30 seconds each). Fmoc-amino acid solution was then added to the resin and mixed for 30 seconds before the addition of activation buffer. The amino acid was allowed to react with the resin for 1-4 hours under N2. Then, 20% piperidine/DMF was added and mixed for 30 min. The procedure was repeated for subsequent amino acid coupling(s). Coupling reactions were monitored by ninhydrin (A: 5% ninhydrin/EtOH; B: 80% phenol/EtOH; C: pyridine) or tetrachlor (A: 2% tetrachlor/DMF; B: 2% aldehyde/DMF 110° C. for 3 min) color test. Subsequently, the resin was washed with DMF 5 times, and then with MeOH 3 times before drying under vacuum.

To cleave the peptide from the resin, 20 mL of cleavage buffer (5.0% DTT/2.5% H₂O/2.5% TIS/90% TFA) was added to the flask containing the side chain protected peptide on resin at room temperature, and the solution was stirred for 3 hrs. The mixture was then filtered and washed with 5 mL TFA. The combined filtrate was triturated with cold methyl tertbutyl ether (MTBE). The mixture was centrifuged (3000 rpm, 3 min) and decanted. The pellet was washed with MTBE and centrifuged. The residue was lyophilized to give Intermediate 9 (451 mg).

Step B—Peptide Cyclization and Purification: Intermediate 9 (451 mg, 0.151 mmol) was dissolved in 20% MeCN/H2O (300 mL). To a stirred solution of the peptide was added iodine in MeOH (0.1M, 1.0 mL) drop-wise until the color of the solution remained yellow. After ˜2 h LCMS showed the reaction was complete. Excess iodine was quenched by the addition of 1M Na₂S₂O₃ in water (15 uL) (turned colorless instantly). MeCN (10-20 mL) was added to reduce turbidity. The solution was purified by Prep-HPLC (A: 0.075% TFA in H₂O, B: ACN) (Note 1: Method H) to give SEQ ID NO: 115 (43.7 mg, 96.1% purity, 8.08% yield for this step; over all yield: 6.08%) obtained as white solid. Analysis was performed using a C18 column with a flow rate of 1 m/min (Note 2).

LCMS Summary: calculated MW: 2994.53, observed MW: 998.89[(M+3H)/3].

### Example 12: Synthesis of SEQ ID NO: 283—MeCO-Pen(3)-N-T-7MeW-K(Ac)-Pen(3)-AEF-6OH2Nal-THP-E-N-3Pya-Sar-K(PEG2PEG2gEC18OH)—CONH2

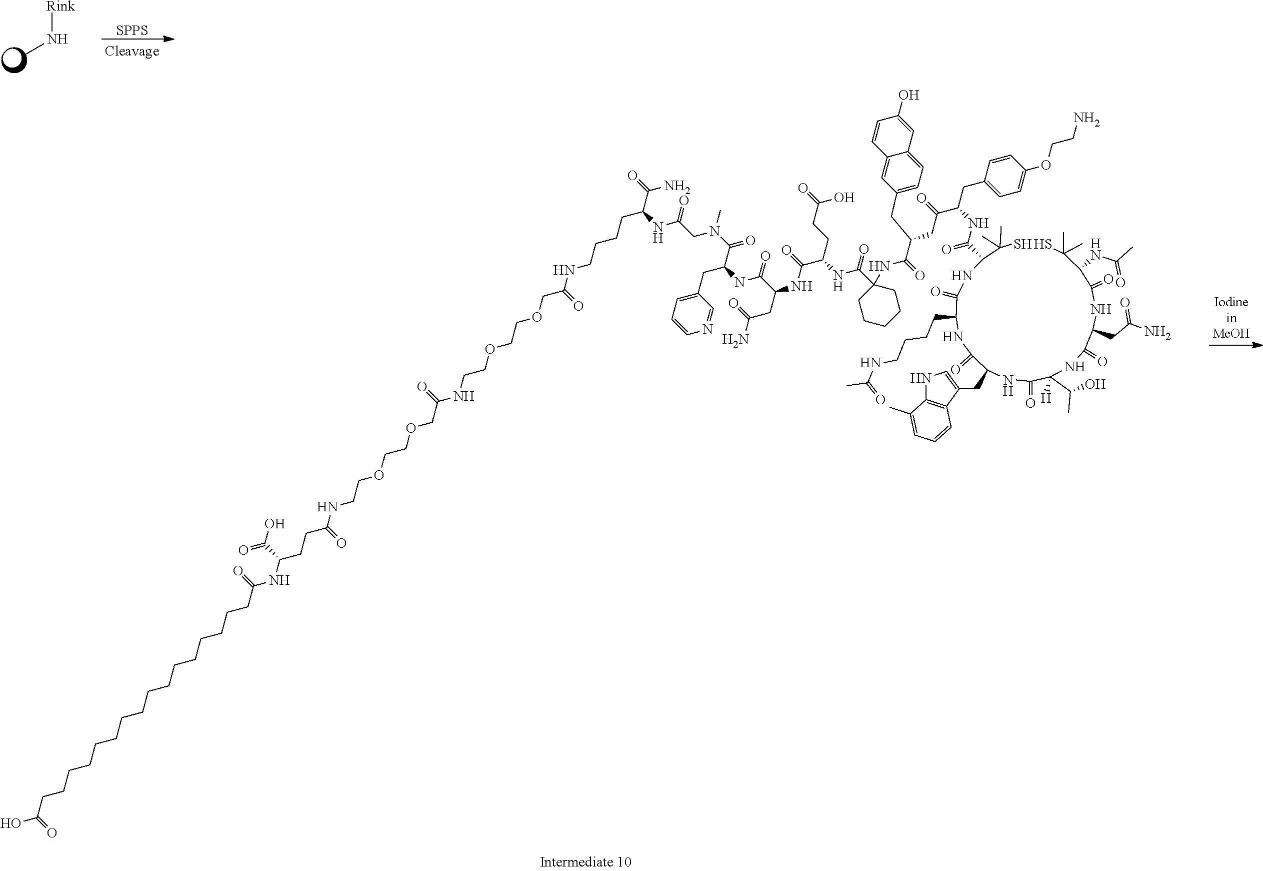

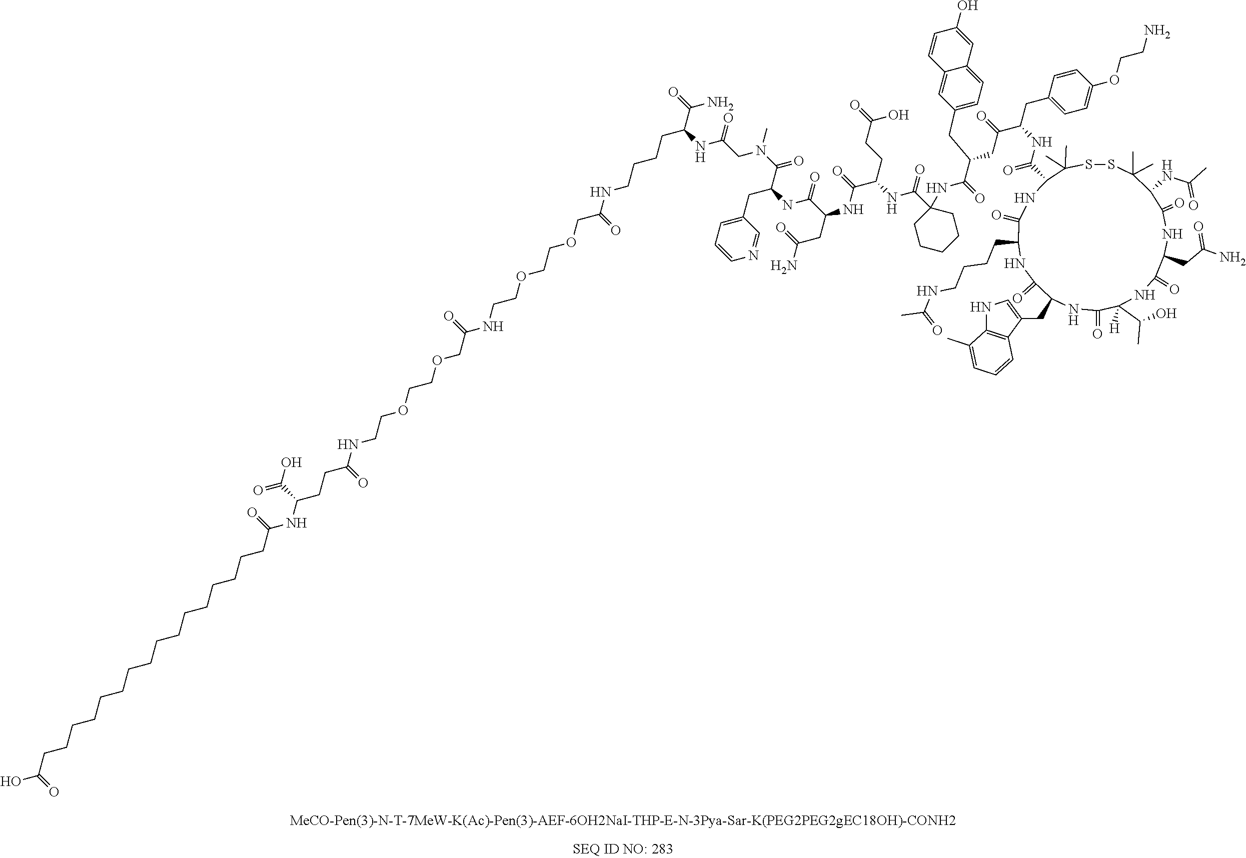 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)C[C@@H](Cc2ccc3cc(O)ccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(=O)N[C@@H](CCCCNC(=O)COCCOCCNC(=O)COCCOCCNC(=O)CC[C@H](NC(=O)CCCCCCCCCCCCCCCCC(=O)O)C(=O)O)C(N)=O)CCCCC2)NC1=O]

Step A—Synthesis of Intermediate 10: The peptide was synthesized by solid-phase peptide synthesis using Fmoc chemistry. DMF was added to a vessel containing MBHA Resin (0.3 mmol, 1.0 g, sub: 0.28 mmol/g), and the resin was allowed to swell for 2 hours. Then, 20% piperidine/DMF was added to the resin and mixed for 30 minutes. The mixture was drained and washed five times with DMF (for 30 seconds each). Fmoc-amino acid solution was then added to the resin and mixed for 30 seconds before the addition of activation buffer. The amino acid was allowed to react with the resin for 1-16 hours under N2. Then, 20% piperidine/DMF was added and mixed for 30 min. The procedure was repeated for subsequent amino acid coupling(s). Coupling reactions were monitored by ninhydrin (A: 500 ninhydrin/EtOH; B: 800% phenol/EtOH; C: pyridine) or tetrachlor (A: 2% tetrachlor/DMF; B: 2% aldehyde/DMF 110° C. for 3 min) color test. Subsequently, the resin was washed with DMF 5 times, and then with MeOH 3 times before drying under vacuum.

To cleave the peptide from the resin, 75 mL of cleavage buffer (5.0% DTT/2.5% H₂O/2.5% TIS/90% TFA) was added to the flask containing the side chain protected peptide on resin at room temperature, and the solution was stirred for 3 hrs. The mixture was then filtered and washed with 5 mL TFA. The combined filtrate was triturated with cold methyl tertbutyl ether (MTBE). The mixture was centrifuged (3000 rpm, 3 min) and decanted. The pellet was washed with MTBE and centrifuged. The residue was lyophilized to give Intermediate 10 (680 mg).

Step B—Peptide Cyclization and Purification: Intermediate 10 (680 mg, 0.246 mmol) was dissolved in 20% MeCN/H2O (300 mL). To a stirred solution of the peptide was added iodine in MeOH (0.1M, 1.5 mL) drop-wise until the color of the solution remained yellow. After ˜2 h LCMS showed the reaction was complete. Excess iodine was quenched by the addition of 1M Na₂S₂O₃ in water (15 uL) (turned colorless instantly). MeCN (10-20 mL) was added to reduce turbidity. The solution was purified by Prep-HPLC (A: 0.075% TFA in H₂O, B: ACN) (Note 1: Method H) to give SEQ ID NO: 283 (99.4 mg, 96.1% purity, 12.9% yield for this step; over all yield: 10.6%) obtained as white solid. Analysis was performed using a C18 column with a flow rate of 1 mL/min (Note 2).

LCMS Summary: calculated MW: 2758.21, observed MW: 1380.2 (M+2H)²⁺.

### Example 13: Synthesis of SEQ ID NO: 282—MeCO-r-Pen(3)-N-T-7MeW-K(Ac)-Pen(3)-AEF-6OH2Nal-THP-E-N-3Pya-Sar-K(PEG2PEG2gEC18OH)—CONH2

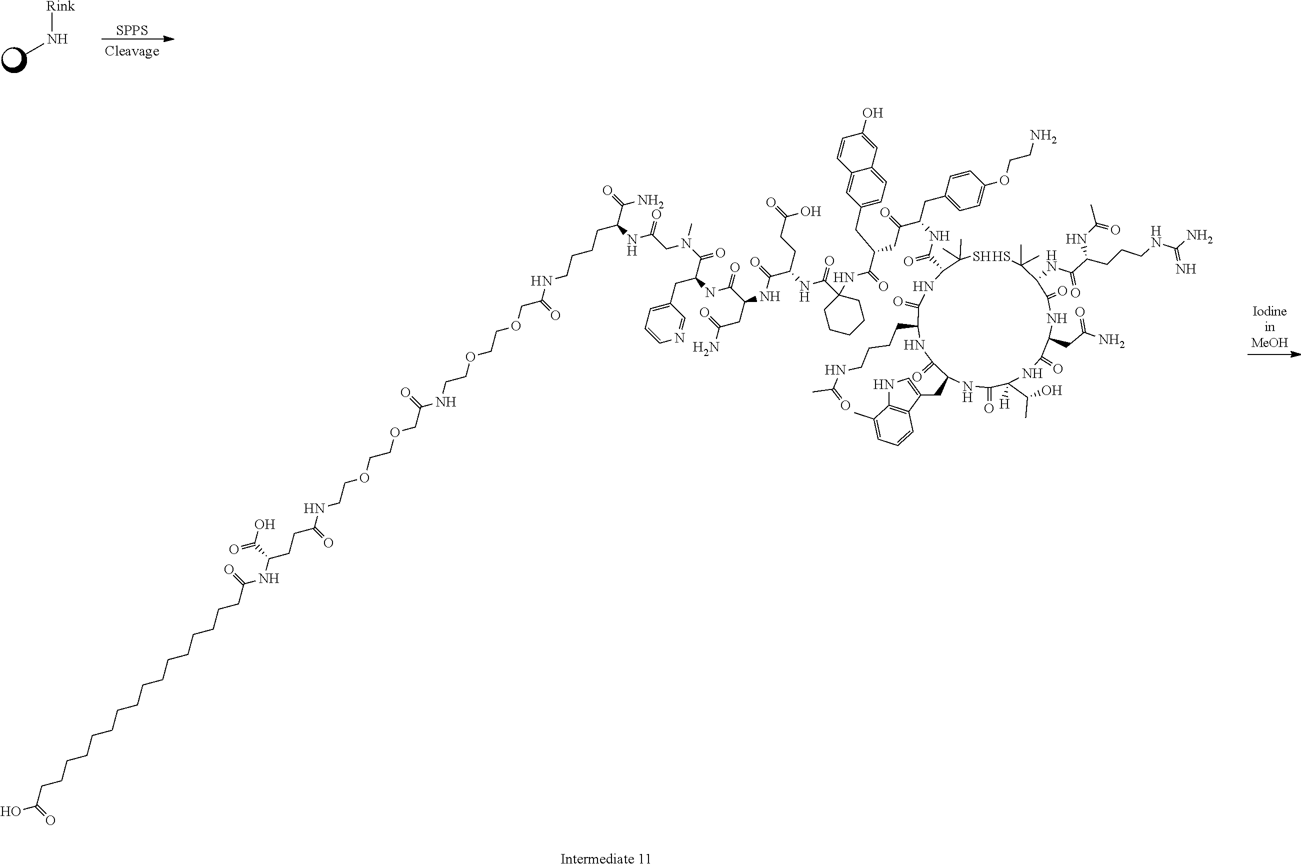

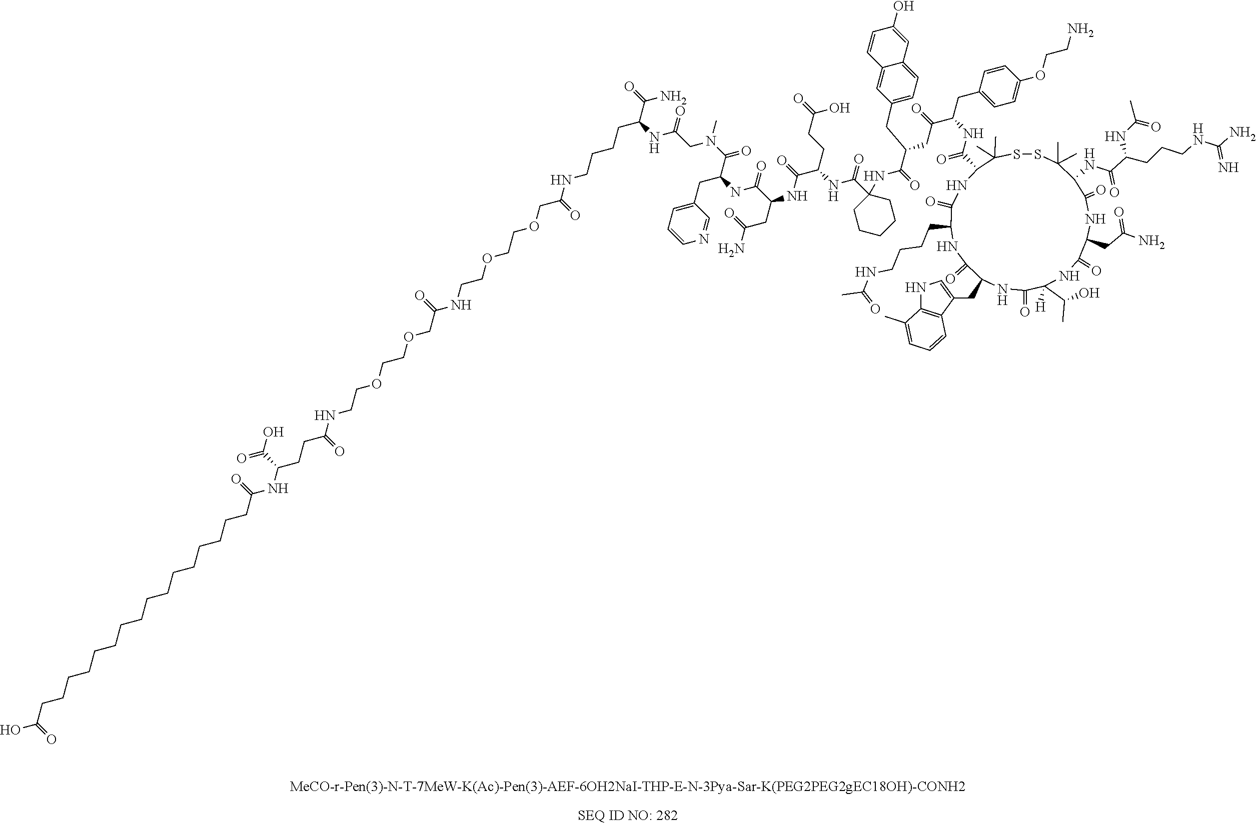 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)[C@@H](CCCNC(=N)N)NC(C)=O)C(C)(C)SSC(C)(C)[C@@H](C(=O)N[C@@H](Cc2ccc(OCCN)cc2)C(=O)C[C@@H](Cc2ccc3cc(O)ccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(=O)N[C@@H](CCCCNC(=O)COCCOCCNC(=O)COCCOCCNC(=O)CC[C@H](NC(=O)CCCCCCCCCCCCCCCCC(=O)O)C(=O)O)C(N)=O)CCCCC2)NC1=O]

Step A—Synthesis of Intermediate 11: The peptide was synthesized by solid-phase peptide synthesis using Fmoc chemistry. DMF was added to a vessel containing MBHA Resin (0.3 mmol, 1.0 g, sub: 0.28 mmol/g), and the resin was allowed to swell for 2 hours. Then, 20% piperidine/DMF was added to the resin and mixed for 30 minutes. The mixture was drained and washed five times with DMF (for 30 seconds each). Fmoc-amino acid solution was then added to the resin and mixed for 30 seconds before the addition of activation buffer. The amino acid was allowed to react with the resin for 1-16 hours under N2. Then, 20% piperidine/DMF was added and mixed for 30 min. The procedure was repeated for subsequent amino acid coupling(s). Coupling reactions were monitored by ninhydrin (A: 5% ninhydrin/EtOH; B: 80% phenol/EtOH; C: pyridine) or tetrachlor (A: 2% tetrachlor/DMF; B: 2% aldehyde/DMF 110° C. for 3 min) color test. Subsequently, the resin was washed with DMF 5 times, and then with MeOH 3 times before drying under vacuum.

To cleave the peptide from the resin, 25 mL of cleavage buffer (5.0% DTT/2.5% H₂O/2.5% TIS/90% TFA) was added to the flask containing the side chain protected peptide on resin at room temperature, and the solution was stirred for 3 hrs. The mixture was then filtered and washed with 5 mL TFA. The combined filtrate was triturated with cold methyl tertbutyl ether (MTBE). The mixture was centrifuged (3000 rpm, 3 min) and decanted. The pellet was washed with MTBE and centrifuged. The residue was lyophilized to give Intermediate 11 (750 mg).

Step B—Peptide Cyclization and Purification: Intermediate 11 (750 mg, 0.257 mmol) was dissolved in 20% MeCN/H2O (300 mL). To a stirred solution of the peptide was added iodine in MeOH (0.1M, 1.5 mL) drop-wise until the color of the solution remained yellow. After ˜2 h LCMS showed the reaction was complete. Excess iodine was quenched by the addition of 1M Na₂S₂O₃ in water (15 uL) (turned colorless instantly). MeCN (10-20 mL) was added to reduce turbidity. The solution was purified by Prep-HPLC (A: 0.075% TFA in H₂O, B: ACN) (Note 1: Method H) to give SEQ ID NO: 282 (109.2 mg, 93.2% purity, 12.1% yield for this step; over all yield: 10.4%) obtained as white solid. Analysis was performed using a C18 column with a flow rate of 1 m/min (Note 2).

LCMS Summary: calculated MW: 2914.4, observed MW: 1458.3 (M+2H)²⁺.

### Example 14: Synthesis of SEQ ID NO: 273—MeCO-Pen(3)-N-T-7MeW-K(Ac)-Pen(3)-AEF-6OH2Nal-THP-K(PEG2PEG2gEC18OH)-N-3Pya-Sar-CONH2

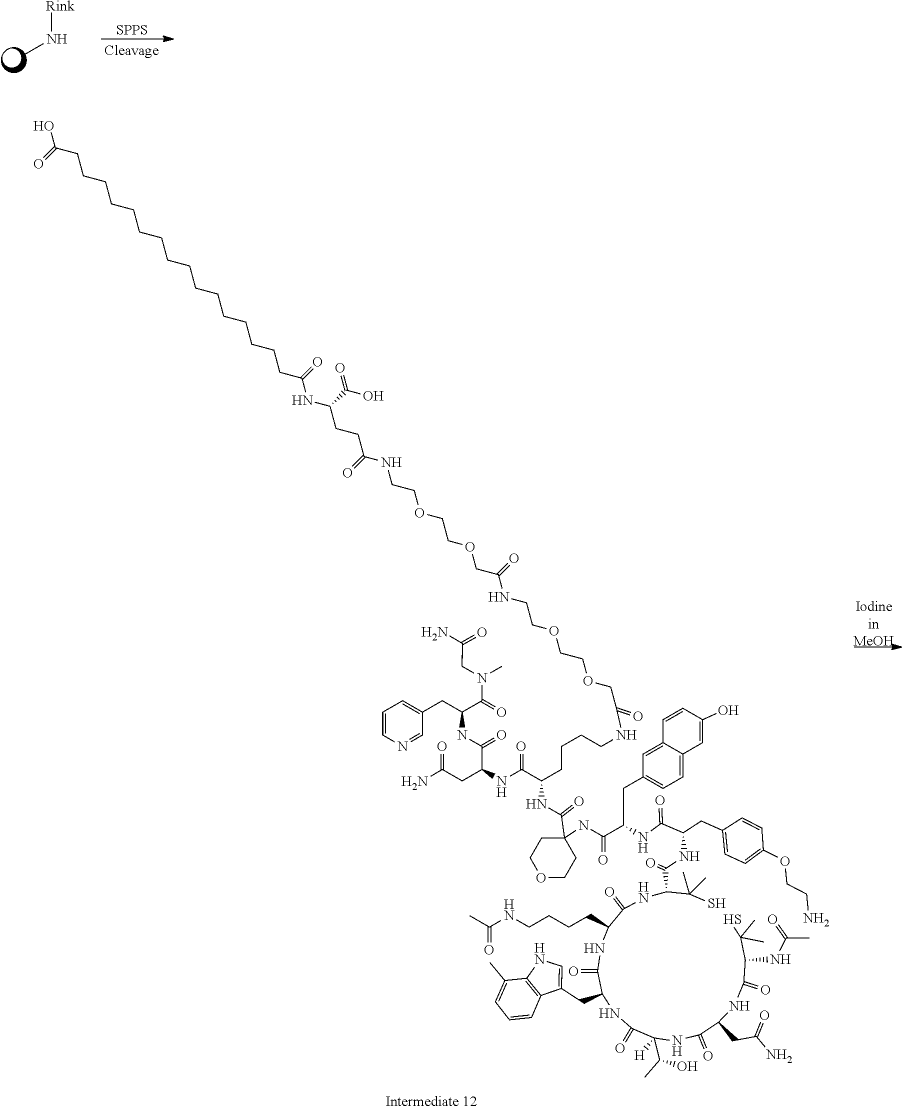

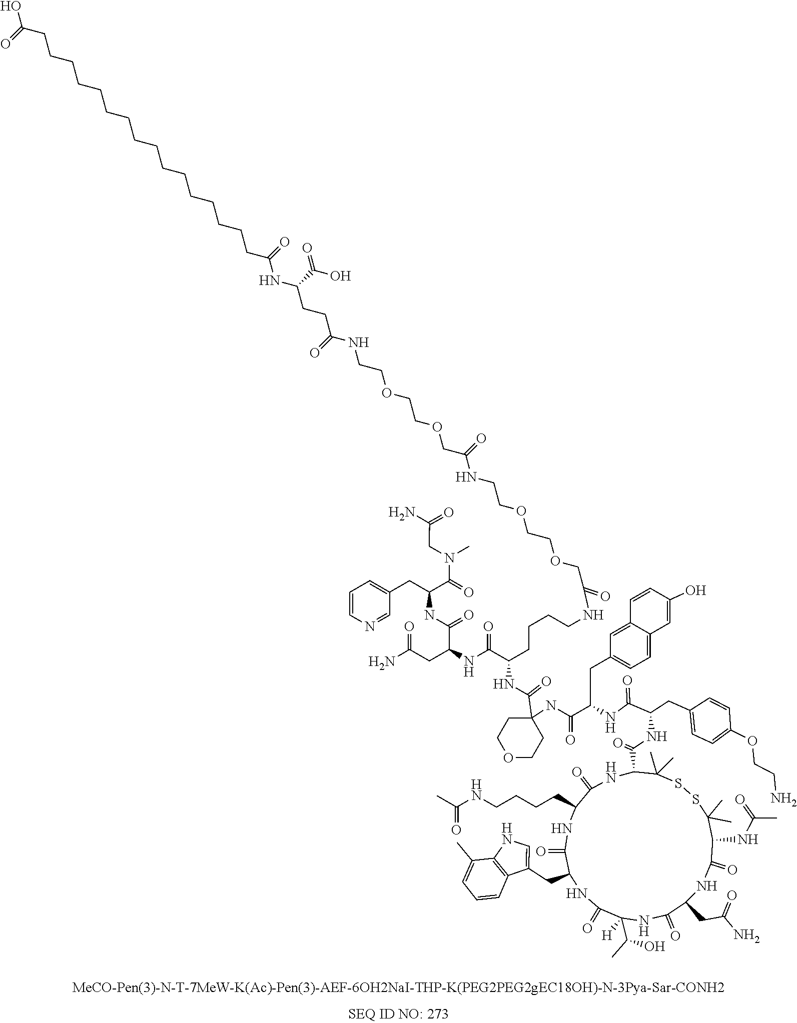

Step A—Synthesis of Intermediate 12: The peptide was synthesized by solid-phase peptide synthesis using Fmoc chemistry. DMF was added to a vessel containing MBHA Resin (1.0 mmol, 2.9 g, sub: 0.35 mmol/g), and the resin was allowed to swell for 2 hours. Then, 20% piperidine/DMF was added to the resin and mixed for 30 minutes. The mixture was drained and washed five times with DMF (for 30 seconds each). Fmoc-amino acid solution was then added to the resin and mixed for 30 seconds before the addition of activation buffer. The amino acid was allowed to react with the resin for 1-16 hours under N2. Then, 20% piperidine/DMF was added and mixed for 30 min. The procedure was repeated for subsequent amino acid coupling(s). Coupling reactions were monitored by ninhydrin (A: 5% ninhydrin/EtOH; B: 80% phenol/EtOH; C: pyridine) or tetrachlor (A: 2% tetrachlor/DMF; B: 2% aldehyde/DMF 110° C. for 3 min) color test. Subsequently, the resin was washed with DMF 5 times, and then with MeOH 3 times before drying under vacuum.

To cleave the peptide from the resin, 90 mL of cleavage buffer (5.0% DTT/2.5% H₂O/2.5% TIS/90% TFA) was added to the flask containing the side chain protected peptide on resin at room temperature, and the solution was stirred for 3 hrs. The mixture was then filtered and washed with 5 mL TFA. The combined filtrate was triturated with cold methyl tertbutyl ether (MTBE). The mixture was centrifuged (3000 rpm, 3 min) and decanted. The pellet was washed with MTBE and centrifuged. The residue was lyophilized to give Intermediate 12 (2.0 g).

Step B—Peptide Cyclization and Purification: Intermediate 12 (2.0 g, 0.76 mmol) was dissolved in 20% MeCN/H2O (1000 mL). To a stirred solution of the peptide was added iodine in MeOH (0.1M, 5.0 mL) drop-wise until the color of the solution remained yellow. After ˜2 h LCMS showed the reaction was complete. Excess iodine was quenched by the addition of 1M Na₂S₂O₃ in water (15 uL) (turned colorless instantly). MeCN (10-20 mL) was added to reduce turbidity. The solution was purified by Prep-HPLC (A: 0.075% TFA in H₂O, B: ACN) (Note 1: Method F) to give SEQ ID NO: 273 (434.5 mg, 97.6% purity, 19.5% yield for this step; over all yield: 14.8%) obtained as white solid. Analysis was performed using a C18 column with a flow rate of 1 mE/min (Note 2).

LCMS Summary: calculated MW: 2629.1, observed MW: 1314.8 (M+2H)²⁺.

### Example 15: Synthesis of SEQ ID NO: 442—MeCO-r-Pen(3)-N-T-7MeW-K(Ac)-Pen(3)-AEF-6OH2Nal-THP-E-N-3Pya-Sar-CONH2

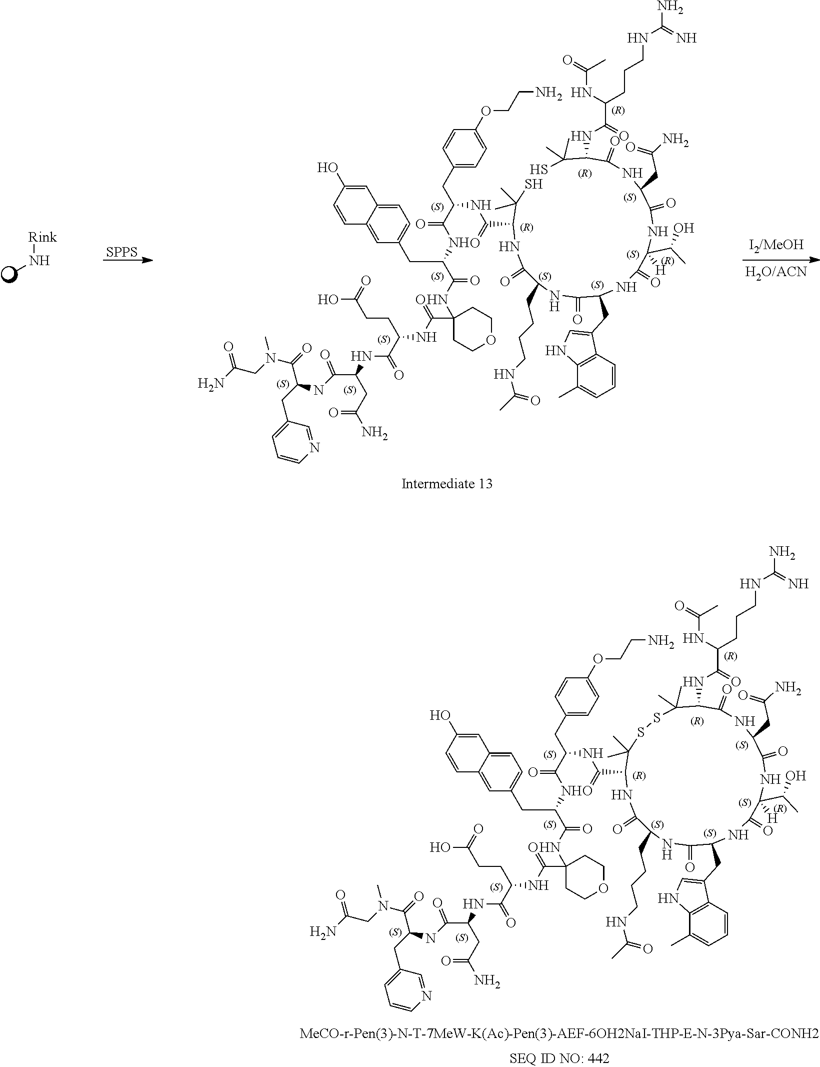

Step A—Synthesis of Intermediate 13: The peptide was synthesized by solid-phase peptide synthesis using Fmoc chemistry. DMF was added to a vessel containing MBHA Resin (0.20 mmol, 0.64 g, sub: 0.31 mmol/g), and the resin was allowed to swell for 2 hours. Then, 20% piperidine/DMF was added to the resin and mixed for 30 minutes. The mixture was drained and washed five times with DMF (for 30 seconds each). Fmoc-amino acid solution was then added to the resin and mixed for 30 seconds before the addition of activation buffer. The amino acid was allowed to react with the resin for 1-4 hours under N2. Then, 20% piperidine/DMF was added and mixed for 30 min. The procedure was repeated for subsequent amino acid coupling(s). Coupling reactions were monitored by ninhydrin (A: 5% ninhydrin/EtOH; B: 80% phenol/EtOH; C: pyridine) or tetrachlor (A: 2% tetrachlor/DMF; B: 2% aldehyde/DMF 110° C. for 3 min) color test. Subsequently, the resin was washed with DMF 5 times, and then with MeOH 3 times before drying under vacuum.

To cleave the peptide from the resin, 12 mL of cleavage buffer (5.0% DTT/2.5% H₂O/2.5% TIS/90% TFA) was added to the flask containing the side chain protected peptide on resin at room temperature, and the solution was stirred for 3 hrs. The mixture was then filtered and washed with 5 mL TFA. The combined filtrate was triturated with cold methyl tertbutyl ether (MTBE). The mixture was centrifuged (3000 rpm, 3 min) and decanted. The pellet was washed with MTBE and centrifuged. The residue was lyophilized to give Intermediate 13 (400 mg).

Step B—Peptide Cyclization and Purification: Intermediate 13 (400 mg, 0.193 mmol) was dissolved in 20% MeCN/H2O (200 mL). To a stirred solution of the peptide was added iodine in MeOH (0.1M, 1.0 mL) drop-wise until the color of the solution remained yellow. After ˜2 h LCMS showed the reaction was complete. Excess iodine was quenched by the addition of 1M Na₂S₂O₃ in water (15 uL) (turned colorless instantly). MeCN (10-20 mL) was added to reduce turbidity. The solution was purified by Prep-HPLC (A: 0.075% TFA in H₂O, B: ACN) (Note 1: Method C) to give SEQ ID NO: 442 (50.8 mg, 94.2% purity, 10.3% yield for this step; over all yield: 9.9%) obtained as white solid. Analysis was performed using a C18 column with a flow rate of 1 mL/min (Note 2).

LCMS Summary: calculated MW: 2070.3, observed MW: 1036.2 (M+2H)²⁺, 691.1 (M+3H)³⁺.

### Example 16: Synthesis of SEQ ID NO: 428—MeCO-Pen(3)-N-T-7MeW-K(Ac)-Pen(3)-AEF-6OH2Nal-THP-E-N-3Pya-Sar-CONH2

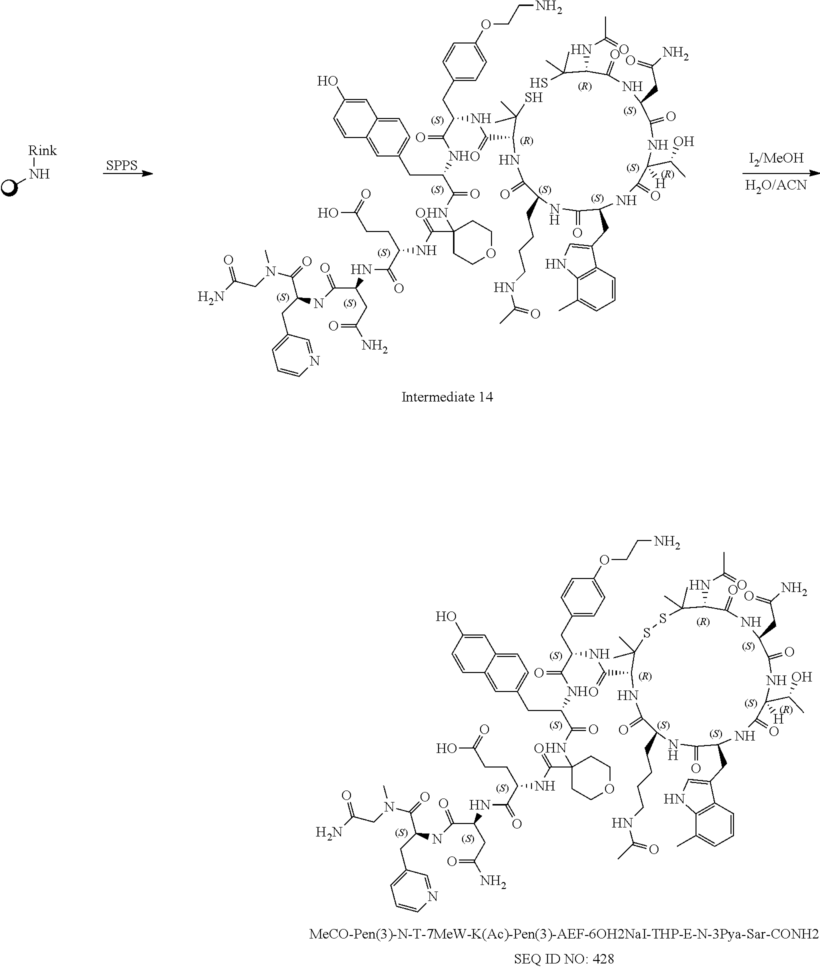

Step A—Synthesis of Intermediate 14: The peptide was synthesized by solid-phase peptide synthesis using Fmoc chemistry. DMF was added to a vessel containing MBHA Resin (0.10 mmol, 0.32 g, sub: 0.31 mmol/g), and the resin was allowed to swell for 2 hours. Then, 20% piperidine/DMF was added to the resin and mixed for 30 minutes. The mixture was drained and washed five times with DMF (for 30 seconds each). Fmoc-amino acid solution was then added to the resin and mixed for 30 seconds before the addition of activation buffer. The amino acid was allowed to react with the resin for 1-4 hours under N2. Then, 20% piperidine/DMF was added and mixed for 30 min. The procedure was repeated for subsequent amino acid coupling(s). Coupling reactions were monitored by ninhydrin (A: 5% ninhydrin/EtOH; B: 80% phenol/EtOH; C: pyridine) or tetrachlor (A: 2% tetrachlor/DMF; B: 2% aldehyde/DMF 110° C. for 3 min) color test. Subsequently, the resin was washed with DMF 5 times, and then with MeOH 3 times before drying under vacuum.

To cleave the peptide from the resin, 8 mL of cleavage buffer (5.0% DTT/2.5% H₂O/2.5% TIS/90% TFA) was added to the flask containing the side chain protected peptide on resin at room temperature, and the solution was stirred for 3 hrs. The mixture was then filtered and washed with 5 mL TFA. The combined filtrate was triturated with cold methyl tertbutyl ether (MTBE). The mixture was centrifuged (3000 rpm, 3 min) and decanted. The pellet was washed with MTBE and centrifuged. The residue was lyophilized to give Intermediate 14 (195 mg).

Step B—Peptide Cyclization and Purification: Intermediate 14 (195 mg, 0.10 mmol) was dissolved in 20% MeCN/H2O (100 mL). To a stirred solution of this peptide was added iodine in MeOH (0.1M, 0.5 mL) drop-wise until the color of the solution remained yellow. After about 2 h LCMS analysis showed that Intermediate 14 was no longer present. The excess iodine was quenched by the addition of 1M Na₂S₂O₃ in water (15 μL). MeCN (10-20 mL) was added to reduce the turbidity of the solution. The peptide was purified by Prep-HPLC (A: 0.075% TFA in H₂O, B: ACN) (Note 1: Method C) to provide SEQ ID NO: 428 (18.9 mg, 99.3% purity, 8.61% yield for this step; over all yield: 8.6%) obtained as white solid. Analysis was performed using a C18 column with a flow rate of 1 mL/min (Note 2).

LCMS Summary: calculated MW: 1914.17, observed MW: 958.0 (M+2H)²⁺.

### Example 17: Synthesis of SEQ ID NO: 444—MeCO-r-Pen(3)-N-T-7MeW-K(Ac)-Pen(3)-AEF-6Br2Nal-THP-E-N-THP-CONH2

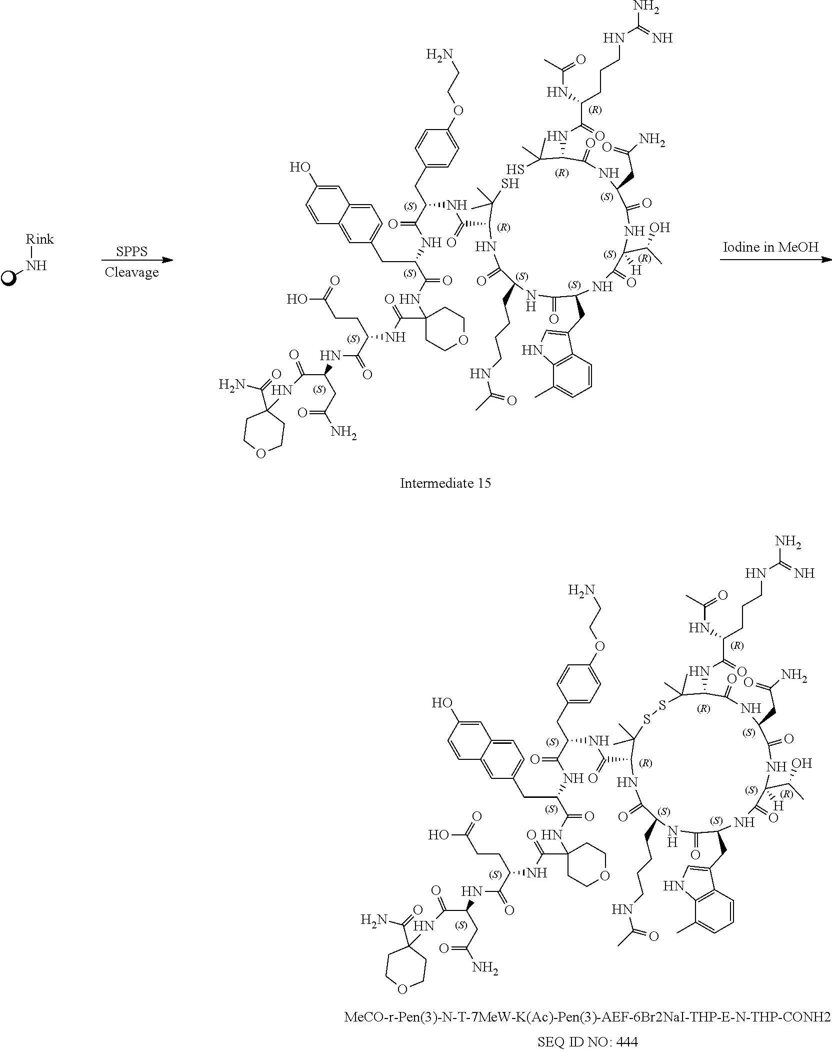

Step A—Synthesis of Intermediate 15: The peptide was synthesized by solid-phase peptide synthesis using Fmoc chemistry. DMF was added to a vessel containing MBHA Resin (3 mmol, 9.6 g, sub: 0.31 mmol/g), and the resin was allowed to swell for 2 hours. Then, 20% piperidine/DMF was added to the resin and mixed for 30 minutes. The mixture was drained and washed five times with DMF (for 30 seconds each). Fmoc-amino acid solution was then added to the resin and mixed for 30 seconds before the addition of activation buffer. The amino acid was allowed to react with the resin for 1-16 hours under N2. Then, 20% piperidine/DMF was added and mixed for 30 min. The procedure was repeated for subsequent amino acid coupling(s). Coupling reactions were monitored by ninhydrin (A: 5% ninhydrin/EtOH; B: 80% phenol/EtOH; C: pyridine) or tetrachlor (A: 2% tetrachlor/DMF; B: 2% aldehyde/DMF 110° C. for 3 min) color test. Subsequently, the resin was washed with DMF 5 times, and then with MeOH 3 times before drying under vacuum.

To cleave the peptide from the resin, 200 mL of cleavage buffer (5.0% DTT/2.5% H₂O/2.5% TIS/90% TFA) was added to the flask containing the side chain protected peptide on resin at room temperature, and the solution was stirred for 3 hrs. The mixture was then filtered and washed with 20 mL TFA. The combined filtrate was triturated with cold methyl tertbutyl ether (MTBE). The mixture was centrifuged (3000 rpm, 3 min) and decanted. The pellet was washed with MTBE and centrifuged. The residue was lyophilized to give Intermediate 15 (6.0 g).

Step B—Peptide Cyclization and Purification: Intermediate 15 (6.0 g, 2.93 mmol) was dissolved in 20% MeCN/H2O (3.0 L). To a stirred solution of the peptide was added iodine in MeOH (0.1M, 15 mL) drop-wise until the color of the solution remained yellow. After ˜2 h LCMS showed the reaction was complete. Excess iodine was quenched by the addition of 1M Na₂S₂O₃ in water (15 uL) (turned colorless instantly). MeCN (150-200 mL) was added to reduce turbidity. The solution was purified by Prep-HPLC (A: 0.075% TFA in H₂O, B: ACN) (Note 1: Method I) to give SEQ ID NO: 444 (2.35 g, 96.7% purity, 34.1% yield for this step; over all yield: 33.4%) obtained as white solid. Analysis was performed using a C18 column with a flow rate of 1 mL/min (Note 2).

LCMS Summary: calculated MW: 2041.1, observed MW: 1021.3 (M+2H)².

### Example 18: Synthesis of SEQ ID NO: 432—MeCO-r-Pen(3)-N-T-7MeW-K(Ac)-Pen(3)-AEF-2Nal6(Ph4(NMorph))-THP-E-N-THP-CONH2

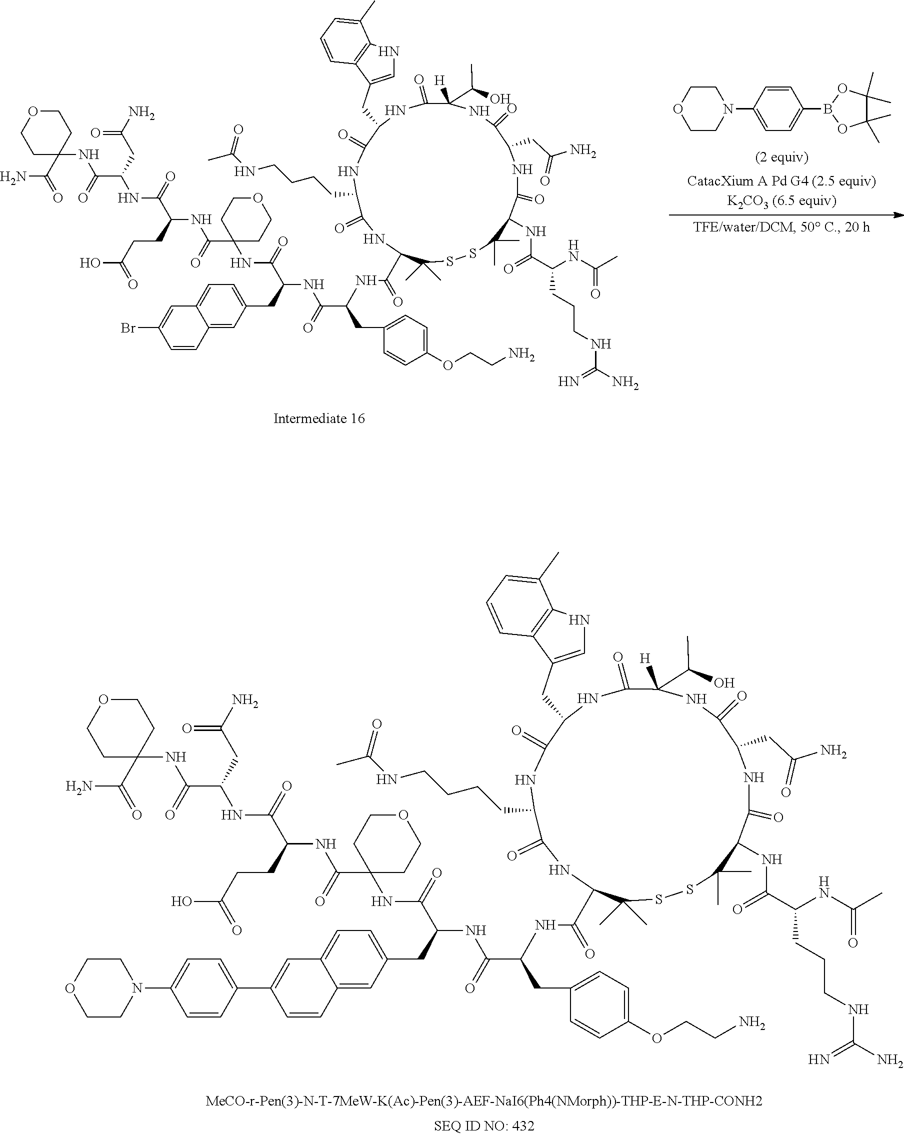

In a glovebox, a 1 dram vial was charged with Intermediate 16 (15 mg, 7.4 μmol), 4-[4-(4,4,5,5-tetramethyl-1,3,2-dioxaborolan-2-yl)phenyl]morpholine (4.3 mg, 15 μmol, 2 equiv), CatacXium A Pd G4 (13.6 mg, 18.4 μmol, 2.5 equiv), and K₂CO₃ (6.6 mg, 48 μmol, 6.5 equiv) before adding TFE (0.37 mL), water (0.37 mL), and DCM (75 μL). The reaction vial was sealed, brought out of the glovebox and heated at 50° C. with stirring for 20 h before cooling to room temperature, adding SiliaMet DMT scavenger (15 mg) and DMF (0.5 mL) and stirring for an additional 3 h. The resulting mixture was acidified by the addition of a MeCN/water (9:1, 1% TFA) mixture and filtered, rinsed with minimal DMF, and concentrated to a volume of approx. 1 mL by rotary evaporation. Purification by mass-directed liquid chromatography (HPLC (Waters XSelect CSH C18, 5 u, 19×100 mm) using a gradient of 20-28% B over 25 minutes at a flow rate of 25 mL/min (mobile phase A: water+0.16% Formic Acid, mobile phase B: acetonitrile+0.16% Formic Acid)) afforded SEQ ID NO: 432 as a white solid 2 (4.5 mg, 27% yield). LCMS calc'd for C101H139N23O24S2: 2123.49, obsvd (m/z): 1062.2 [M+2H]²⁺.

### Example 19: IL23R Reporter Assay

Compounds were serially diluted in 100% (v/v) DMSO) and plated using an Echo acoustic dispenser (Labcyte) into 1536-well non-treated black assay plates (Corning #9146). 3 μL of HEK293 cells containing IL-23R, IL-12Rβ1 and a firefly luciferase reporter gene driven by a STAT-inducible promoter (Promega) were added to the plates (4000 cells/well), followed by 3 μL of 10 ng/mL IL-23 (equivalent to EC₉₀ concentration). After 5 h at 37° C., 5% CO₂, 95% relative humidity, cells were placed at 20° C. and treated with BioGlo reagent (Promega) according to the Manufacturer's instructions. Luminescence was measured on a Pherastar FSX (BMG LabTech). Data were normalized to IL-23 treatment (0% inhibition) and 30 μM of control inhibitor (100% inhibition), and IC₅₀ values were determined using a 4-parameter Hill equation. Data for exemplary compounds are shown below.
A: IC₅₀<0.01 μM;
B: 0.01 μM≤IC₅₀<0.1 μM;
C: 0.1 μM≤IC₅₀
ND: Not determined

TABLE 8
IL-23 Binding Data

| SEQ ID NO | IC₅₀ |
|---|---|
| 1 | A |
| 2 | A |
| 3 | A |
| 4 | A |
| 5 | A |
| 6 | A |
| 7 | A |
| 8 | A |
| 9 | A |
| 10 | A |
| 11 | A |
| 12 | A |
| 13 | A |
| 14 | A |
| 15 | A |
| 16 | A |
| 17 | A |
| 18 | A |
| 19 | A |
| 20 | A |
| 21 | A |
| 22 | A |
| 23 | A |
| 24 | A |
| 25 | A |
| 26 | A |
| 27 | A |
| 28 | A |
| 29 | A |
| 30 | A |
| 31 | A |
| 32 | A |
| 33 | A |
| 34 | A |
| 35 | A |
| 36 | A |
| 37 | A |
| 38 | A |
| 39 | A |
| 40 | A |
| 41 | A |
| 42 | A |
| 43 | B |
| 44 | A |
| 45 | B |
| 46 | B |
| 47 | B |
| 48 | A |
| 49 | A |
| 50 | A |
| 51 | A |
| 52 | A |
| 53 | A |
| 54 | B |
| 55 | B |
| 56 | A |
| 57 | B |
| 58 | A |
| 59 | A |
| 60 | A |
| 61 | A |
| 62 | A |
| 63 | A |
| 64 | B |
| 65 | A |
| 66 | A |
| 67 | A |
| 68 | A |
| 69 | A |
| 70 | B |
| 71 | A |
| 72 | A |
| 73 | A |
| 74 | A |
| 75 | A |
| 76 | A |
| 77 | A |
| 78 | A |
| 79 | A |
| 80 | A |
| 81 | A |
| 82 | A |
| 83 | B |
| 84 | A |
| 85 | A |
| 86 | A |
| 87 | A |
| 88 | B |
| 89 | A |
| 90 | A |
| 91 | A |
| 92 | A |
| 93 | A |
| 94 | A |
| 95 | A |
| 96 | B |
| 97 | B |
| 98 | A |
| 99 | A |
| 100 | A |
| 101 | A |
| 102 | A |
| 103 | A |
| 104 | A |
| 105 | B |
| 106 | A |
| 107 | B |
| 108 | A |
| 109 | A |
| 110 | B |
| 111 | A |
| 112 | A |
| 113 | A |
| 114 | A |
| 115 | B |
| 116 | B |
| 117 | B |
| 118 | B |
| 119 | B |
| 120 | C |
| 121 | B |
| 122 | B |
| 123 | B |
| 124 | B |
| 125 | B |
| 126 | B |
| 127 | B |
| 128 | B |
| 129 | B |
| 130 | C |
| 131 | ND |
| 132 | A |
| 133 | A |
| 134 | B |
| 135 | B |
| 136 | A |
| 137 | A |
| 138 | B |
| 139 | B |
| 140 | B |
| 141 | B |
| 142 | B |
| 143 | A |
| 144 | B |
| 145 | B |
| 146 | A |
| 147 | B |
| 148 | B |
| 149 | A |
| 150 | B |
| 151 | B |
| 152 | C |
| 153 | B |
| 154 | C |
| 155 | B |
| 156 | B |
| 157 | A |
| 158 | A |
| 159 | A |
| 160 | A |
| 161 | ND |
| 162 | ND |
| 163 | ND |
| 164 | B |
| 165 | ND |
| 166 | B |
| 167 | C |
| 168 | C |
| 169 | B |
| 170 | B |
| 171 | A |
| 172 | A |
| 173 | B |
| 174 | B |
| 175 | C |
| 176 | C |
| 177 | C |
| 178 | C |
| 179 | C |
| 180 | C |
| 181 | B |
| 182 | B |
| 183 | C |
| 184 | B |
| 185 | B |
| 186 | B |
| 187 | C |
| 188 | C |
| 189 | C |
| 190 | B |
| 191 | B |
| 192 | B |
| 193 | B |
| 194 | B |
| 195 | B |
| 196 | B |
| 197 | B |
| 198 | ND |
| 199 | ND |
| 200 | C |
| 201 | B |
| 202 | B |
| 203 | B |
| 204 | B |
| 205 | B |
| 206 | B |
| 207 | B |
| 208 | B |
| 209 | B |
| 210 | B |
| 211 | C |
| 212 | B |
| 213 | B |
| 214 | B |
| 215 | B |
| 216 | B |
| 217 | B |
| 218 | B |
| 219 | A |
| 220 | B |
| 221 | B |
| 222 | C |
| 223 | B |
| 224 | B |
| 225 | B |
| 226 | A |
| 227 | B |
| 228 | B |
| 229 | B |
| 230 | A |
| 231 | B |
| 232 | B |
| 233 | B |
| 234 | B |
| 235 | B |
| 236 | B |
| 237 | B |
| 238 | B |
| 239 | B |
| 240 | B |
| 241 | B |
| 242 | B |
| 243 | B |
| 244 | B |
| 245 | B |
| 246 | B |
| 247 | ND |
| 248 | ND |
| 249 | B |
| 250 | B |
| 251 | B |
| 252 | B |
| 253 | B |
| 254 | B |
| 255 | B |
| 256 | B |
| 257 | B |
| 258 | B |
| 259 | B |
| 260 | B |
| 261 | B |
| 262 | B |
| 263 | B |
| 264 | B |
| 265 | B |
| 266 | B |
| 267 | B |
| 268 | B |
| 269 | B |
| 270 | B |
| 271 | B |
| 272 | C |
| 273 | B |
| 274 | C |
| 275 | C |
| 276 | C |
| 277 | B |
| 278 | C |
| 279 | C |
| 280 | B |
| 281 | A |
| 282 | B |
| 283 | B |
| 284 | B |
| 285 | B |
| 286 | B |
| 287 | A |
| 288 | C |
| 289 | C |
| 290 | A |
| 291 | A |
| 292 | A |
| 293 | A |
| 294 | A |
| 295 | A |
| 296 | A |
| 297 | A |
| 298 | B |
| 299 | A |
| 300 | A |
| 301 | A |
| 302 | A |
| 303 | A |
| 304 | A |
| 305 | A |
| 306 | A |
| 307 | A |
| 308 | A |
| 309 | A |
| 310 | A |
| 311 | A |
| 312 | A |
| 313 | A |
| 314 | B |
| 315 | A |
| 316 | A |
| 317 | ND |
| 318 | A |
| 319 | A |
| 320 | A |
| 321 | A |
| 322 | A |
| 323 | A |
| 324 | A |
| 325 | A |
| 326 | A |
| 327 | A |
| 328 | A |
| 329 | B |
| 330 | A |
| 331 | A |
| 332 | A |
| 333 | A |
| 334 | A |
| 335 | A |
| 336 | A |
| 337 | A |
| 338 | A |
| 339 | A |
| 340 | A |
| 341 | B |
| 342 | A |
| 343 | A |
| 344 | A |
| 345 | A |
| 346 | A |
| 347 | A |
| 348 | A |
| 349 | A |
| 350 | A |
| 351 | A |
| 352 | A |
| 353 | A |
| 354 | A |
| 355 | A |
| 356 | A |
| 357 | A |
| 358 | A |
| 359 | A |
| 360 | A |
| 361 | A |
| 362 | A |
| 363 | A |
| 364 | A |
| 365 | A |
| 366 | A |
| 367 | ND |
| 368 | A |
| 369 | A |
| 370 | A |
| 371 | A |
| 372 | A |
| 373 | A |
| 374 | A |
| 375 | A |
| 376 | A |
| 377 | A |
| 378 | A |
| 379 | A |
| 380 | A |
| 381 | A |
| 382 | A |
| 383 | A |
| 384 | A |
| 385 | A |
| 386 | A |
| 387 | A |
| 388 | A |
| 389 | A |
| 390 | A |
| 391 | A |
| 392 | A |
| 393 | A |
| 394 | A |
| 395 | B |
| 396 | A |
| 397 | A |
| 398 | A |
| 399 | A |
| 400 | A |
| 401 | A |
| 402 | A |
| 403 | A |
| 404 | A |
| 405 | A |
| 406 | A |
| 407 | A |
| 408 | A |
| 409 | A |
| 410 | A |
| 411 | A |
| 412 | A |
| 413 | A |
| 414 | A |
| 415 | A |
| 416 | A |
| 417 | A |
| 418 | ND |
| 419 | B |
| 420 | B |
| 421 | B |
| 422 | A |
| 423 | B |
| 424 | C |
| 425 | B |
| 426 | B |
| 427 | B |
| 428 | A |
| 429 | C |
| 430 | C |
| 431 | C |
| 432 | C |
| 433 | ND |
| 434 | ND |
| 435 | B |
| 436 | B |
| 437 | C |
| 438 | ND |
| 439 | C |
| 440 | B |
| 441 | A |
| 442 | A |
| 443 | B |
| 444 | C |
| 445 | C |
| 446 | C |
| 447 | C |

### Example 19: PBMC pSTAT3 Assay

Cryopreserved peripheral blood mononuclear cells (PBMCs) from healthy donors were thawed and washed twice in ImmunoCult-XF T cell expansion medium (XF-TCEM) supplemented with CTL anti-aggregate wash. The cells were counted, resuspended at 2-6×10⁵ cells per mL XF-TCEM supplemented with penicillin/streptomycin and 100 ng/mL IL-1β (BioLegend, 579404), and cultured in tissue culture flasks coated with anti-CD3 (eBioscience, 16-0037-85 or BD Pharmingen, 555329) at 37° C. in 5% CO2. On day 4 of culture, PBMCs were collected, washed twice in RPMI-1640 supplemented with 0.1% BSA (RPMI-BSA), and incubated in RPMI-BSA in upright tissue culture flasks for ˜4 hours at 37° C. in 5% CO₂. Following this ‘starvation,’ a total of 6×104 cells in 30 μL RPMI-BSA was transferred into each well of a 384-well plate pre-spotted with peptide or DMSO. The cells were incubated for 30 minutes prior to the addition of IL-23 at a final concentration of 5 ng/mL. The cells were stimulated with cytokine for 30 minutes at 37° C. in 5% CO2, transferred onto ice for 10 minutes, and lysed. Cell lysates were stored at −80° C. until phosphorylated STAT3 was measured using the phospho-STAT panel kit (Meso Scale Discovery, K15202D). Results are provided below.
A: IC₅₀<0.01 nM;
B: 0.01 nM≤IC₅₀<0.1 nM;
C: 0.1 nM<IC₅₀<1 nM;
D: 1 nM<IC₅₀;
ND: Not determined

TABLE 9
PBMC pSTAT3 Assay Results

| SEQ ID NO | PBMC pSTAT3 IC₅₀ |
|---|---|
| 1 | ND |
| 2 | ND |
| 3 | ND |
| 4 | ND |
| 5 | A |
| 6 | B |
| 7 | B |
| 8 | A |
| 9 | D |
| 10 | A |
| 11 | A |
| 12 | A |
| 13 | A |
| 14 | A |
| 15 | A |
| 16 | A |
| 17 | A |
| 18 | A |
| 19 | B |
| 20 | B |
| 21 | A |
| 22 | A |
| 23 | B |
| 24 | B |
| 25 | B |
| 26 | B |
| 27 | A |
| 28 | ND |
| 29 | B |
| 30 | B |
| 31 | B |
| 32 | A |
| 33 | A |
| 34 | B |
| 35 | B |
| 36 | ND |
| 37 | ND |
| 38 | ND |
| 39 | ND |
| 40 | A |
| 41 | ND |
| 42 | D |
| 43 | ND |
| 44 | ND |
| 45 | ND |
| 46 | ND |
| 47 | ND |
| 48 | ND |
| 49 | ND |
| 50 | ND |
| 51 | ND |
| 52 | ND |
| 53 | ND |
| 54 | ND |
| 55 | ND |
| 56 | A |
| 57 | C |
| 58 | B |
| 59 | B |
| 60 | B |
| 61 | B |
| 62 | C |
| 63 | B |
| 64 | C |
| 65 | C |
| 66 | B |
| 67 | B |
| 68 | B |
| 69 | A |
| 70 | ND |
| 71 | ND |
| 72 | ND |
| 73 | ND |
| 74 | B |
| 75 | ND |
| 76 | ND |
| 77 | A |
| 78 | A |
| 79 | A |
| 80 | ND |
| 81 | ND |
| 82 | ND |
| 83 | ND |
| 84 | ND |
| 85 | ND |
| 86 | ND |
| 87 | ND |
| 88 | ND |
| 89 | B |
| 90 | B |
| 91 | B |
| 92 | B |
| 93 | B |
| 94 | C |
| 95 | B |
| 96 | B |
| 97 | C |
| 98 | A |
| 99 | A |
| 100 | A |
| 101 | A |
| 102 | A |
| 103 | A |
| 104 | B |
| 105 | C |
| 106 | B |
| 107 | B |
| 108 | A |
| 109 | A |
| 110 | C |
| 111 | B |
| 112 | A |
| 113 | B |
| 114 | A |
| 115 | C |
| 116 | C |
| 117 | ND |
| 118 | C |
| 119 | C |
| 120 | C |
| 121 | ND |
| 122 | C |
| 123 | ND |
| 124 | C |
| 125 | ND |
| 126 | C |
| 127 | C |
| 128 | C |
| 129 | C |
| 130 | C |
| 131 | C |
| 132 | A |
| 133 | A |
| 134 | C |
| 135 | C |
| 136 | A |
| 137 | A |
| 138 | ND |
| 139 | B |
| 140 | C |
| 141 | B |
| 142 | B |
| 143 | A |
| 144 | C |
| 145 | B |
| 146 | A |
| 147 | C |
| 148 | B |
| 149 | B |
| 150 | C |
| 151 | C |
| 152 | C |
| 153 | B |
| 154 | C |
| 155 | ND |
| 156 | C |
| 157 | A |
| 158 | A |
| 159 | A |
| 160 | A |
| 161 | A |
| 162 | A |
| 163 | A |
| 164 | C |
| 165 | D |
| 166 | C |
| 167 | C |
| 168 | C |
| 169 | C |
| 170 | C |
| 171 | A |
| 172 | A |
| 173 | ND |
| 174 | C |
| 175 | ND |
| 176 | D |
| 177 | D |
| 178 | D |
| 179 | D |
| 180 | D |
| 181 | C |
| 182 | C |
| 183 | C |
| 184 | C |
| 185 | C |
| 186 | C |
| 187 | C |
| 188 | C |
| 189 | C |
| 190 | C |
| 191 | C |
| 192 | C |
| 193 | C |
| 194 | B |
| 195 | B |
| 196 | C |
| 197 | C |
| 198 | C |
| 199 | ND |
| 200 | C |
| 201 | C |
| 202 | C |
| 203 | C |
| 204 | C |
| 205 | B |
| 206 | C |
| 207 | C |
| 208 | C |
| 209 | B |
| 210 | C |
| 211 | C |
| 212 | C |
| 213 | C |
| 214 | C |
| 215 | B |
| 216 | B |
| 217 | B |
| 218 | D |
| 219 | B |
| 220 | C |
| 221 | D |
| 222 | D |
| 223 | C |
| 224 | C |
| 225 | C |
| 226 | C |
| 227 | C |
| 228 | C |
| 229 | C |
| 230 | A |
| 231 | C |
| 232 | C |
| 233 | C |
| 234 | C |
| 235 | C |
| 236 | C |
| 237 | C |
| 238 | C |
| 239 | C |
| 240 | C |
| 241 | C |
| 242 | C |
| 243 | B |
| 244 | C |
| 245 | C |
| 246 | C |
| 247 | C |
| 248 | C |
| 249 | C |
| 250 | C |
| 251 | C |
| 252 | B |
| 253 | C |
| 254 | C |
| 255 | C |
| 256 | C |
| 257 | B |
| 258 | C |
| 259 | C |
| 260 | C |
| 261 | A |
| 262 | C |
| 263 | C |
| 264 | A |
| 265 | C |
| 266 | B |
| 267 | C |
| 268 | C |
| 269 | C |
| 270 | C |
| 271 | C |
| 272 | C |
| 273 | B |
| 274 | C |
| 275 | C |
| 276 | C |
| 277 | C |
| 278 | C |
| 279 | C |
| 280 | ND |
| 281 | ND |
| 282 | C |
| 283 | C |
| 284 | C |
| 285 | C |
| 286 | C |
| 287 | B |
| 288 | ND |
| 289 | ND |
| 290 | B |
| 291 | ND |
| 292 | B |
| 293 | ND |
| 294 | B |
| 295 | B |
| 296 | ND |
| 297 | ND |
| 298 | ND |
| 299 | B |
| 300 | B |
| 301 | B |
| 302 | B |
| 303 | ND |
| 304 | ND |
| 305 | ND |
| 306 | ND |
| 307 | ND |
| 308 | ND |
| 309 | B |
| 310 | B |
| 311 | ND |
| 312 | ND |
| 313 | ND |
| 314 | D |
| 315 | B |
| 316 | ND |
| 317 | ND |
| 318 | B |
| 319 | B |
| 320 | B |
| 321 | B |
| 322 | B |
| 323 | ND |
| 324 | B |
| 325 | ND |
| 326 | ND |
| 327 | ND |
| 328 | ND |
| 329 | ND |
| 330 | ND |
| 331 | ND |
| 332 | ND |
| 333 | ND |
| 334 | ND |
| 335 | A |
| 336 | B |
| 337 | A |
| 338 | ND |
| 339 | B |
| 340 | A |
| 341 | ND |
| 342 | B |
| 343 | B |
| 344 | B |
| 345 | B |
| 346 | B |
| 347 | B |
| 348 | ND |
| 349 | A |
| 350 | B |
| 351 | ND |
| 352 | B |
| 353 | ND |
| 354 | ND |
| 355 | B |
| 356 | A |
| 357 | ND |
| 358 | A |
| 359 | ND |
| 360 | B |
| 361 | B |
| 362 | ND |
| 363 | B |
| 364 | A |
| 365 | A |
| 366 | A |
| 367 | ND |
| 368 | A |
| 369 | B |
| 370 | A |
| 371 | A |
| 372 | A |
| 373 | A |
| 374 | ND |
| 375 | ND |
| 376 | ND |
| 377 | A |
| 378 | A |
| 379 | ND |
| 380 | ND |
| 381 | A |
| 382 | A |
| 383 | A |
| 384 | A |
| 385 | B |
| 386 | A |
| 387 | A |
| 388 | A |
| 389 | A |
| 390 | A |
| 391 | A |
| 392 | A |
| 393 | A |
| 394 | A |
| 395 | C |
| 396 | A |
| 397 | A |
| 398 | B |
| 399 | C |
| 400 | B |
| 401 | B |
| 402 | A |
| 403 | B |
| 404 | B |
| 405 | B |
| 406 | B |
| 407 | B |
| 408 | B |
| 409 | B |
| 410 | B |
| 411 | A |
| 412 | B |
| 413 | B |
| 414 | A |
| 415 | B |
| 416 | A |
| 417 | A |
| 418 | ND |
| 419 | B |
| 420 | B |
| 421 | C |
| 422 | A |
| 423 | C |
| 424 | C |
| 425 | C |
| 426 | ND |
| 427 | C |
| 428 | A |
| 429 | D |
| 430 | D |
| 431 | D |
| 432 | ND |
| 433 | D |
| 434 | D |
| 435 | B |
| 436 | C |
| 437 | D |
| 438 | D |
| 439 | C |
| 440 | D |
| 441 | B |
| 442 | A |
| 443 | D |
| 444 | D |
| 445 | D |
| 446 | D |
| 447 | C |

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

LENGTHY TABLES
The patent application contains a lengthy table section. A copy of the table is available in electronic form from the USPTO web site (https://seqdata.uspto.gov/?pageRequest=docDetail&DocID=US20240279281A1). An electronic copy of the table will also be available from the USPTO upon request and payment of the fee set forth in 37 CFR 1.19(b)(3).

1-93. (canceled)

94. A peptide having a structure selected from the group consisting of: 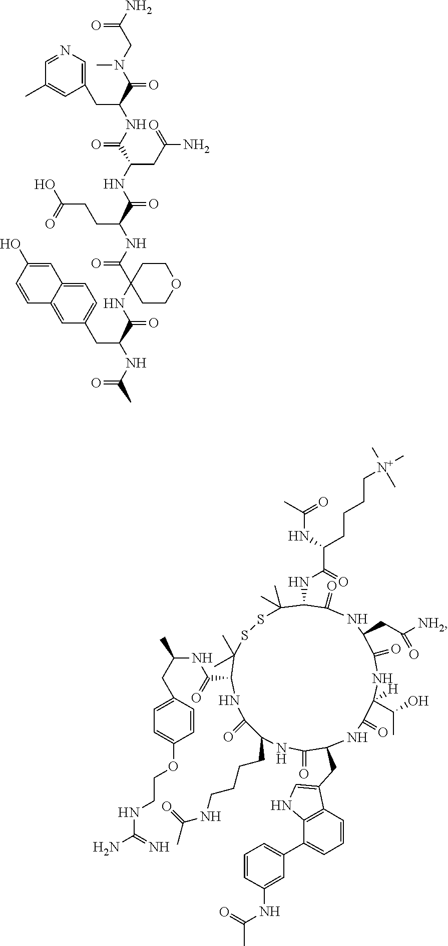 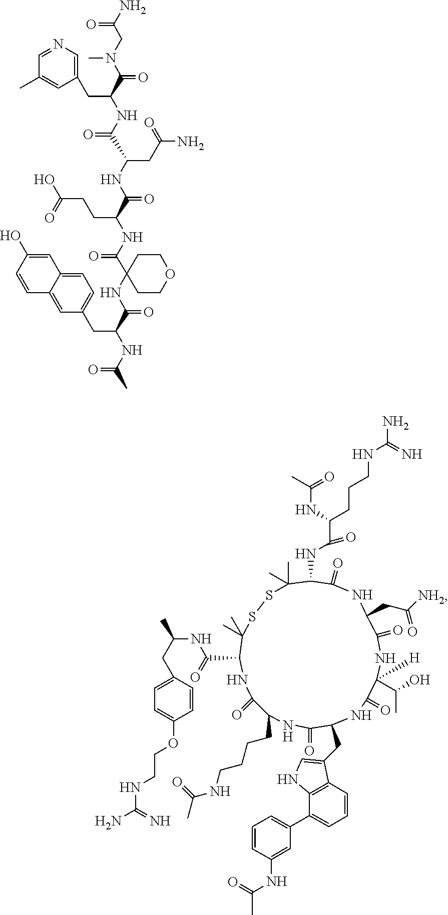 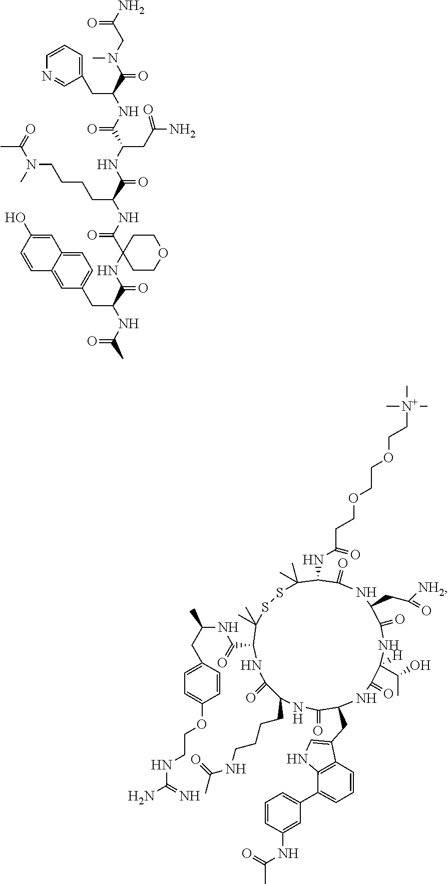 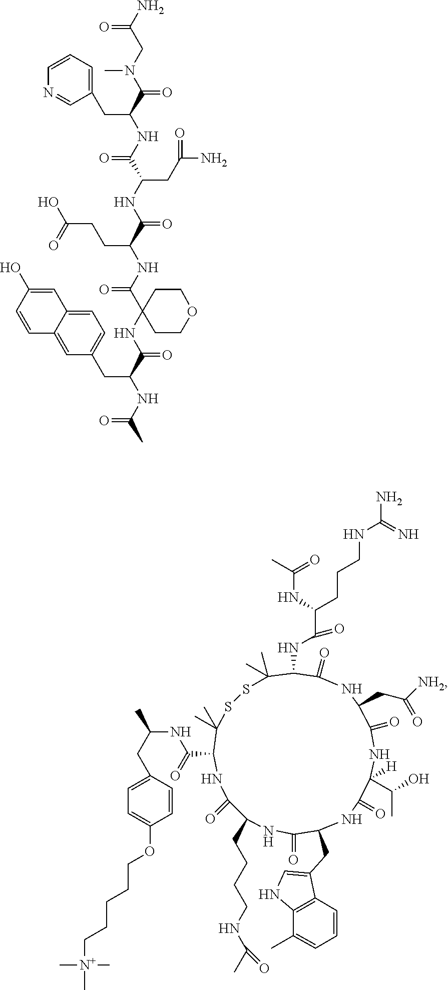 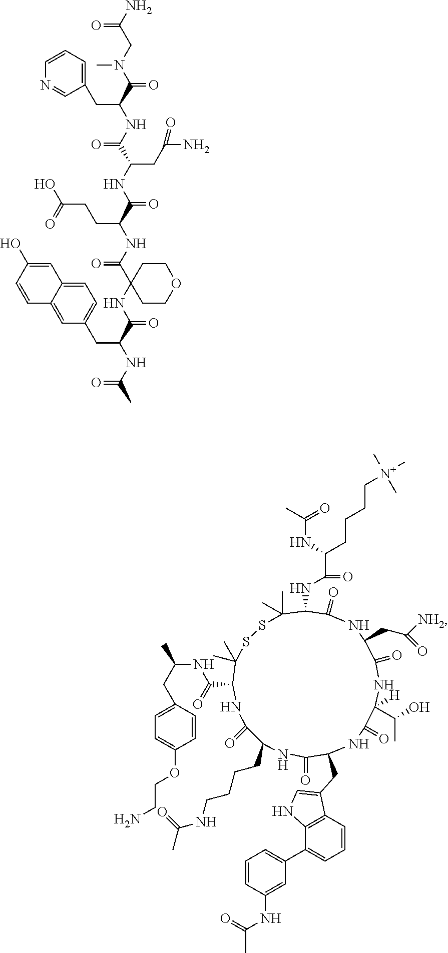 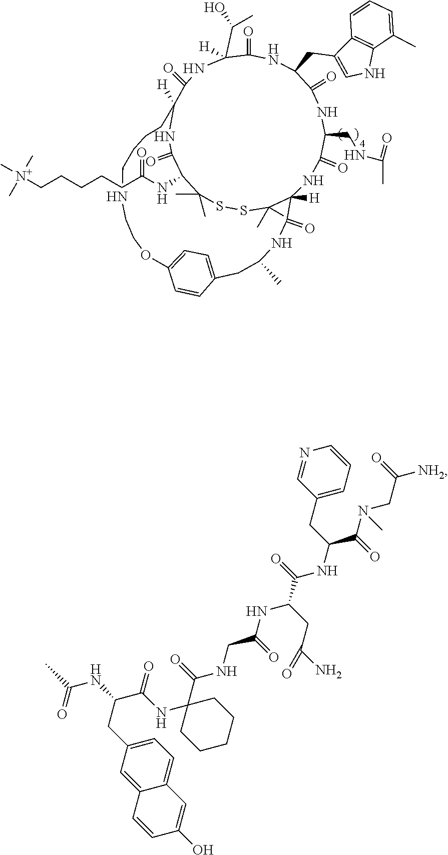 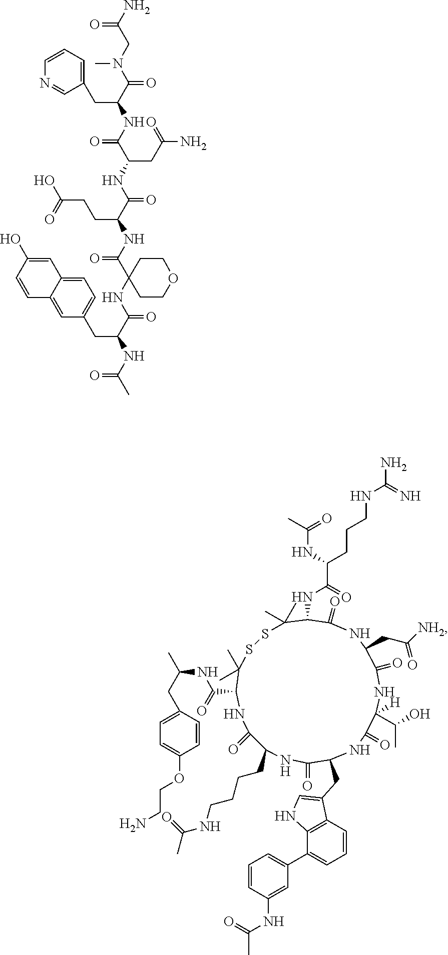 [SMILES: CC(=O)N[C@@H](Cc1ccc2cc(O)ccc2c1)C(=O)NC1(C(=O)N[C@@H](CCC(=O)O)C(=O)N[C@@H](CC(N)=O)C(=O)N[C@@H](Cc2cccnc2)C(=O)N(C)CC(N)=O)CCOCC1 ; CC(=O)NCCCC[C@@H]1NC(=O)[C@H](Cc2c[nH]c3c(-c4cccc(NC(C)=O)c4)cccc23)NC(=O)[C@H]([C@@H](C)O)NC(=O)[C@H](CC(N)=O)NC(=O)[C@@H](NC(=O)[C@@H](CCCNC(=N)N)NC(C)=O)C(C)(C)SSC(C)(C)[C@@H](C(=O)N[C@H](C)Cc2ccc(OCCN)cc2)NC1=O] 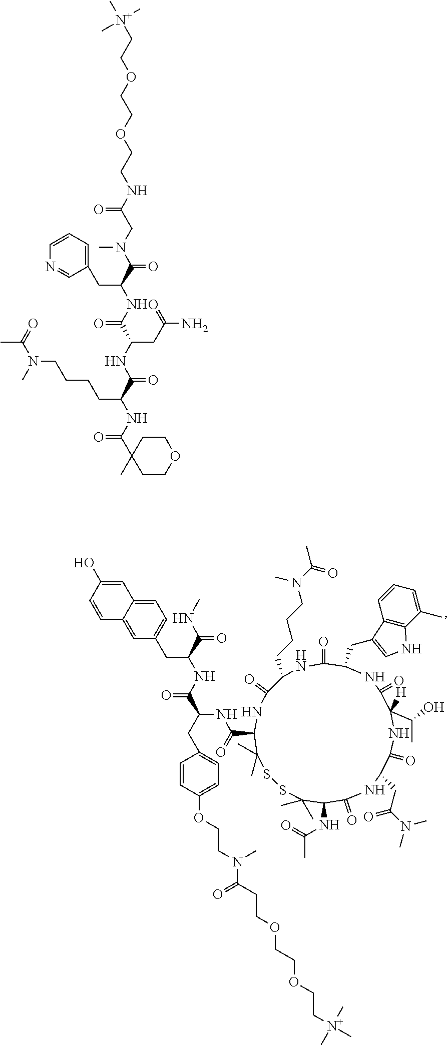 [SMILES: CC(=O)N(C)CCCC[C@H](NC(=O)C1(C)CCOCC1)C(=O)N[C@@H](CC(N)=O)C(=O)N[C@@H](Cc1cccnc1)C(=O)N(C)CC(=O)NCCOCCOCC[N+](C)(C)C ; CNC(=O)[C@H](Cc1ccc2cc(O)ccc2c1)NC(=O)[C@H](Cc1ccc(OCCN(C)C(=O)CCOCCOCC[N+](C)(C)C)cc1)NC(=O)[C@H]1NC(=O)[C@H](CCCCN(C)C(C)=O)NC(=O)[C@H](Cc2c[nH]c3c(C)cccc23)NC(=O)[C@H]([C@@H](C)O)NC(=O)[C@H](CC(=O)N(C)C)NC(=O)[C@@H](NC(C)=O)C(C)(C)SSC1(C)C] 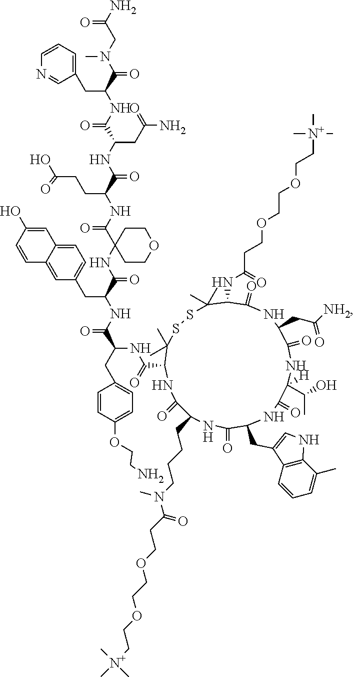 [SMILES: Cc1cccc2c(C[C@@H]3NC(=O)[C@H]([C@@H](C)O)NC(=O)[C@H](CC(N)=O)NC(=O)[C@@H](NC(=O)CCOCCOCC[N+](C)(C)C)C(C)(C)SSC(C)(C)[C@@H](C(=O)N[C@@H](Cc4ccc(OCCN)cc4)C(=O)N[C@@H](Cc4ccc5cc(O)ccc5c4)C(=O)NC4(C(=O)N[C@@H](CCC(=O)O)C(=O)N[C@@H](CC(N)=O)C(=O)N[C@@H](Cc5cccnc5)C(=O)N(C)CC(N)=O)CCOCC4)NC(=O)[C@H](CCCCN(C)C(=O)CCOCCOCC[N+](C)(C)C)NC3=O)c[nH]c12] 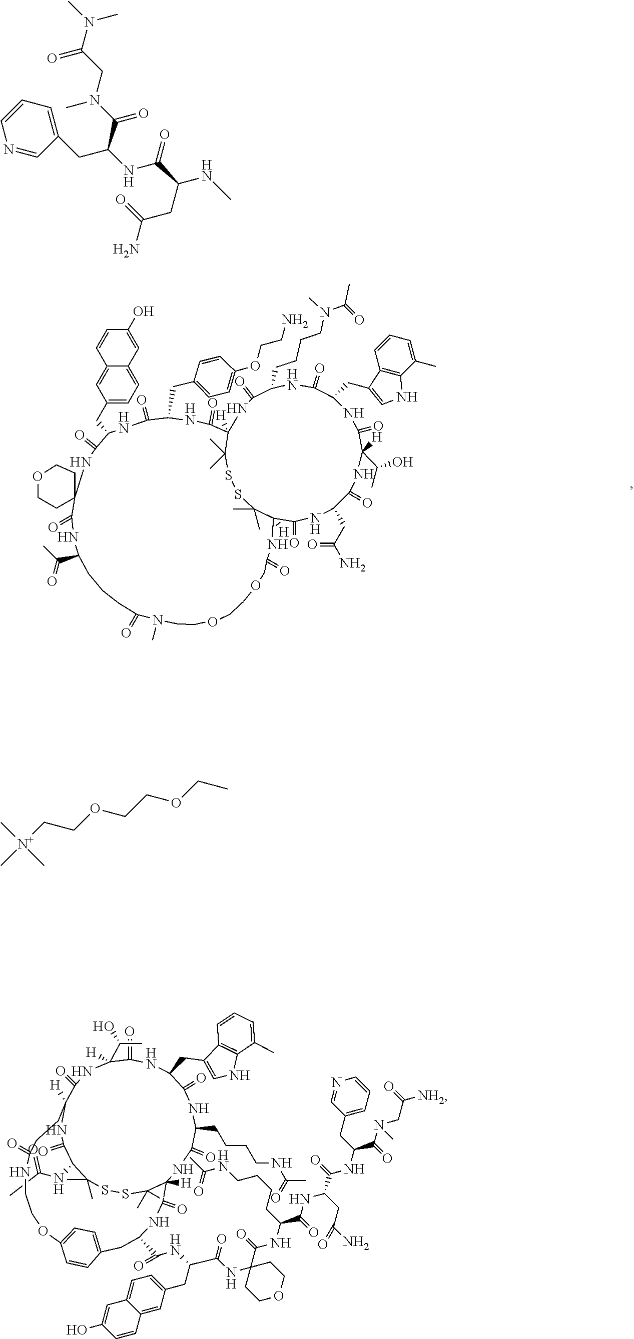 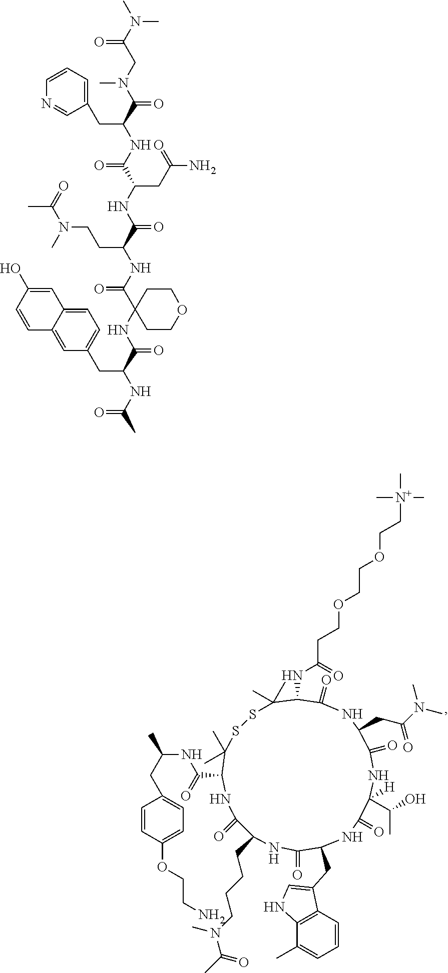 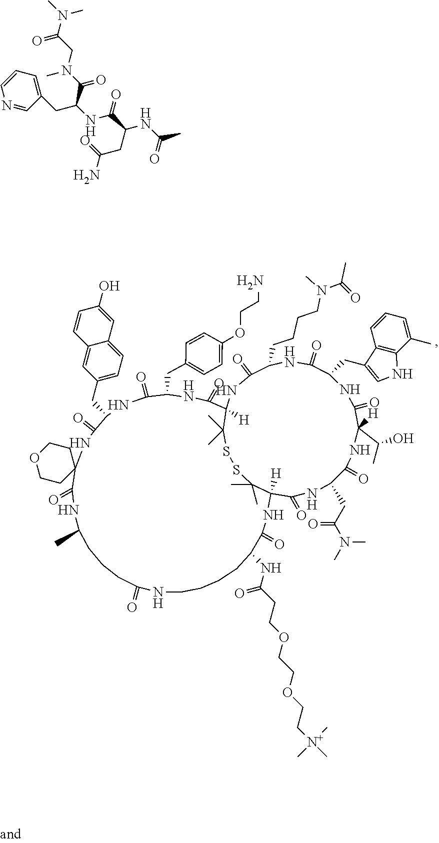 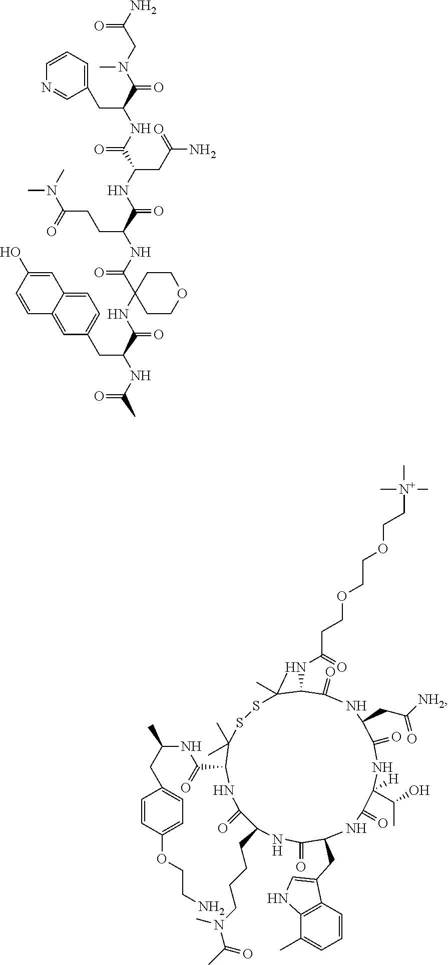
or a pharmaceutically acceptable salt thereof.

95. The peptide of claim 94, having the following structure: 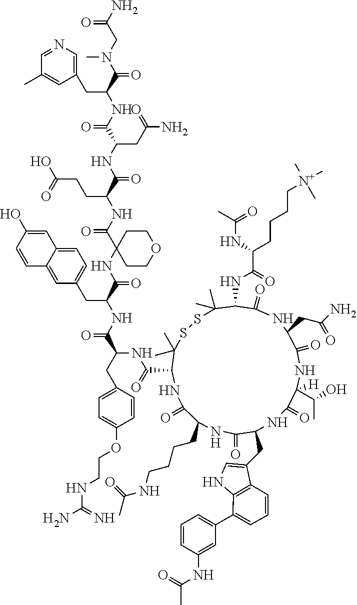
or a pharmaceutically acceptable salt thereof.

96. The peptide of claim 94, having the following structure: 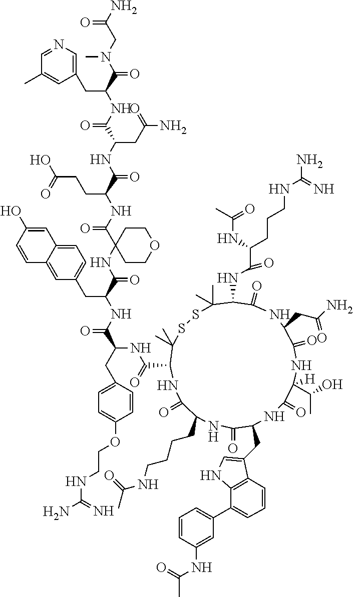
or a pharmaceutically acceptable salt thereof.

97. The peptide of claim 94, having the following structure: 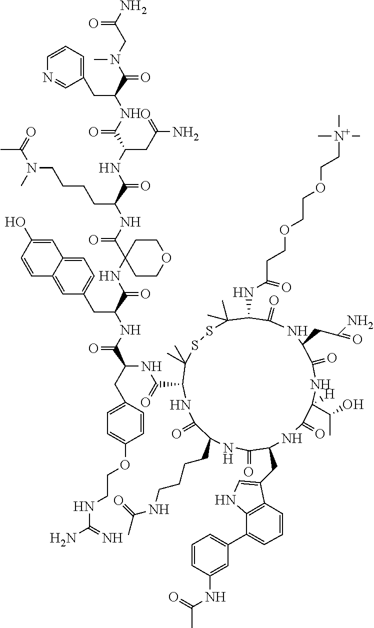
or a pharmaceutically acceptable salt thereof.

98. The peptide of claim 94, having the following structure: 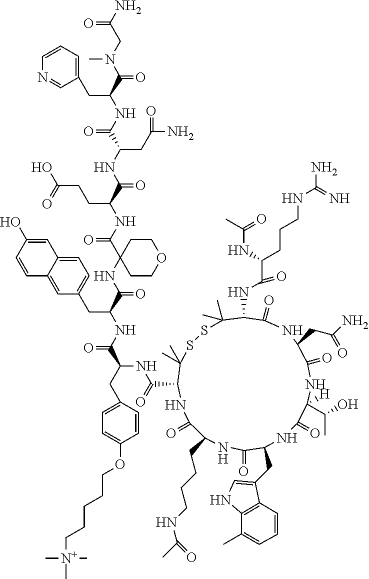
or a pharmaceutically acceptable salt thereof.

99. The peptide of claim 94, having the following structure: 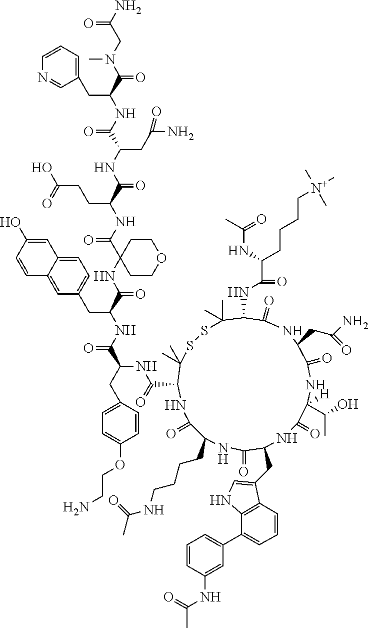
or a pharmaceutically acceptable salt thereof.

100. The peptide of claim 94, having the following structure: 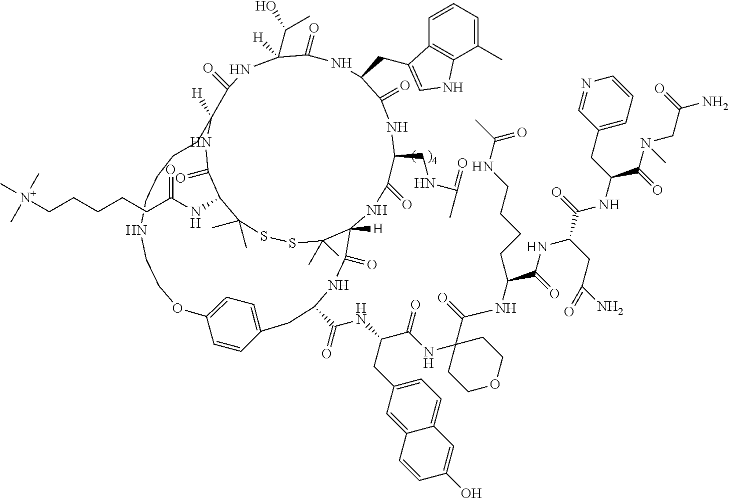
or a pharmaceutically acceptable salt thereof.

101. The peptide of claim 94, having the following structure: 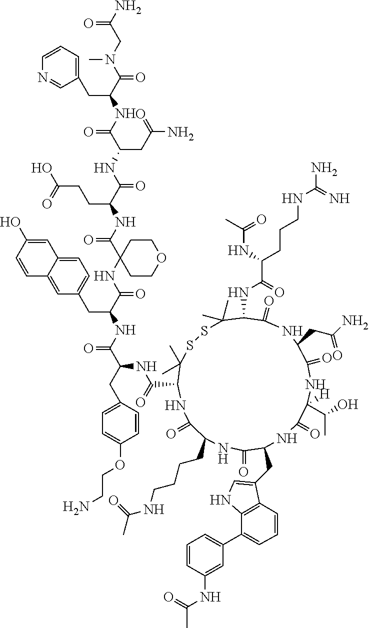
or a pharmaceutically acceptable salt thereof.

102. The peptide of claim 94, having the following structure: 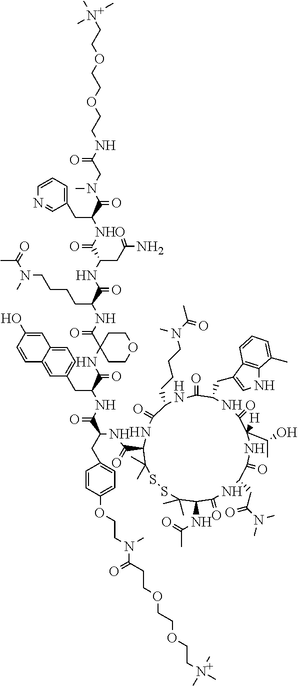
or a pharmaceutically acceptable salt thereof.

103. The peptide of claim 94, having the following structure: 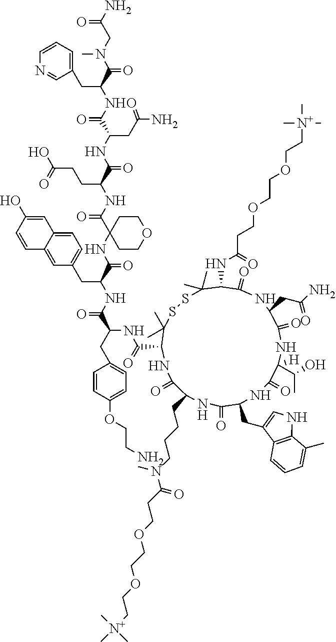
or a pharmaceutically acceptable salt thereof.

104. The peptide of claim 94, having the following structure: 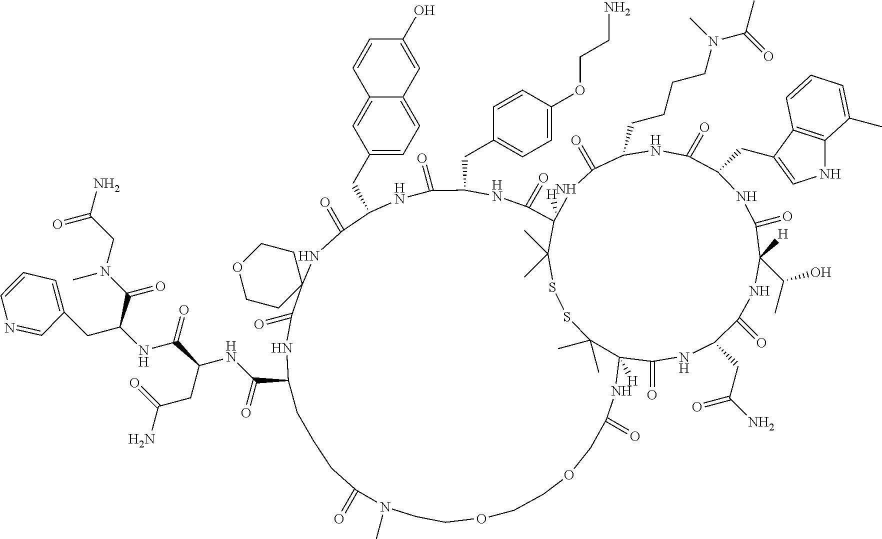
or a pharmaceutically acceptable salt thereof.

105. The peptide of claim 94, having the following structure: 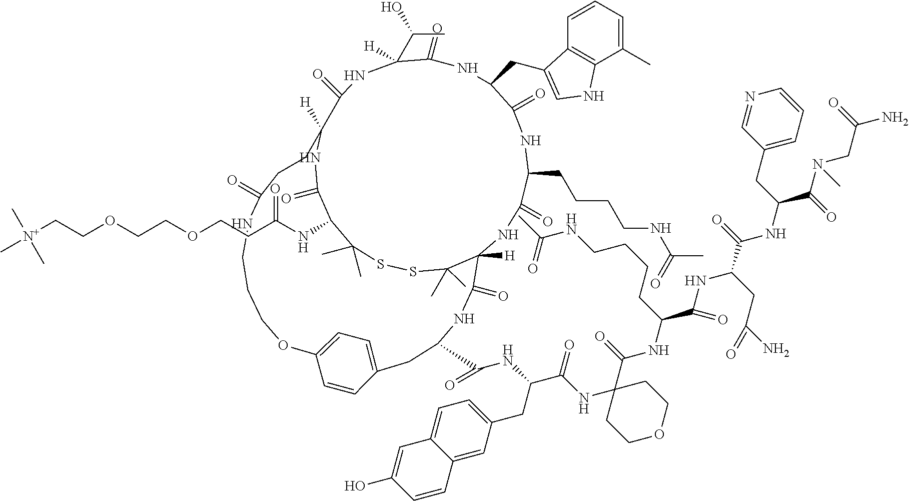 [SMILES: CC(=O)NCCCC[C@@H]1NC(=O)[C@H](Cc2c[nH]c3c(C)cccc23)NC(=O)[C@H]([C@@H](C)O)NC(=O)[C@@H]2CCC(=O)NCCCOc3ccc(cc3)C[C@@H](C(=O)N[C@@H](Cc3ccc4cc(O)ccc4c3)C(=O)NC3(C(=O)N[C@@H](CCCCNC(C)=O)C(=O)N[C@@H](CC(N)=O)C(=O)N[C@@H](Cc4cccnc4)C(=O)N(C)CC(N)=O)CCOCC3)NC(=O)[C@@H](NC1=O)C(C)(C)SSC(C)(C)[C@H](NC(=O)CCOCCOCC[N+](C)(C)C)C(=O)N2]
or a pharmaceutically acceptable salt thereof.

106. The peptide of claim 94, having the following structure: 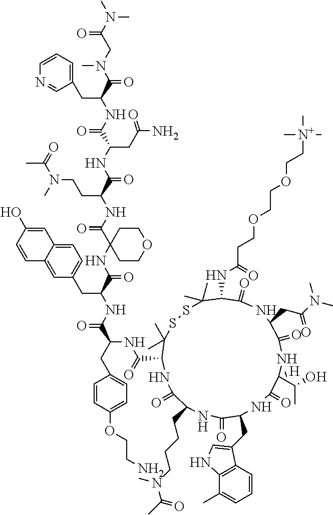
or a pharmaceutically acceptable salt thereof.

107. The peptide of claim 94, having the following structure: 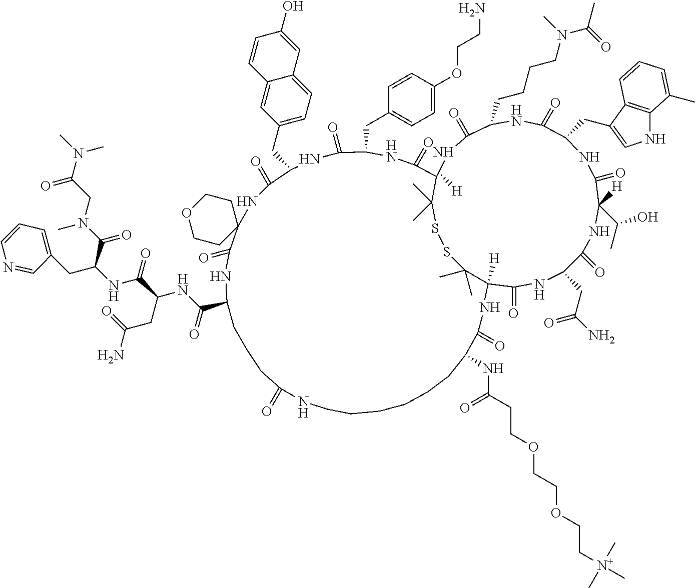
or a pharmaceutically acceptable salt thereof.

108. The peptide of claim 94, having the following structure: 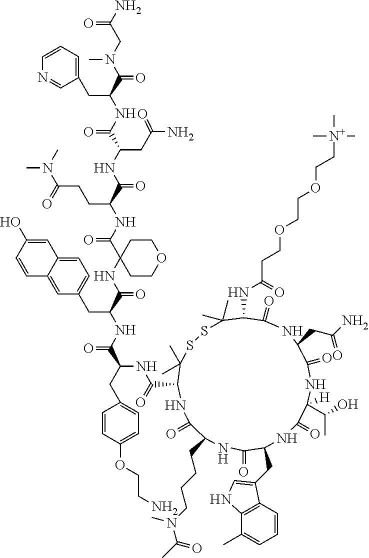
or a pharmaceutically acceptable salt thereof.

109. A pharmaceutical composition comprising the peptide of claim 94, or a pharmaceutically acceptable salt thereof, and a pharmaceutically acceptable carrier.

110. A method for treating a disease or disorder associated with Interleukin 23 (IL-23)/Interleukin 23 Receptor (IL-23R), comprising administering to a subject in need thereof a therapeutically effective amount of the peptide of claim 94.

111. The method of claim 110, wherein the disease or disorder is selected from multiple sclerosis, asthma, rheumatoid arthritis, inflammation of the gut, inflammatory bowel diseases (IBDs), juvenile IBD, adolescent IBD, Crohn's disease, ulcerative colitis, Celiac disease (nontropical Sprue), microscopic colitis, collagenous colitis, eosinophilic gastroenteritis/esophagitis, colitis associated with radio- or chemo-therapy, colitis associated with disorders of innate immunity as in leukocyte adhesion deficiency-1, sarcoidosis, Systemic Lupus Erythematosus, ankylosing spondylitis (axial spondyloarthritis), psoriatic arthritis, psoriasis (e.g., plaque psoriasis, guttate psoriasis, inverse psoriasis, pustular psoriasis, Palmo-Plantar Pustulosis, psoriasis vulgaris, or erythrodermic psoriasis), atopic dermatitis, acne ectopica, enteropathy associated with seronegative arthropathies, chronic granulomatous disease, glycogen storage disease type 1b, Hermansky-Pudlak syndrome, Chediak-Higashi syndrome, Wiskott-Aldrich Syndrome, pouchitis, pouchitis resulting after proctocolectomy and ileoanal anastomosis, gastrointestinal cancer, pancreatitis, insulin-dependent diabetes mellitus, mastitis, cholecystitis, cholangitis, primary biliary cirrhosis, viral-associated enteropathy, pericholangitis, chronic bronchitis, chronic sinusitis, asthma, uveitis, or graft versus host disease.

112. The method of claim 110, wherein the disease or disorder is selected from ulcerative colitis (UC), Crohn's disease (CD), psoriasis (PsO), or psoriatic arthritis (PsA).


SEQUENCE LISTING (447 sequences)

SEQ ID NO: 1 - protein, 14 aa, synthetic construct
  SITE 1: note = N,N,N-trimethyl-D-lysine
  MOD_RES 1: note = N,N,N-trimethyl-D-lysine
  MOD_RES 2: note = Abu
  MOD_RES 6: note = N-acetyllysine
  MOD_RES 7: note = Alpha-methylcysteine
  MOD_RES 8: note = 2-amino-3-(4-(2-guanidinoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
XXNTWXXXXXENXX

SEQ ID NO: 2 - protein, 14 aa, synthetic construct
  MOD_RES 5: note = 3-(7-(3-acetamidophenyl)-1H-indol-3-yl)-2-aminoprop anoic acid
  MOD_RES 6: note = N-acetyllysine
  MOD_RES 7: note = Alpha-methylcysteine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RCNTXXXXXXENXX

SEQ ID NO: 3 - protein, 13 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 12: note = 3-pyridinylalanine
  MOD_RES 13: note = MeGly
XNTXXXXXXENXX

SEQ ID NO: 4 - protein, 14 aa, synthetic construct
  MOD_RES 5: note = 7-methyltryptophan
  MOD_RES 6: note = N-acetyllysine
  MOD_RES 7: note = Alpha-methylcysteine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RCNTXXXXXXENXX

SEQ ID NO: 5 - protein, 13 aa, synthetic construct
  MOD_RES 4: note = 7-methyltryptophan
  MOD_RES 5: note = N-acetyllysine
  MOD_RES 6: note = Alpha-methylcysteine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 10: note = N-acetyllysine
  MOD_RES 12: note = 3-pyridinylalanine
  MOD_RES 13: note = MeGly
CNTXXXXXXXNXX

SEQ ID NO: 6 - protein, 14 aa, synthetic construct
  MOD_RES 2: note = Abu
  MOD_RES 5: note = 3-(7-(3-acetamidophenyl)-1H-indol-3-yl)-2-aminoprop anoic acid
  MOD_RES 6: note = N-acetyllysine
  MOD_RES 7: note = Alpha-methylcysteine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXNTXXXXXXENXX

SEQ ID NO: 7 - protein, 14 aa, synthetic construct
  MOD_RES 2: note = Abu
  MOD_RES 6: note = N-acetyllysine
  MOD_RES 7: note = Alpha-methylcysteine
  MOD_RES 8: note = 2-amino-3-(4-(2-guanidinoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 11: note = N-acetyllysine
  MOD_RES 13: note = 3-pyridinylalanine
  MOD_RES 14: note = MeGly
RXNTWXXXXXXNXX

SEQ ID NO: 8 - protein, 14 aa, synthetic construct
  MOD_RES 1: note = N,N,N-trimethyl-D-lysine
  MOD_RES 5: note = 7-methyltryptophan
  MOD_RES 6: note = N-acetyllysine
  MOD_RES 7: note = Alpha-methylcysteine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
  SITE 1: note = N,N,N-trimethyl-D-lysine
XCNTXXXXXXENXX

SEQ ID NO: 9 - protein, 13 aa, synthetic construct
  MOD_RES 1: note = Penicillamine
  MOD_RES 4: note = 3-(7-(3-acetamidophenyl)-1H-indol-3-yl)-2-aminoprop anoic acid
  MOD_RES 5: note = N-acetyllysine
  MOD_RES 6: note = Penicillamine
  MOD_RES 7: note = 2-amino-3-(4-(2-guanidinoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 12: note = 3-pyridinylalanine
  MOD_RES 13: note = MeGly
XNTXXXXXXENXX

SEQ ID NO: 10 - protein, 14 aa, synthetic construct
  MOD_RES 1: note = N-propionyl-D-lysine
  MOD_RES 2: note = Abu
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
  SITE 1: note = N-propionyl-D-lysine
XXNTWQCXXXCNXX

SEQ ID NO: 11 - protein, 14 aa, synthetic construct
  MOD_RES 1: note = N,N,N-trimethyl-D-lysine
  MOD_RES 2: note = Penicillamine
  MOD_RES 5: note = 3-(7-(3-acetamidophenyl)-1H-indol-3-yl)-2-aminoprop anoic acid
  MOD_RES 6: note = N-acetyllysine
  MOD_RES 7: note = Penicillamine
  MOD_RES 8: note = 2-amino-3-(4-(2-guanidinoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 2-amino-3-(5-methylpyridin-3-yl)propanoic acid
  MOD_RES 14: note = MeGly
  SITE 1: note = N,N,N-trimethyl-D-lysine
XXNTXXXXXXENXX

SEQ ID NO: 12-14 - skipped (3 entries)

SEQ ID NO: 15 - protein, 14 aa, synthetic construct
  MOD_RES 2: note = Penicillamine
  MOD_RES 5: note = 3-(7-(3-acetamidophenyl)-1H-indol-3-yl)-2-aminoprop anoic acid
  MOD_RES 6: note = N-acetyllysine
  MOD_RES 7: note = Penicillamine
  MOD_RES 8: note = 2-amino-3-(4-(2-guanidinoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 2-amino-3-(5-methylpyridin-3-yl)propanoic acid
  MOD_RES 14: note = MeGly
RXNTXXXXXXENXX

SEQ ID NO: 16 - protein, 13 aa, synthetic construct
  MOD_RES 1: note = Penicillamine
  MOD_RES 4: note = 3-(7-(3-acetamidophenyl)-1H-indol-3-yl)-2-aminoprop anoic acid
  MOD_RES 5: note = N-acetyllysine
  MOD_RES 6: note = Penicillamine
  MOD_RES 7: note = 2-amino-3-(4-(2-guanidinoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 12: note = 2-amino-3-(5-methylpyridin-3-yl)propanoic acid
  MOD_RES 13: note = MeGly
XNTXXXXXXENXX

SEQ ID NO: 17 - protein, 14 aa, synthetic construct
  MOD_RES 1: note = N,N,N-trimethyl-D-lysine
  MOD_RES 2: note = Penicillamine
  MOD_RES 5: note = 3-(7-(3-acetamidophenyl)-1H-indol-3-yl)-2-aminoprop anoic acid
  MOD_RES 6: note = N-acetyllysine
  MOD_RES 7: note = Penicillamine
  MOD_RES 8: note = 5-(4-(2-amino-2-carboxyethyl)phenoxy)-N,N,N-trimeth ylpentan-1-aminium
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 2-amino-3-(5-methylpyridin-3-yl)propanoic acid
  MOD_RES 14: note = MeGly
  SITE 1: note = N,N,N-trimethyl-D-lysine
XXNTXXXXXXENXX

SEQ ID NO: 18-19 - skipped (2 entries)

SEQ ID NO: 20 - protein, 14 aa, synthetic construct
  MOD_RES 2: note = Penicillamine
  MOD_RES 5: note = 3-(7-(3-acetamidophenyl)-1H-indol-3-yl)-2-aminoprop anoic acid
  MOD_RES 6: note = N-acetyllysine
  MOD_RES 7: note = Penicillamine
  MOD_RES 8: note = 5-(4-(2-amino-2-carboxyethyl)phenoxy)-N,N,N-trimeth ylpentan-1-aminium
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 2-amino-3-(5-methylpyridin-3-yl)propanoic acid
  MOD_RES 14: note = MeGly
RXNTXXXXXXENXX

SEQ ID NO: 21 - skipped

SEQ ID NO: 22 - protein, 13 aa, synthetic construct
  MOD_RES 1: note = Penicillamine
  MOD_RES 4: note = 3-(7-(3-acetamidophenyl)-1H-indol-3-yl)-2-aminoprop anoic acid
  MOD_RES 5: note = N-acetyllysine
  MOD_RES 6: note = Penicillamine
  MOD_RES 7: note = 5-(4-(2-amino-2-carboxyethyl)phenoxy)-N,N,N-trimeth ylpentan-1-aminium
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 12: note = 2-amino-3-(5-methylpyridin-3-yl)propanoic acid
  MOD_RES 13: note = MeGly
XNTXXXXXXENXX

SEQ ID NO: 23 - skipped

SEQ ID NO: 24 - protein, 13 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-guanidinoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 12: note = 3-pyridinylalanine
  MOD_RES 13: note = MeGly
XNTXXXXXXENXX

SEQ ID NO: 25-27 - skipped (3 entries)

SEQ ID NO: 28 - protein, 14 aa, synthetic construct
  MOD_RES 2: note = Abu
  MOD_RES 6: note = N-acetyllysine
  MOD_RES 7: note = Alpha-methylcysteine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 11: note = N-acetyllysine
  MOD_RES 13: note = 3-pyridinylalanine
  MOD_RES 14: note = MeGly
RXNTWXXXXXXNXX

SEQ ID NO: 29 - protein, 14 aa, synthetic construct
  MOD_RES 2: note = Abu
  MOD_RES 6: note = N-acetyllysine
  MOD_RES 7: note = Alpha-methylcysteine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 11: note = N-acetyllysine
  MOD_RES 13: note = 3-pyridinylalanine
  MOD_RES 14: note = MeGly
RXNTWXXXXXXNXX

SEQ ID NO: 30 - protein, 14 aa, synthetic construct
  MOD_RES 1: note = N,N,N-trimethyl-D-lysine
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-guanidinoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
  SITE 1: note = N,N,N-trimethyl-D-lysine
XXNTXXXXXXENXX

SEQ ID NO: 31 - skipped

SEQ ID NO: 32 - protein, 14 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-guanidinoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXNTXXXXXXENXX

SEQ ID NO: 33 - protein, 14 aa, synthetic construct
  MOD_RES 1: note = N,N,N-trimethyl-D-lysine
  MOD_RES 2: note = Penicillamine
  MOD_RES 5: note = 3-(7-(3-acetamidophenyl)-1H-indol-3-yl)-2-aminoprop anoic acid
  MOD_RES 6: note = N-acetyllysine
  MOD_RES 7: note = Penicillamine
  MOD_RES 8: note = 2-amino-3-(4-(2-guanidinoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
  SITE 1: note = N,N,N-trimethyl-D-lysine
XXNTXXXXXXENXX

SEQ ID NO: 34 - skipped

SEQ ID NO: 35 - protein, 14 aa, synthetic construct
  MOD_RES 2: note = Penicillamine
  MOD_RES 5: note = 3-(7-(3-acetamidophenyl)-1H-indol-3-yl)-2-aminoprop anoic acid
  MOD_RES 6: note = N-acetyllysine
  MOD_RES 7: note = Penicillamine
  MOD_RES 8: note = 2-amino-3-(4-(2-guanidinoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXNTXXXXXXENXX

SEQ ID NO: 36 - skipped

SEQ ID NO: 37 - protein, 14 aa, synthetic construct
  MOD_RES 1: note = N,N,N-trimethyl-D-lysine
  MOD_RES 2: note = Penicillamine
  MOD_RES 5: note = 3-(7-(3-acetamidophenyl)-1H-indol-3-yl)-2-aminoprop anoic acid
  MOD_RES 6: note = N-acetyllysine
  MOD_RES 7: note = Penicillamine
  MOD_RES 8: note = 5-(4-(2-amino-2-carboxyethyl)phenoxy)-N,N,N-trimeth ylpentan-1-aminium
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
  SITE 1: note = N,N,N-trimethyl-D-lysine
XXNTXXXXXXENXX

SEQ ID NO: 38 - skipped

SEQ ID NO: 39 - protein, 14 aa, synthetic construct
  MOD_RES 2: note = Penicillamine
  MOD_RES 5: note = 3-(7-(3-acetamidophenyl)-1H-indol-3-yl)-2-aminoprop anoic acid
  MOD_RES 6: note = N-acetyllysine
  MOD_RES 7: note = Penicillamine
  MOD_RES 8: note = 5-(4-(2-amino-2-carboxyethyl)phenoxy)-N,N,N-trimeth ylpentan-1-aminium
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXNTXXXXXXENXX

SEQ ID NO: 40-42 - skipped (3 entries)

SEQ ID NO: 43 - protein, 14 aa, synthetic construct
  MOD_RES 1: note = N,N,N-trimethyl-D-lysine
  MOD_RES 2: note = Abu
  MOD_RES 6: note = N-acetyllysine
  MOD_RES 7: note = Alpha-methylcysteine
  MOD_RES 8: note = 5-(4-(2-amino-2-carboxyethyl)phenoxy)-N,N,N-trimeth ylpentan-1-aminium
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
  SITE 1: note = N,N,N-trimethyl-D-lysine
XXNTWXXXXXENXX

SEQ ID NO: 44 - protein, 14 aa, synthetic construct
  MOD_RES 2: note = Abu
  MOD_RES 6: note = N-acetyllysine
  MOD_RES 7: note = Alpha-methylcysteine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXNTWXXXXXENXX

SEQ ID NO: 45 - protein, 14 aa, synthetic construct
  MOD_RES 2: note = Abu
  MOD_RES 6: note = N-acetyllysine
  MOD_RES 7: note = Alpha-methylcysteine
  MOD_RES 8: note = 5-(4-(2-amino-2-carboxyethyl)phenoxy)-N,N,N-trimeth ylpentan-1-aminium
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 11: note = N-acetyllysine
  MOD_RES 13: note = 3-pyridinylalanine
  MOD_RES 14: note = MeGly
RXNTWXXXXXXNXX

SEQ ID NO: 46 - protein, 14 aa, synthetic construct
  MOD_RES 2: note = Abu
  MOD_RES 6: note = N-acetyllysine
  MOD_RES 7: note = Alpha-methylcysteine
  MOD_RES 8: note = 5-(4-(2-amino-2-carboxyethyl)phenoxy)-N,N,N-trimeth ylpentan-1-aminium
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXNTWXXXXXENXX

SEQ ID NO: 47 - protein, 14 aa, synthetic construct
  MOD_RES 2: note = Abu
  MOD_RES 6: note = N-acetyllysine
  MOD_RES 7: note = Alpha-methylcysteine
  MOD_RES 8: note = 5-(4-(2-amino-2-carboxyethyl)phenoxy)-N,N,N-trimeth ylpentan-1-aminium
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXNTWXXXXXENXX

SEQ ID NO: 48 - protein, 14 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 5-(4-(2-amino-2-carboxyethyl)phenoxy)-N,N,N-trimeth ylpentan-1-aminium
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 2-amino-3-(5-methylpyridin-3-yl)propanoic acid
  MOD_RES 14: note = MeGly
RXNTXXXXXXENXX

SEQ ID NO: 49 - protein, 14 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 11: note = N-acetyllysine
  MOD_RES 13: note = 2-amino-3-(5-methylpyridin-3-yl)propanoic acid
  MOD_RES 14: note = MeGly
RXNTXXXXXXXNXX

SEQ ID NO: 50 - protein, 14 aa, synthetic construct
  MOD_RES 1: note = N,N,N-trimethyl-D-lysine
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 2-amino-3-(5-methylpyridin-3-yl)propanoic acid
  MOD_RES 14: note = MeGly
  SITE 1: note = N,N,N-trimethyl-D-lysine
XXNTXXXXXXENXX

SEQ ID NO: 51 - protein, 14 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 2-amino-3-(5-methylpyridin-3-yl)propanoic acid
  MOD_RES 14: note = MeGly
RXNTXXXXXXENXX

SEQ ID NO: 52 - protein, 14 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 2-amino-3-(5-methylpyridin-3-yl)propanoic acid
  MOD_RES 14: note = MeGly
RXNTXXXXXXENXX

SEQ ID NO: 53 - protein, 14 aa, synthetic construct
  MOD_RES 2: note = Penicillamine
  MOD_RES 5: note = 3-(7-(3-acetamidophenyl)-1H-indol-3-yl)-2-aminoprop anoic acid
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 2-amino-3-(5-methylpyridin-3-yl)propanoic acid
  MOD_RES 14: note = MeGly
RXNTXXXXXXENXX

SEQ ID NO: 54 - protein, 14 aa, synthetic construct
  MOD_RES 2: note = Penicillamine
  MOD_RES 5: note = 7-methyltryptophan
  LIPID 6: note = Lipidated Lysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 11: note = N-acetyllysine
  MOD_RES 13: note = 3-pyridinylalanine
  MOD_RES 14: note = MeGly
RXNTXKXXXXXNXX

SEQ ID NO: 55 - protein, 13 aa, synthetic construct
  MOD_RES 1: note = Penicillamine
  MOD_RES 4: note = 7-methyltryptophan
  LIPID 5: note = Lipidated Lysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 10: note = N-acetyllysine
  MOD_RES 12: note = 3-pyridinylalanine
  MOD_RES 13: note = MeGly
XNTXKXXXXXNXX

SEQ ID NO: 56 - protein, 13 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 4-(2-(4-(2-amino-2-carboxyethyl)phenoxy)ethyl)-4-me thylmorpholin-4-ium
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 12: note = 3-pyridinylalanine
  MOD_RES 13: note = MeGly
XNTXXXXXXENXX

SEQ ID NO: 57 - protein, 14 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 14: note = MeGly
RXNTXXXXXXENHX

SEQ ID NO: 58 - protein, 14 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 5-(4-(2-amino-2-carboxyethyl)phenoxy)-N,N,N-trimeth ylpentan-1-aminium
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 11: note = N-acetyllysine
  MOD_RES 13: note = 3-pyridinylalanine
  MOD_RES 14: note = MeGly
RXNTXXXXXXXNXX

SEQ ID NO: 59 - protein, 14 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 5-(4-(2-amino-2-carboxyethyl)phenoxy)-N,N,N-trimeth ylpentan-1-aminium
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXNTXXXXXXENXX

SEQ ID NO: 60 - protein, 14 aa, synthetic construct
  MOD_RES 1: note = N,N,N-trimethyl-D-lysine
  MOD_RES 2: note = Abu
  MOD_RES 6: note = N-acetyllysine
  MOD_RES 7: note = Alpha-methylcysteine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 11: note = N-acetyllysine
  MOD_RES 13: note = 3-pyridinylalanine
  MOD_RES 14: note = MeGly
  SITE 1: note = N,N,N-trimethyl-D-lysine
XXNTWXXXXXXNXX

SEQ ID NO: 61 - protein, 14 aa, synthetic construct
  MOD_RES 1: note = N,N,N-trimethyl-D-lysine
  MOD_RES 2: note = Abu
  MOD_RES 6: note = N-acetyllysine
  MOD_RES 7: note = Alpha-methylcysteine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
  SITE 1: note = N,N,N-trimethyl-D-lysine
XXNTWXXXXXENXX

SEQ ID NO: 62 - protein, 13 aa, synthetic construct
  MOD_RES 1: note = Penicillamine
  MOD_RES 4: note = 3-(7-(3-acetamidophenyl)-1H-indol-3-yl)-2-aminoprop anoic acid
  MOD_RES 5: note = N-acetyllysine
  MOD_RES 6: note = Penicillamine
  MOD_RES 7: note = 4-(2-(4-(2-amino-2-carboxyethyl)phenoxy)ethyl)-4-me thylmorpholin-4-ium
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 10: note = N-methyl-N-acetyllysine
  MOD_RES 13: note = MeGly
XNTXXXXXXXNHX

SEQ ID NO: 63 - protein, 13 aa, synthetic construct
  MOD_RES 1: note = Penicillamine
  MOD_RES 4: note = 3-(7-(3-acetamidophenyl)-1H-indol-3-yl)-2-aminoprop anoic acid
  MOD_RES 5: note = N-acetyllysine
  MOD_RES 6: note = Penicillamine
  MOD_RES 7: note = 5-(4-(2-amino-2-carboxyethyl)phenoxy)-N,N,N-trimeth ylpentan-1-aminium
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 12: note = 3-pyridinylalanine
  MOD_RES 13: note = MeGly
XNTXXXXXXENXX

SEQ ID NO: 64 - protein, 14 aa, synthetic construct
  MOD_RES 2: note = Penicillamine
  MOD_RES 5: note = 3-(7-(3-acetamidophenyl)-1H-indol-3-yl)-2-aminoprop anoic acid
  MOD_RES 6: note = N-acetyllysine
  MOD_RES 7: note = Penicillamine
  MOD_RES 8: note = 4-(2-(4-(2-amino-2-carboxyethyl)phenoxy)ethyl)-4-me thylmorpholin-4-ium
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 14: note = MeGly
RXNTXXXXXXENHX

SEQ ID NO: 65 - protein, 14 aa, synthetic construct
  MOD_RES 2: note = Penicillamine
  MOD_RES 5: note = 3-(7-(3-acetamidophenyl)-1H-indol-3-yl)-2-aminoprop anoic acid
  MOD_RES 6: note = N-acetyllysine
  MOD_RES 7: note = Penicillamine
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXNTXXXYXXENXX

SEQ ID NO: 66 - skipped

SEQ ID NO: 67 - protein, 14 aa, synthetic construct
  MOD_RES 1: note = N,N,N-trimethyl-D-lysine
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 5-(4-(2-amino-2-carboxyethyl)phenoxy)-N,N,N-trimeth ylpentan-1-aminium
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
  SITE 1: note = N,N,N-trimethyl-D-lysine
XXNTXXXXXXENXX

SEQ ID NO: 68 - protein, 14 aa, synthetic construct
  MOD_RES 1: note = N,N,N-trimethyl-D-lysine
  MOD_RES 2: note = Penicillamine
  MOD_RES 5: note = 3-(7-(3-acetamidophenyl)-1H-indol-3-yl)-2-aminoprop anoic acid
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
  SITE 1: note = N,N,N-trimethyl-D-lysine
XXNTXXXXXXENXX

SEQ ID NO: 69 - protein, 13 aa, synthetic construct
  MOD_RES 1: note = Penicillamine
  MOD_RES 4: note = 3-(7-(3-acetamidophenyl)-1H-indol-3-yl)-2-aminoprop anoic acid
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 12: note = 3-pyridinylalanine
  MOD_RES 13: note = MeGly
XNTXXXXXXENXX

SEQ ID NO: 70-71 - skipped (2 entries)

SEQ ID NO: 72 - protein, 14 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 10: note = N-acetyllysine
  MOD_RES 12: note = 3-pyridinylalanine
  MOD_RES 13: note = MeGly
  LIPID 14: note = Lipidated Lysine
XNTXXXXXXXNXXK

SEQ ID NO: 73 - protein, 14 aa, synthetic construct
  MOD_RES 2: note = Penicillamine
  MOD_RES 6: note = N-acetyllysine
  MOD_RES 7: note = Penicillamine
  MOD_RES 8: note = 4-(2-(4-(2-amino-2-carboxyethyl)phenoxy)ethyl)-4-me thylmorpholin-4-ium
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXNTWXXXXXENXX

SEQ ID NO: 74 - protein, 14 aa, synthetic construct
  MOD_RES 2: note = Penicillamine
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXNTWXXXXXENXX

SEQ ID NO: 75 - protein, 13 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 5-(4-(2-amino-2-carboxyethyl)phenoxy)-N,N,N-trimeth ylpentan-1-aminium
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 12: note = 3-pyridinylalanine
  MOD_RES 13: note = MeGly
XNTXXXXXXENXX

SEQ ID NO: 76 - skipped

SEQ ID NO: 77 - protein, 14 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 4-(2-(4-(2-amino-2-carboxyethyl)phenoxy)ethyl)-4-me thylmorpholin-4-ium
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXNTXXXXXXENXX

SEQ ID NO: 78 - skipped

SEQ ID NO: 79 - protein, 13 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 5-(4-(2-amino-2-carboxyethyl)phenoxy)-N,N,N-trimeth ylpentan-1-aminium
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 12: note = 3-pyridinylalanine
  MOD_RES 13: note = MeGly
XNTXXXXXXENXX

SEQ ID NO: 80 - protein, 13 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  LIPID 10: note = Lipidated Lysine
  MOD_RES 12: note = 3-pyridinylalanine
  MOD_RES 13: note = MeGly
XNTXXXXXXKNXX

SEQ ID NO: 81 - protein, 13 aa, synthetic construct
  MOD_RES 1: note = Penicillamine
  MOD_RES 4: note = 7-methyltryptophan
  LIPID 5: note = Lipidated Lysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 10: note = N-acetyllysine
  MOD_RES 12: note = 3-pyridinylalanine
  MOD_RES 13: note = MeGly
XNTXKXXXXXNXX

SEQ ID NO: 82 - skipped

SEQ ID NO: 83 - protein, 14 aa, synthetic construct
  MOD_RES 2: note = Penicillamine
  MOD_RES 5: note = 7-methyltryptophan
  LIPID 6: note = Lipidated Lysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXNTXKXXXXENXX

SEQ ID NO: 84 - protein, 14 aa, synthetic construct
  MOD_RES 2: note = Penicillamine
  MOD_RES 5: note = 7-methyltryptophan
  LIPID 6: note = Lipidated Lysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXNTXKXXXXENXX

SEQ ID NO: 85 - protein, 14 aa, synthetic construct
  LIPID 1: note = Lipidated Lysine
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 11: note = N-acetyllysine
  MOD_RES 13: note = 3-pyridinylalanine
  MOD_RES 14: note = MeGly
KXNTXXXXXXXNXX

SEQ ID NO: 86 - protein, 14 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  LIPID 11: note = Lipidated Lysine
  MOD_RES 13: note = 3-pyridinylalanine
  MOD_RES 14: note = MeGly
RXNTXXXXXXKNXX

SEQ ID NO: 87 - protein, 14 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  LIPID 11: note = Lipidated Lysine
  MOD_RES 13: note = 3-pyridinylalanine
  MOD_RES 14: note = MeGly
RXNTXXXXXXKNXX

SEQ ID NO: 88 - protein, 14 aa, synthetic construct
  MOD_RES 2: note = Penicillamine
  LIPID 3: note = Lipidated Lysine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXKTXXXXXXENXX

SEQ ID NO: 89 - protein, 14 aa, synthetic construct
  MOD_RES 2: note = Penicillamine
  LIPID 3: note = Lipidated Lysine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXKTXXXXXXENXX

SEQ ID NO: 90 - protein, 14 aa, synthetic construct
  LIPID 1: note = Lipidated Lysine
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
KXNTXXXXXXENXX

SEQ ID NO: 91 - protein, 14 aa, synthetic construct
  LIPID 1: note = Lipidated Lysine
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
KXNTXXXXXXENXX

SEQ ID NO: 92 - protein, 15 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
  LIPID 15: note = Lipidated Lysine
RXNTXXXXXXENXXK

SEQ ID NO: 93 - skipped

SEQ ID NO: 94 - protein, 14 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXNTXXXXXXENXX

SEQ ID NO: 95 - protein, 13 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  LIPID 10: note = Lipidated Lysine
  MOD_RES 12: note = 3-pyridinylalanine
  MOD_RES 13: note = MeGly
XNTXXXXXXKNXX

SEQ ID NO: 96 - protein, 13 aa, synthetic construct
  MOD_RES 1: note = Penicillamine
  MOD_RES 4: note = 7-methyltryptophan
  LIPID 5: note = Lipidated Lysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 10: note = N-acetyllysine
  MOD_RES 12: note = 3-pyridinylalanine
  MOD_RES 13: note = MeGly
XNTXKXXXXXNXX

SEQ ID NO: 97 - skipped

SEQ ID NO: 98 - protein, 13 aa, synthetic construct
  MOD_RES 1: note = Abu
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 12: note = 3-pyridinylalanine
  MOD_RES 13: note = MeGly
XNTWQCXXXENXX

SEQ ID NO: 99 - skipped

SEQ ID NO: 100 - protein, 13 aa, synthetic construct
  MOD_RES 1: note = Penicillamine
  MOD_RES 4: note = 3-(7-(3-acetamidophenyl)-1H-indol-3-yl)-2-aminoprop anoic acid
  MOD_RES 5: note = N-acetyllysine
  MOD_RES 6: note = Penicillamine
  MOD_RES 7: note = 4-(2-(4-(2-amino-2-carboxyethyl)phenoxy)ethyl)-4-me thylmorpholin-4-ium
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 12: note = 3-pyridinylalanine
  MOD_RES 13: note = MeGly
XNTXXXXXXENXX

SEQ ID NO: 101-109 - skipped (9 entries)

SEQ ID NO: 110 - protein, 13 aa, synthetic construct
  MOD_RES 2: note = Penicillamine
  MOD_RES 5: note = 3-(7-(3-acetamidophenyl)-1H-indol-3-yl)-2-aminoprop anoic acid
  MOD_RES 6: note = N-acetyllysine
  MOD_RES 7: note = Penicillamine
  MOD_RES 8: note = 4-(2-(4-(2-amino-2-carboxyethyl)phenoxy)ethyl)-4-me thylmorpholin-4-ium
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 4-aminotetrahydro-2H-pyran-4-carboxylic acid
RXNTXXXXXXENX

SEQ ID NO: 111-114 - skipped (4 entries)

SEQ ID NO: 115 - protein, 15 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
  LIPID 15: note = Lipidated Lysine
RXNTXXXXXXENXXK

SEQ ID NO: 116 - protein, 15 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
  LIPID 15: note = Lipidated Lysine
RXNTXXXXXXENXXK

SEQ ID NO: 117 - protein, 15 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
  LIPID 15: note = Lipidated Lysine
RXNTXXXXXXENXXK

SEQ ID NO: 118 - protein, 14 aa, synthetic construct
  LIPID 1: note = Lipidated Lysine
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 11: note = N-acetyllysine
  MOD_RES 13: note = 3-pyridinylalanine
  MOD_RES 14: note = MeGly
KXNTXXXXXXXNXX

SEQ ID NO: 119 - protein, 14 aa, synthetic construct
  MOD_RES 1: note = N,N,N-trimethyl-D-homolysine
  MOD_RES 2: note = Penicillamine
  MOD_RES 5: note = 7-methyltryptophan
  LIPID 6: note = Lipidated Lysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 11: note = N-acetyllysine
  MOD_RES 13: note = 3-pyridinylalanine
  MOD_RES 14: note = MeGly
  SITE 1: note = N,N,N-trimethyl-D-homolysine
XXNTXKXXXXXNXX

SEQ ID NO: 120 - protein, 14 aa, synthetic construct
  MOD_RES 2: note = Penicillamine
  MOD_RES 5: note = 7-methyltryptophan
  LIPID 6: note = Lipidated Lysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXNTXKXXXXENXX

SEQ ID NO: 121-124 - skipped (4 entries)

SEQ ID NO: 125 - protein, 15 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
  LIPID 15: note = Lipidated Lysine
RXNTXXXXXXENXXK

SEQ ID NO: 126 - protein, 14 aa, synthetic construct
  LIPID 1: note = Lipidated Lysine
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
KXNTXXXXXXENXX

SEQ ID NO: 127 - skipped

SEQ ID NO: 128 - protein, 13 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  LIPID 10: note = Lipidated Lysine
  MOD_RES 12: note = 3-pyridinylalanine
  MOD_RES 13: note = MeGly
XNTXXXXXXKNXX

SEQ ID NO: 129 - protein, 13 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  LIPID 10: note = Lipidated Lysine
  MOD_RES 12: note = 3-pyridinylalanine
  MOD_RES 13: note = MeGly
XNTXXXXXXKNXX

SEQ ID NO: 130 - protein, 13 aa, synthetic construct
  MOD_RES 1: note = Penicillamine
  MOD_RES 4: note = 7-methyltryptophan
  LIPID 5: note = Lipidated Lysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 10: note = N-acetyllysine
  MOD_RES 12: note = 3-pyridinylalanine
  MOD_RES 13: note = MeGly
XNTXKXXXXXNXX

SEQ ID NO: 131 - protein, 13 aa, synthetic construct
  MOD_RES 1: note = Penicillamine
  MOD_RES 4: note = 7-methyltryptophan
  LIPID 5: note = Lipidated Lysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 10: note = N-acetyllysine
  MOD_RES 12: note = 3-pyridinylalanine
  MOD_RES 13: note = MeGly
XNTXKXXXXXNXX

SEQ ID NO: 132 - protein, 13 aa, synthetic construct
  MOD_RES 1: note = Penicillamine
  MOD_RES 4: note = 3-(7-(3-acetamidophenyl)-1H-indol-3-yl)-2-aminoprop anoic acid
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 12: note = 3-pyridinylalanine
  MOD_RES 13: note = MeGly
XNTXXXXXXENXX

SEQ ID NO: 133 - skipped

SEQ ID NO: 134 - protein, 14 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 11: note = N-acetyllysine
  MOD_RES 13: note = 3-pyridinylalanine
  MOD_RES 14: note = MeGly
KXETXXXXXXXNXX

SEQ ID NO: 135 - skipped

SEQ ID NO: 136 - protein, 14 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXETXXXXXXENXX

SEQ ID NO: 137 - protein, 14 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXETXXXXXXENXX

SEQ ID NO: 138 - protein, 15 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
  LIPID 15: note = Lipidated Lysine
RXNTXXXXXXENXXK

SEQ ID NO: 139 - protein, 14 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 10: note = N-acetyllysine
  MOD_RES 12: note = 3-pyridinylalanine
  MOD_RES 13: note = MeGly
  LIPID 14: note = Lipidated Lysine
XNTXXXXXXXNXXK

SEQ ID NO: 140 - protein, 14 aa, synthetic construct
  MOD_RES 2: note = Penicillamine
  MOD_RES 5: note = 7-methyltryptophan
  LIPID 6: note = Lipidated Lysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXETXKXXXXENXX

SEQ ID NO: 141 - protein, 14 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXETXXXXXXENXX

SEQ ID NO: 142 - protein, 15 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
  LIPID 15: note = Lipidated Lysine
RXETXXXXXXENXXK

SEQ ID NO: 143 - skipped

SEQ ID NO: 144 - protein, 14 aa, synthetic construct
  MOD_RES 1: note = N,N,N-trimethyl-D-homolysine
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
  SITE 1: note = N,N,N-trimethyl-D-homolysine
XXNTXXXXXXENXX

SEQ ID NO: 145 - protein, 14 aa, synthetic construct
  MOD_RES 1: note = (R)-2-(2-(3-((5-carboxy-5-hydrazineylpentyl)amino)- 3-oxopropoxy)ethoxy)-N,N,N-trimethylethan-1-aminium
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
XXNTXXXXXXENXX

SEQ ID NO: 146 - skipped

SEQ ID NO: 147 - protein, 15 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
  LIPID 15: note = Lipidated Lysine
RXETXXXXXXENXXK

SEQ ID NO: 148 - protein, 15 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
  LIPID 15: note = Lipidated Lysine
RXETXXXXXXENXXK

SEQ ID NO: 149 - protein, 13 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(2-hydroxyquinolin-6-yl)propanoic acid
  MOD_RES 9: note = 4-aminotetrahydro-2H-pyran-4-carboxylic acid
  MOD_RES 12: note = 3-pyridinylalanine
  MOD_RES 13: note = MeGly
XNTXXXXXXENXX

SEQ ID NO: 150 - protein, 14 aa, synthetic construct
  MOD_RES 2: note = Penicillamine
  LIPID 3: note = Lipidated Lysine
  MOD_RES 5: note = 3-(7-(3-acetamidophenyl)-1H-indol-3-yl)-2-aminoprop anoic acid
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXKTXXXXXXENXX

SEQ ID NO: 151 - protein, 14 aa, synthetic construct
  MOD_RES 2: note = Penicillamine
  MOD_RES 5: note = 3-(7-(3-acetamidophenyl)-1H-indol-3-yl)-2-aminoprop anoic acid
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXNTXXXXXXENXX

SEQ ID NO: 152 - protein, 13 aa, synthetic construct
  MOD_RES 1: note = Penicillamine
  MOD_RES 4: note = 3-(7-(3-acetamidophenyl)-1H-indol-3-yl)-2-aminoprop anoic acid
  LIPID 5: note = Lipidated Lysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 10: note = N-acetyllysine
  MOD_RES 12: note = 3-pyridinylalanine
  MOD_RES 13: note = MeGly
XNTXKXXXXXNXX

SEQ ID NO: 153 - protein, 13 aa, synthetic construct
  MOD_RES 1: note = Penicillamine
  MOD_RES 4: note = 3-(7-(3-acetamidophenyl)-1H-indol-3-yl)-2-aminoprop anoic acid
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  LIPID 10: note = Lipidated Lysine
  MOD_RES 12: note = 3-pyridinylalanine
  MOD_RES 13: note = MeGly
XNTXXXXXXKNXX

SEQ ID NO: 154 - protein, 14 aa, synthetic construct
  MOD_RES 2: note = Penicillamine
  MOD_RES 5: note = 3-(7-(3-acetamidophenyl)-1H-indol-3-yl)-2-aminoprop anoic acid
  LIPID 6: note = Lipidated Lysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXNTXKXXXXENXX

SEQ ID NO: 155 - protein, 14 aa, synthetic construct
  MOD_RES 1: note = Penicillamine
  MOD_RES 4: note = 3-(7-(3-acetamidophenyl)-1H-indol-3-yl)-2-aminoprop anoic acid
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 10: note = N-acetyllysine
  MOD_RES 12: note = 3-pyridinylalanine
  MOD_RES 13: note = MeGly
  LIPID 14: note = Lipidated Lysine
XNTXXXXXXXNXXK

SEQ ID NO: 156 - protein, 15 aa, synthetic construct
  MOD_RES 2: note = Penicillamine
  MOD_RES 5: note = 3-(7-(3-acetamidophenyl)-1H-indol-3-yl)-2-aminoprop anoic acid
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
  LIPID 15: note = Lipidated Lysine
RXNTXXXXXXENXXK

SEQ ID NO: 157 - protein, 14 aa, synthetic construct
  MOD_RES 2: note = Penicillamine
  MOD_RES 5: note = 3-(7-(3-acetamidophenyl)-1H-indol-3-yl)-2-aminoprop anoic acid
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXETXXXXXXENXX

SEQ ID NO: 158 - protein, 14 aa, synthetic construct
  MOD_RES 2: note = Penicillamine
  MOD_RES 5: note = 3-(7-(3-acetamidophenyl)-1H-indol-3-yl)-2-aminoprop anoic acid
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXETXXXXXXENXX

SEQ ID NO: 159-160 - skipped (2 entries)

SEQ ID NO: 161 - protein, 14 aa, synthetic construct
  MOD_RES 2: note = Penicillamine
  MOD_RES 5: note = 3-(7-(3-acetamidophenyl)-1H-indol-3-yl)-2-aminoprop anoic acid
  MOD_RES 6: note = N-acetyllysine
  MOD_RES 7: note = Penicillamine
  MOD_RES 8: note = 4-(2-(4-(2-amino-2-carboxyethyl)phenoxy)ethyl)-4-me thylmorpholin-4-ium
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXNTXXXXXXENXX

SEQ ID NO: 162-163 - skipped (2 entries)

SEQ ID NO: 164 - protein, 14 aa, synthetic construct
  LIPID 1: note = Lipidated Lysine
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 11: note = N-acetyllysine
  MOD_RES 13: note = 3-pyridinylalanine
  MOD_RES 14: note = MeGly
KXNTXXXXXXXNXX

SEQ ID NO: 165 - protein, 14 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 10: note = N-acetyllysine
  MOD_RES 12: note = 3-pyridinylalanine
  MOD_RES 13: note = MeGly
  LIPID 14: note = Lipidated Lysine
XNTXXXXXXXNXXK

SEQ ID NO: 166 - protein, 15 aa, synthetic construct
  MOD_RES 1: note = (R)-3-carboxy-2-(4-(((R)-5-carboxy-5-hydrazineylpen tyl)amino)-4-oxobutanamido)-N,N,N-trimethylpropan-1-aminiu m
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
  LIPID 15: note = Lipidated Lysine
XXNTXXXXXXENXXK

SEQ ID NO: 167 - protein, 14 aa, synthetic construct
  MOD_RES 1: note = (R)-3-carboxy-2-(4-(((R)-5-carboxy-5-hydrazineylpen tyl)amino)-4-oxobutanamido)-N,N,N-trimethylpropan-1-aminiu m
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = N-Methyl-Lysine(LIPID)
  LIPID 14: note = Lipidated N-Methyl-Lysine
XXNTXXXXXXENXX

SEQ ID NO: 168 - protein, 14 aa, synthetic construct
  MOD_RES 1: note = (R)-3-carboxy-2-(4-(((R)-5-carboxy-5-hydrazineylpen tyl)amino)-4-oxobutanamido)-N,N,N-trimethylpropan-1-aminiu m
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = N-Methyl-Lysine(LIPID)
  LIPID 14: note = Lipidated N-Methyl-Lysine
XXNTXXXXXXENXX

SEQ ID NO: 169 - protein, 14 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 10: note = N-acetyllysine
  MOD_RES 12: note = 3-pyridinylalanine
  MOD_RES 13: note = MeGly
  LIPID 14: note = Lipidated Lysine
XETXXXXXXXNXXK

SEQ ID NO: 170 - protein, 14 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 10: note = N-acetyllysine
  MOD_RES 12: note = 3-pyridinylalanine
  MOD_RES 13: note = MeGly
  LIPID 14: note = Lipidated Lysine
XETXXXXXXXNXXK

SEQ ID NO: 171 - protein, 14 aa, synthetic construct
  MOD_RES 1: note = 5-aminovaleric acid
  MOD_RES 2: note = Abu
  MOD_RES 5: note = 7-methyltryptophan
  MOD_RES 6: note = N-acetyllysine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
XXNTXXCXXXENXX

SEQ ID NO: 172 - protein, 14 aa, synthetic construct
  MOD_RES 1: note = 5-aminovaleric acid
  MOD_RES 2: note = Abu
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
XXNTWQCXXXENXX

SEQ ID NO: 173 - protein, 14 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 5-(4-(2-amino-2-carboxyethyl)phenoxy)-N,N,N-trimeth ylpentan-1-aminium
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 10: note = N-acetyllysine
  MOD_RES 12: note = 3-pyridinylalanine
  MOD_RES 13: note = MeGly
  LIPID 14: note = Lipidated Lysine
XNTXXXXXXXNXXK

SEQ ID NO: 174 - protein, 14 aa, synthetic construct
  MOD_RES 2: note = Penicillamine
  MOD_RES 5: note = 7-methyltryptophan
  MOD_RES 6: note = N-methyl-N-acetyllysine
  MOD_RES 7: note = Penicillamine
  MOD_RES 8: note = 5-(4-(2-amino-2-carboxyethyl)phenoxy)-N,N,N-trimeth ylpentan-1-aminium
  MOD_RES 9: note = 2-amino-3-(6-hydroxynaphthalen-2-yl)propanoic acid
  MOD_RES 10: note = 4-aminotetrahydro-2H-pyran-4-carboxylic acid
  LIPID 11: note = Lipidated Lysine
  MOD_RES 13: note = 3-pyridinylalanine
  MOD_RES 14: note = MeGly
RXNTXXXXXXKNXX

SEQ ID NO: 175-176 - skipped (2 entries)

SEQ ID NO: 177 - protein, 13 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  LIPID 10: note = Lipidated Lysine
  MOD_RES 12: note = 3-pyridinylalanine
  MOD_RES 13: note = MeGly
XNTXXXXXXKNXX

SEQ ID NO: 178 - protein, 13 aa, synthetic construct
  MOD_RES 1: note = Penicillamine
  MOD_RES 4: note = 7-methyltryptophan
  LIPID 5: note = Lipidated Lysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 10: note = N-acetyllysine
  MOD_RES 12: note = 3-pyridinylalanine
  MOD_RES 13: note = MeGly
XNTXKXXXXXNXX

SEQ ID NO: 179-180 - skipped (2 entries)

SEQ ID NO: 181 - protein, 15 aa, synthetic construct
  MOD_RES 1: note = N,N,N-trimethyl-D-homolysine
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 11: note = N-acetyllysine
  MOD_RES 13: note = 3-pyridinylalanine
  MOD_RES 14: note = MeGly
  SITE 1: note = N,N,N-trimethyl-D-homolysine
  LIPID 15: note = Lipidated Lysine
XXNTXXXXXXXNXXK

SEQ ID NO: 182 - protein, 14 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 10: note = N-acetyllysine
  MOD_RES 12: note = 3-pyridinylalanine
  MOD_RES 13: note = MeGly
  LIPID 14: note = Lipidated Lysine
XNTXXXXXXXNXXK

SEQ ID NO: 183 - protein, 13 aa, synthetic construct
  MOD_RES 1: note = Penicillamine
  MOD_RES 4: note = 7-methyltryptophan
  LIPID 5: note = Lipidated Lysine
  MOD_RES 6: note = Penicillamine
  MOD_RES 7: note = 5-(4-(2-amino-2-carboxyethyl)phenoxy)-N,N,N-trimeth ylpentan-1-aminium
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 10: note = N-acetyllysine
  MOD_RES 12: note = 3-pyridinylalanine
  MOD_RES 13: note = MeGly
XNTXKXXXXXNXX

SEQ ID NO: 184 - protein, 14 aa, synthetic construct
  MOD_RES 1: note = N,N,N-trimethyl-D-homolysine
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  LIPID 11: note = Lipidated Lysine
  MOD_RES 13: note = 3-pyridinylalanine
  MOD_RES 14: note = MeGly
  SITE 1: note = N,N,N-trimethyl-D-homolysine
XXNTXXXXXXKNXX

SEQ ID NO: 185 - protein, 13 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  LIPID 10: note = Lipidated Lysine
  MOD_RES 12: note = 3-pyridinylalanine
  MOD_RES 13: note = MeGly
XNTXXXXXXKNXX

SEQ ID NO: 186 - protein, 13 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  LIPID 10: note = Lipidated Lysine
  MOD_RES 12: note = 3-pyridinylalanine
  MOD_RES 13: note = MeGly
XNTXXXXXXKNXX

SEQ ID NO: 187 - protein, 14 aa, synthetic construct
  MOD_RES 1: note = N,N,N-trimethyl-D-homolysine
  MOD_RES 2: note = Penicillamine
  MOD_RES 5: note = 7-methyltryptophan
  LIPID 6: note = Lipidated Lysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 11: note = N-acetyllysine
  MOD_RES 13: note = 3-pyridinylalanine
  MOD_RES 14: note = MeGly
  SITE 1: note = N,N,N-trimethyl-D-homolysine
XXNTXKXXXXXNXX

SEQ ID NO: 188 - protein, 13 aa, synthetic construct
  MOD_RES 1: note = Penicillamine
  MOD_RES 4: note = 7-methyltryptophan
  LIPID 5: note = Lipidated Lysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 10: note = N-acetyllysine
  MOD_RES 12: note = 3-pyridinylalanine
  MOD_RES 13: note = MeGly
XNTXKXXXXXNXX

SEQ ID NO: 189 - protein, 13 aa, synthetic construct
  MOD_RES 1: note = Penicillamine
  MOD_RES 4: note = 7-methyltryptophan
  LIPID 5: note = Lipidated Lysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 10: note = N-acetyllysine
  MOD_RES 12: note = 3-pyridinylalanine
  MOD_RES 13: note = MeGly
XNTXKXXXXXNXX

SEQ ID NO: 190 - protein, 14 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = N-Methyl-Lysine(LIPID)
  LIPID 14: note = Lipidated N-Methyl-Lysine
RXNTXXXXXXENXX

SEQ ID NO: 191 - protein, 15 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
  LIPID 15: note = Lipidated Lysine
RXNTXXXXXXENXXK

SEQ ID NO: 192 - protein, 13 aa, synthetic construct
  MOD_RES 1: note = Penicillamine
  MOD_RES 4: note = 7-methyltryptophan
  LIPID 5: note = Lipidated Lysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 10: note = N-acetyllysine
  MOD_RES 12: note = 3-pyridinylalanine
  MOD_RES 13: note = MeGly
XETXKXXXXXNXX

SEQ ID NO: 193 - protein, 13 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  LIPID 10: note = Lipidated Lysine
  MOD_RES 12: note = 3-pyridinylalanine
  MOD_RES 13: note = MeGly
XETXXXXXXKNXX

SEQ ID NO: 194-197 - skipped (4 entries)

SEQ ID NO: 198 - protein, 13 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 5-(4-(2-amino-2-carboxyethyl)phenoxy)-N,N,N-trimeth ylpentan-1-aminium
  MOD_RES 8: note = 2-amino-3-(6-hydroxynaphthalen-2-yl)propanoic acid
  MOD_RES 9: note = 4-aminotetrahydro-2H-pyran-4-carboxylic acid
  LIPID 10: note = Lipidated Lysine
  MOD_RES 12: note = 3-pyridinylalanine
  MOD_RES 13: note = MeGly
XNTXXXXXXKNXX

SEQ ID NO: 199 - protein, 13 aa, synthetic construct
  MOD_RES 1: note = Penicillamine
  MOD_RES 4: note = 7-methyltryptophan
  MOD_RES 5: note = N-polyethyleneglycollysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  LIPID 10: note = Lipidated Lysine
  MOD_RES 12: note = 3-pyridinylalanine
  MOD_RES 13: note = MeGly
XNTXXXXXXKNXX

SEQ ID NO: 200 - protein, 13 aa, synthetic construct
  MOD_RES 1: note = Penicillamine
  MOD_RES 4: note = 7-methyltryptophan
  LIPID 5: note = Lipidated Lysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 10: note = N-polyethyleneglycollysine
  MOD_RES 12: note = 3-pyridinylalanine
  MOD_RES 13: note = MeGly
XNTXKXXXXXNXX

SEQ ID NO: 201-203 - skipped (3 entries)

SEQ ID NO: 204 - protein, 14 aa, synthetic construct
  MOD_RES 1: note = (R)-3-carboxy-2-(4-(((R)-5-carboxy-5-hydrazineylpen tyl)amino)-4-oxobutanamido)-N,N,N-trimethylpropan-1-aminiu m
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = N-Methyl-Lysine(LIPID)
  LIPID 14: note = Lipidated N-Methyl-Lysine
XXNTXXXXXXENXX

SEQ ID NO: 205-209 - skipped (5 entries)

SEQ ID NO: 210 - protein, 14 aa, synthetic construct
  MOD_RES 1: note = (R)-3-carboxy-2-(4-(((R)-5-carboxy-5-hydrazineylpen tyl)amino)-4-oxobutanamido)-N,N,N-trimethylpropan-1-aminiu m
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = N-Methyl-Lysine(LIPID)
  LIPID 14: note = Lipidated N-Methyl-Lysine
XXNTXXXXXXENXX

SEQ ID NO: 211-213 - skipped (3 entries)

SEQ ID NO: 214 - protein, 13 aa, synthetic construct
  MOD_RES 1: note = Penicillamine
  MOD_RES 4: note = 7-methyltryptophan
  LIPID 5: note = Lipidated Lysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 10: note = N-acetyllysine
  MOD_RES 12: note = 3-pyridinylalanine
  MOD_RES 13: note = MeGly
XETXKXXXXXNXX

SEQ ID NO: 215 - protein, 13 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  LIPID 10: note = Lipidated Lysine
  MOD_RES 12: note = 3-pyridinylalanine
  MOD_RES 13: note = MeGly
XETXXXXXXKNXX

SEQ ID NO: 216 - protein, 14 aa, synthetic construct
  LIPID 1: note = Lipidated Lysine
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 11: note = N-acetyllysine
  MOD_RES 13: note = 3-pyridinylalanine
  MOD_RES 14: note = MeGly
KXNTXXXXXXXNXX

SEQ ID NO: 217 - protein, 14 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXNTXXXXXXENXX

SEQ ID NO: 218 - protein, 15 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
  LIPID 15: note = Lipidated Lysine
RXNTXXXXXXENXXK

SEQ ID NO: 219 - protein, 14 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 11: note = N-acetyllysine
  MOD_RES 13: note = 3-pyridinylalanine
  MOD_RES 14: note = MeGly
RXNTXXXXXXXNXX

SEQ ID NO: 220 - protein, 14 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 11: note = N-acetyllysine
  MOD_RES 13: note = 3-pyridinylalanine
  MOD_RES 14: note = MeGly
RXNTXXXXXXXNXX

SEQ ID NO: 221 - protein, 15 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 11: note = N-acetyllysine
  MOD_RES 13: note = 3-pyridinylalanine
  MOD_RES 14: note = MeGly
  LIPID 15: note = Lipidated Lysine
RXNTXXXXXXXNXXK

SEQ ID NO: 222 - protein, 15 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 11: note = N-acetyllysine
  MOD_RES 13: note = 3-pyridinylalanine
  MOD_RES 14: note = MeGly
  LIPID 15: note = Lipidated Lysine
RXNTXXXXXXXNXXK

SEQ ID NO: 223 - skipped

SEQ ID NO: 224 - protein, 14 aa, synthetic construct
  MOD_RES 1: note = Penicillamine
  MOD_RES 4: note = 7-methyltryptophan
  MOD_RES 5: note = N-methyl-N-acetyllysine
  MOD_RES 6: note = Penicillamine
  MOD_RES 7: note = 5-(4-(2-amino-2-carboxyethyl)phenoxy)-N,N,N-trimeth ylpentan-1-aminium
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 10: note = N-methyl-N-acetyllysine
  MOD_RES 12: note = 3-pyridinylalanine
  MOD_RES 13: note = MeGly
  LIPID 14: note = Lipidated Lysine
XNTXXXXXXXNXXK

SEQ ID NO: 225 - protein, 13 aa, synthetic construct
  MOD_RES 1: note = Penicillamine
  MOD_RES 4: note = 7-methyltryptophan
  LIPID 5: note = Lipidated Lysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 10: note = N-methyl-N-acetyllysine
  MOD_RES 12: note = 3-pyridinylalanine
  MOD_RES 13: note = MeGly
XNTXKXXXXXNXX

SEQ ID NO: 226 - skipped

SEQ ID NO: 227 - protein, 14 aa, synthetic construct
  LIPID 1: note = Lipidated Lysine
  MOD_RES 2: note = Penicillamine
  MOD_RES 5: note = 7-methyltryptophan
  MOD_RES 6: note = N-methyl-N-acetyllysine
  MOD_RES 7: note = Penicillamine
  MOD_RES 8: note = 5-(4-(2-amino-2-carboxyethyl)phenoxy)-N,N,N-trimeth ylpentan-1-aminium
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 11: note = N-methyl-N-acetyllysine
  MOD_RES 13: note = 3-pyridinylalanine
  MOD_RES 14: note = MeGly
KXNTXXXXXXXNXX

SEQ ID NO: 228 - protein, 14 aa, synthetic construct
  MOD_RES 1: note = Penicillamine
  MOD_RES 4: note = 7-methyltryptophan
  MOD_RES 5: note = N-methyl-N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 10: note = N-methyl-N-acetyllysine
  MOD_RES 12: note = 3-pyridinylalanine
  MOD_RES 13: note = MeGly
  LIPID 14: note = Lipidated Lysine
XNTXXXXXXXNXXK

SEQ ID NO: 229 - protein, 14 aa, synthetic construct
  LIPID 1: note = Lipidated Lysine
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
KXNTXXXXXXENXX

SEQ ID NO: 230-232 - skipped (3 entries)

SEQ ID NO: 233 - protein, 14 aa, synthetic construct
  MOD_RES 1: note = (R)-3-carboxy-2-(4-(((R)-5-carboxy-5-hydrazineylpen tyl)amino)-4-oxobutanamido)-N,N,N-trimethylpropan-1-aminiu m
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = N-Methyl-Lysine(LIPID)
  LIPID 14: note = Lipidated N-Methyl-Lysine
XXNTXXXXXXENXX

SEQ ID NO: 234-238 - skipped (5 entries)

SEQ ID NO: 239 - protein, 14 aa, synthetic construct
  MOD_RES 1: note = N,N,N-trimethyl-D-homolysine
  MOD_RES 2: note = Penicillamine
  MOD_RES 3: note = N,N-dimethylasparagine
  MOD_RES 5: note = 7-methyltryptophan
  LIPID 6: note = Lipidated Lysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
  SITE 1: note = N,N,N-trimethyl-D-homolysine
XXXTXKXXXXENXX

SEQ ID NO: 240 - protein, 14 aa, synthetic construct
  MOD_RES 1: note = N,N,N-trimethyl-D-homolysine
  MOD_RES 2: note = Penicillamine
  LIPID 3: note = Lipidated Lysine
  MOD_RES 5: note = 7-methyltryptophan
  MOD_RES 6: note = N-methyl-N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
  SITE 1: note = N,N,N-trimethyl-D-homolysine
XXKTXXXXXXENXX

SEQ ID NO: 241 - protein, 14 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 10: note = N-acetyllysine
  MOD_RES 12: note = 3-pyridinylalanine
  MOD_RES 13: note = MeGly
  LIPID 14: note = Lipidated Lysine
XNTXXXXXXXNXXK

SEQ ID NO: 242-243 - skipped (2 entries)

SEQ ID NO: 244 - protein, 15 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
  LIPID 15: note = Lipidated Lysine
RXNTXXXXXXENXXK

SEQ ID NO: 245 - protein, 14 aa, synthetic construct
  LIPID 1: note = Lipidated Lysine
  MOD_RES 2: note = Penicillamine
  MOD_RES 5: note = 7-methyltryptophan
  MOD_RES 6: note = N-methyl-N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 11: note = N-methyl-N-acetyllysine
  MOD_RES 13: note = 3-pyridinylalanine
  MOD_RES 14: note = MeGly
KXNTXXXXXXXNXX

SEQ ID NO: 246 - protein, 14 aa, synthetic construct
  MOD_RES 1: note = Penicillamine
  MOD_RES 4: note = 7-methyltryptophan
  MOD_RES 5: note = N-methyl-N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 10: note = N-methyl-N-acetyllysine
  MOD_RES 12: note = 3-pyridinylalanine
  MOD_RES 13: note = MeGly
  LIPID 14: note = Lipidated Lysine
XNTXXXXXXXNXXK

SEQ ID NO: 247 - skipped

SEQ ID NO: 248 - protein, 14 aa, synthetic construct
  LIPID 1: note = Lipidated Lysine
  MOD_RES 2: note = Penicillamine
  MOD_RES 5: note = 7-methyltryptophan
  MOD_RES 6: note = N-methyl-N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 11: note = N-methyl-N-acetyllysine
  MOD_RES 13: note = 3-pyridinylalanine
  MOD_RES 14: note = MeGly
KXNTXXXXXXXNXX

SEQ ID NO: 249 - protein, 13 aa, synthetic construct
  MOD_RES 1: note = Penicillamine
  MOD_RES 4: note = 7-methyltryptophan
  MOD_RES 5: note = N-methyl-N-acetyllysine
  MOD_RES 6: note = Penicillamine
  MOD_RES 7: note = 5-(4-(2-amino-2-carboxyethyl)phenoxy)-N,N,N-trimeth ylpentan-1-aminium
  MOD_RES 8: note = 2-amino-3-(6-hydroxynaphthalen-2-yl)propanoic acid
  MOD_RES 9: note = 4-aminotetrahydro-2H-pyran-4-carboxylic acid
  LIPID 10: note = Lipidated Lysine
  MOD_RES 12: note = 3-pyridinylalanine
  MOD_RES 13: note = MeGly
XNTXXXXXXKNXX

SEQ ID NO: 250 - protein, 13 aa, synthetic construct
  MOD_RES 1: note = Penicillamine
  MOD_RES 4: note = 7-methyltryptophan
  LIPID 5: note = Lipidated Lysine
  MOD_RES 6: note = Penicillamine
  MOD_RES 7: note = 5-(4-(2-amino-2-carboxyethyl)phenoxy)-N,N,N-trimeth ylpentan-1-aminium
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 10: note = N-methyl-N-acetyllysine
  MOD_RES 12: note = 3-pyridinylalanine
  MOD_RES 13: note = MeGly
XNTXKXXXXXNXX

SEQ ID NO: 251 - skipped

SEQ ID NO: 252 - protein, 13 aa, synthetic construct
  MOD_RES 1: note = Penicillamine
  MOD_RES 4: note = 7-methyltryptophan
  MOD_RES 5: note = N-methyl-N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  LIPID 10: note = Lipidated Lysine
  MOD_RES 12: note = 3-pyridinylalanine
  MOD_RES 13: note = MeGly
XNTXXXXXXKNXX

SEQ ID NO: 253 - protein, 13 aa, synthetic construct
  MOD_RES 1: note = Penicillamine
  MOD_RES 4: note = 7-methyltryptophan
  LIPID 5: note = Lipidated Lysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 10: note = N-methyl-N-acetyllysine
  MOD_RES 12: note = 3-pyridinylalanine
  MOD_RES 13: note = MeGly
XNTXKXXXXXNXX

SEQ ID NO: 254 - protein, 15 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 11: note = N-acetyllysine
  MOD_RES 13: note = 3-pyridinylalanine
  MOD_RES 14: note = MeGly
  LIPID 15: note = Lipidated Lysine
RXNTXXXXXXXNXXK

SEQ ID NO: 255 - protein, 15 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 11: note = N-acetyllysine
  MOD_RES 13: note = 3-pyridinylalanine
  MOD_RES 14: note = MeGly
  LIPID 15: note = Lipidated Lysine
RXNTXXXXXXXNXXK

SEQ ID NO: 256 - protein, 14 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 10: note = N-acetyllysine
  MOD_RES 12: note = 3-pyridinylalanine
  MOD_RES 13: note = MeGly
  LIPID 14: note = Lipidated Lysine
XNTXXXXXXXNXXK

SEQ ID NO: 257-262 - skipped (6 entries)

SEQ ID NO: 263 - protein, 14 aa, synthetic construct
  MOD_RES 1: note = (R)-3-carboxy-2-(4-(((R)-5-carboxy-5-hydrazineylpen tyl)amino)-4-oxobutanamido)-N,N,N-trimethylpropan-1-aminiu m
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
XXNTXXXXXXENXX

SEQ ID NO: 264 - protein, 14 aa, synthetic construct
  MOD_RES 1: note = (R)-3-carboxy-2-(4-(((R)-5-carboxy-5-hydrazineylpen tyl)amino)-4-oxobutanamido)-N,N,N-trimethylpropan-1-aminiu m
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
XXNTXXXXXXENXX

SEQ ID NO: 265 - protein, 14 aa, synthetic construct
  MOD_RES 1: note = (R)-3-carboxy-2-(4-(((R)-5-carboxy-5-hydrazineylpen tyl)amino)-4-oxobutanamido)-N,N,N-trimethylpropan-1-aminiu m
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
XXNTXXXXXXENXX

SEQ ID NO: 266 - protein, 14 aa, synthetic construct
  MOD_RES 1: note = (R)-3-carboxy-2-(4-(((R)-5-carboxy-5-hydrazineylpen tyl)amino)-4-oxobutanamido)-N,N,N-trimethylpropan-1-aminiu m
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
XXNTXXXXXXENXX

SEQ ID NO: 267 - protein, 14 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 10: note = N-acetyllysine
  MOD_RES 12: note = 3-pyridinylalanine
  MOD_RES 13: note = MeGly
  LIPID 14: note = Lipidated Lysine
XNTXXXXXXXNXXK

SEQ ID NO: 268 - protein, 14 aa, synthetic construct
  LIPID 1: note = Lipidated Lysine
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
KXNTXXXXXXENXX

SEQ ID NO: 269 - protein, 15 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
  LIPID 15: note = Lipidated Lysine
RXNTXXXXXXENXXK

SEQ ID NO: 270 - skipped

SEQ ID NO: 271 - protein, 14 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 10: note = N-acetyllysine
  MOD_RES 12: note = 3-pyridinylalanine
  MOD_RES 13: note = MeGly
  LIPID 14: note = Lipidated Lysine
XNTXXXXXXXNXXK

SEQ ID NO: 272 - skipped

SEQ ID NO: 273 - protein, 13 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  LIPID 10: note = Lipidated Lysine
  MOD_RES 12: note = 3-pyridinylalanine
  MOD_RES 13: note = MeGly
XNTXXXXXXKNXX

SEQ ID NO: 274 - skipped

SEQ ID NO: 275 - protein, 14 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 10: note = N-acetyllysine
  MOD_RES 12: note = 3-pyridinylalanine
  MOD_RES 13: note = MeGly
  LIPID 14: note = Lipidated Lysine
XNTXXXXXXXNXXK

SEQ ID NO: 276 - protein, 13 aa, synthetic construct
  MOD_RES 1: note = Penicillamine
  LIPID 2: note = Lipidated Lysine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 12: note = 3-pyridinylalanine
  MOD_RES 13: note = MeGly
XKTXXXXXXENXX

SEQ ID NO: 277 - protein, 14 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 10: note = N-acetyllysine
  MOD_RES 12: note = 3-pyridinylalanine
  MOD_RES 13: note = MeGly
  LIPID 14: note = Lipidated Lysine
XNTXXXXXXXNXXK

SEQ ID NO: 278 - protein, 14 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 10: note = N-acetyllysine
  MOD_RES 12: note = 3-pyridinylalanine
  MOD_RES 13: note = MeGly
  LIPID 14: note = Lipidated Lysine
XNTXXXXXXXNXXK

SEQ ID NO: 279 - protein, 14 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = N-Methyl-Lysine(LIPID)
  LIPID 14: note = Lipidated N-Methyl-Lysine
RXNTXXXXXXENXX

SEQ ID NO: 280 - protein, 14 aa, synthetic construct
  MOD_RES 1: note = (R)-3-carboxy-2-(4-(((R)-5-carboxy-5-hydrazineylpen tyl)amino)-4-oxobutanamido)-N,N,N-trimethylpropan-1-aminiu m
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-(2-(2-(4-(2-amino-2-carboxyethyl)phenoxy)ethoxy)e thoxy)-N,N,N-trimethylethan-1-aminium
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
XXNTXXXXXXENXX

SEQ ID NO: 281 - skipped

SEQ ID NO: 282 - protein, 15 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
  LIPID 15: note = Lipidated Lysine
RXNTXXXXXXENXXK

SEQ ID NO: 283 - protein, 14 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 12: note = 3-pyridinylalanine
  MOD_RES 13: note = MeGly
  LIPID 14: note = Lipidated Lysine
XNTXXXXXXENXXK

SEQ ID NO: 284-286 - skipped (3 entries)

SEQ ID NO: 287 - protein, 14 aa, synthetic construct
  MOD_RES 2: note = Penicillamine
  MOD_RES 5: note = 3-(7-(3-acetamidophenyl)-1H-indol-3-yl)-2-aminoprop anoic acid
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXNTXXXXXXENXX

SEQ ID NO: 288 - protein, 13 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(2-hydroxyquinolin-6-yl)propanoic acid
  MOD_RES 9: note = 4-aminotetrahydro-2H-pyran-4-carboxylic acid
  MOD_RES 12: note = 3-pyridinylalanine
  MOD_RES 13: note = MeGly
XNTXXXXXXENXX

SEQ ID NO: 289 - protein, 13 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(2-methoxyquinolin-6-yl)propanoic acid
  MOD_RES 9: note = 4-aminotetrahydro-2H-pyran-4-carboxylic acid
  MOD_RES 12: note = 3-pyridinylalanine
  MOD_RES 13: note = MeGly
XNTXXXXXXENXX

SEQ ID NO: 290 - protein, 14 aa, synthetic construct
  MOD_RES 1: note = N-amino-O-(2-(2-(methylamino)ethoxy)ethyl)-D-serine
  MOD_RES 2: note = Penicillamine
  MOD_RES 5: note = 7-methyltryptophan
  MOD_RES 6: note = N-methyl-N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
  SITE 1: note = N-amino-O-(2-(2-(methylamino)ethoxy)ethyl)-D-serine
XXNTXXXXXXDNXX

SEQ ID NO: 291-295 - skipped (5 entries)

SEQ ID NO: 296 - protein, 13 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(2-methoxyquinolin-6-yl)propanoic acid
  MOD_RES 9: note = 4-aminotetrahydro-2H-pyran-4-carboxylic acid
  MOD_RES 12: note = 3-pyridinylalanine
  MOD_RES 13: note = MeGly
XNTXXXXXXENXX

SEQ ID NO: 297 - skipped

SEQ ID NO: 298 - protein, 13 aa, synthetic construct
  MOD_RES 1: note = Penicillamine
  MOD_RES 4: note = 7-methyltryptophan
  MOD_RES 5: note = N-polyethyleneglycollysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 12: note = 3-pyridinylalanine
  MOD_RES 13: note = MeGly
XETXXXXXXENXX

SEQ ID NO: 299-302 - skipped (4 entries)

SEQ ID NO: 303 - protein, 13 aa, synthetic construct
  MOD_RES 1: note = Penicillamine
  MOD_RES 4: note = 7-methyltryptophan
  LIPID 5: note = Lipidated Lysine
  MOD_RES 6: note = Penicillamine
  MOD_RES 8: note = 2-amino-3-(6-hydroxynaphthalen-2-yl)propanoic acid
  MOD_RES 9: note = Alpha-methyllysine
  MOD_RES 12: note = 3-pyridinylalanine
  MOD_RES 13: note = MeGly
XLTXKXFXXLNXX

SEQ ID NO: 304 - protein, 13 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 8: note = 2-amino-3-(6-hydroxynaphthalen-2-yl)propanoic acid
  MOD_RES 9: note = Alpha-methyllysine
  MOD_RES 12: note = 3-pyridinylalanine
  MOD_RES 13: note = MeGly
XLTXXXFXXLNXX

SEQ ID NO: 305 - protein, 13 aa, synthetic construct
  MOD_RES 1: note = Penicillamine
  MOD_RES 4: note = 7-methyltryptophan
  LIPID 5: note = Lipidated Lysine
  MOD_RES 6: note = Penicillamine
  MOD_RES 8: note = 2-amino-3-(6-hydroxynaphthalen-2-yl)propanoic acid
  MOD_RES 9: note = Alpha-methyllysine
  MOD_RES 12: note = 3-pyridinylalanine
  MOD_RES 13: note = MeGly
XNTXKXFXXLNXX

SEQ ID NO: 306 - protein, 13 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 4-methoxyphenylalanine
  MOD_RES 8: note = 2-amino-3-(6-hydroxynaphthalen-2-yl)propanoic acid
  MOD_RES 9: note = Alpha-methyllysine
  MOD_RES 12: note = 3-pyridinylalanine
  MOD_RES 13: note = MeGly
XNTXXXXXXLNXX

SEQ ID NO: 307 - protein, 13 aa, synthetic construct
  MOD_RES 1: note = Penicillamine
  MOD_RES 4: note = 7-methyltryptophan
  LIPID 5: note = Lipidated Lysine
  MOD_RES 6: note = Penicillamine
  MOD_RES 7: note = 4-methoxyphenylalanine
  MOD_RES 8: note = 2-amino-3-(6-hydroxynaphthalen-2-yl)propanoic acid
  MOD_RES 9: note = Alpha-methyllysine
  MOD_RES 12: note = 3-pyridinylalanine
  MOD_RES 13: note = MeGly
XNTXKXXXXLNXX

SEQ ID NO: 308 - protein, 13 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 4-methoxyphenylalanine
  MOD_RES 8: note = 2-amino-3-(6-hydroxynaphthalen-2-yl)propanoic acid
  MOD_RES 9: note = Alpha-methyllysine
  MOD_RES 12: note = 3-pyridinylalanine
  MOD_RES 13: note = MeGly
XNTXXXXXXLNXX

SEQ ID NO: 309-310 - skipped (2 entries)

SEQ ID NO: 311 - protein, 13 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(7-hydroxynaphthalen-2-yl)propanoic acid
  MOD_RES 9: note = 4-aminotetrahydro-2H-pyran-4-carboxylic acid
  MOD_RES 12: note = 3-pyridinylalanine
  MOD_RES 13: note = MeGly
XNTXXXXXXENXX

SEQ ID NO: 312 - protein, 14 aa, synthetic construct
  MOD_RES 2: note = Penicillamine
  MOD_RES 5: note = 7-bromotryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXNTXXXXXXENXX

SEQ ID NO: 313 - protein, 13 aa, synthetic construct
  MOD_RES 1: note = Penicillamine
  MOD_RES 4: note = 7-bromotryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 12: note = 3-pyridinylalanine
  MOD_RES 13: note = MeGly
XNTXXXXXXENXX

SEQ ID NO: 314-317 - skipped (4 entries)

SEQ ID NO: 318 - protein, 13 aa, synthetic construct
  MOD_RES 1: note = Penicillamine
  MOD_RES 4: note = 7-methyltryptophan
  MOD_RES 5: note = N-methyl-N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 12: note = 3-pyridinylalanine
  MOD_RES 13: note = MeGly
XETXXXXXXENXX

SEQ ID NO: 319 - protein, 13 aa, synthetic construct
  MOD_RES 1: note = Penicillamine
  MOD_RES 4: note = 7-methyltryptophan
  MOD_RES 5: note = N-polyethyleneglycollysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 12: note = 3-pyridinylalanine
  MOD_RES 13: note = MeGly
XNTXXXXXXENXX

SEQ ID NO: 320-325 - skipped (6 entries)

SEQ ID NO: 326 - protein, 13 aa, synthetic construct
  MOD_RES 1: note = Penicillamine
  MOD_RES 4: note = 7-methyltryptophan
  MOD_RES 5: note = N-polyethyleneglycollysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 12: note = 3-pyridinylalanine
  MOD_RES 13: note = MeGly
XETXXXXXXENXX

SEQ ID NO: 327-330 - skipped (4 entries)

SEQ ID NO: 331 - protein, 13 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 12: note = 3-pyridinylalanine
  MOD_RES 13: note = MeGly
XETXXXXXXENXX

SEQ ID NO: 332 - protein, 13 aa, synthetic construct
  MOD_RES 1: note = Penicillamine
  MOD_RES 4: note = 7-methyltryptophan
  MOD_RES 5: note = N-methyl-N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 12: note = 3-pyridinylalanine
  MOD_RES 13: note = MeGly
XETXXXXXXENXX

SEQ ID NO: 333 - skipped

SEQ ID NO: 334 - protein, 13 aa, synthetic construct
  MOD_RES 1: note = Penicillamine
  MOD_RES 4: note = 2-amino-3-(benzo[b]thiophen-3-yl)propanoic acid
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 12: note = 3-pyridinylalanine
  MOD_RES 13: note = MeGly
XNTXXXXXXENXX

SEQ ID NO: 335-337 - skipped (3 entries)

SEQ ID NO: 338 - protein, 13 aa, synthetic construct
  MOD_RES 1: note = Penicillamine
  MOD_RES 4: note = 7-methyltryptophan
  MOD_RES 5: note = 2-amino-4-(N-methylacetamido)butanoic acid
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 12: note = 3-pyridinylalanine
  MOD_RES 13: note = MeGly
XNTXXXXXXENXX

SEQ ID NO: 339 - skipped

SEQ ID NO: 340 - protein, 14 aa, synthetic construct
  MOD_RES 1: note = (R)-3-carboxy-2-(4-(((R)-5-carboxy-5-hydrazineylpen tyl)amino)-4-oxobutanamido)-N,N,N-trimethylpropan-1-aminiu m
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
XXNTXXXXXXENXX

SEQ ID NO: 341-346 - skipped (6 entries)

SEQ ID NO: 347 - protein, 13 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 12: note = 3-pyridinylalanine
  MOD_RES 13: note = MeGly
XNTXXXXXXENXX

SEQ ID NO: 348 - protein, 14 aa, synthetic construct
  MOD_RES 2: note = Penicillamine
  MOD_RES 5: note = 7-methyltryptophan
  MOD_RES 6: note = N-methyl-N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 11: note = Homoglutamic acid
  MOD_RES 13: note = 3-pyridinylalanine
  MOD_RES 14: note = MeGly
KXNTXXXXXXXNXX

SEQ ID NO: 349 - protein, 14 aa, synthetic construct
  MOD_RES 1: note = (R)-3-carboxy-2-(4-(((R)-5-carboxy-5-hydrazineylpen tyl)amino)-4-oxobutanamido)-N,N,N-trimethylpropan-1-aminiu m
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
XXNTXXXXXXENXX

SEQ ID NO: 350-364 - skipped (15 entries)

SEQ ID NO: 365 - protein, 13 aa, synthetic construct
  MOD_RES 1: note = Penicillamine
  MOD_RES 4: note = 7-methyltryptophan
  MOD_RES 5: note = N-methyl-N-acetyllysine
  MOD_RES 6: note = Penicillamine
  MOD_RES 7: note = 2-amino-3-(4-(2-(methylamino)ethoxy)phenyl)propanoi c acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 12: note = 3-pyridinylalanine
  MOD_RES 13: note = MeGly
XETXXXXXXENXX

SEQ ID NO: 366-371 - skipped (6 entries)

SEQ ID NO: 372 - protein, 13 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 12: note = 3-pyridinylalanine
  MOD_RES 13: note = MeGly
XETXXXXXXENXX

SEQ ID NO: 373-374 - skipped (2 entries)

SEQ ID NO: 375 - protein, 13 aa, synthetic construct
  MOD_RES 1: note = Penicillamine
  MOD_RES 4: note = 7-methyltryptophan
  MOD_RES 5: note = N-methyl-N-acetyllysine
  MOD_RES 6: note = Penicillamine
  MOD_RES 7: note = 2-amino-3-(4-(2-(methylamino)ethoxy)phenyl)propanoi c acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 12: note = 3-pyridinylalanine
  MOD_RES 13: note = MeGly
XETXXXXXXENXX

SEQ ID NO: 376-377 - skipped (2 entries)

SEQ ID NO: 378 - protein, 13 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 12: note = 3-pyridinylalanine
  MOD_RES 13: note = MeGly
XNTXXXXXXENXX

SEQ ID NO: 379 - protein, 13 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 12: note = 3-pyridinylalanine
  MOD_RES 13: note = MeGly
XNTXXXXXXENXX

SEQ ID NO: 380 - protein, 13 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 12: note = 3-pyridinylalanine
  MOD_RES 13: note = MeGly
XNTXXXXXXENXX

SEQ ID NO: 381 - skipped

SEQ ID NO: 382 - protein, 13 aa, synthetic construct
  MOD_RES 1: note = Penicillamine
  MOD_RES 4: note = 7-methyltryptophan
  MOD_RES 5: note = N,N,N-trimethylhomolysine
  MOD_RES 6: note = Penicillamine
  MOD_RES 7: note = 2-amino-3-(4-(2-(methylamino)ethoxy)phenyl)propanoi c acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 12: note = 3-pyridinylalanine
  MOD_RES 13: note = MeGly
XETXXXXXXENXX

SEQ ID NO: 383-387 - skipped (5 entries)

SEQ ID NO: 388 - protein, 14 aa, synthetic construct
  MOD_RES 1: note = N,N,N-trimethyl-D-lysine
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
  SITE 1: note = N,N,N-trimethyl-D-lysine
XXNTXXXXXXENXX

SEQ ID NO: 389-394 - skipped (6 entries)

SEQ ID NO: 395 - protein, 14 aa, synthetic construct
  LIPID 1: note = Lipidated Lysine
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
KXETXXXXXXENXX

SEQ ID NO: 396 - skipped

SEQ ID NO: 397 - protein, 13 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 12: note = 3-pyridinylalanine
  MOD_RES 13: note = MeGly
XETXXXXXXENXX

SEQ ID NO: 398-406 - skipped (9 entries)

SEQ ID NO: 407 - protein, 14 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-fluoronaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXNTXXXXXXENXX

SEQ ID NO: 408-412 - skipped (5 entries)

SEQ ID NO: 413 - protein, 14 aa, synthetic construct
  MOD_RES 1: note = (R)-4-acetamido-2-aminobutanoic acid
  MOD_RES 2: note = Abu
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
XXNTWQCXXXCNXX

SEQ ID NO: 414-416 - skipped (3 entries)

SEQ ID NO: 417 - protein, 14 aa, synthetic construct
  MOD_RES 1: note = 7-aminoheptanoic acid
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
XXNTXXXXXXENXX

SEQ ID NO: 418 - protein, 14 aa, synthetic construct
  MOD_RES 1: note = Acp
  MOD_RES 2: note = Abu
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
XXNTWQCXXXENXX

SEQ ID NO: 419 - protein, 14 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 2-amino-3-(4-carbamoyl-1H-imidazol-1-yl)propanoic acid
  MOD_RES 14: note = MeGly
RXNTXXXXXXENXX

SEQ ID NO: 420 - protein, 14 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  LIPID 11: note = Lipidated Lysine
  MOD_RES 13: note = 3-pyridinylalanine
  MOD_RES 14: note = MeGly
RXETXXXXXXKNXX

SEQ ID NO: 421 - protein, 14 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 11: note = N-acetyllysine
  MOD_RES 13: note = 3-pyridinylalanine
  MOD_RES 14: note = MeGly
RXETXXXXXXXNXX

SEQ ID NO: 422 - protein, 14 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 11: note = N-acetyllysine
  MOD_RES 13: note = 3-pyridinylalanine
  MOD_RES 14: note = MeGly
RXETXXXXXXXNXX

SEQ ID NO: 423 - protein, 14 aa, synthetic construct
  LIPID 1: note = Lipidated Lysine
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 11: note = N-acetyllysine
  MOD_RES 13: note = 3-pyridinylalanine
  MOD_RES 14: note = MeGly
KXETXXXXXXXNXX

SEQ ID NO: 424 - protein, 14 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  LIPID 11: note = Lipidated Lysine
  MOD_RES 13: note = 3-pyridinylalanine
  MOD_RES 14: note = MeGly
RXNTXXXXXXKNXX

SEQ ID NO: 425 - protein, 14 aa, synthetic construct
  MOD_RES 2: note = Penicillamine
  LIPID 3: note = Lipidated Lysine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXKTXXXXXXENXX

SEQ ID NO: 426 - protein, 14 aa, synthetic construct
  LIPID 1: note = Lipidated Lysine
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
KXNTXXXXXXENXX

SEQ ID NO: 427 - protein, 14 aa, synthetic construct
  LIPID 1: note = Lipidated Lysine
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 11: note = N-acetyllysine
  MOD_RES 13: note = 3-pyridinylalanine
  MOD_RES 14: note = MeGly
KXNTXXXXXXXNXX

SEQ ID NO: 428 - protein, 13 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(6-hydroxynaphthalen-2-yl)propanoic acid
  MOD_RES 9: note = 4-aminotetrahydro-2H-pyran-4-carboxylic acid
  MOD_RES 12: note = 3-pyridinylalanine
  MOD_RES 13: note = MeGly
XNTXXXXXXENXX

SEQ ID NO: 429 - protein, 14 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 14: note = MeGly
RXNTXXXXXXENAX

SEQ ID NO: 430 - protein, 13 aa, synthetic construct
  MOD_RES 2: note = Penicillamine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 4-aminotetrahydro-2H-pyran-4-carboxylic acid
RXQTWQXXXXENX

SEQ ID NO: 431 - protein, 13 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-(2-hydroxyphenyl)naphthalen-2-yl)propa noic acid
  MOD_RES 10: note = 4-aminotetrahydro-2H-pyran-4-carboxylic acid
  MOD_RES 13: note = 4-aminotetrahydro-2H-pyran-4-carboxylic acid
RXNTXXXXXXENX

SEQ ID NO: 432 - protein, 13 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-(4-morpholinophenyl)naphthalen-2-yl)pr opanoic acid
  MOD_RES 10: note = 4-aminotetrahydro-2H-pyran-4-carboxylic acid
  MOD_RES 13: note = 4-aminotetrahydro-2H-pyran-4-carboxylic acid
RXNTXXXXXXENX

SEQ ID NO: 433 - protein, 13 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 3-(6-(1H-pyrazol-4-yl)naphthalen-2-yl)-2-aminopropa noic acid
  MOD_RES 10: note = 4-aminotetrahydro-2H-pyran-4-carboxylic acid
  MOD_RES 13: note = 4-aminotetrahydro-2H-pyran-4-carboxylic acid
RXNTXXXXXXENX

SEQ ID NO: 434 - protein, 13 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-(3-(trifluoromethyl)-1H-pyrazol-4-yl)n aphthalen-2-yl)propanoic acid
  MOD_RES 10: note = 4-aminotetrahydro-2H-pyran-4-carboxylic acid
  MOD_RES 13: note = 4-aminotetrahydro-2H-pyran-4-carboxylic acid
RXNTXXXXXXENX

SEQ ID NO: 435 - protein, 14 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = N,N,N-trimethylethanaminium
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXNTXXXXXXENXX

SEQ ID NO: 436 - protein, 14 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = N,N,N-trimethylethanaminium
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXNTXXXXXXENXX

SEQ ID NO: 437 - protein, 13 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(5-methoxynaphthalen-2-yl)propanoic acid
  MOD_RES 10: note = 4-aminotetrahydro-2H-pyran-4-carboxylic acid
  MOD_RES 13: note = Alpha-methylphenylalanine
RXNTXXXXXXENX

SEQ ID NO: 438 - protein, 13 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-(4-methoxyphenyl)naphthalen-2-yl)propa noic acid
  MOD_RES 10: note = 4-aminotetrahydro-2H-pyran-4-carboxylic acid
  MOD_RES 13: note = 4-aminotetrahydro-2H-pyran-4-carboxylic acid
RXNTXXXXXXENX

SEQ ID NO: 439 - protein, 13 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(5-carbamoylnaphthalen-2-yl)propanoic acid
  MOD_RES 10: note = 4-aminotetrahydro-2H-pyran-4-carboxylic acid
  MOD_RES 13: note = Alpha-methylphenylalanine
RXNTXXXXXXENX

SEQ ID NO: 440 - protein, 14 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 2-amino-3-(3-carbamoyl-1H-pyrazol-4-yl)propanoic acid
  MOD_RES 14: note = MeGly
RXNTXXXXXXENXX

SEQ ID NO: 441 - protein, 14 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 2-amino-3-(5-carbamoylpyridin-3-yl)propanoic acid
  MOD_RES 14: note = MeGly
RXNTXXXXXXENXX

SEQ ID NO: 442 - protein, 14 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXNTXXXXXXENXX

SEQ ID NO: 443 - protein, 13 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(5-bromonaphthalen-2-yl)propanoic acid
  MOD_RES 10: note = 4-aminotetrahydro-2H-pyran-4-carboxylic acid
  MOD_RES 13: note = 4-aminotetrahydro-2H-pyran-4-carboxylic acid
RXNTXXXXXXENX

SEQ ID NO: 444 - protein, 13 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-bromonaphthalen-2-yl)propanoic acid
  MOD_RES 10: note = 4-aminotetrahydro-2H-pyran-4-carboxylic acid
  MOD_RES 13: note = 4-aminotetrahydro-2H-pyran-4-carboxylic acid
RXNTXXXXXXENX

SEQ ID NO: 445 - protein, 13 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(5-methylnaphthalen-2-yl)propanoic acid
  MOD_RES 10: note = 4-aminotetrahydro-2H-pyran-4-carboxylic acid
  MOD_RES 13: note = Alpha-methylphenylalanine
RXNTXXXXXXENX

SEQ ID NO: 446 - skipped

SEQ ID NO: 447 - protein, 14 aa, synthetic construct
  MOD_RES 1: note = 7-aminoheptanoic acid
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(6-hydroxynaphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
XXNTXXXXXXENXX